(19) United States
(12) Patent Application Publication
(10) Pub. No.: US 2011/0251077 A1
(43) Pub. Date: Oct. 13, 2011

(54) Methods and Systems for Diagnosis of Non-Central Nervous System (CNS) Diseases in CNS Samples

(76) Inventors: Osvaldo L. Podhajcer, Buenos Aires (AR); Fernando Juan Pitossi, Buenos Aires (AR); Marcelo Rubinstein, Buenos Aires (AR)

(21) Appl. No.: 13/036,536

(22) Filed: Feb. 28, 2011

Related U.S. Application Data
(63) Continuation of application No. 10/563,049, filed on Jul. 12, 2006, now abandoned.

(51) Int. Cl. C40B 30/00; C12Q 1/02; C12Q 1/68

(52) U.S. Cl. 506/7; 435/6.1; 435/6.17; 435/29; 435/6.14

(57) ABSTRACT

The invention features methods, systems, and compositions for diagnosing non-central nervous system (non-CNS) disorders by detecting changes in gene expression in the CNS, e.g., in cerebrospinal fluid, in brain or spinal cord tissue samples, or other bodily fluid samples.

### CROSS-REFERENCE TO RELATED APPLICATIONS

This applications is a continuation of U.S. patent application Ser. No. 10/563,049, filed on Dec. 30, 2005, which claims priority from U.S. Provisional Patent Application Nos. 60/484,683 and 60/484,726, both filed on Jul. 3, 2003. The entire contents of these two applications, including figures, are incorporated herein by reference.

### FIELD OF THE INVENTION

The invention relates to methods and compositions for risk assessment, identification, diagnosis, prognosis, and/or monitoring of disease, and for early therapeutic intervention.

### BACKGROUND OF THE INVENTION

It is axiomatic that early diagnosis and concomitant early therapeutic intervention is the key to successful treatment and/or management of most human disorders. However, many disorders cannot be diagnosed until the pathological process is already advanced. For example, many solid tumors are usually not clinically detectable before they can be palpated or visualized by tissue imaging techniques (i.e., when they are at least 0.5 cm in size), at which time neoplasia may have been present for years. Similarly, the diagnostic criterion for diabetes mellitus (increased fasting plasma glucose levels or hyperglycemia) identifies the disorder when glucose intolerance (the underlying cause of hyperglycemia) is already present. In another example, rheumatoid arthritis (RA) is diagnosed by the presence of joint stiffness and soreness and the presence of positive rheumatoid factor, all factors that indicate RA is already present and may be advanced.

### Diagnostic Disease Markers

In cancer, progression from preneoplasia to malignancy is accompanied by the accumulation of genetic changes in the neoplastic cells that lead to histopathological modifications. In some circumstances, when such a genetic change corresponds to an increase in a protein made by the tumor cells, such a protein can be detected in the tumor or in body fluids (if secreted from the tumor), and used as a biological tumor marker. Most tumors have been associated with one or more such tumor markers. Such markers have been evaluated as potential tools to diagnose cancer, determine prognosis, and/or monitor cancer progression. However, many tumor markers are detectable only after neoplasia has already progressed to the stage of formation of a tumor. In some cases, a tumor marker may not be detectable until a tumor is already malignant. Thus, many of the most widely used tumor markers are used primarily to monitor disease progression or response to treatment rather than for early diagnosis.

In rheumatoid arthritis, anti-cyclic citrullinated peptide (anti-CCP) antibodies, anti-keratin antibodies (AKA) and IgM rheumatoid factors have been suggested as markers for rheumatoid arthritis (Bas et al., Rheumatology (Oxford), 2002, 41(7):809-14). However, the value of such markers remains inconclusive (Scott, Rheumatology (Oxford), 2000, 39(Supp) 1:24-9). Similarly, while several protein and gene markers have been found to correlate with the presence of active diabetes, the use of markers as diagnostic or predictive has not been proven valuable at this time for either type I or type 2 diabetes (see the National Academy of Clinical Biochemistry (NACB) Laboratory Medicine Practice Guidelines: Guidelines and Recommendations for Laboratory Analysis in the Diagnosis and Management of Diabetes Mellitus, 2002, available online at the NACB web site).

### Genomics and Proteomics Tools for Disease Diagnosis

The development of high throughput screening approaches such as functional genomics and proteomics has provided a new biological platform to search for molecules associated with different disorders. Gene-expression profiles based on microarray analysis have been of some use to predict survival of patients with lung carcinoma (Beer et al., 2002, Nat. Med., 8(8):816-24). A similar approach identified a group of genes that were said to be useful to predict the clinical outcome of diffuse large B-cell lymphoma following combination chemotherapy (Shipp et al., 2002, Nat. Med., 8(1):68-74). In addition, comparison of the proteomic profile of patients with ovary or prostate cancer compared to non cancerous volunteers was said to have provided a set of serum proteins that might be useful for early cancer detection (Petricoin et al., 2002, Lancet, 2002, 359(9306):572-7; Petricoin et al., 2002, J. Natl. Cancer Inst., 94(20):1576-8).

At present, most functional genomics studies in cancer have used cancer samples obtained from patients to generate cancer-associated gene expression profiles (either by a genomics or a proteomics approach).

A need remains for methods to detect and diagnose disease. Particularly needed are predictive methods and markers for early stage or very early stage disease detection and risk assessment.

### SUMMARY OF THE INVENTION

The methods and systems described herein are based, at least in part, on the discovery that the central nervous system (CNS) exhibits specific changes in gene expression (e.g., changes in patterns of gene expression) in response to the presence of a peripheral (non-CNS) disease or disorder (e.g., a hyperproliferative disorder such as a non-CNS tumor or cancer, an immunological disorder, an inflammatory disorder, a metabolic disorder, or a pathogenic infection). While not bound by any theory, the inventors believe that specific changes in gene expression in the CNS, e.g., in the brain, occur in response to the presence of peripheral disease at an early stage in the development of the disease, e.g., before the disorder is clinically detectable and/or before the subject is symptomatic. Thus, peripheral disorders can be diagnosed at an early stage and targeted for early therapeutic intervention by analyzing changes or patterns in gene expression in the CNS.

Accordingly, in one aspect, the invention features methods of diagnosing a non-CNS disorder in a subject, such as a human. The non-CNS disorder can be, e.g., a hyperproliferative disorder, e.g., a non-CNS tumor or cancer; an immunological disorder, e.g., rheumatoid arthritis; an inflammatory or allergic disorder, e.g., asthma; a metabolic disorder, e.g., diabetes or obesity; or a pathogenic infection, e.g., a viral infection. The methods include detecting expression of a gene in a CNS sample of the subject, e.g., a brain tissue or cell (such as a tissue or cell of the hypothalamus, the cerebellum, the midbrain, the hippocampus, the prefrontal cortex or the striatum) or a sample of cerebrospinal fluid (CSF) or any other bodily fluid where the CNS gene product (or derivatives from it) could be detected. The method optionally includes a step of obtaining the CNS sample. A change in gene expression compared to a reference value, e.g., a control or basal value, is correlated with the presence of a non-CNS disorder. The method is not limiting in that it can be used to detect the risk or presence of any non-CNS disorder. In one embodiment, the non-CNS disorder is not lymphoma.

The subject can be a human. In one embodiment, the human is not symptomatic for the disorder to be diagnosed. In another embodiment, the disorder is not clinically detectable, e.g., it is not detectable by a routine general clinical exam.

Detecting expression of a gene in a CNS sample, or any other bodily fluid where the CNS gene product (or derivatives from it) could be detected, can include detecting or determining a value for one or more of the level of mRNA, rate of transcription, amount of a gene product, and activity of a gene product. In some embodiments, expression of a single gene in the CNS may be detected, where a change in gene expression in that gene is associated with the presence of a non-CNS disorder. In other embodiments, expression of a plurality of genes (e.g., a panel or cluster of genes) may be evaluated, where a specific profile of gene expression of the plurality of genes is associated with the presence of a particular non-CNS disorder.

The method can include correlating the result of the detecting step to the presence or absence of a non-CNS disorder. “Correlating” means identifying the probability, based on the result of a detecting step, that the subject has or does not have, or will develop or will not develop at some future time, a non-CNS disorder. Correlating can include generating a dataset from, or providing a record of, the detecting step, e.g., a printed or computer readable record such as a laboratory record or dataset. The record can include other information, such as a specific subject identifier, a sample identifier for the CNS sample, a date, the identity of the operator of the method, and/or other information. The record can be used to provide or store information about the subject. For example, the record can be used to provide information (e.g., to the subject, a health care provider, the government, or insurance company). The record or information derived from the record can be used, e.g., to identify the subject as suitable or unsuitable for a particular therapy or a particular clinical trial group.

In the methods described herein, gene expression of a CNS gene can be detected by any technique available to the skilled artisan, e.g., genomics or proteomics microarray analysis of a CNS biological sample, such as brain tissue, CSF, or any other bodily fluid where the CNS gene product (or derivatives from it) could be detected; or brain imaging techniques that detect changes in gene expression. In one embodiment, the method involves detecting a CNS gene product released or secreted into the CSF. In such embodiments, an agent (such as an antibody, e.g., a labeled antibody) for detecting the gene product can be immobilized on a solid phase, e.g., in a dipstick format.

The gene or genes to be evaluated will depend on the specific gene or profile of gene expression associated with a particular disorder (reference gene expression profile). For example, exemplary genes (or profiles or clusters of genes) that are regulated in response to the presence of cancer cells (or particular types of cancer cells) are shown in FIGS. 1-29, infra. Such genes are also referred to herein as CNS “marker genes” or “disease surveillance genes” for non-CNS disorders. The exemplary CNS marker genes are not limiting, as the methods described herein can include the detection of other genes or gene products determined to exhibit a change in expression associated with the presence of a peripheral non-CNS disorder. CNS marker genes can include, inter alia, genes encoding hormones, growth factors, immune system components, and cytokines.

In another aspect, the invention features systems for diagnosing non-CNS disorders in a subject. The systems include a sampling device to obtain a CNS sample; a gene expression detection device that generates gene expression data for one or more genes in the CNS sample; a reference gene expression profile for a specific non-CNS disorder; and a comparator that receives and compares the gene expression data with the reference gene expression profile. The invention also includes kits that can be used with such systems. The kits include the sampling device or containers for the sample, and the reference gene expression profile for a specific disorder. The profile can be in the form of a digital data set in a computer-readable medium, or an analog profile in electronic form.

Other systems included herein for diagnosing non-CNS disorders include an imaging device (e.g., PET or MRI device) to obtain an image of gene expression of one or more genes in the CNS and generate gene expression data for the one or more genes; a reference gene expression profile for a specific non-CNS disorders; and a comparator that receives and compares the gene expression data with the reference gene expression profile.

In other aspects, the invention also includes methods of diagnosing non-CNS disorders in a subject, by detecting expression of one or more genes in a CNS sample of the subject; generating gene expression data from the detected expression; obtaining a reference gene expression profile for a specific non-CNS disorders; and comparing the gene expression data with the reference gene expression profile, wherein a match of the CNS sample gene expression data to the reference gene expression profile indicates the subject has or will develop the non-CNS disorder.

In these systems and methods, the CNS sample can be a cerebrospinal fluid (CSF) sample, and the gene expression data can corresponds to a protein in the CSF. Alternatively, the CNS sample can be a bodily fluid sample that contains a protein expressed by a gene in the CNS, and the gene expression data corresponds to the presence or level of the protein in the sample. The CNS sample can also be a bodily fluid sample that contains a protein whose presence or level in the sample is affected by a gene expressed in the CNS, and the gene expression data corresponds to the presence or level of the protein in the sample. For example, the protein can be selected from a hormone, a growth factor, an immune system component, and a cytokine. The protein can be encoded by any of the genes listed in any of FIGS. 1, 50, and 54, or a human or other mammalian homolog thereof. Human homologs of the genes named herein can be easily obtained from publicly available databases, e.g., on the Internet, such as GenBank.

Specific genes encode a gene product (e.g., protein) selected from the group consisting of hepatocyte growth factor (HGF), apherin A3, chemokine (C—C motif) ligand 4, growth differentiation factor-9b (GDF-9b); bone morphogenetic protein 15 (BMP 15), neuroblastoma suppressor of tumorigenicity 1, melanocyte proliferating gene 1, and fibroblast growth factor 22 (FGF 22).

The CNS sample can also be one or more cells from the brain, and the gene expression data can correspond to a nucleic acid molecule (e.g., mRNA corresponding to the gene) or protein in the sample. The brain cells can be selected from the hypothalamus, the midbrain, the prefrontal cortex, or the striatum.

In these systems and methods, two or more reference gene expression profiles can be used, each specific for a different non-CNS disorder. The non-CNS disorder can be, for example, cancer, rheumatoid arthritis, asthma, diabetes, or obesity. For example, the non-CNS disorder can be a solid tumor less than 0.5 cm in diameter. The gene expression data can contain data for a plurality of genes in the CNS sample, and comprises a gene expression profile.

The methods herein can also include obtaining a control gene expression profile corresponding to one or more healthy subjects; and comparing the gene expression data with the control gene expression profile, wherein a match of the CNS sample gene expression data to the control gene expression profile indicates the subject does not have and will not develop the non-CNS disorder.

In the new systems and methods, gene expression can be detected using a microarray assay, and the subject can be a human.

In another aspect, the invention includes methods of diagnosing non-CNS disorders by obtaining a test gene expression profile for two or more CNS genes from the subject; obtaining a reference gene expression profile for a specific non-CNS disorder; and comparing the test gene expression profile with a reference gene expression profile, wherein a test gene expression profile that matches the reference gene expression profile indicates the subject has or will develop the non-CNS disorder.

The methods and systems herein can include generating a record of the result of the comparing step; and optionally transmitting the record to the subject, health care provider, or other party.

In yet another aspect, the invention features a computer-readable medium that contains a data set corresponding to a reference gene expression profile including expression data of 5 or more genes (e.g., 10, 15, 20, 50, or more), wherein each of the 5 or more genes is differentially expressed in a central nervous system (CNS) sample of a mammal having a specific non-CNS disorder compared to the same 5 or more genes in a mammal not having the specific non-CNS disorder; wherein the data set is used to diagnose a non-CNS disorder.

For example, in some embodiments, the computer-readable medium contains a reference gene expression profile that includes expression data of 5 or more (e.g., 10, 15, 20, 50, or more) genes selected from any of the genes listed in one or more of FIGS. 29-1 to 29-6; 32-1 to 32-6; or 35-1 to 35-6 for breast cancer; FIGS. 30-1 to 30-6; 33-1 to 33-6; or 36-1 to 36-6 for colon cancer; FIGS. 31-1 to 31-6; 34-1 to 34-6; or 37-1 to 37-6 for lung cancer; FIG. 50 for arthritis; or FIG. 54 for asthma.

The genes can also be selected from any one of the following groups of genes:

Breast Cancer Nedd8 (FIG. 29-1), Col4a3 bp (FIG. 29-2), Bgn (FIG. 29-4), Sox5 (FIG. 29-5), Slc38a4 (FIG. 32-1), Tom1 (FIG. 32-2), Calr (FIG. 32-4), Itgae (FIG. 32-5),

Ttrap (FIG. 35-1), P ex11b (FIG. 35-2), Sema7a (FIG. 35-4), and Stam2 (FIG. 35-5);

Colon Cancer: Nmb (FIG. 30-1), Ryr2 (FIG. 30-2), Trfr (FIG. 30-4), Mfap5 (FIG. 30-5), Prrg2 (FIG. 33-1), Faim (FIG. 33-2), Mgrn1 (FIG. 33-4), Stch (FIG. 33-5), Lhb (FIG. 36-1), Prm3 (FIG. 36-2), Crry (FIG. 36-4), and Timp4 (FIG. 36-5);

Lung cancer: Nmb (FIG. 31-1), Pcdh8 (FIG. 31-2), Rock2 (FIG. 31-4), Angpt13 (FIG. 31-5), Sqstm1 (FIG. 34-1), Kcnip2 (FIG. 34-2), Oxt (FIG. 34-4), Myh4 (FIG. 34-5), Enc1 (FIG. 37-1), Gsg1 (FIG. 37-2), Srr (FIG. 37-4), and Ndph (FIG. 37-5);

Arthritis: Bcl21 (FIG. 51A), P2rx1 (FIG. 51B), Pafah1b1 (FIG. 51B), Kcna3 (FIG. 51C), Taf1b (FIG. 51C), Slc38a3 (FIG. 51D), Hprt (FIG. 52A), C1d (FIG. 52B), Car11 (FIG. 52D), Dusp3 (FIG. 52D), Gabrr2 (FIG. 53C), and Aatk (FIG. 53D); and

Asthma: Rasa3 (FIG. 55B), Tnk2 (FIG. 55B), H28 (FIG. 55C), Diap2 (FIG. 55C), Lgals6 (FIG. 56A), Reck (FIG. 56A), Whrn (FIG. 56A), Stk22s1 (FIG. 56B), CD47 (FIG. 57A), Jund1 (FIG. 57A), Cstb (FIG. 57B), and Desrt (FIG. 57B).

In another embodiment, the invention includes methods of identifying a disease surveillance gene for non-CNS disorders in a human, by inducing a non-CNS disorder in a test experimental animal; comparing expression of a gene in a CNS sample from the test experimental animal to expression of the gene in a CNS sample from a control experimental animal; and selecting as a disease surveillance gene a human homolog of a gene that is differentially expressed in the CNS sample from the test experimental animal compared to the CNS sample from the control experimental animal. In some embodiments, a non-CNS neoplasm is induced by chemical or radiation mutagenesis, or by administering a neoplastic cell to the experimental animal, and the experimental animal is an animal model (e.g., a mouse or non-human primate) of rheumatoid arthritis, diabetes, asthma, obesity, or diabetes.

In the new systems and methods, the subject can lack a clinical sign of a disorder as evaluated by imaging analysis, can have a family history of the disorder, and/or can be a carrier of a gene associated with an increased risk of developing the disorder (such as the BRCA1, BRCA2, hMSH2, hMLH1, or hMSH6 gene).

In another aspect, the invention features methods of generating a reference gene expression profile of one or more genes that are differentially expressed in a CNS sample of a mammal having a specific non-CNS disorder, by obtaining a control mammal not having the specific non-CNS disorder; obtaining a diseased mammal of the same type as the control mammal that has the specific non-CNS disorder; obtaining a first CNS sample from the control mammal and a second CNS sample from the diseased mammal; generating a first gene expression profile from the first CNS sample and a second genetic expression profile from the second CNS sample; comparing the first and second genetic expression profiles; selecting a set of genes from the second genetic expression profile that are differentially expressed; and preparing the reference gene expression profile from expression data from the selected genes.

The invention also features, e.g., in electronic digital or analog format a reference gene expression profile corresponding to the presence of a non-central nervous system (non-CNS) disorder in a mammal, comprising expression data of 5 or more genes, wherein each of the 5 or more genes is differentially expressed in a central nervous system (CNS) sample of a mammal having a specific non-CNS disorder compared to the same 5 or more genes in a mammal not having the specific non-CNS disorder.

The invention also includes methods of treating a subject by diagnosing a non-central nervous system (non-CNS) disorder according to the methods or using the systems described herein; and administering to the subject a therapeutic agent for the disorder. For example, the therapeutic agent can be a chemotherapeutic agent, such as an antitubulin/antimicrotubule drug, a topoisomerase I inhibitor, an antimetabolite, and an alkylating agent.

In another aspect, the invention features methods of determining whether a subject (e.g., a human) has, or is at risk for developing, a peripheral (non-CNS) disorder. The method involves providing or obtaining a test gene expression profile for one, two, or more CNS genes in the subject; and comparing the test gene expression profile with a reference gene expression profile (e.g., a reference gene expression profile described herein), wherein the reference gene expression profile is associated with the presence of a particular non-CNS disorder. Non-limiting examples of reference gene expression profiles (e.g., associated with colon, breast or lung carcinoma), are disclosed herein. In one embodiment, the method includes generating a record of the result (e.g., a laboratory record or dataset) of the comparing step; and, optionally; transmitting the record (e.g., by print or computer readable material) to the subject, the subject's health care provider or another party. As with other methods described herein, various techniques can be used to provide a gene expression profile and various types of disorder scan be detected.

The methods described herein are useful, inter alia, for risk assessment for a variety of disorders, for early detection and diagnosis of disease, for monitoring of progression of disease, for monitoring efficacy of treatment for a disease, and/or evaluation of clinical status.

As used herein a “disorder” or “disease” is an alteration in the state of the body or of some of its cells, tissues, or organs, that threatens health. The two terms are meant to encompass all stages of an illness, including the very early stages of an illness (e.g., early alterations in the body that may not be detectable by the subject or a health care provider, but nonetheless set in motion a disease process). For example, the terms “disorder” and “disease” encompass the state of neoplasia, before a neoplasm or tumor is formed; early immunological reactions to an antigen, e.g., in the development of rheumatoid arthritis or asthma, before inflammation or allergy are symptomatic; and early changes in energy metabolism that promote weight gain, before weight gain is produced.

As used herein, “neoplasia” is an unregulated and progressive proliferation of cells under conditions that would not elicit, or would cause cessation of, proliferation of normal cells. Neoplasia can result in the formation of a “neoplasm,” a new and abnormal growth of tissue. If the abnormally proliferating cells form a mass, a neoplasm is generally referred to as a “tumor.” A neoplasm may be benign or malignant (cancerous).

As used herein, the term “matches”, “matching” or “match” if at least 75% of the genes in a test gene expression profile are either up- or down-regulated in the same manner as the genes in the reference expression profile. For example, if genes 1 through 5 are up regulated and genes 6 through 10 are down regulated in the reference expression profile, then a test profile where genes 1 through 10 are down regulated would not be a match, whereas a test profile where genes 1, 2, 3, 4 & 6 are up-regulated and genes 5, 7, 8, 9 & 10 are down-regulated would be a match. A “high level match” would mean that at least 75% of the genes come within at least plus or minus 50% of the expression level (or Log2 ratio of expression level) of the gene in the reference expression profile. For example, in the reference expression profile: for gene A the Log2 ratio of expression level in the presence of a disorder to the expression level in the absence of the disorder is +0.4; for gene B the ratio is −0.4; for gene C the ratio is +0.2; and for gene D the ratio is −0.2. A test profile with the following values (A=+0.3; B=□0.3; C=+0.1; D=+0.3) is a high level match because genes A, B, C in the test profile (75% of the genes in the reference profile) are within ±50% of the ratios for those genes in the reference profile.

A “subject” is a human or animal that is tested for the presence of a possible disorder. The animal can be a mammal, e.g., a domesticated animal such as a dog, cat, horse, pig, cow or goat; an experimental animal such as an experimental rodent (e.g., a mouse, rat, guinea pig, or hamster); a rabbit; or an experimental primate, e.g., a chimpanzee or monkey.

Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, the drawings, and the claims.

### BRIEF DESCRIPTION OF THE DRAWINGS

FIGS. 1-1 to 1-35 are a table showing all the cancer disease surveillance genes (differentially expressed at p<0.01) identified in prefrontal cortex, hypothalamus, and midbrain of relevant animal models for breast, colon, and lung carcinoma. Data corresponds to genes differentially expressed in mice harboring tumors compared to control mice. Samples correspond to 18, 72, and 192 hours post tumor cell injection.

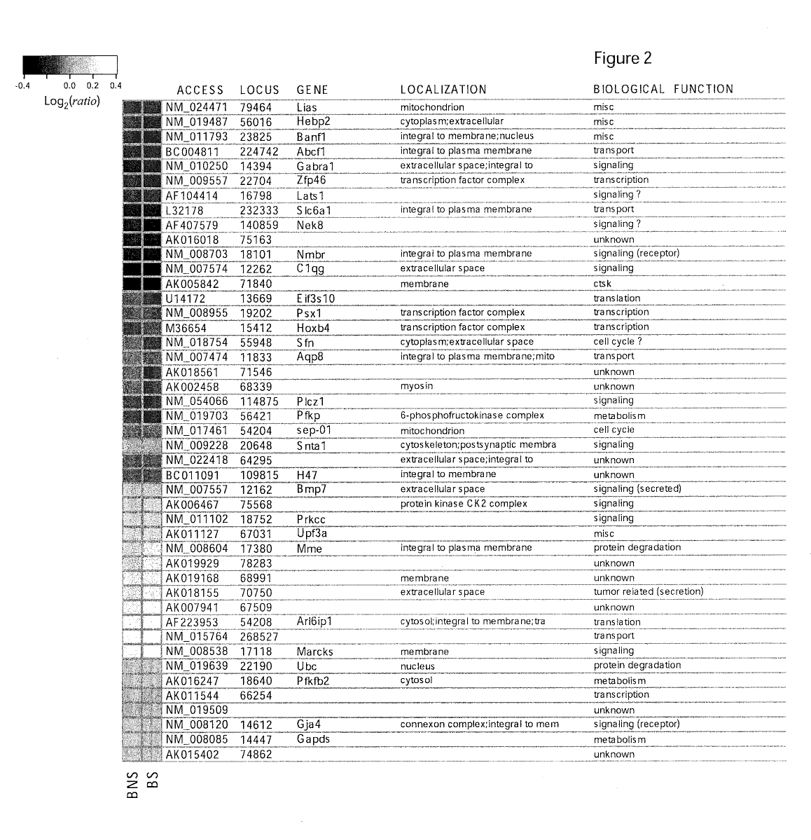

The following FIGS. 2 to 28 are tables showing the differentially expressed genes (p<0.01) in either the prefrontal cortex, the hypothalamus, or the midbrain of mice harboring either breast, lung or colon carcinoma. Samples correspond to either 18, 72, or 192 hours post tumor cell injection. Differentially expressed genes were identified by a mixed model ANalysis Of VAriance (ANOVA), with tumor (or control) as fixed effect. The base 2 logarithm of tumor vs. control ratio is shown as a gray scale. BNS corresponds to data obtained without background subtraction, and BS corresponds to data obtained after background subtraction.

FIG. 2 shows the differentially expressed genes in the prefrontal cortex of mice harboring breast carcinoma at 18 hours.

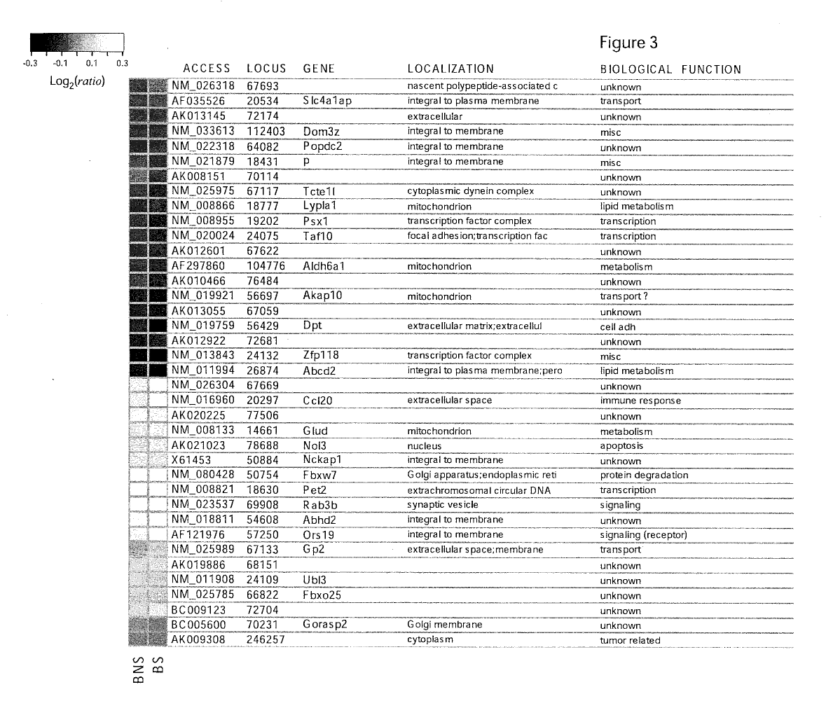

FIG. 3 shows the differentially expressed genes in prefrontal cortex of mice harboring breast carcinoma at 72 hours.

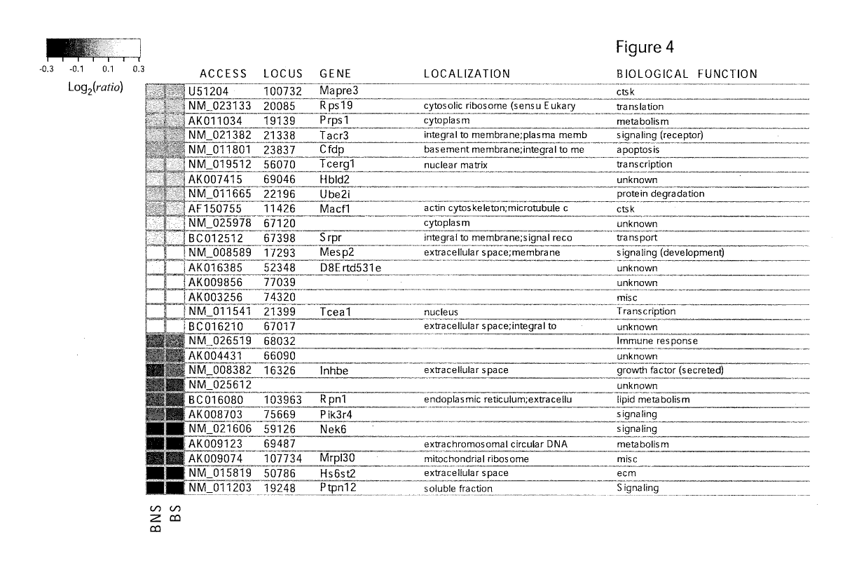

FIG. 4 shows the differentially expressed genes in prefrontal cortex of mice harboring breast carcinoma at 192 hours.

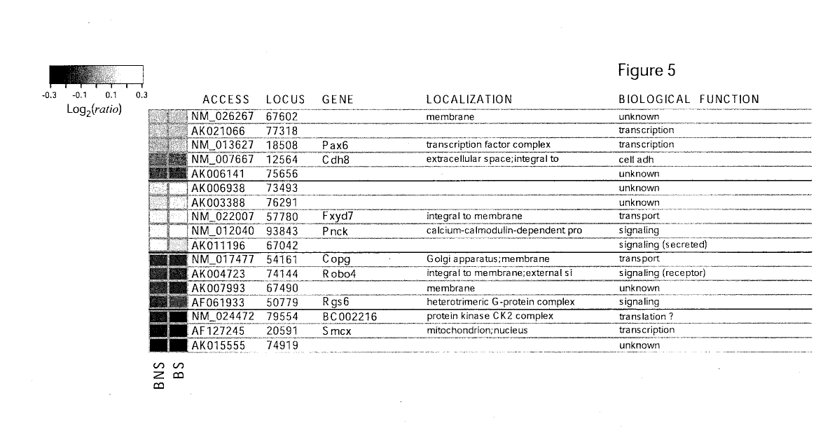

FIG. 5 shows the differentially expressed genes in prefrontal cortex of mice harboring colon carcinoma at 18 hours.

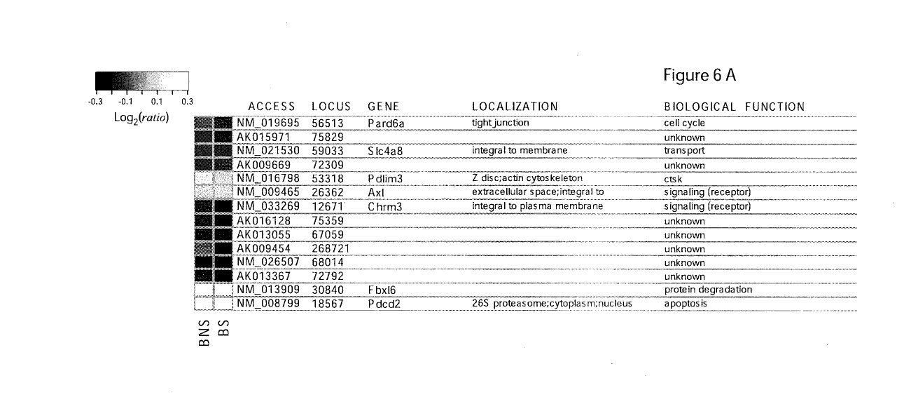

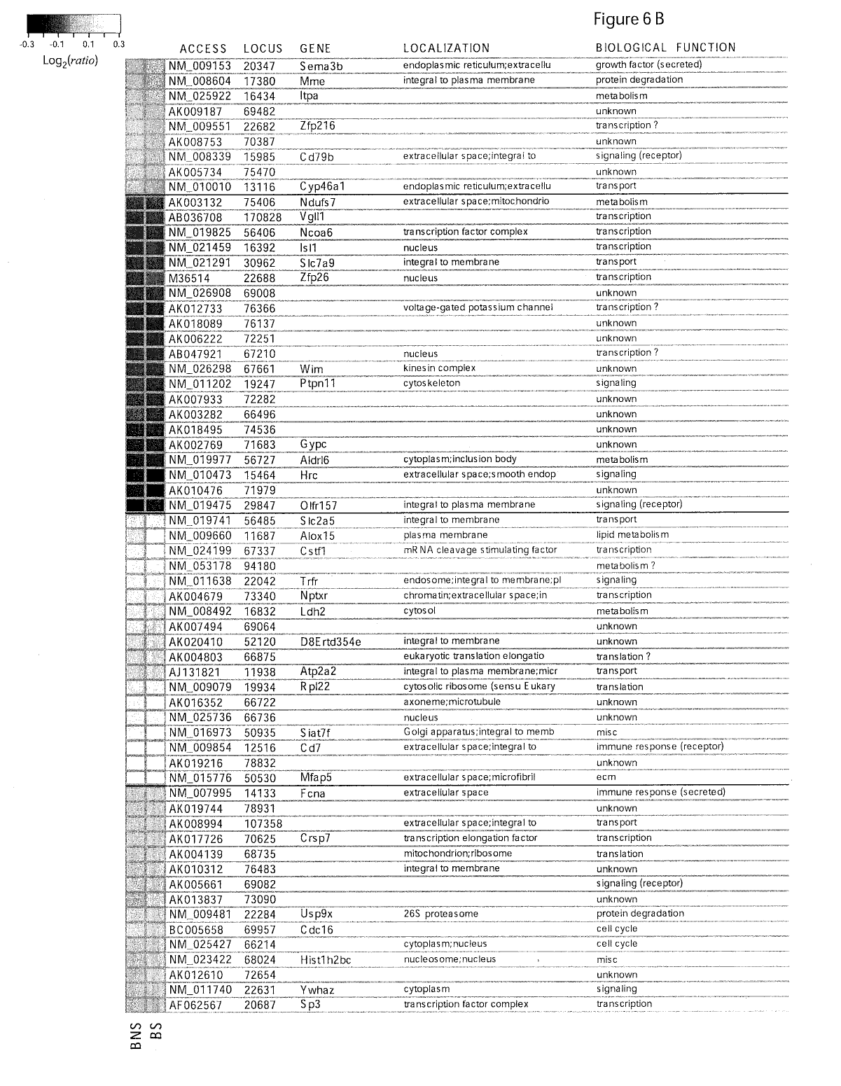

FIGS. 6A & 6B show the differentially expressed genes in prefrontal cortex of mice harboring colon carcinoma at 72 hours.

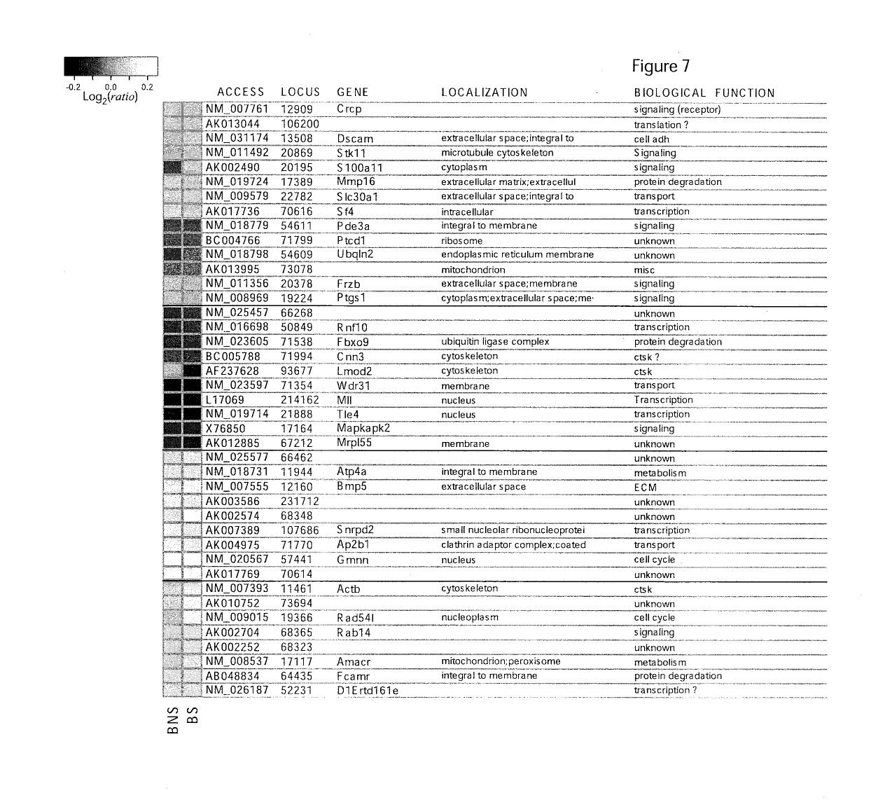

FIG. 7 shows the differentially expressed genes in prefrontal cortex of mice harboring colon carcinoma at 192 hours.

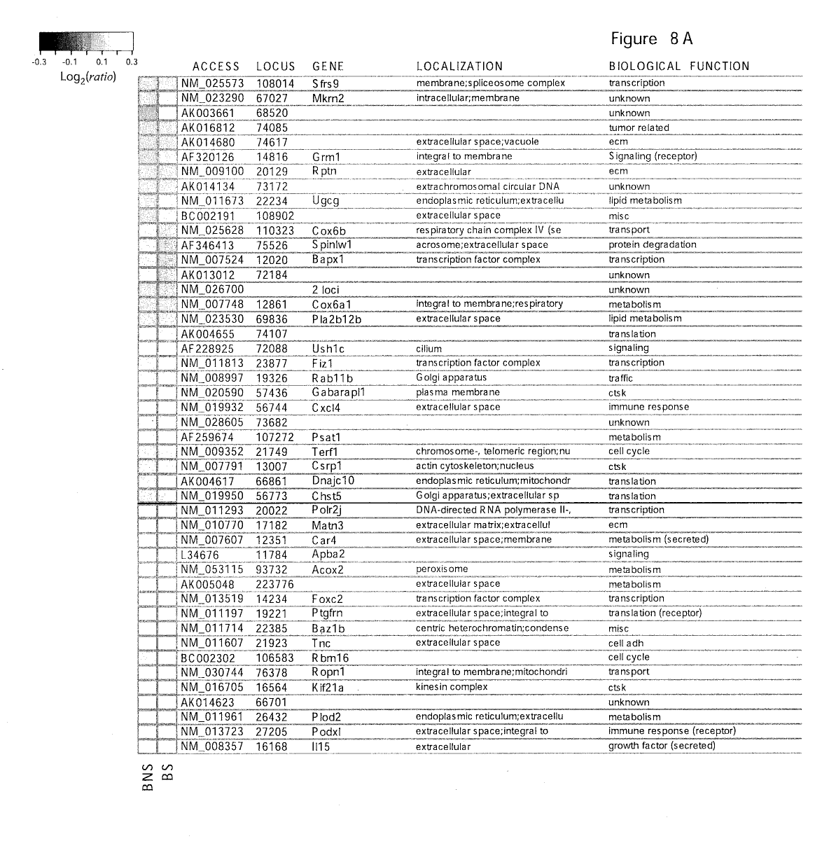

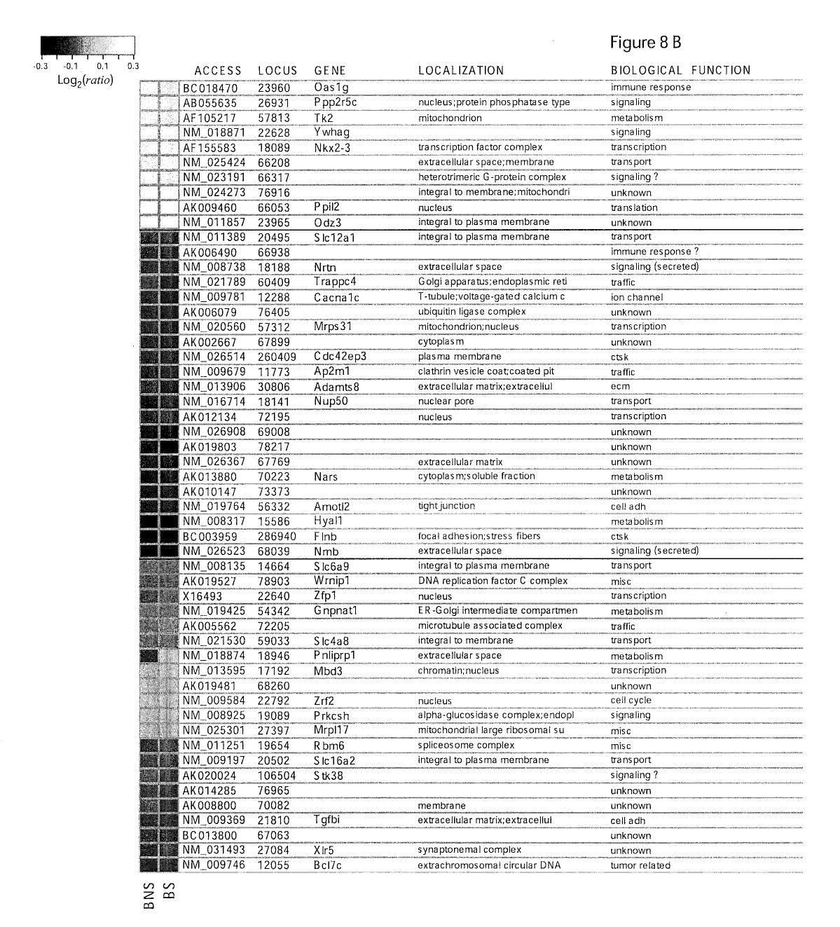

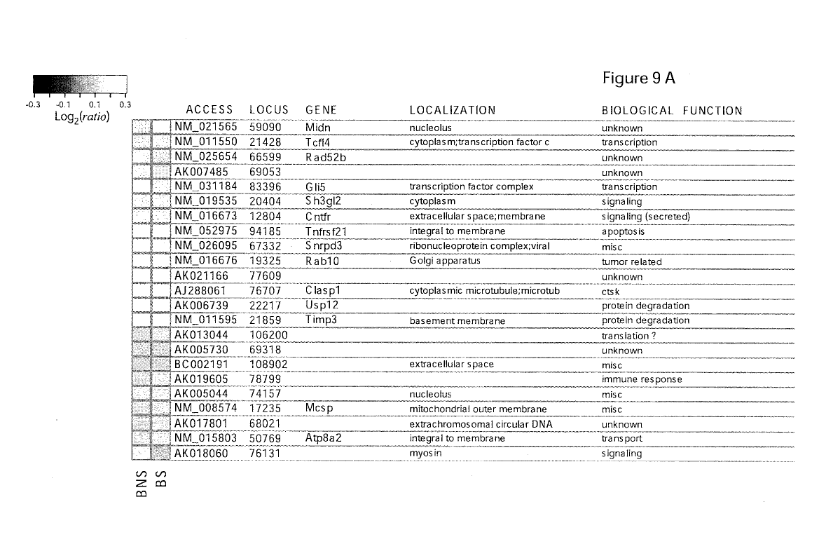

FIGS. 8A & 8B show the differentially expressed genes in prefrontal cortex of mice harboring lung carcinoma at 18 hours.

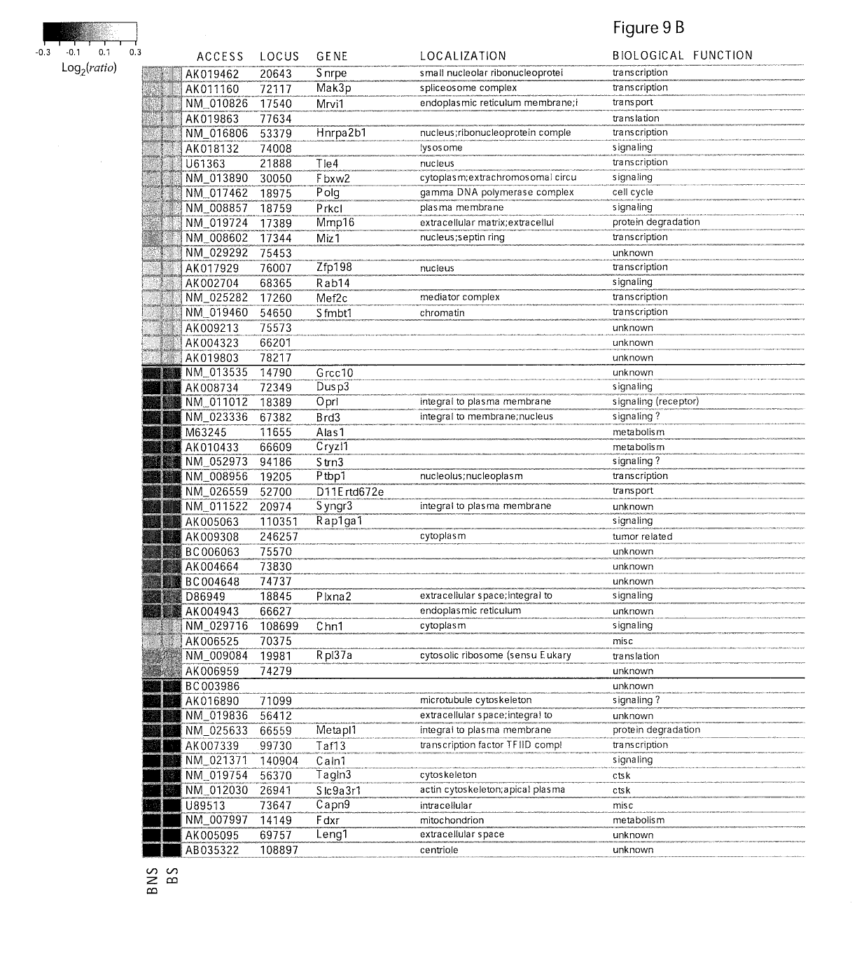

FIGS. 9A & 9B show the differentially expressed genes in prefrontal cortex of mice harboring lung carcinoma at 72 hours.

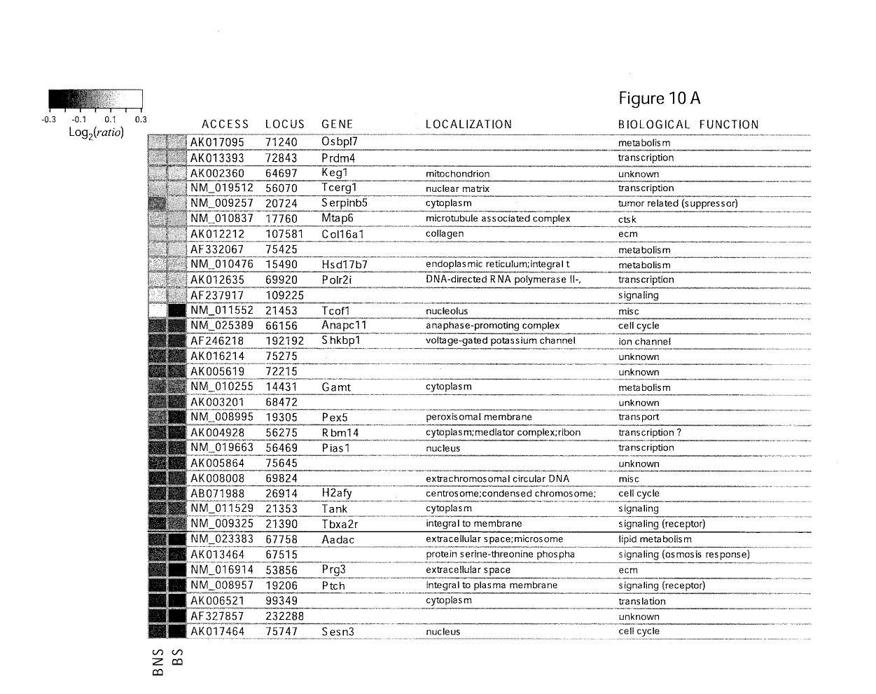

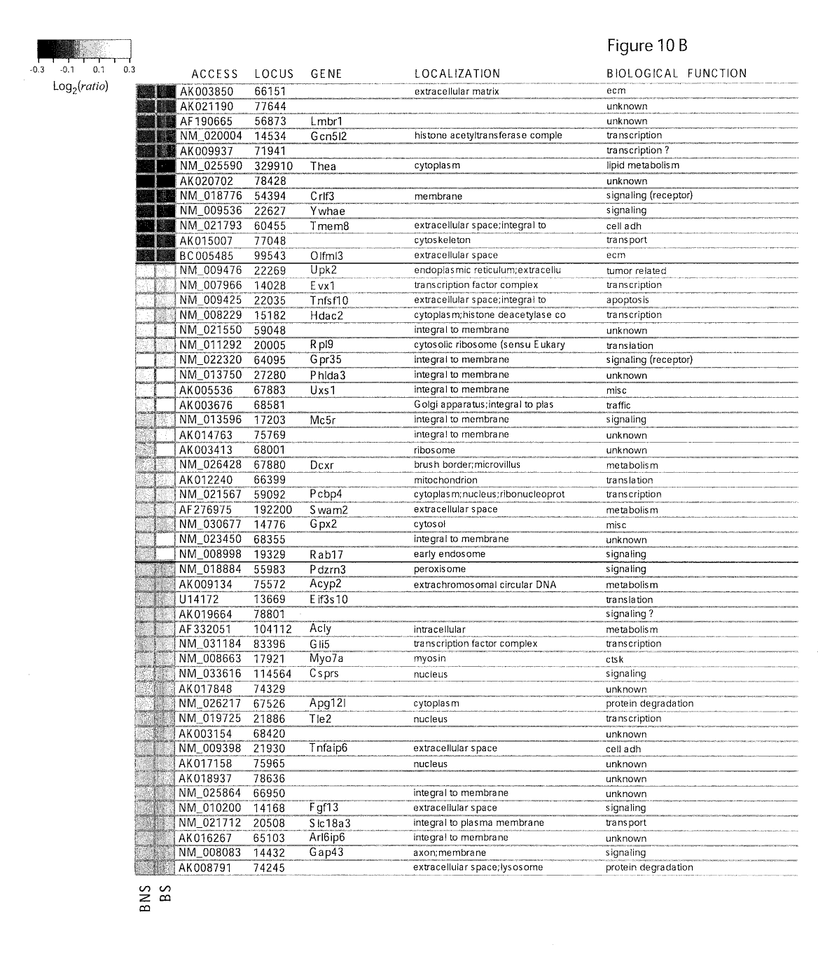

FIGS. 10A & 10B show the differentially expressed genes in prefrontal cortex of mice harboring lung carcinoma at 192 hours.

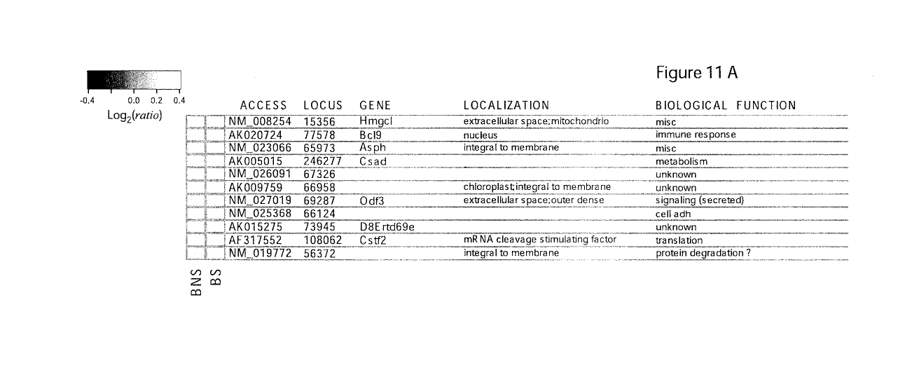

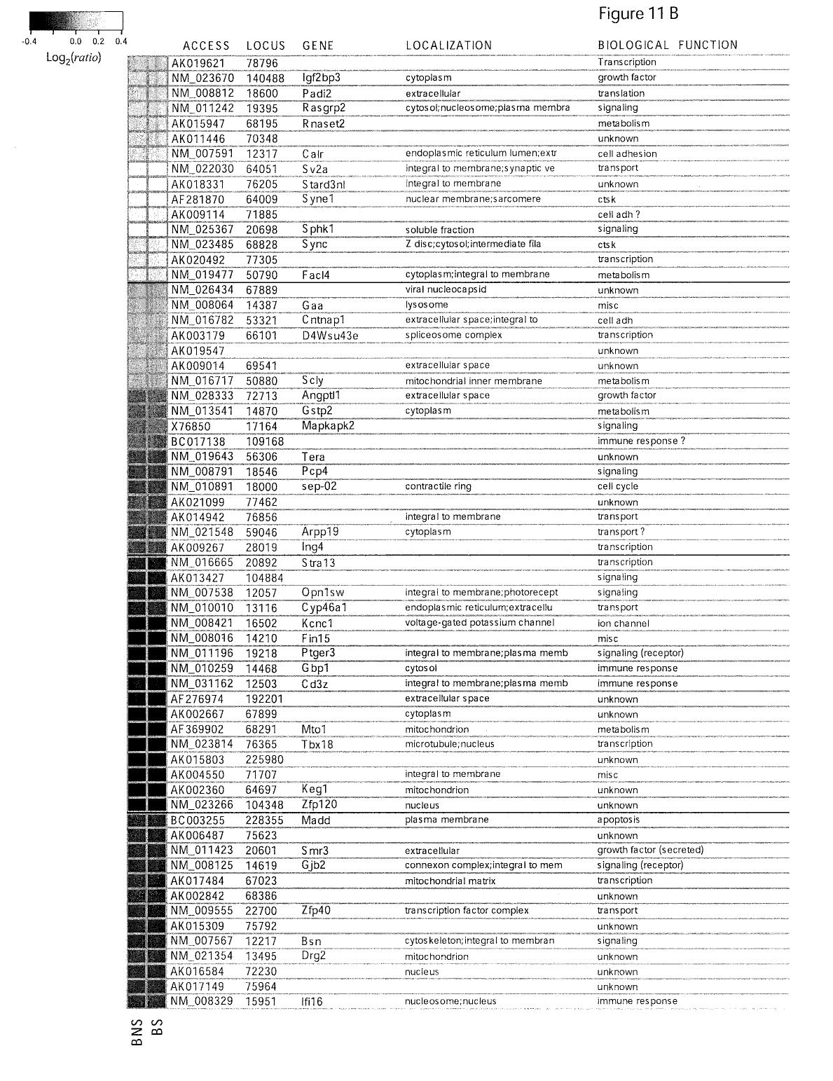

FIGS. 11A & 11B show the differentially expressed genes in hypothalamus of mice harboring breast carcinoma at 18 hours.

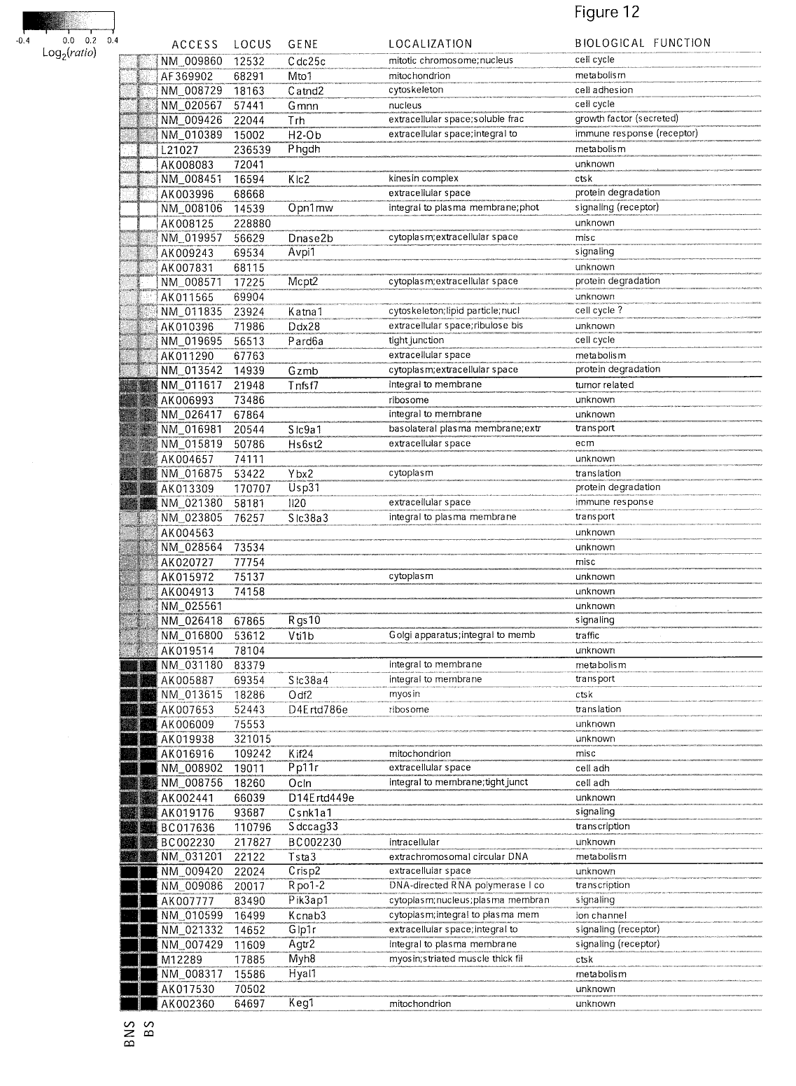

FIG. 12 shows the differentially expressed genes in hypothalamus of mice harboring breast carcinoma at 72 hours.

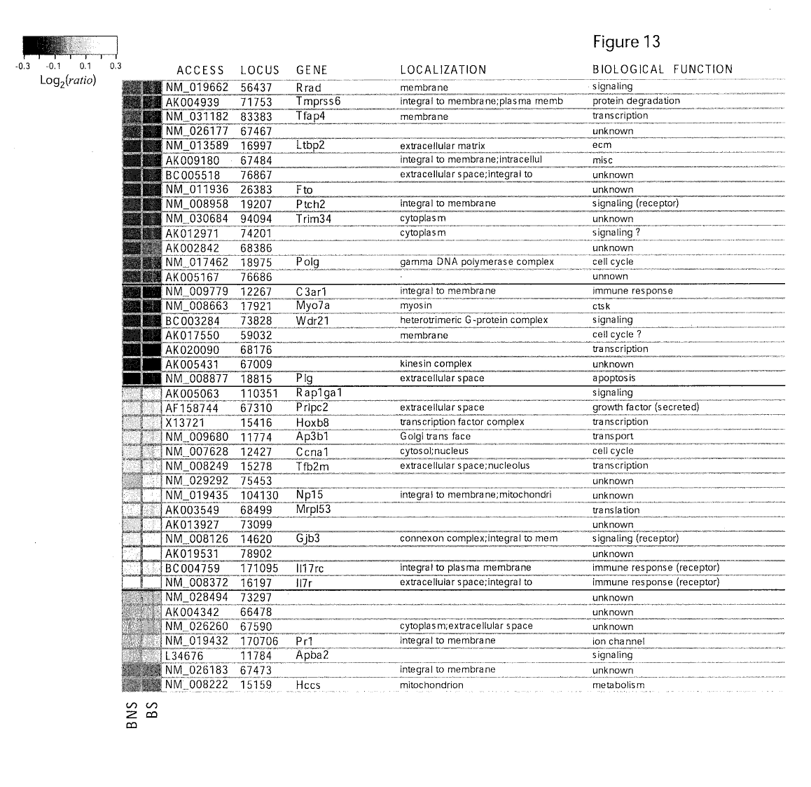

FIG. 13 shows the differentially expressed genes in hypothalamus of mice harboring breast carcinoma at 192 hours.

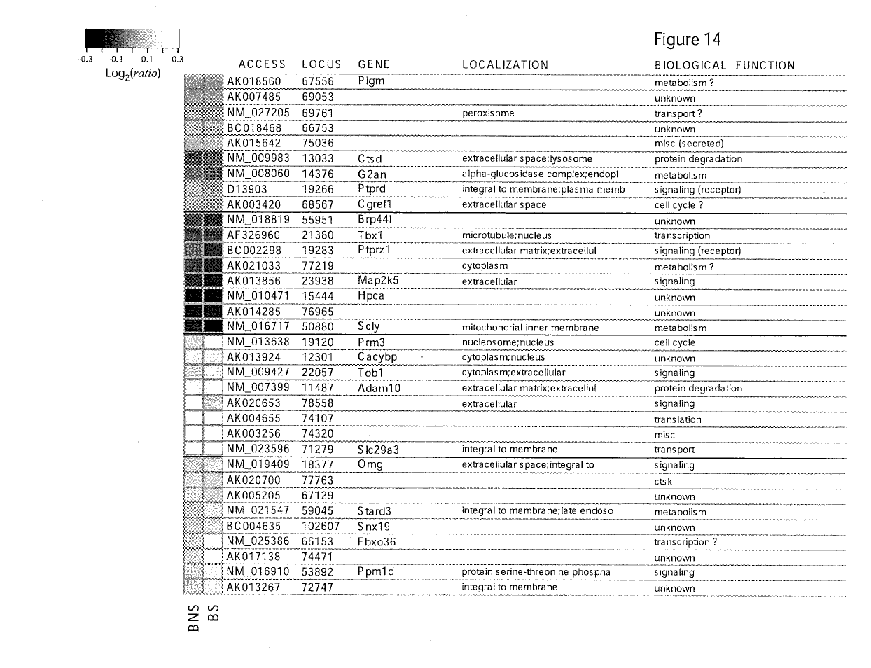

FIG. 14 shows the differentially expressed genes in hypothalamus of mice harboring colon carcinoma at 18 hours.

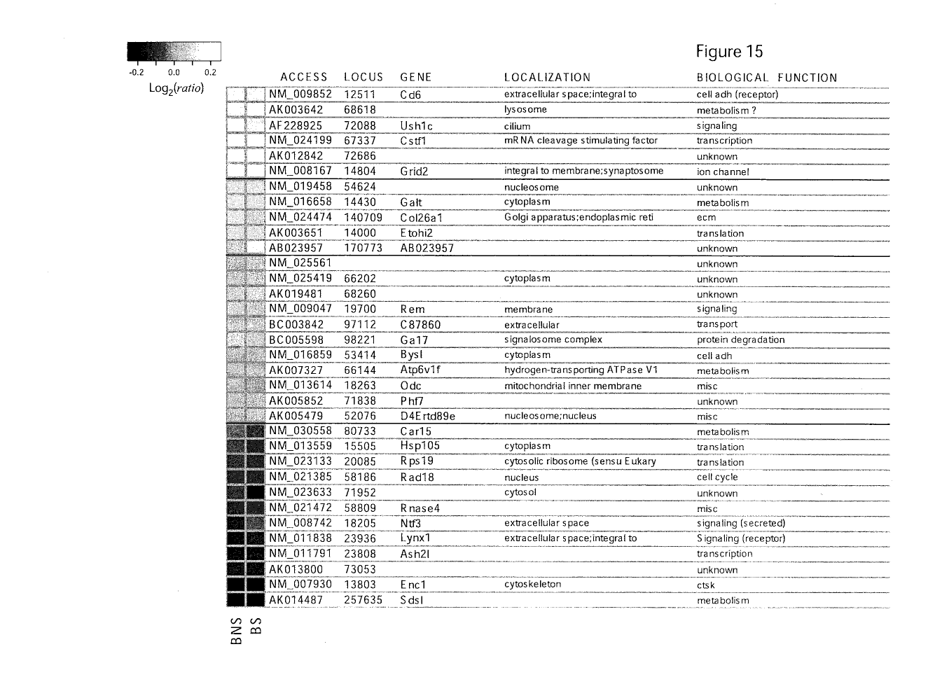

FIG. 15 shows the differentially expressed genes in hypothalamus of mice harboring colon carcinoma at 72 hours.

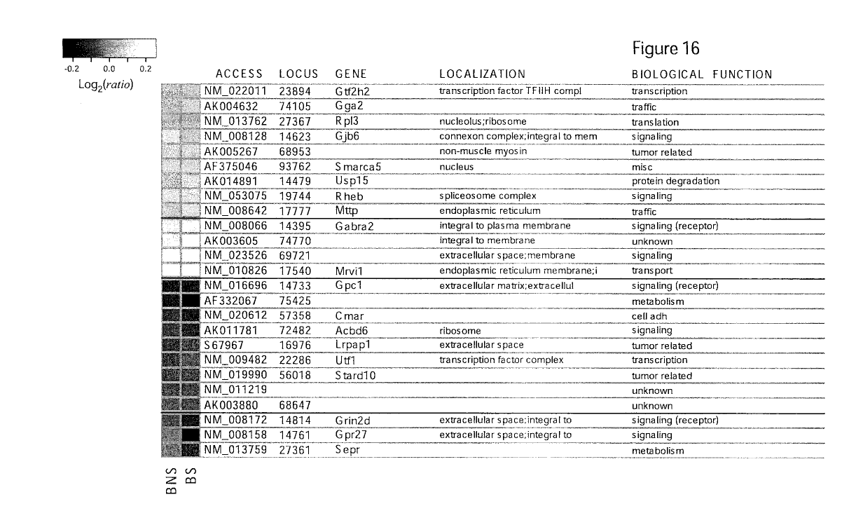

FIG. 16 shows the differentially expressed genes in hypothalamus of mice harboring colon carcinoma at 192 hours.

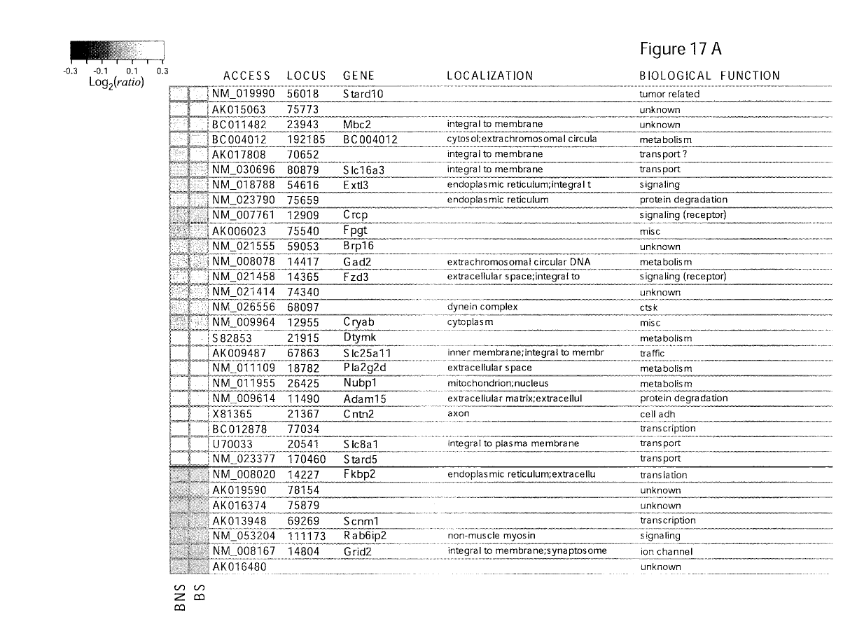

FIGS. 17A & 17B show the differentially expressed genes in hypothalamus of mice harboring lung carcinoma at 18 hours.

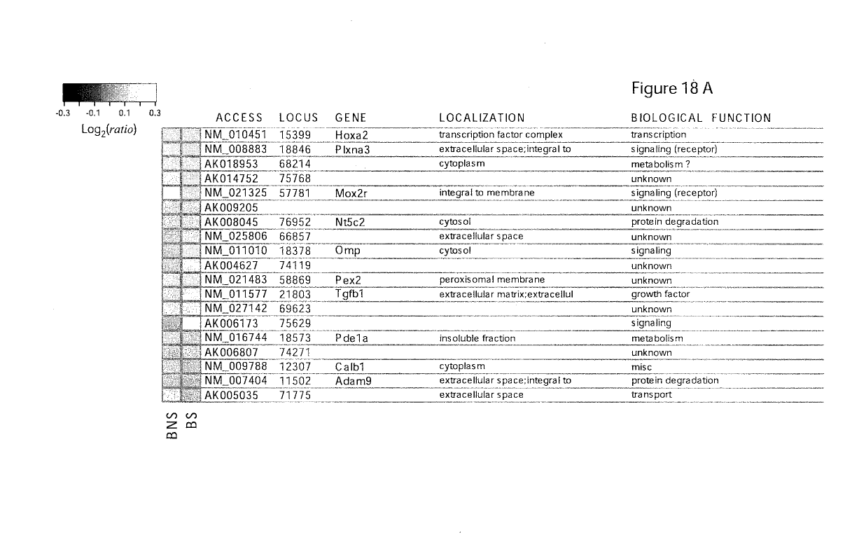

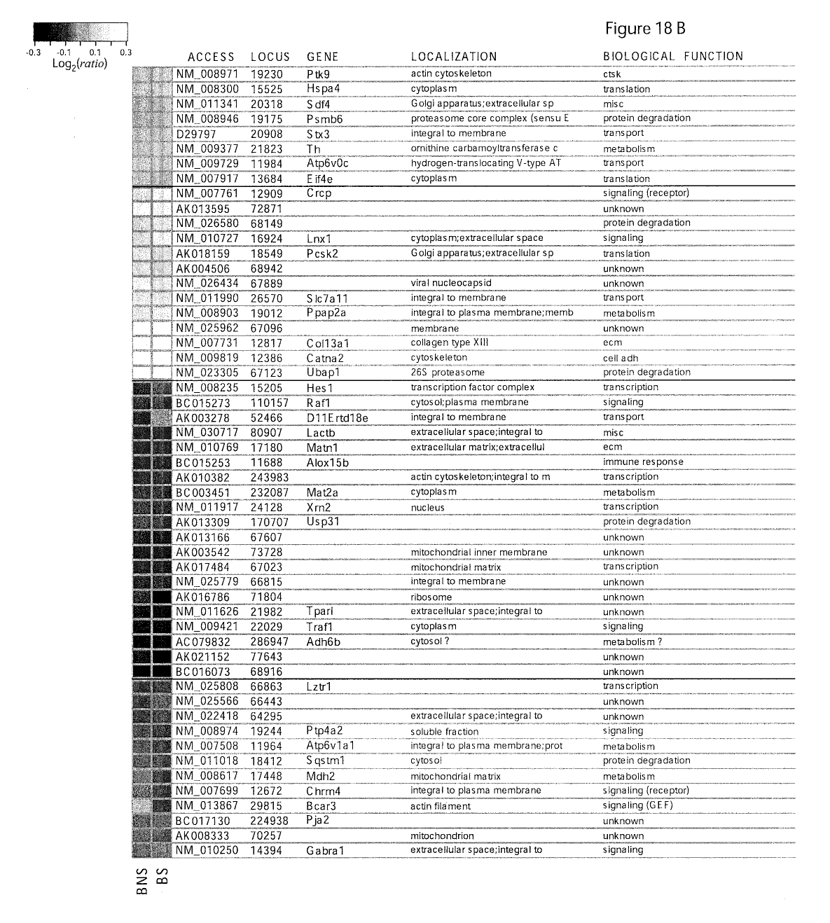

FIGS. 18A & 18B show the differentially expressed genes in hypothalamus of mice harboring lung carcinoma at 72 hours.

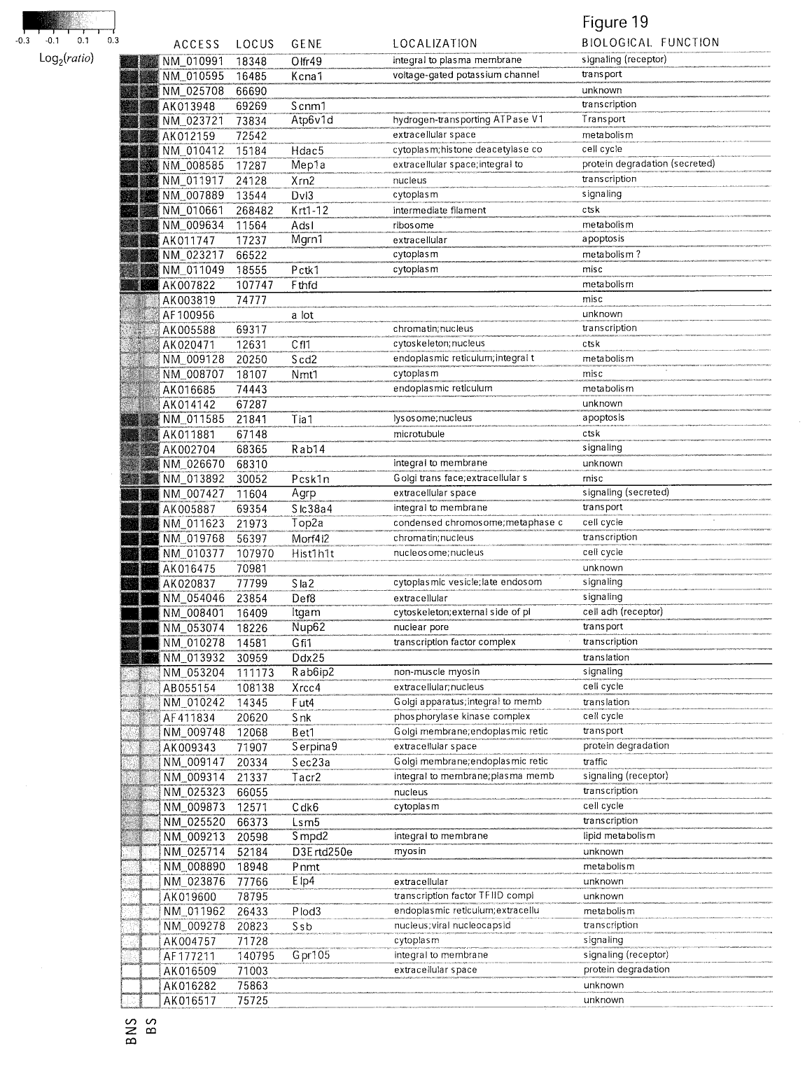

FIG. 19 shows the differentially expressed genes in hypothalamus of mice harboring lung carcinoma at 192 hours.

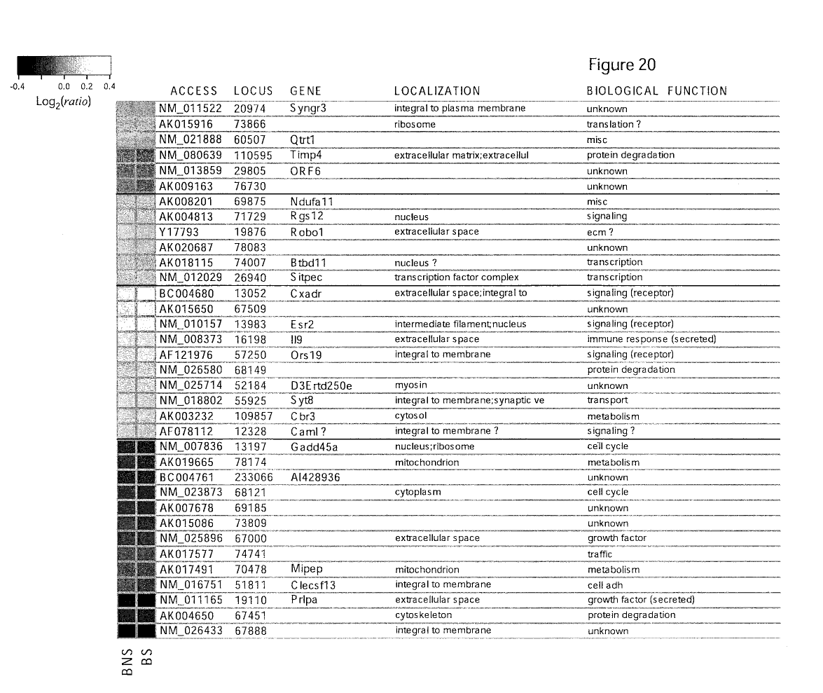

FIG. 20 shows the differentially expressed genes in midbrain of mice harboring breast carcinoma at 18 hours.

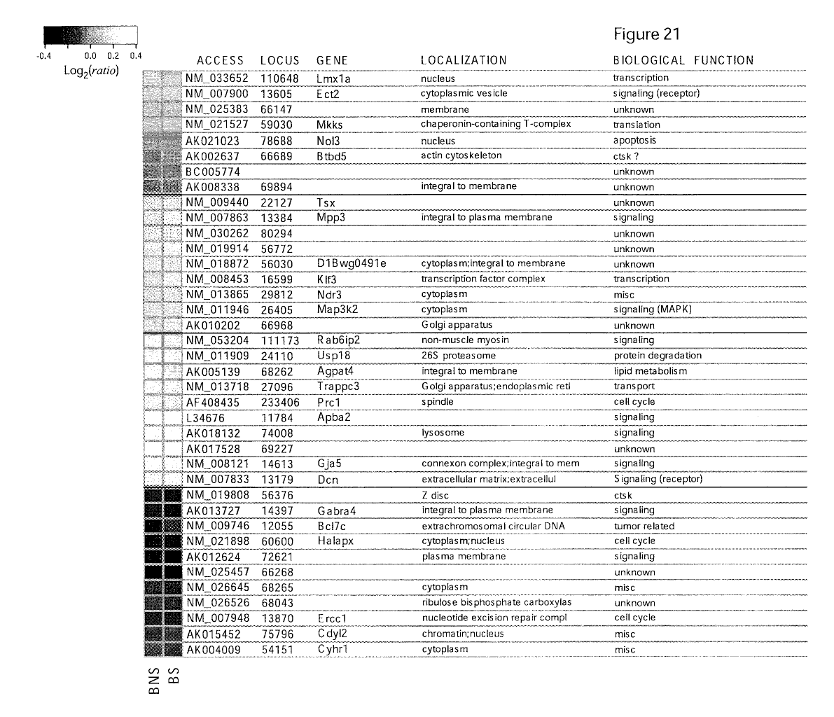

FIG. 21 shows the differentially expressed genes in midbrain of mice harboring breast carcinoma at 72 hours.

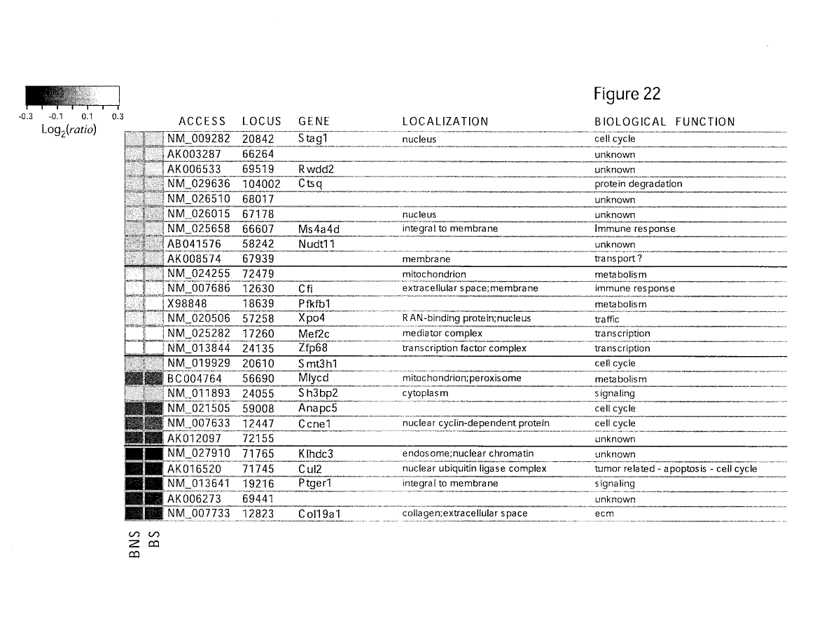

FIG. 22 shows the differentially expressed genes in midbrain of mice harboring breast carcinoma at 192 hours.

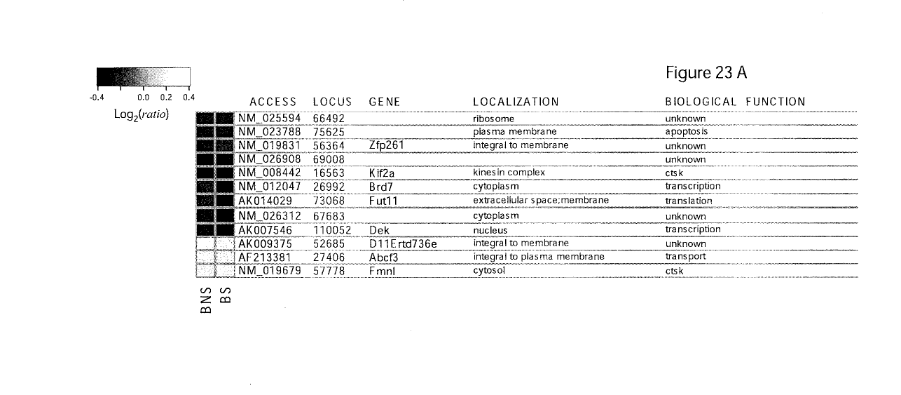

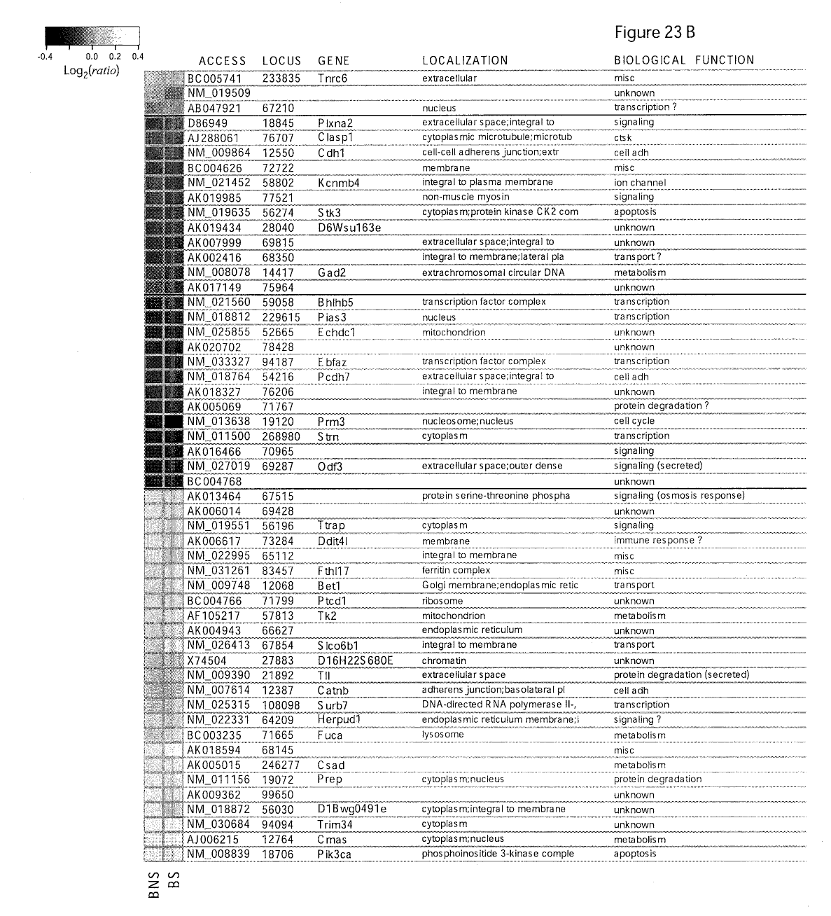

FIGS. 23A & 23B show the differentially expressed genes in midbrain of mice harboring colon carcinoma at 18 hours.

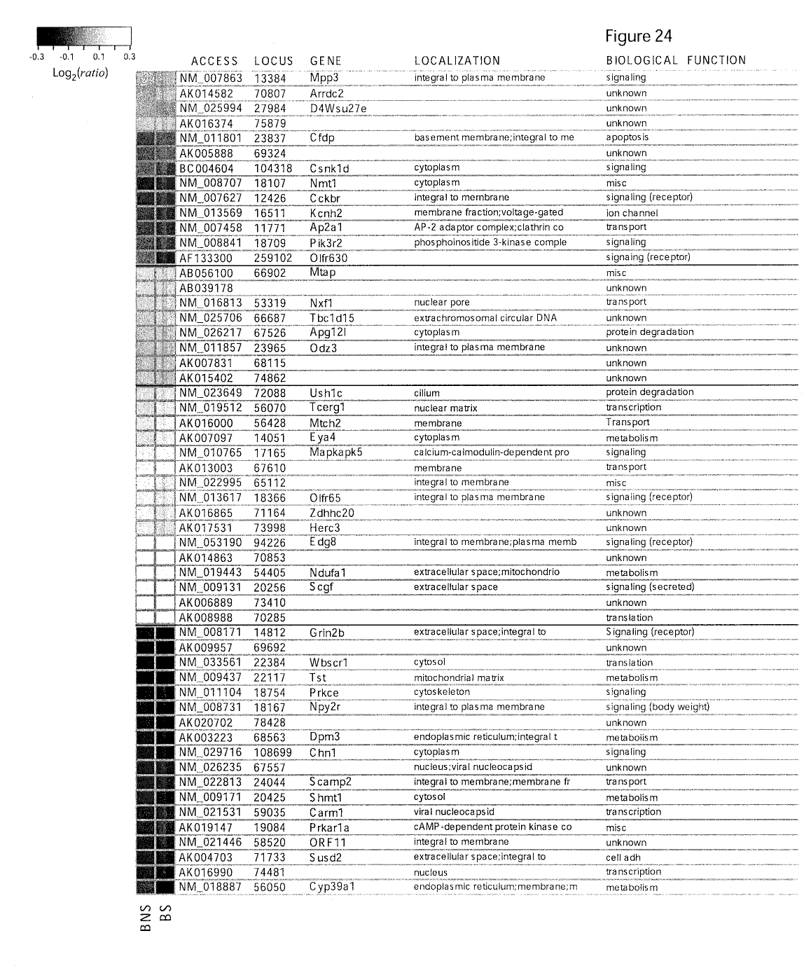

FIG. 24 shows the differentially expressed genes in midbrain of mice harboring colon carcinoma at 72 hours.

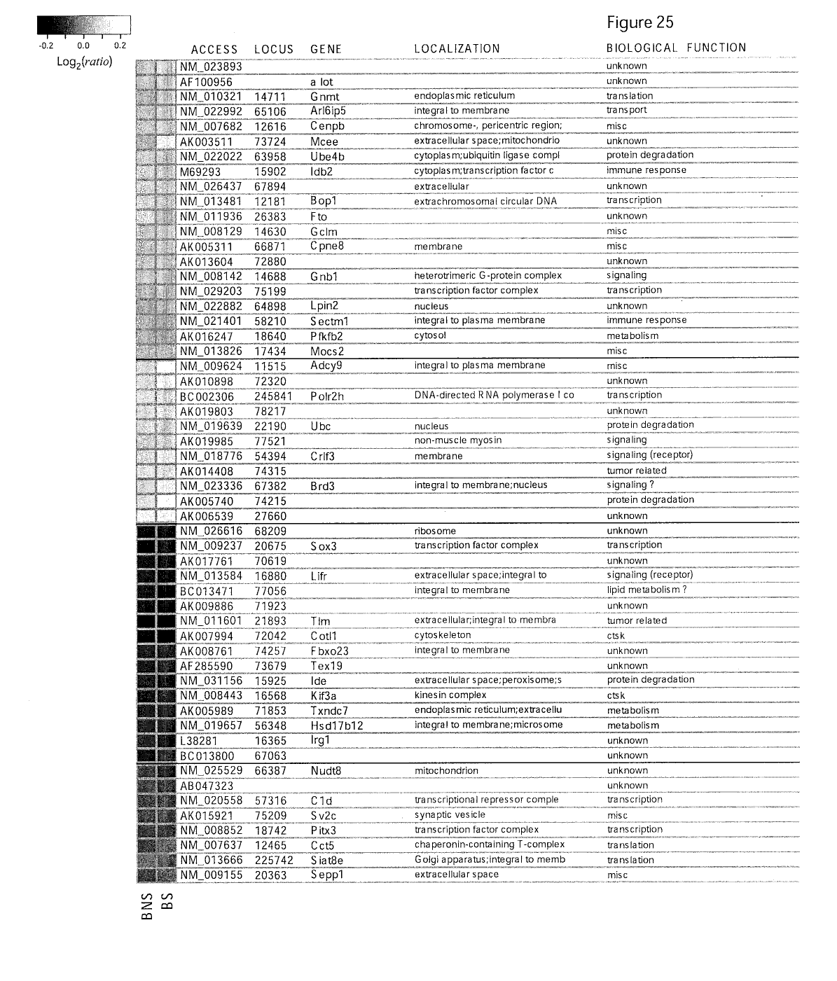

FIG. 25 shows the differentially expressed genes in midbrain of mice harboring colon carcinoma at 192 hours.

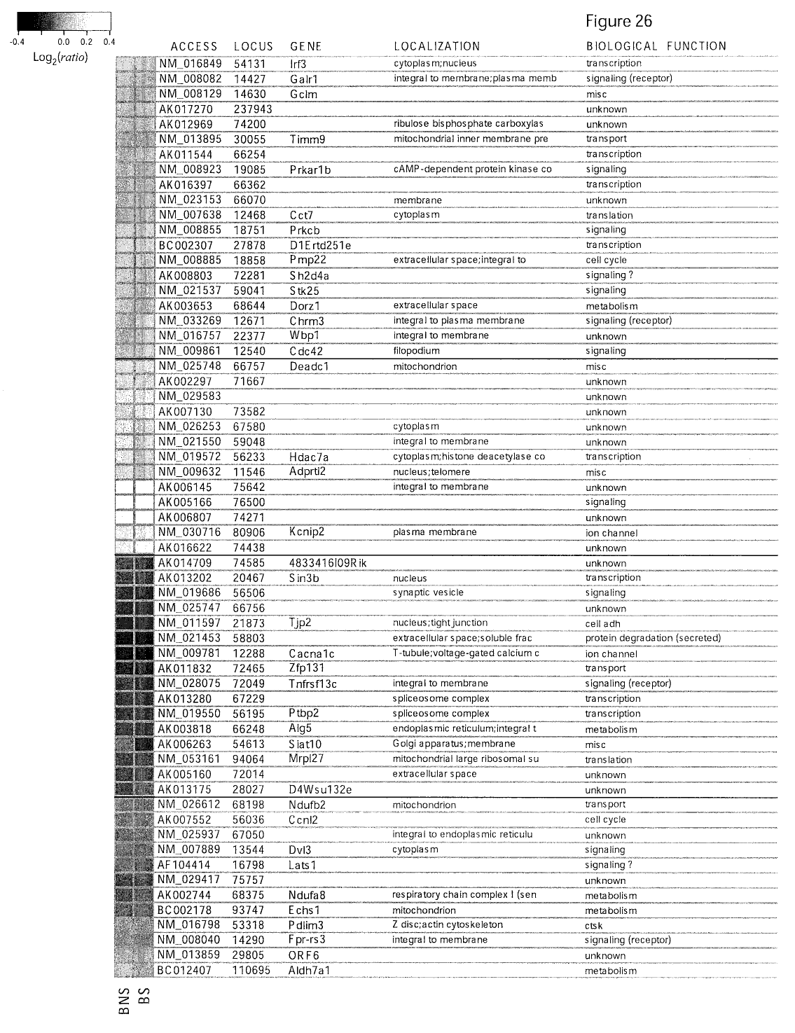

FIG. 26 shows the differentially expressed genes in midbrain of mice harboring lung carcinoma at 18 hours.

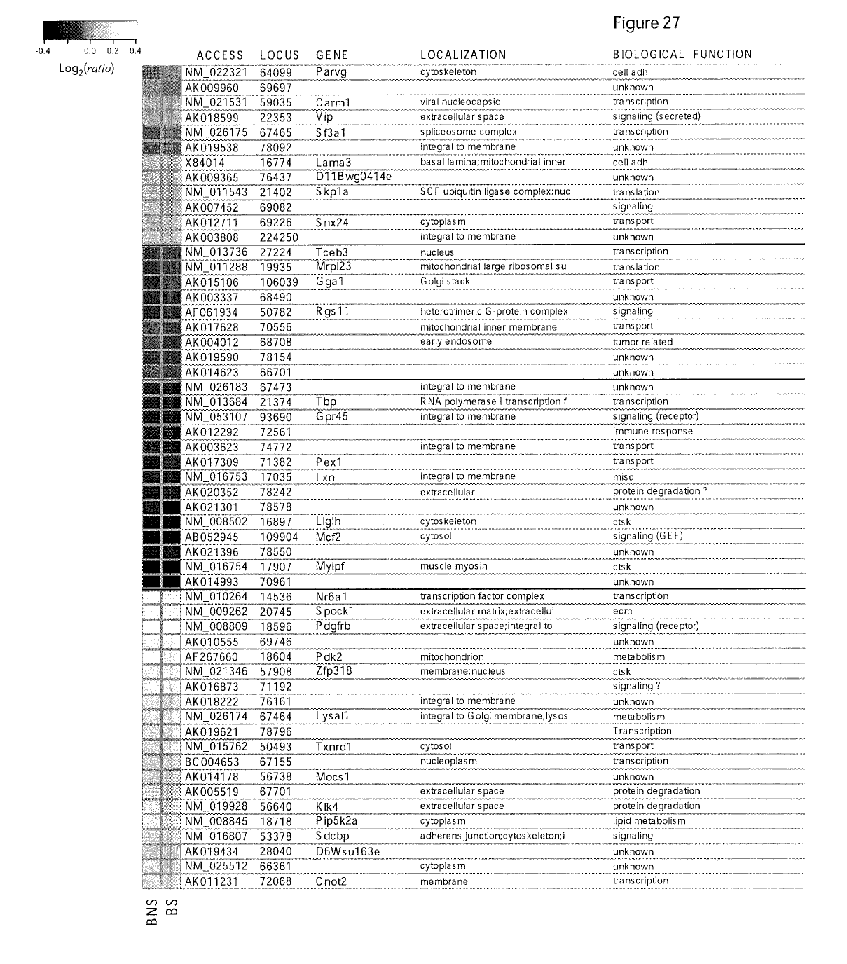

FIG. 27 shows the differentially expressed genes in midbrain of mice harboring lung carcinoma at 72 hours.

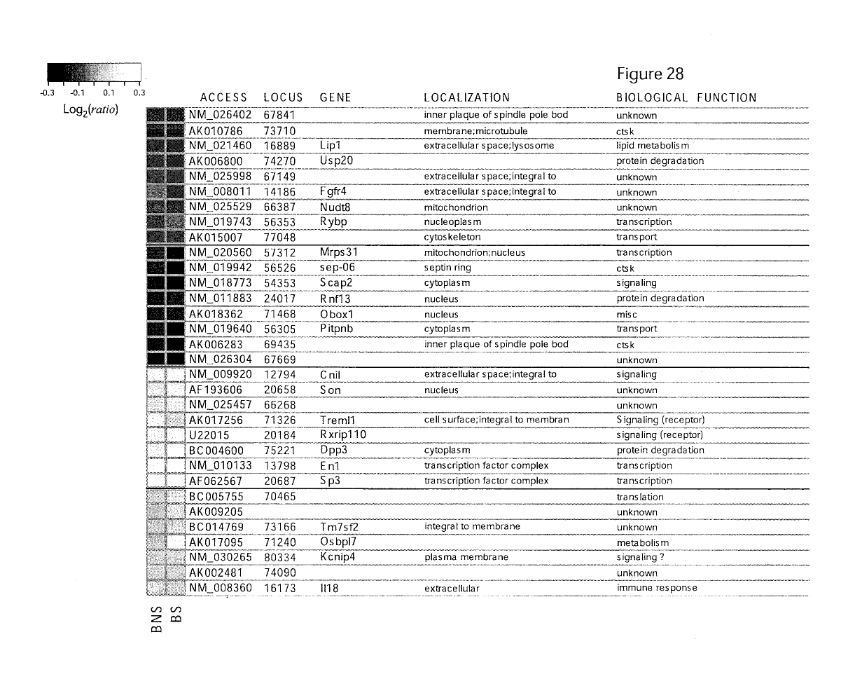

FIG. 28 shows the differentially expressed genes in midbrain of mice harboring lung carcinoma at 192 hours.

The following FIGS. 29 to 37-6 are tables showing genes differentially expressed genes in mice harboring either breast, colon, or lung carcinoma compared to control mice (p<0.01) after performing a hierarchical cluster analysis. Samples were obtained from either the prefrontal cortex, the hypothalamus, or the midbrain at 18, 72 and 192 hours post tumor cell injection. Differentially expressed genes were identified by a mixed model ANOVA, with tumor (or control) and time points as fixed effects. The base 2 logarithm of tumor vs. control ratio is shown as a gray scale. BNS corresponds to data obtained without background subtraction, and BS corresponds to data obtained after background subtraction.

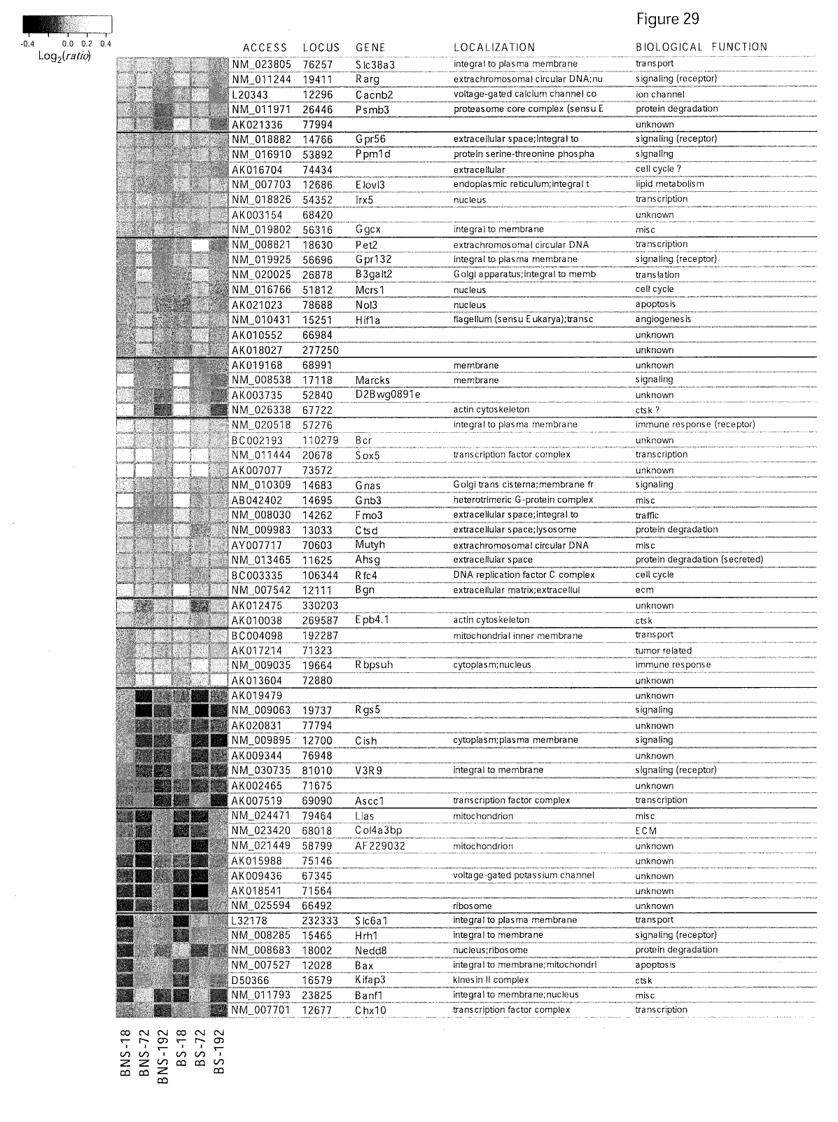

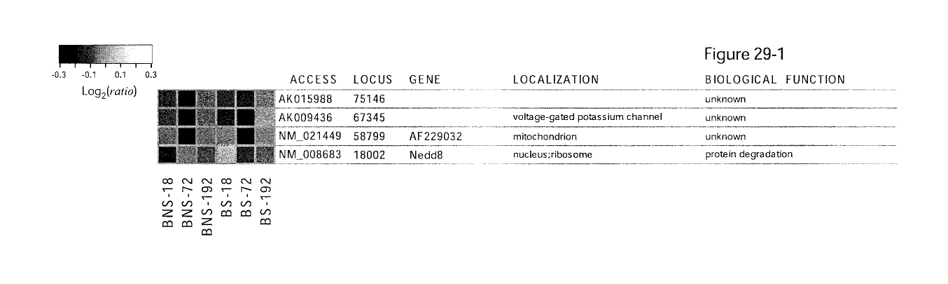

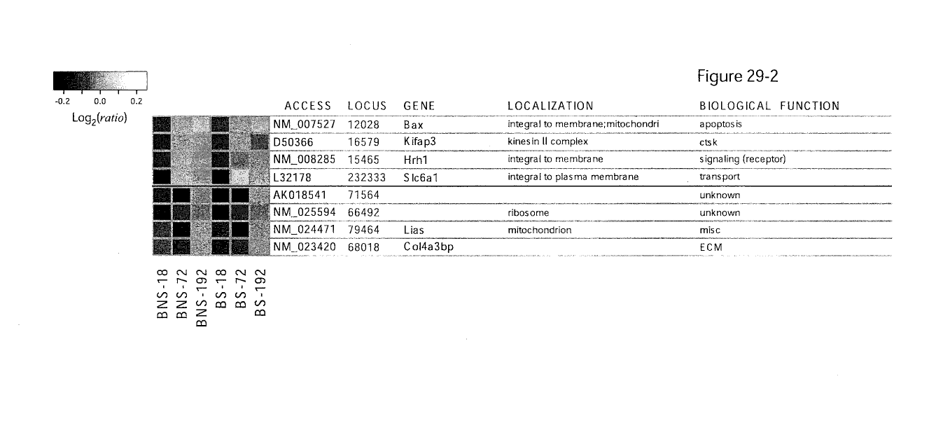

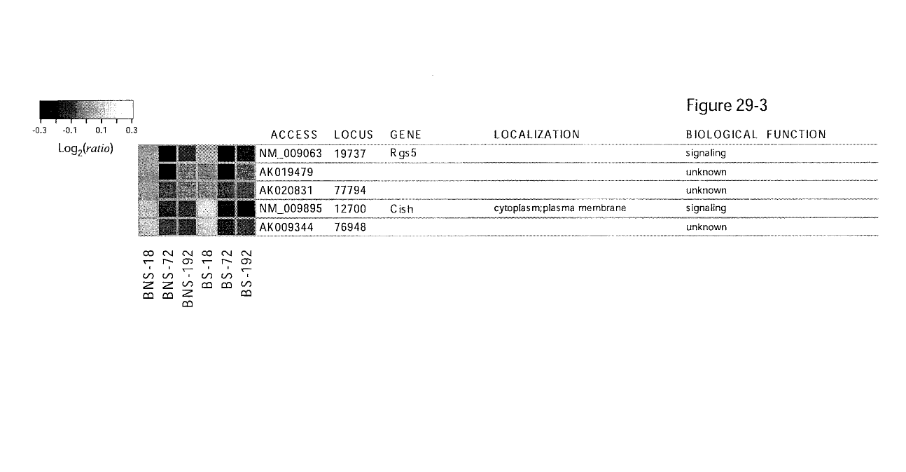

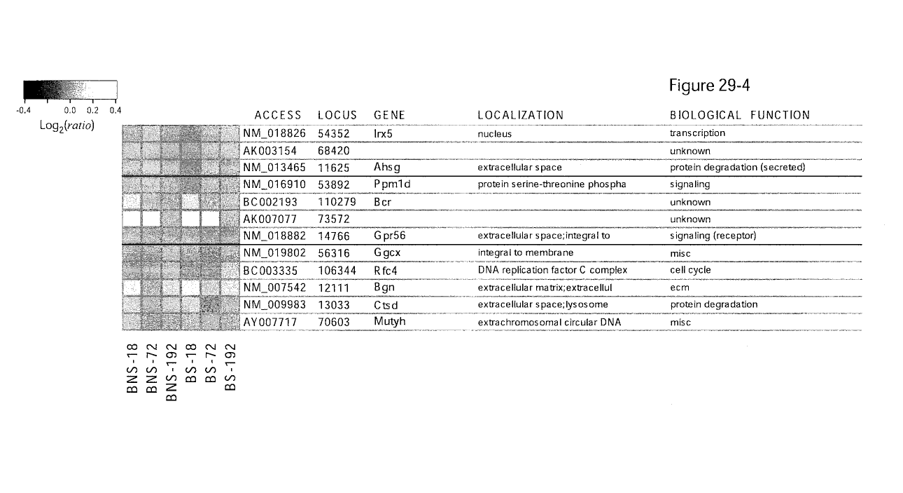

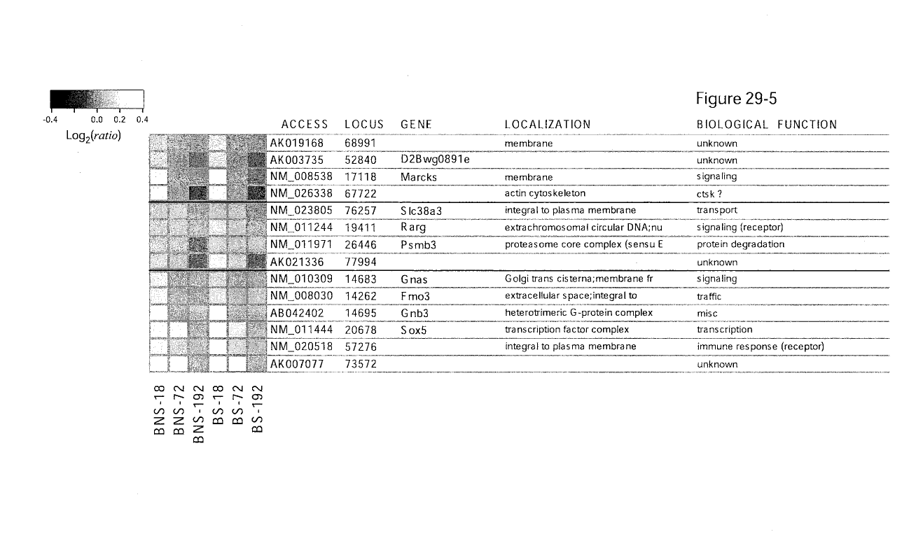

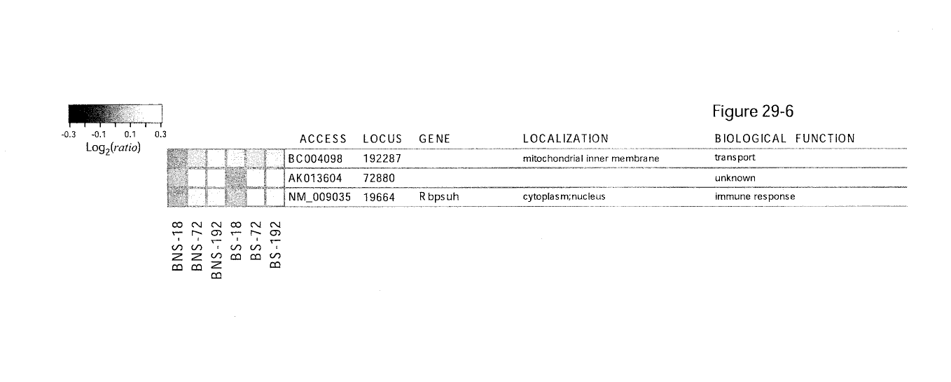

FIG. 29 shows differentially expressed genes in mice harboring breast carcinoma after performing a hierarchical cluster analysis. Samples were obtained from prefrontal cortex.

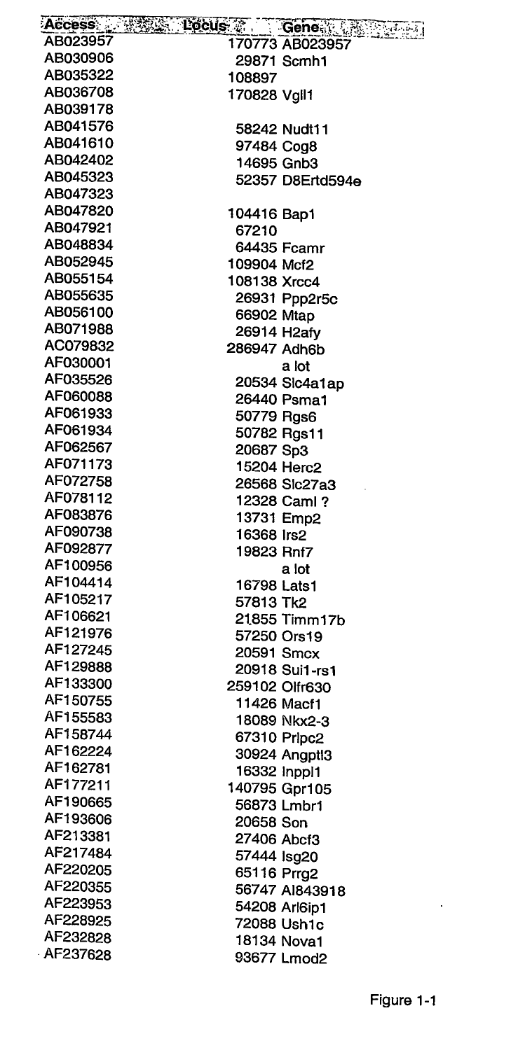

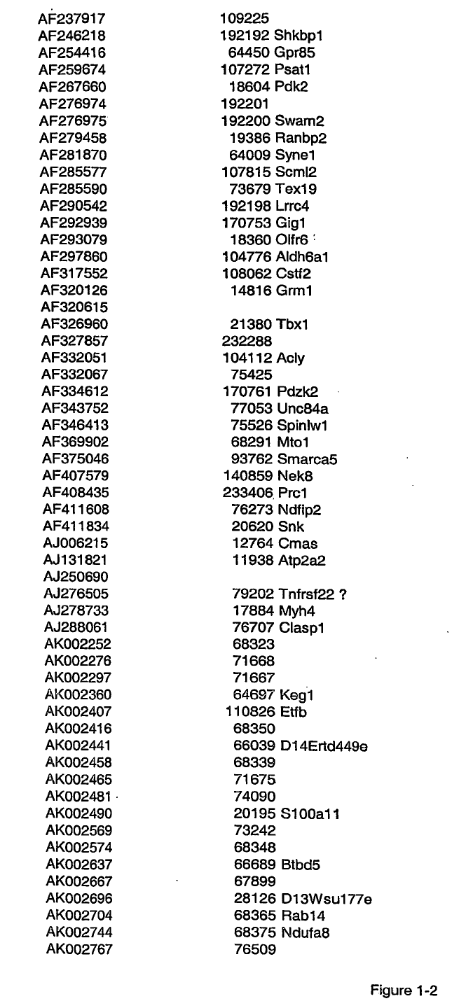

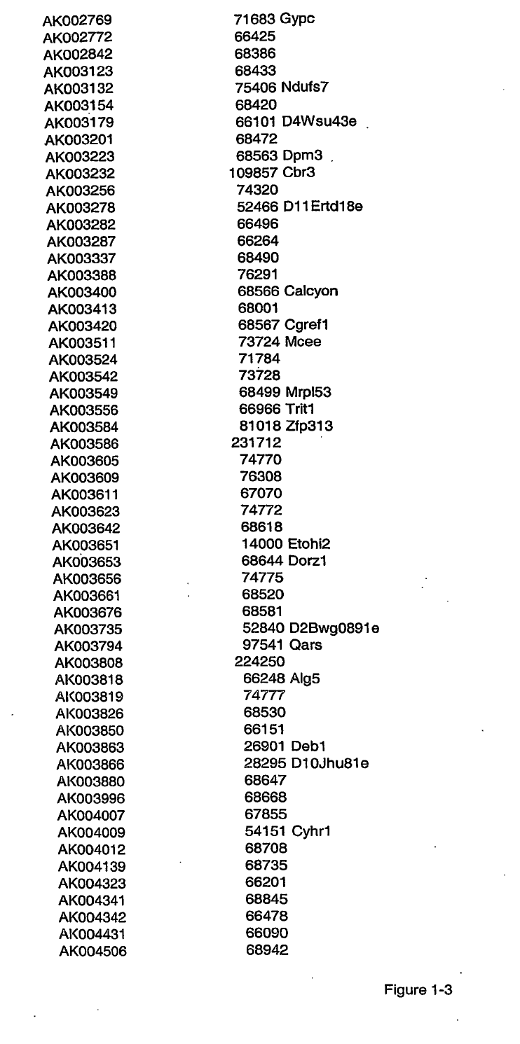

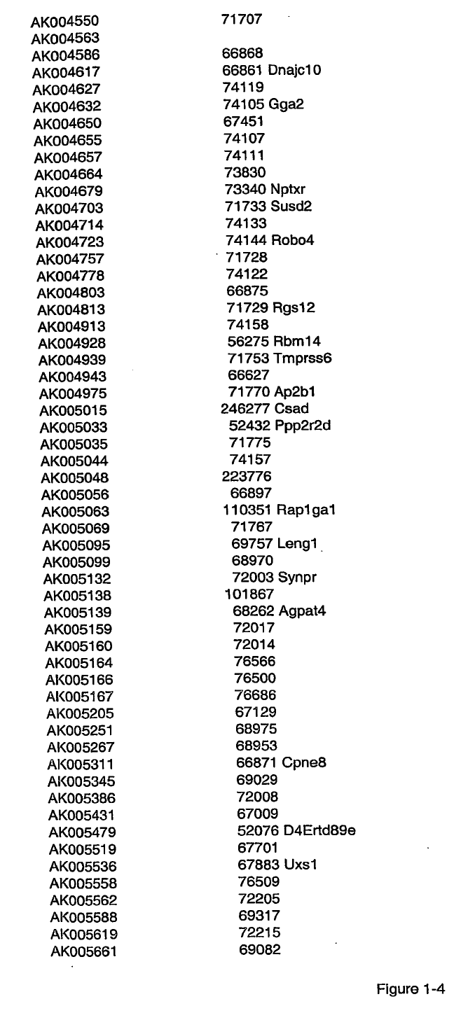

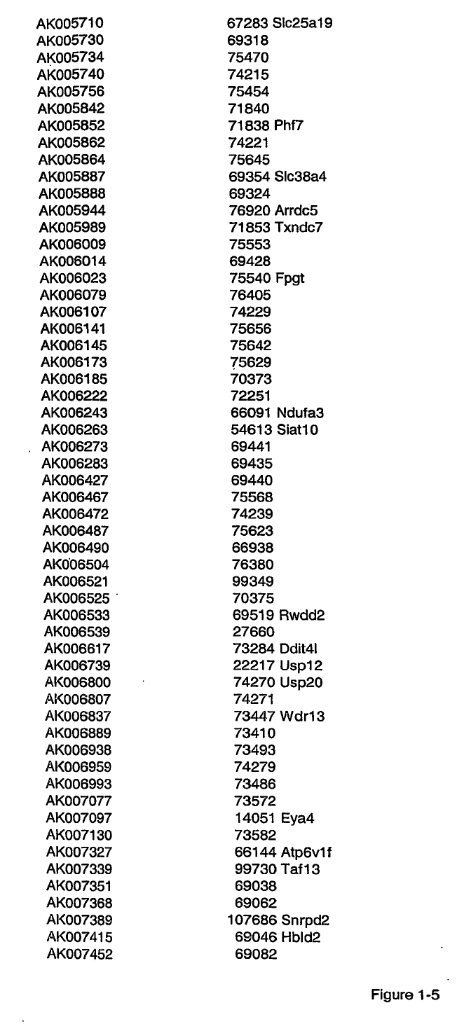

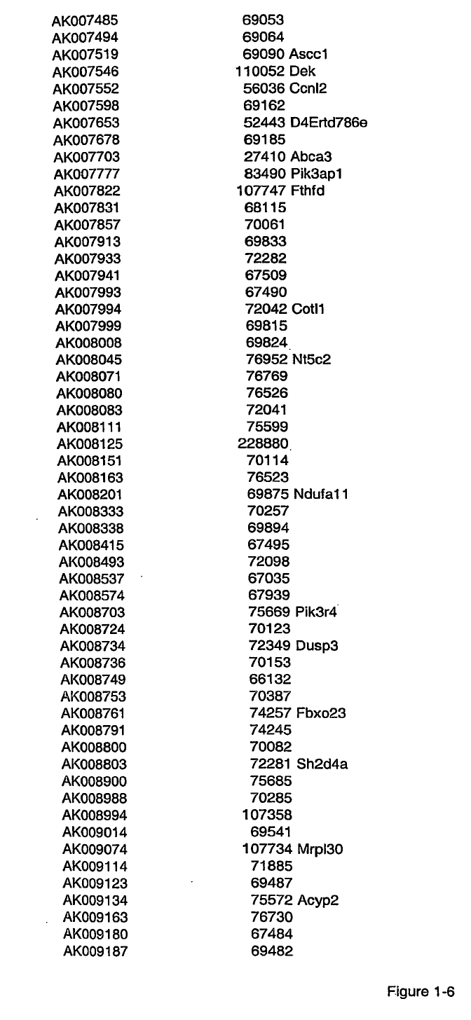

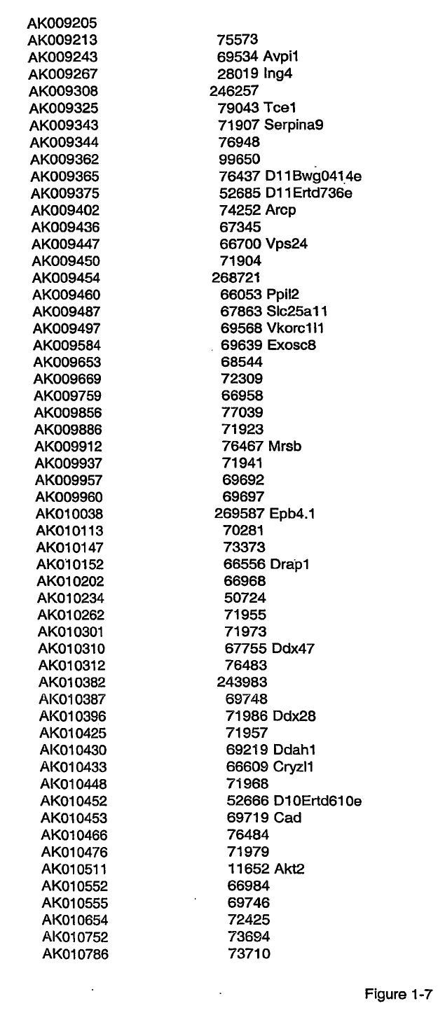

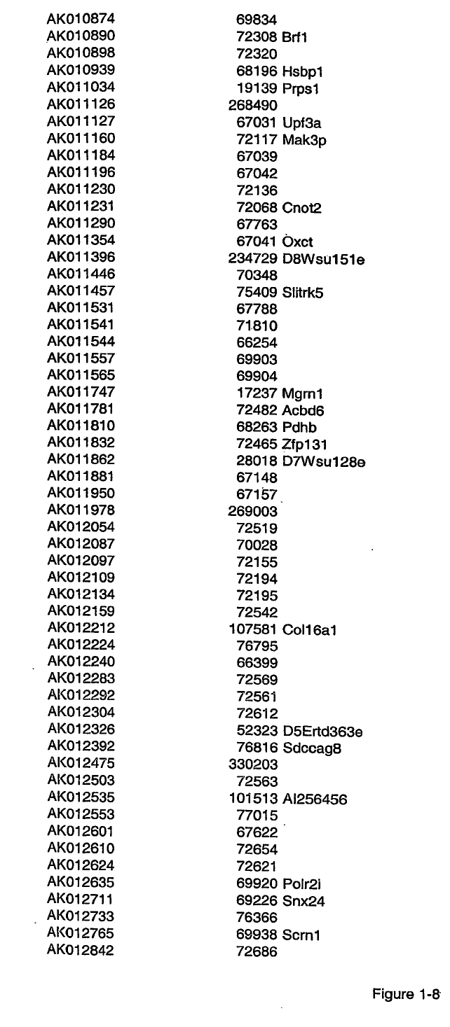

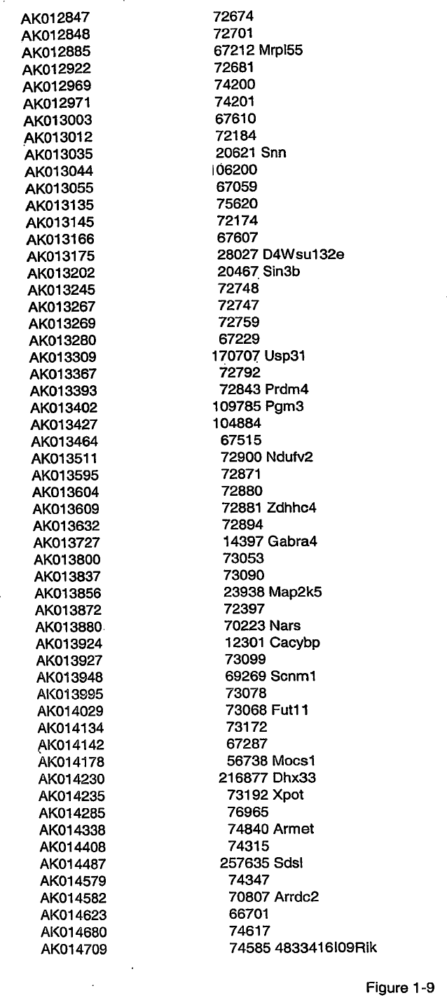

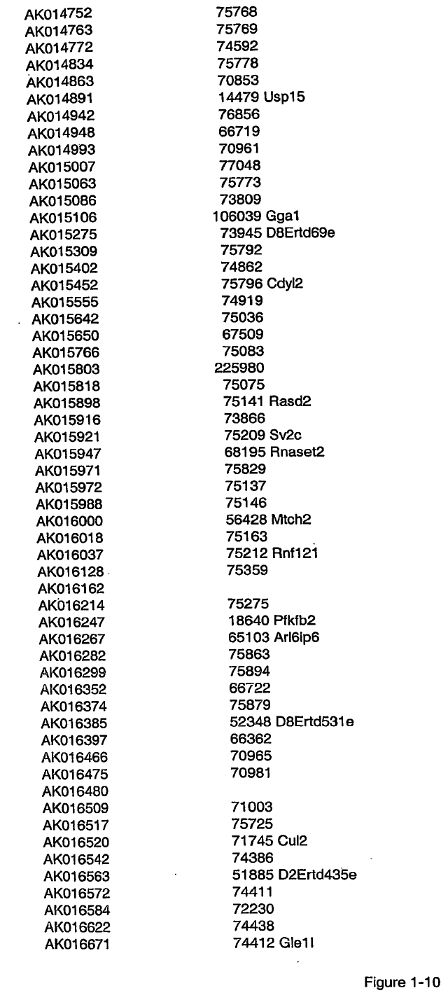

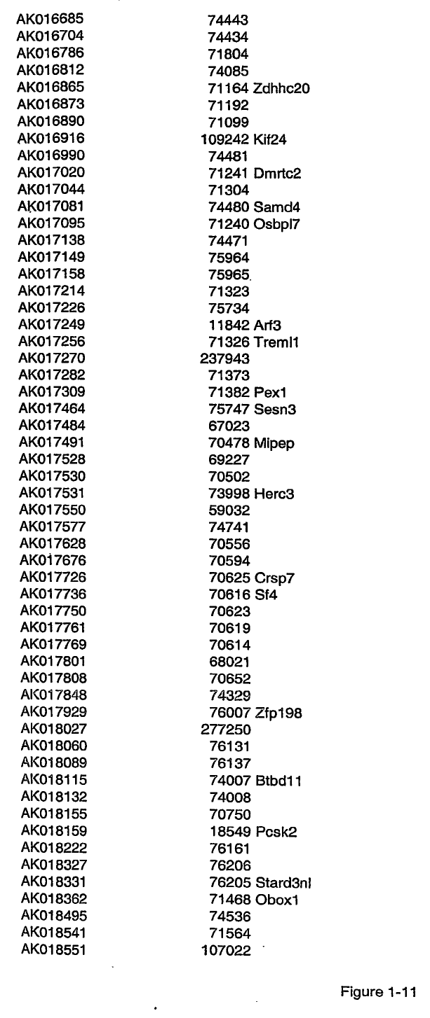

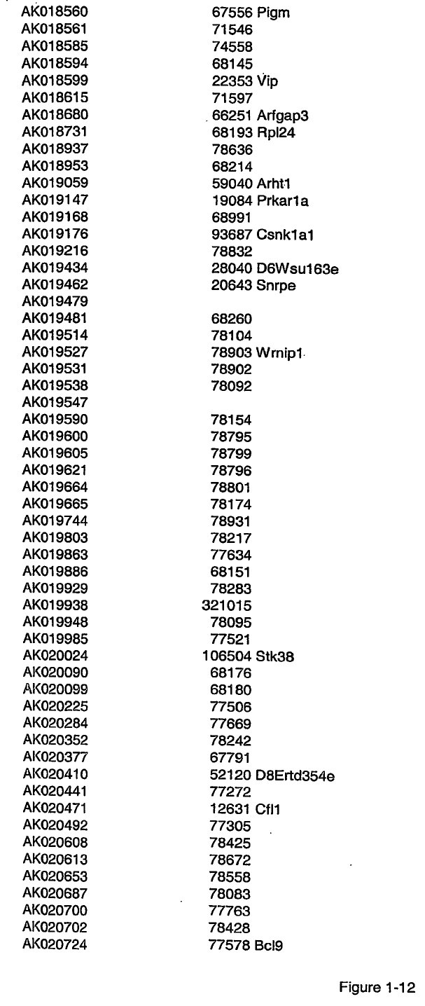

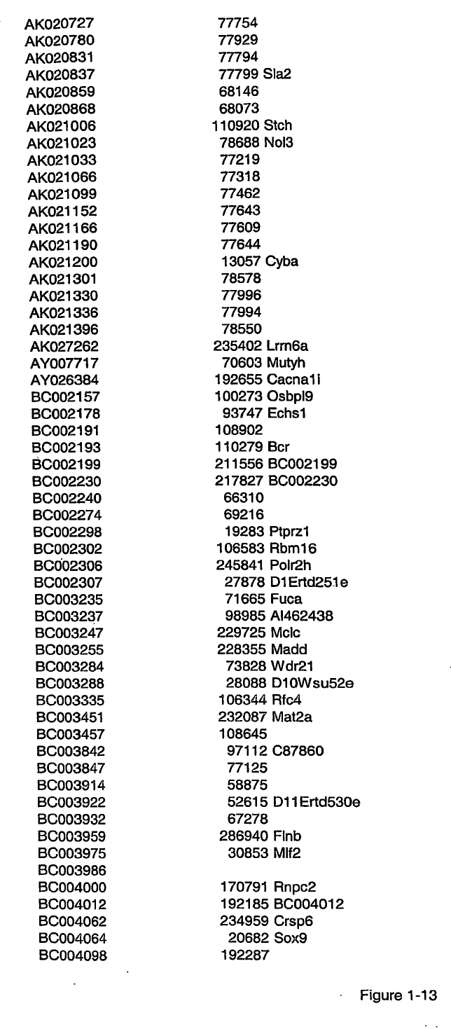

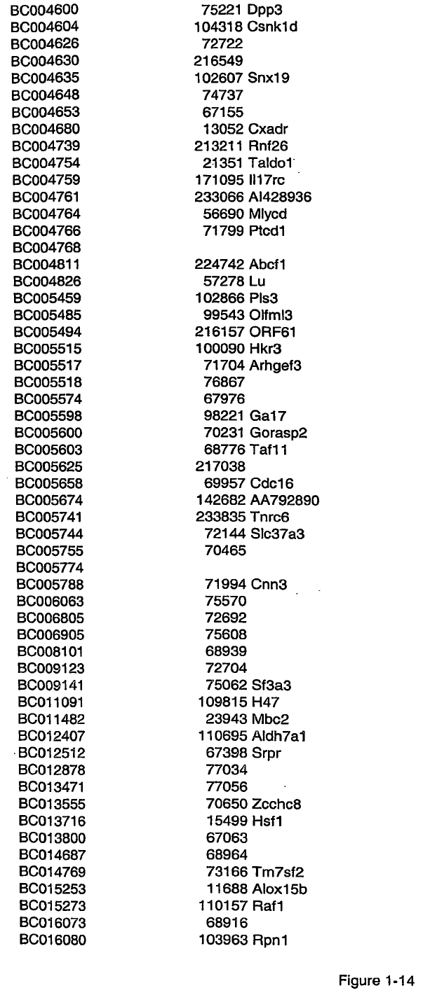

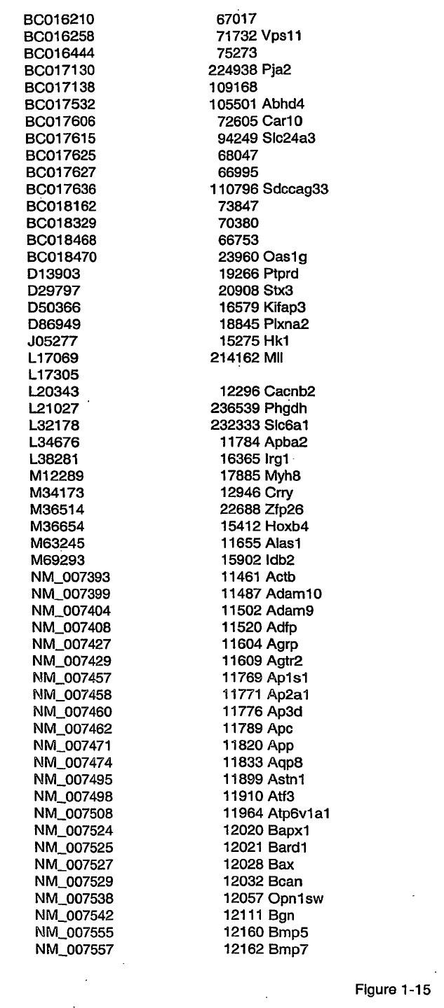

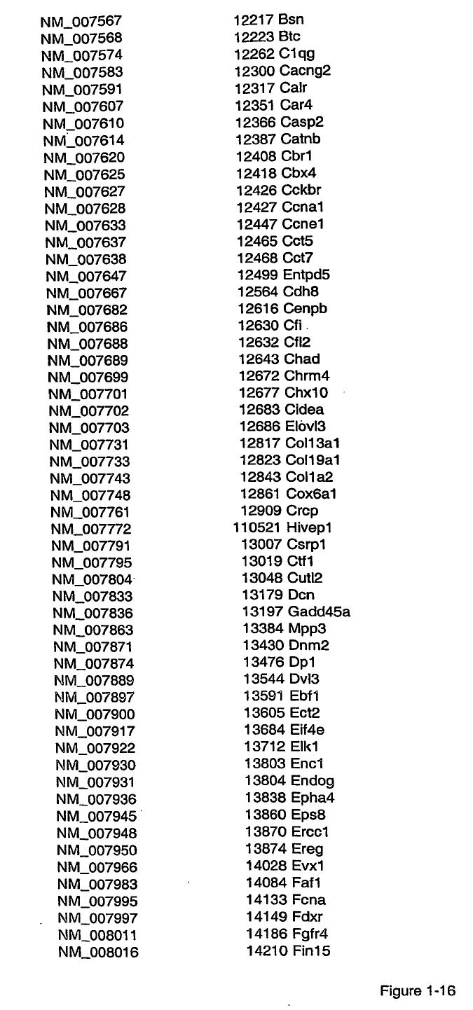

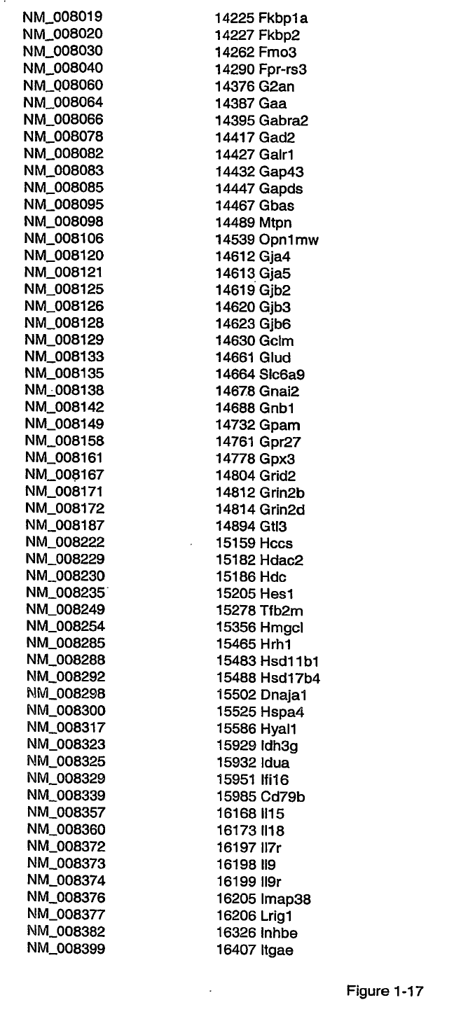

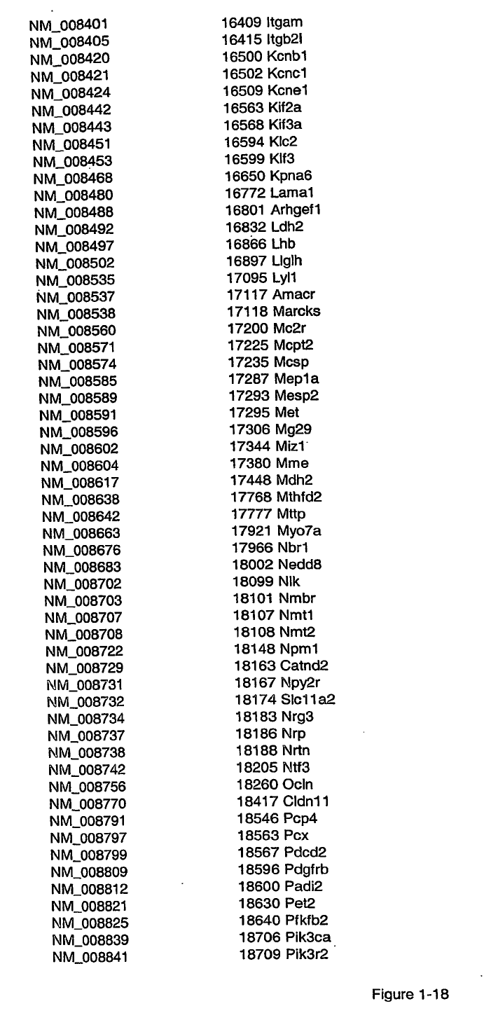

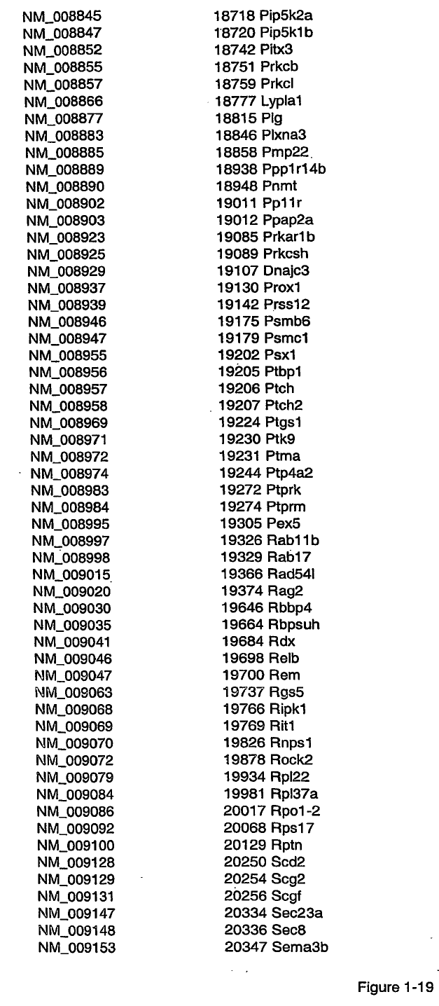

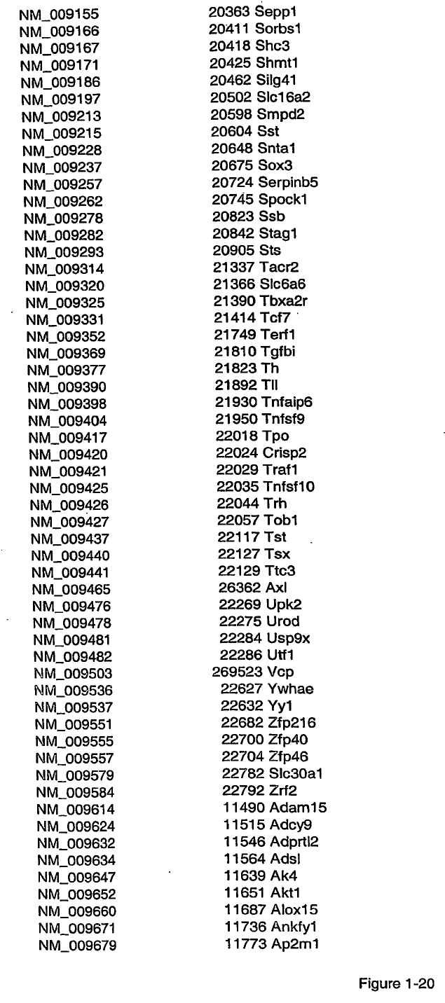

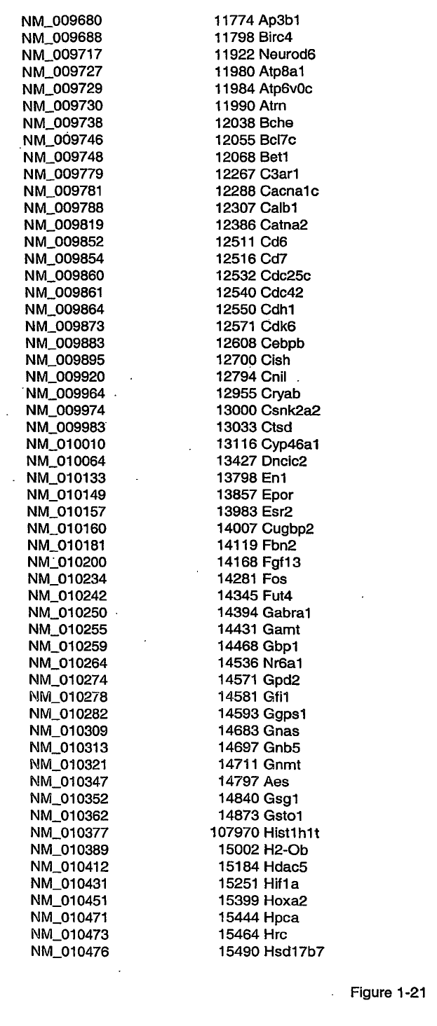

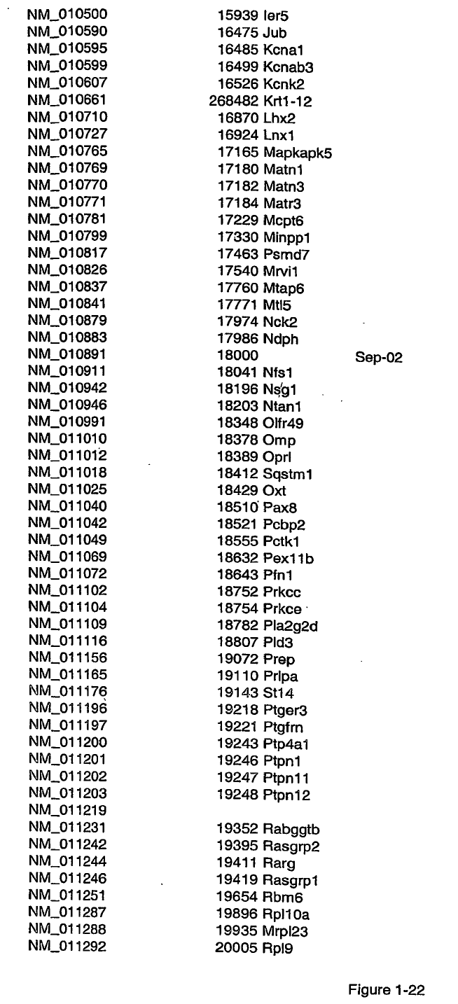

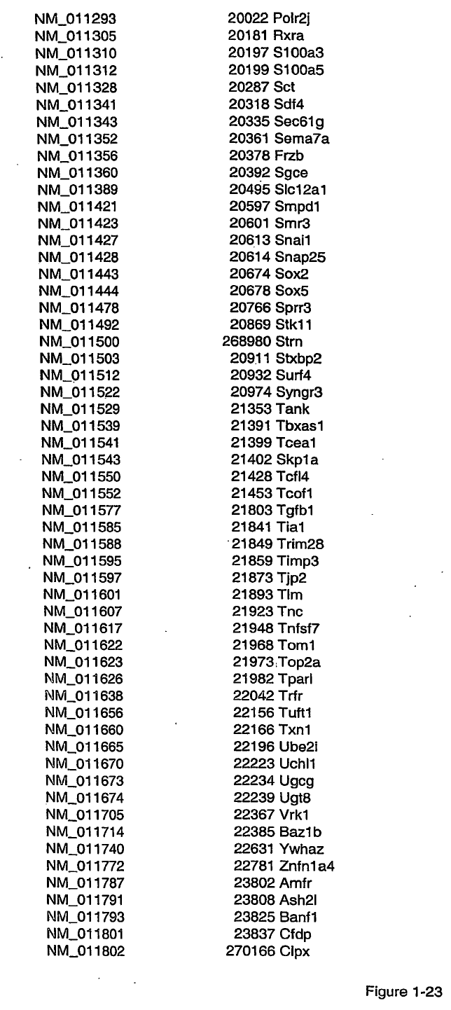

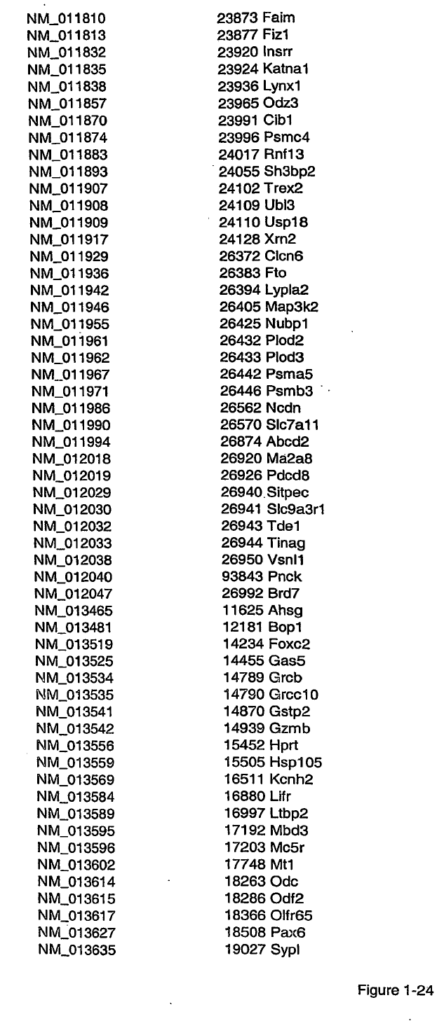

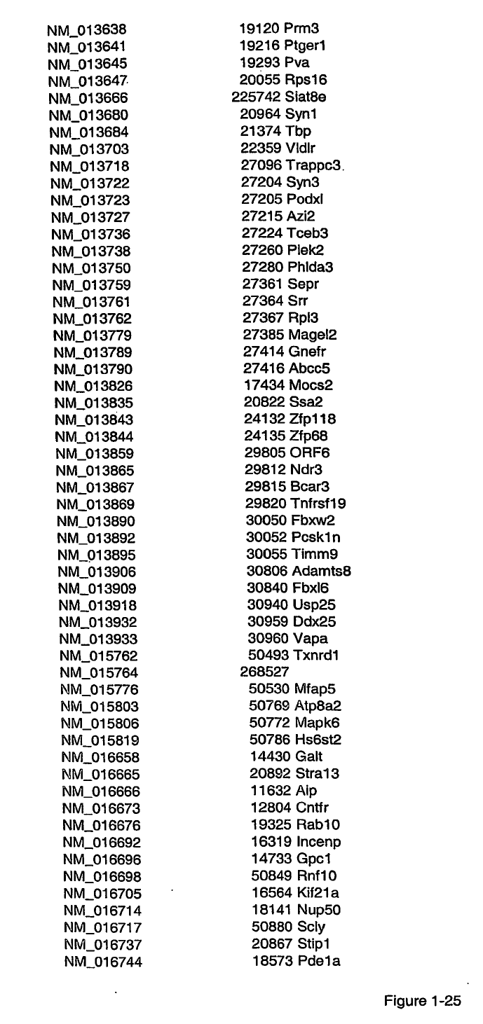

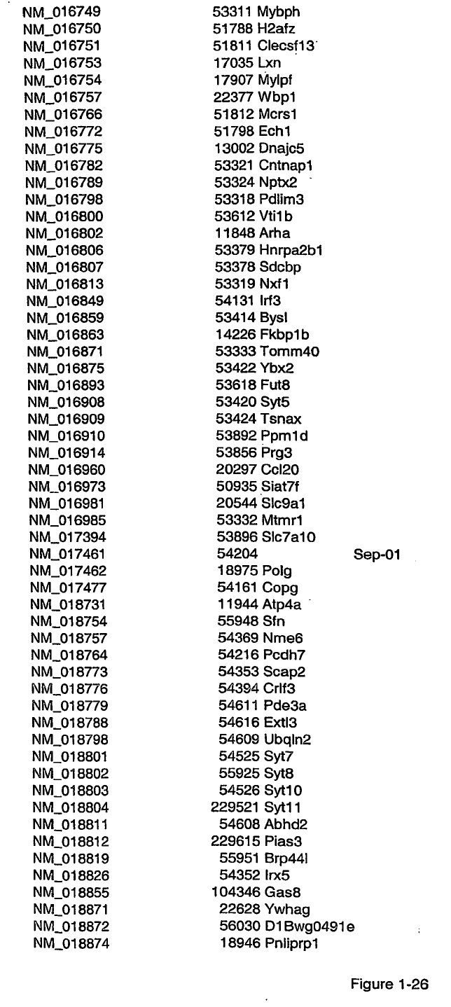

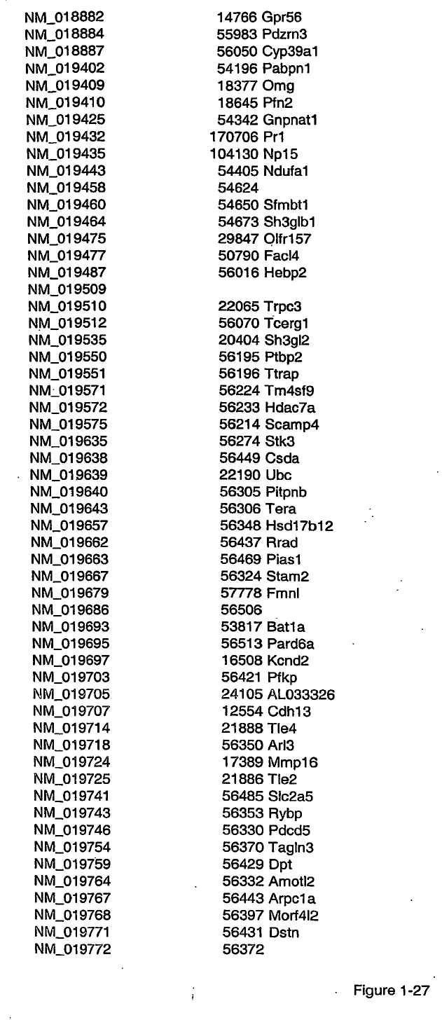

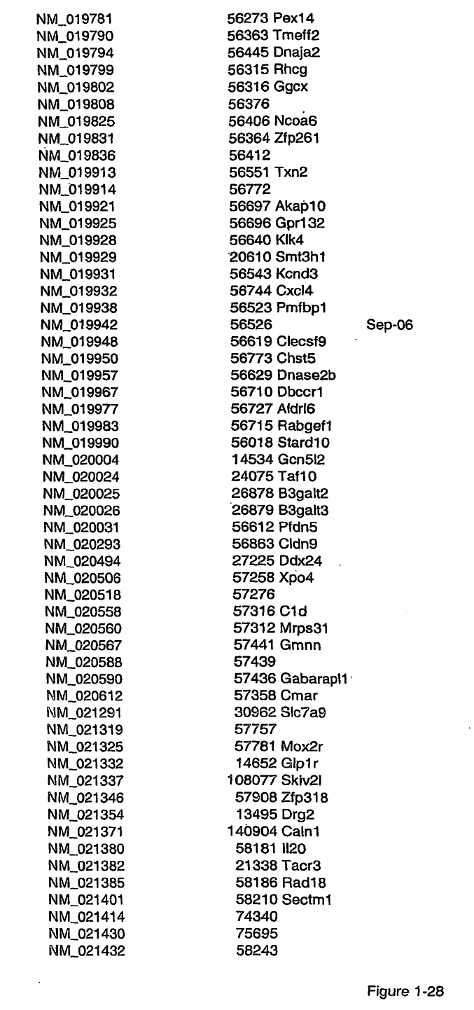

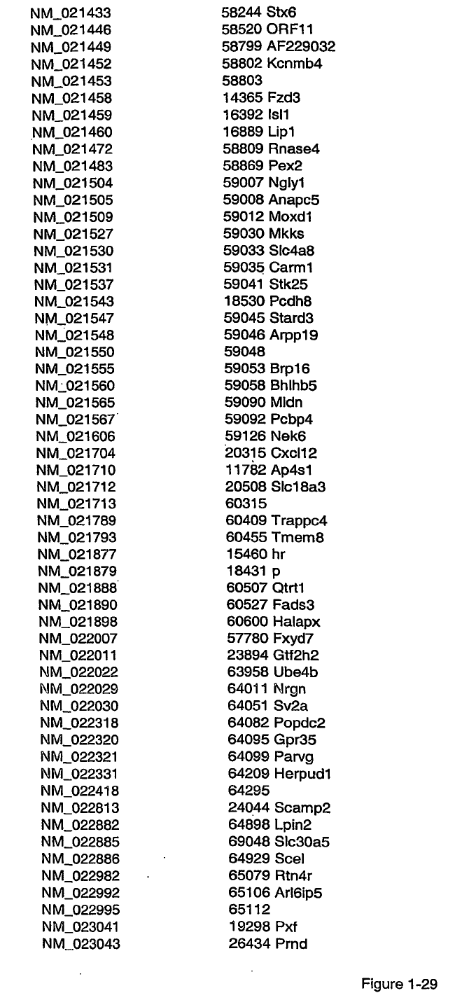

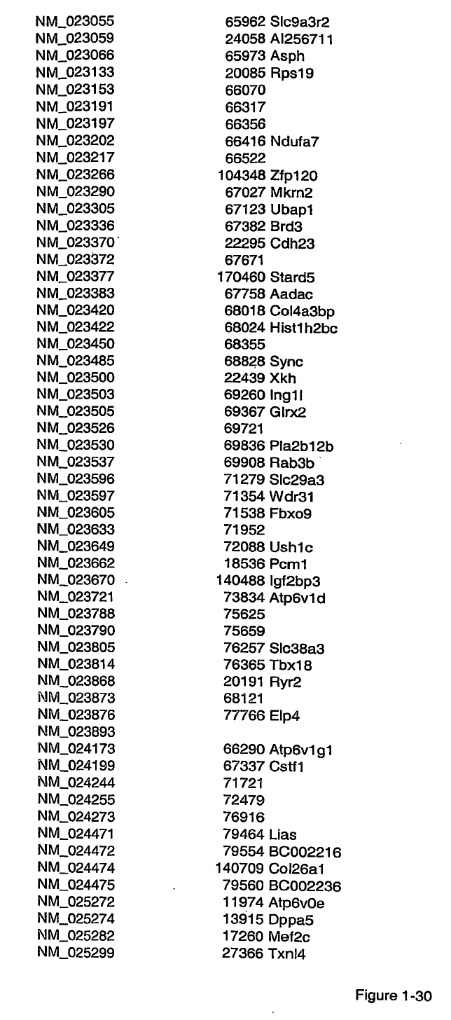

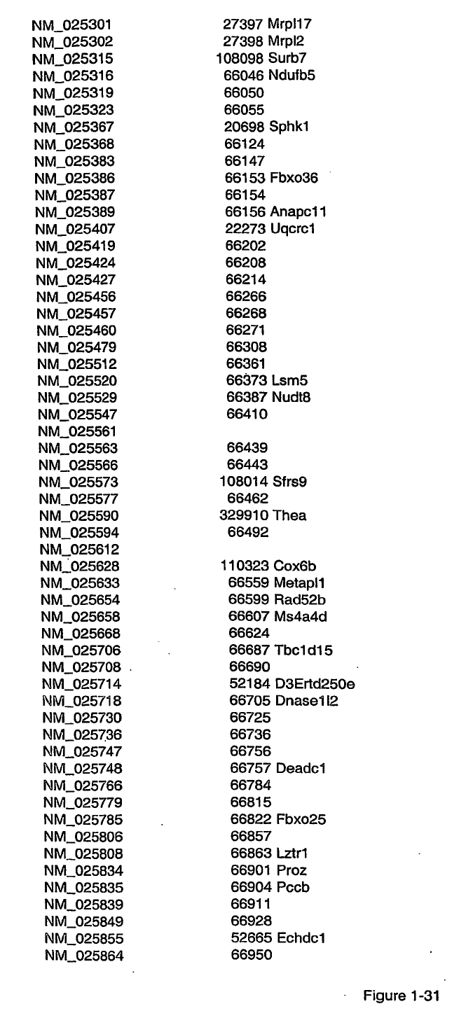

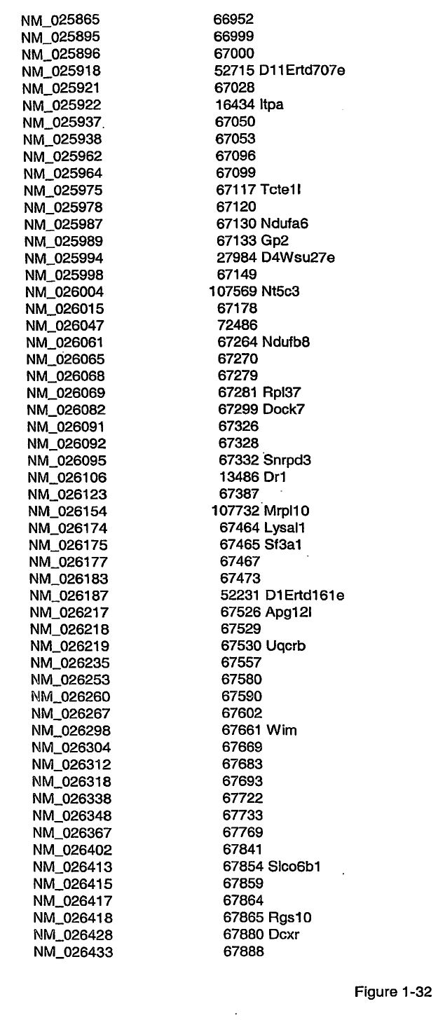

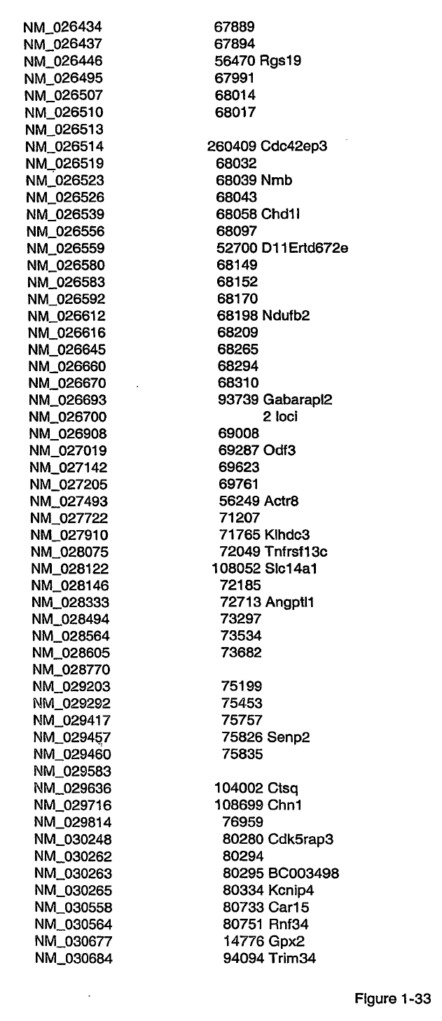

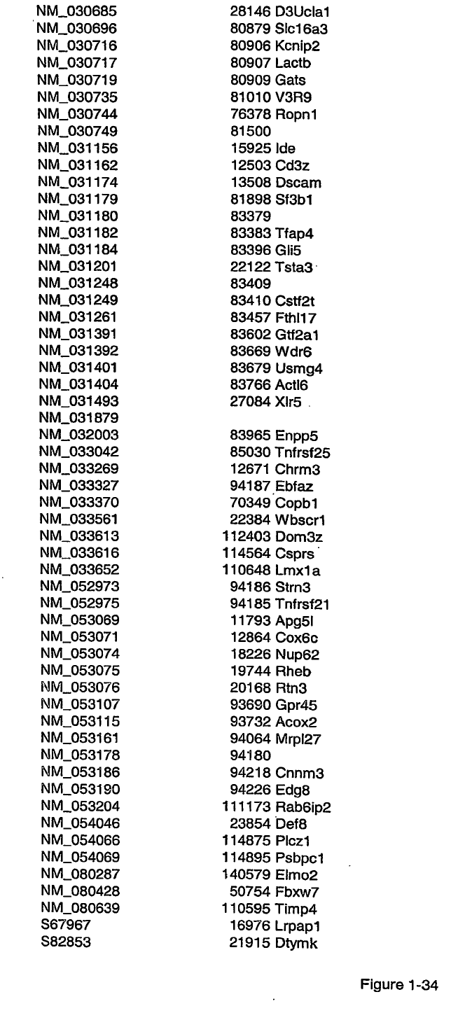

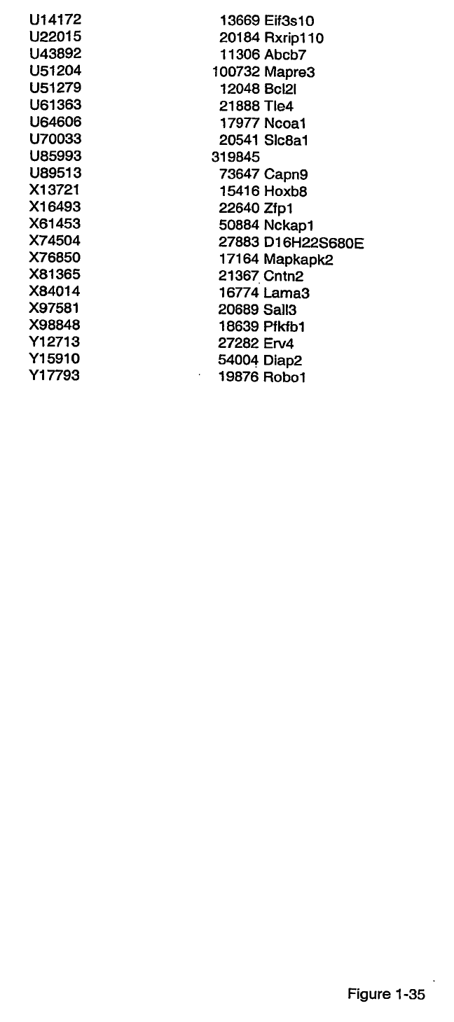

FIG. 29-1 shows down-regulated genes in mice harboring breast carcinoma. Samples were obtained from prefrontal cortex. The list of genes corresponds to those that are down-regulated at all the time points.

FIG. 29-2 shows down-regulated genes in mice harboring breast carcinoma. Samples were obtained from prefrontal cortex. The list of genes corresponds to those that are down-regulated at 18 hours, or at 18 hours and 72 hours post tumor injection.

FIG. 29-3 shows down-regulated genes in mice harboring breast carcinoma. Samples were obtained from prefrontal cortex. The list of genes corresponds to those that are down-regulated at 72 hours and 192 hours post tumor injection.

FIG. 29-4 shows up-regulated genes in mice harboring breast carcinoma. Samples were obtained from prefrontal cortex. The list of genes corresponds to those that are up-regulated at all the time points

FIG. 29-5 shows up-regulated genes in mice harboring breast carcinoma. Samples were obtained from prefrontal cortex. The list of genes corresponds to those that are up regulated at 18 hours, or at 18 hours and 72 hours post tumor injection.

FIG. 29-6 shows up-regulated genes in mice harboring breast carcinoma. Samples were obtained from prefrontal cortex. The list of genes corresponds to those that are up-regulated at 72 hours and 192 hours post tumor injection.

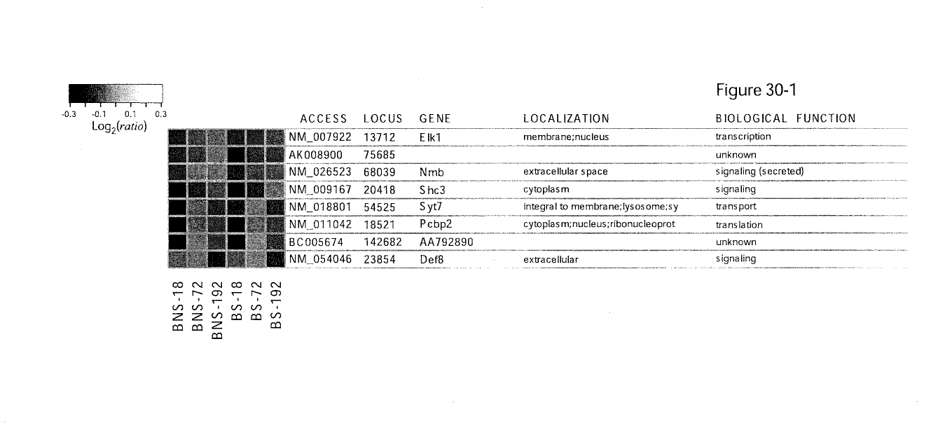

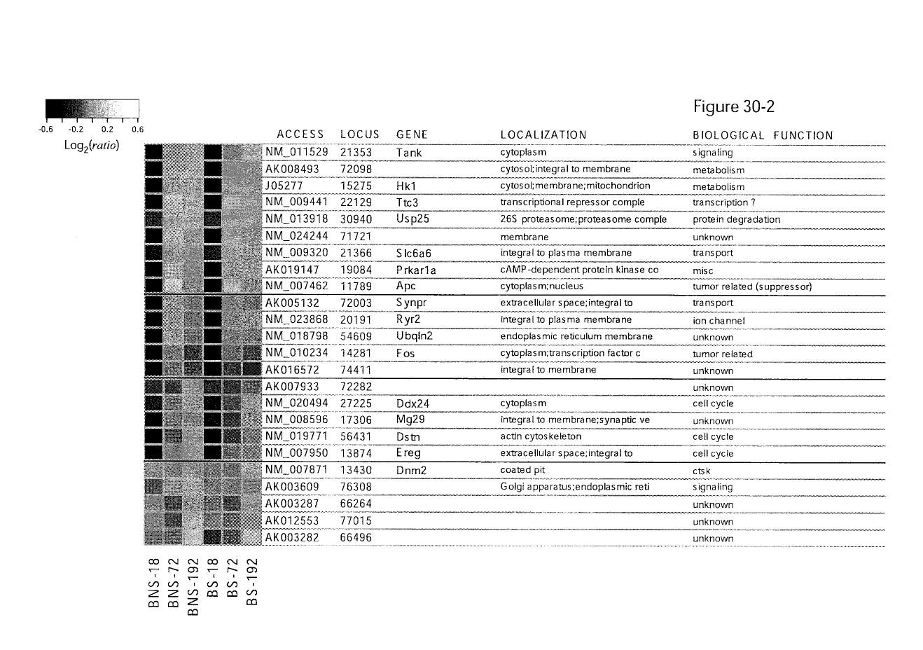

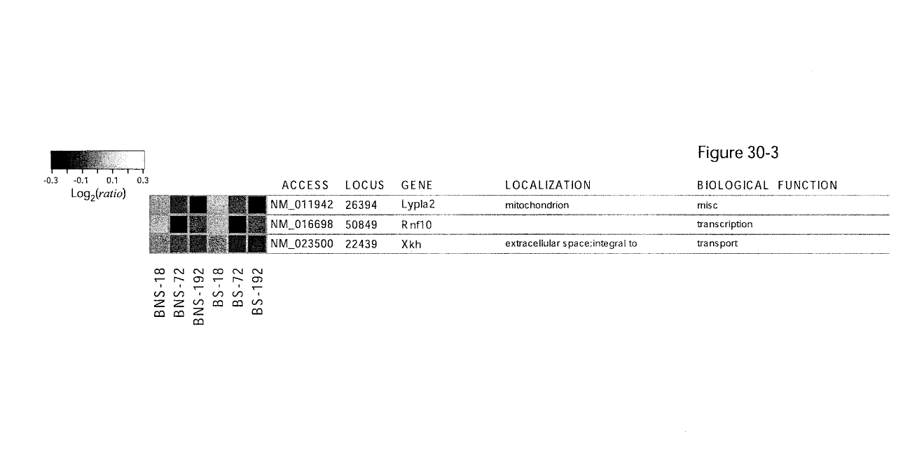

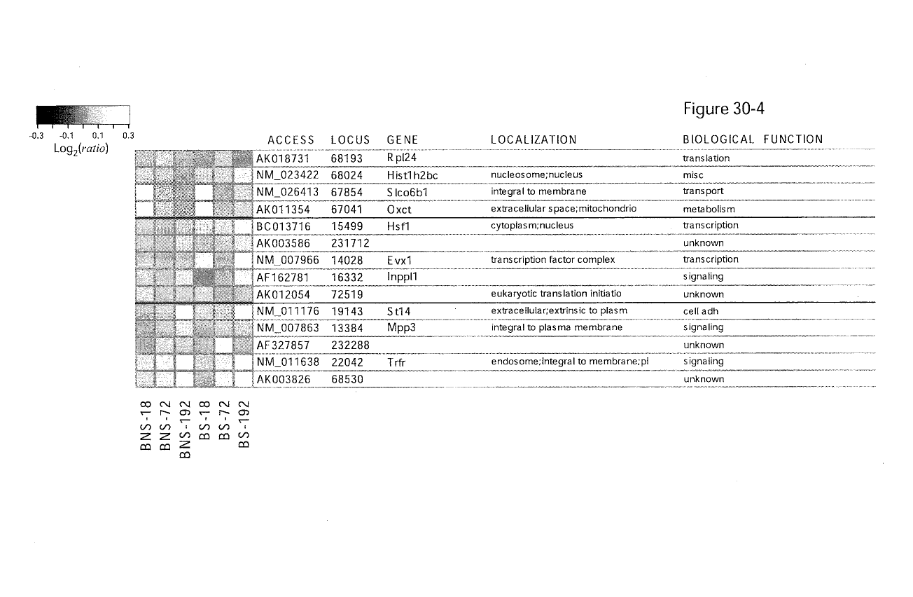

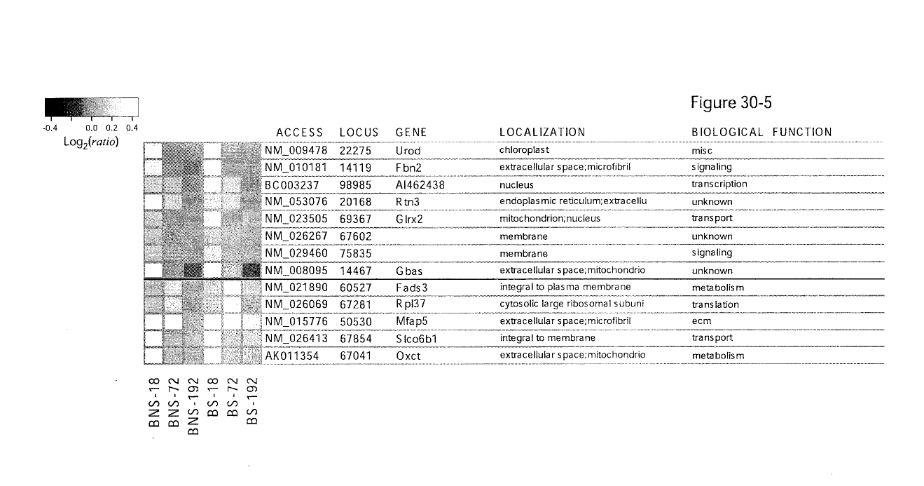

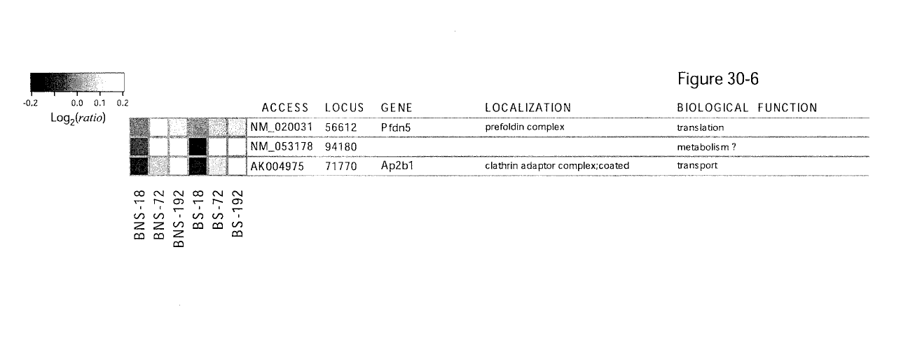

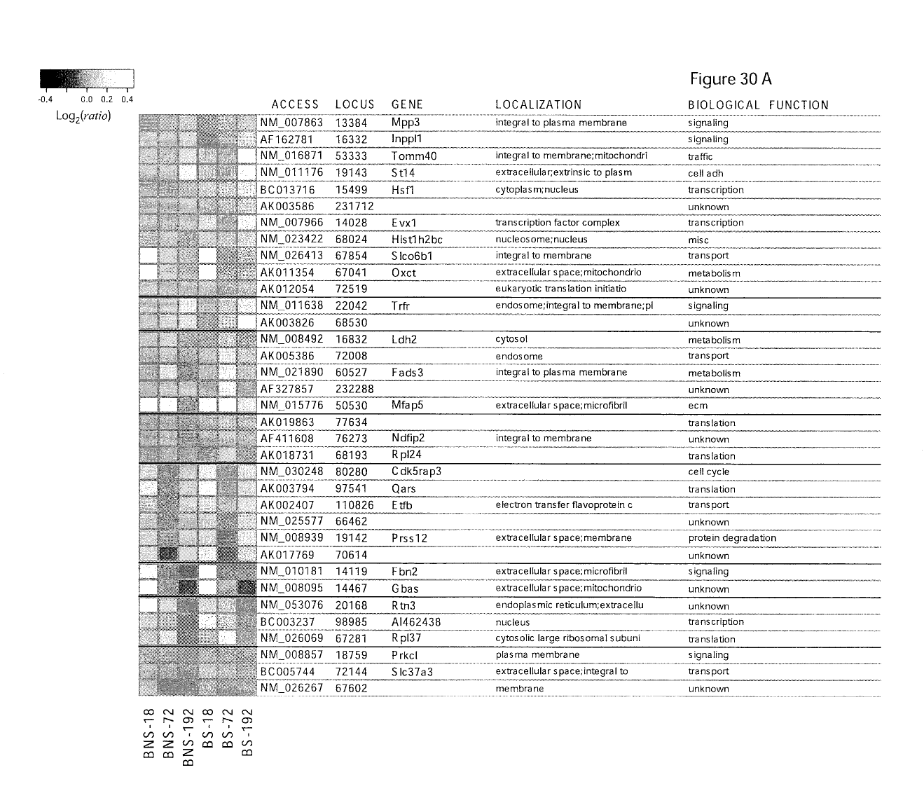

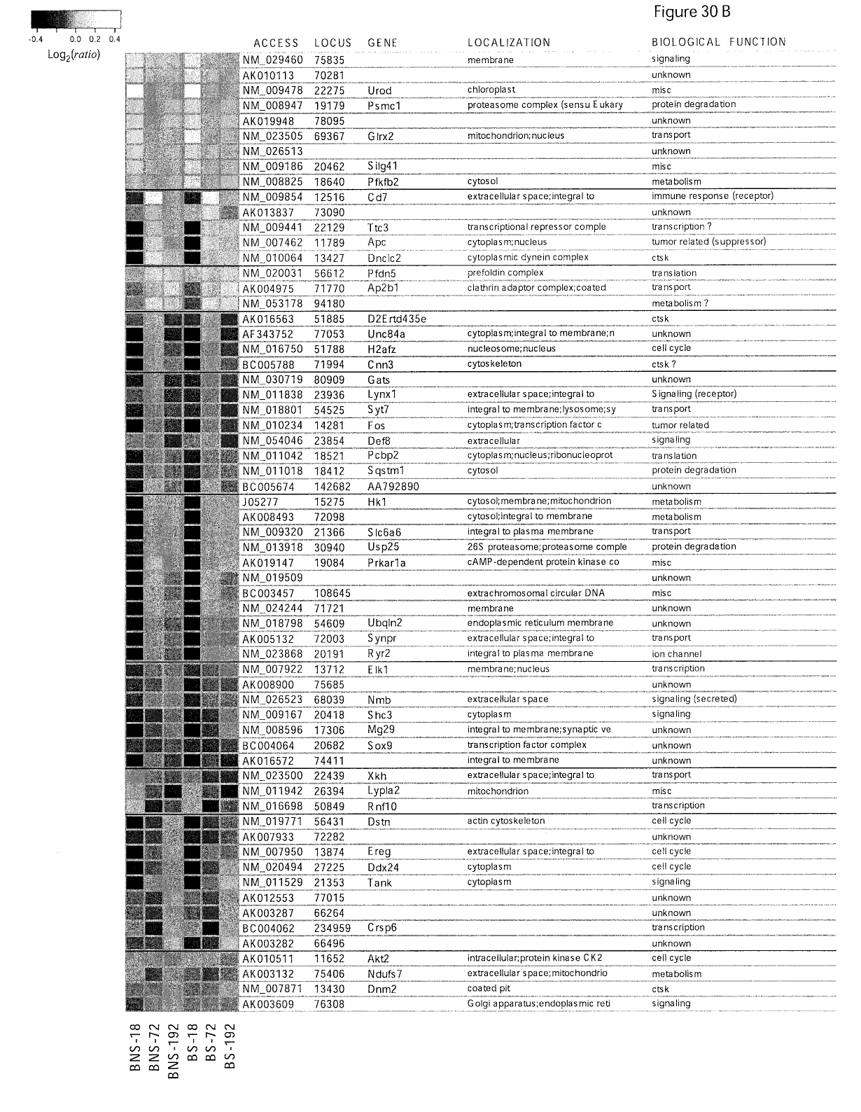

FIGS. 30A & 30B are tables showing genes differentially expressed genes in mice harboring colon carcinoma after performing a hierarchical cluster analysis. Samples were obtained from prefrontal cortex.

FIG. 30-1 shows down-regulated genes in mice harboring colon carcinoma. Samples were obtained from prefrontal cortex. The list of genes corresponds to those that are down-regulated at all the time points.

FIG. 30-2 shows down-regulated genes in mice harboring colon carcinoma. Samples were obtained from prefrontal cortex. The list of genes corresponds to those that are down-regulated at 18 hours, or at 18 hours and 72 hours post tumor injection.

FIG. 30-3 shows down-regulated genes in mice harboring colon carcinoma. Samples were obtained from prefrontal cortex. The list of genes corresponds to those that are down-regulated at 72 hours and 192 hours post tumor injection.

FIG. 30-4 shows up-regulated in mice harboring colon carcinoma. Samples were obtained from prefrontal cortex. The list of genes corresponds to those that are up-regulated at all the time points

FIG. 30-5 shows up regulated genes in mice harboring colon carcinoma. Samples were obtained from prefrontal cortex. The list of genes corresponds to those that are up regulated at 18 hours, or at 18 hours and 72 hours post tumor injection.

FIG. 30-6 shows up regulated genes in mice harboring colon carcinoma. Samples were obtained from prefrontal cortex. The list of genes corresponds to those that are up regulated at 72 hours and 192 hours post tumor injection.

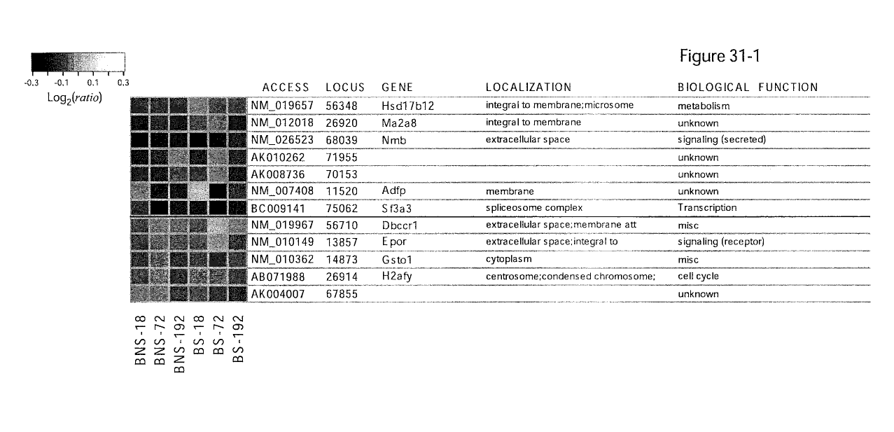

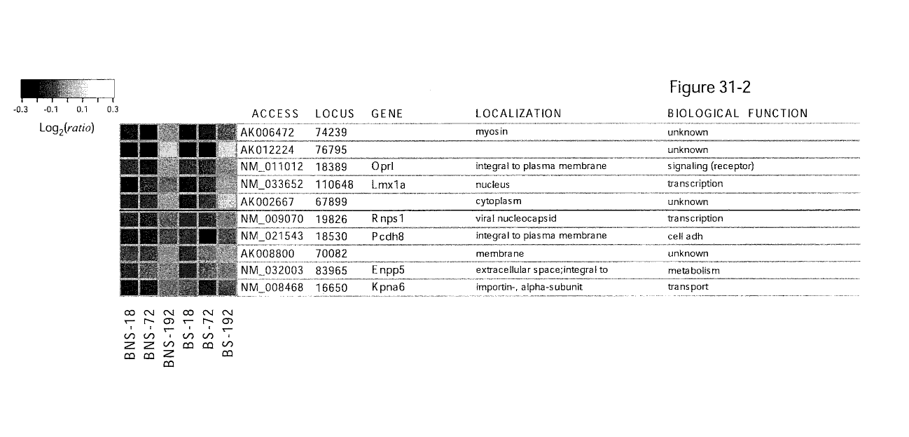

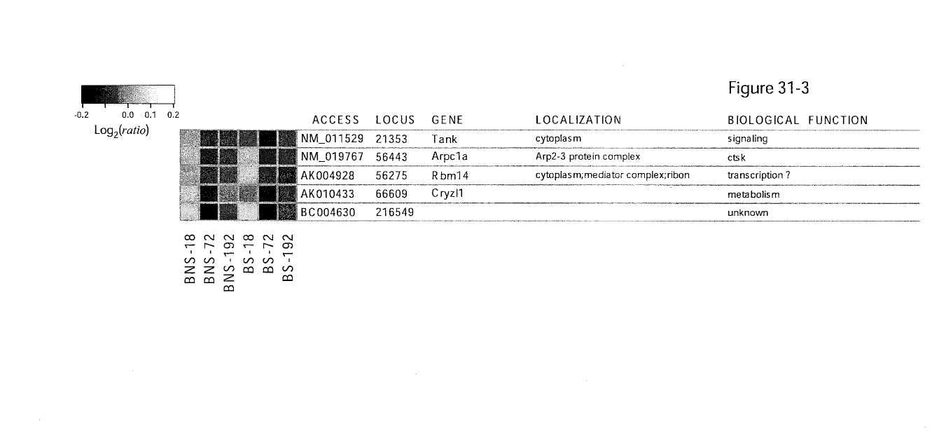

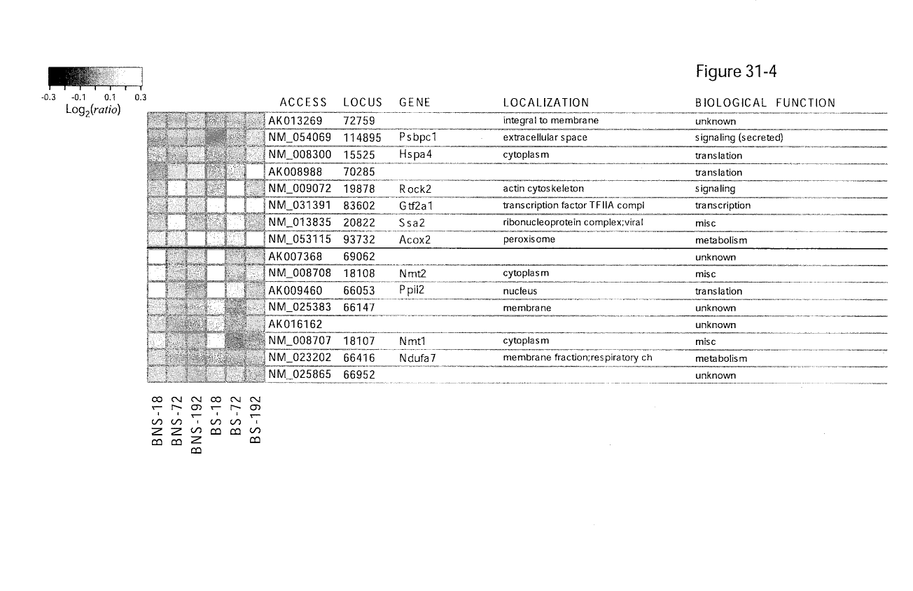

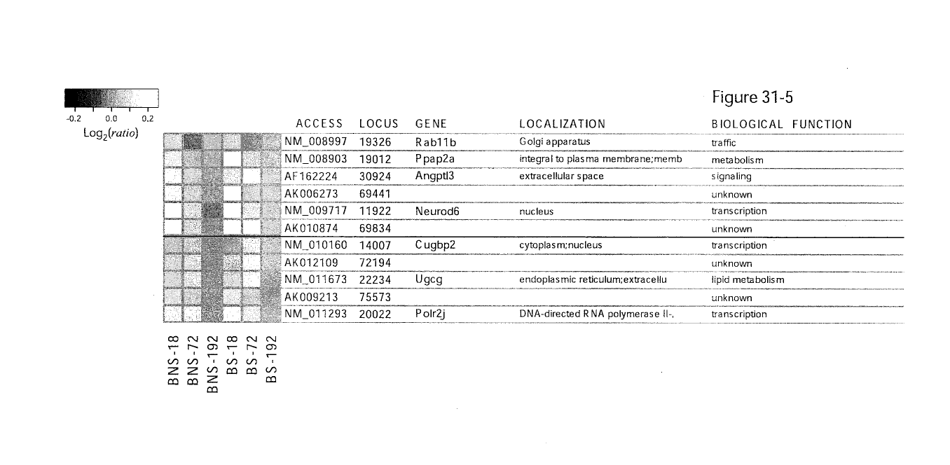

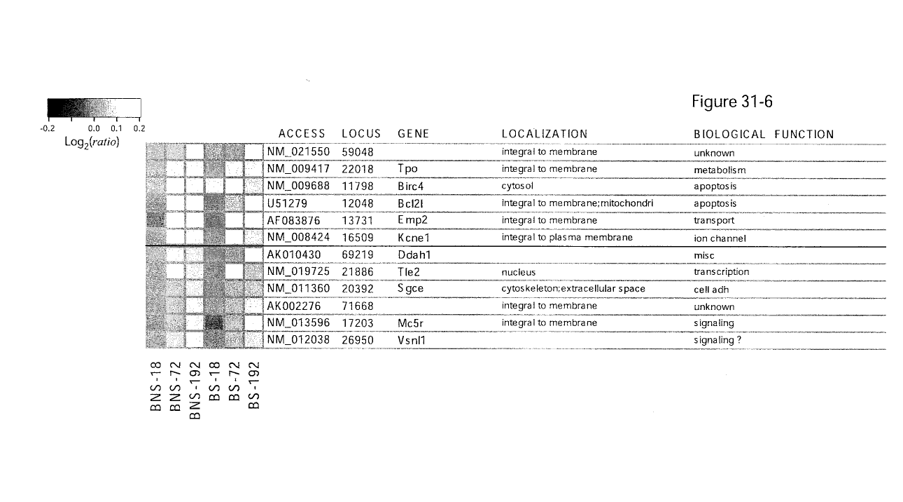

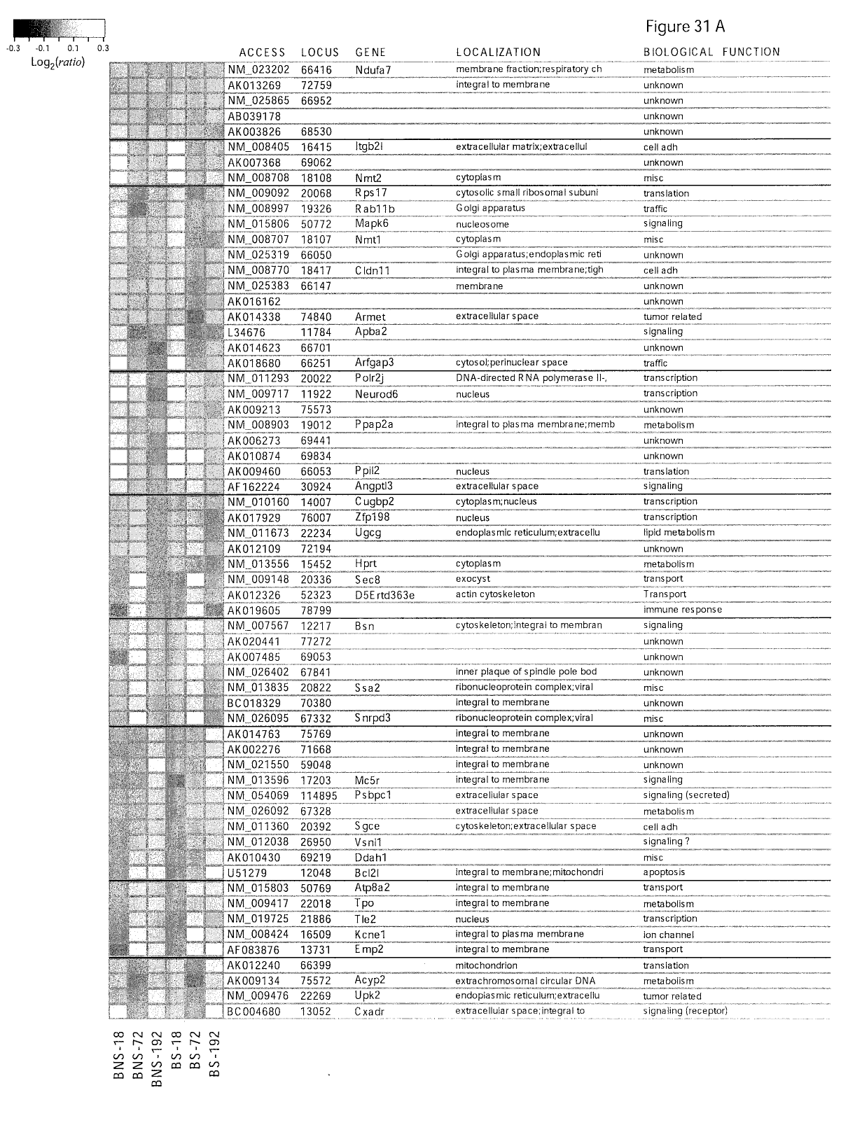

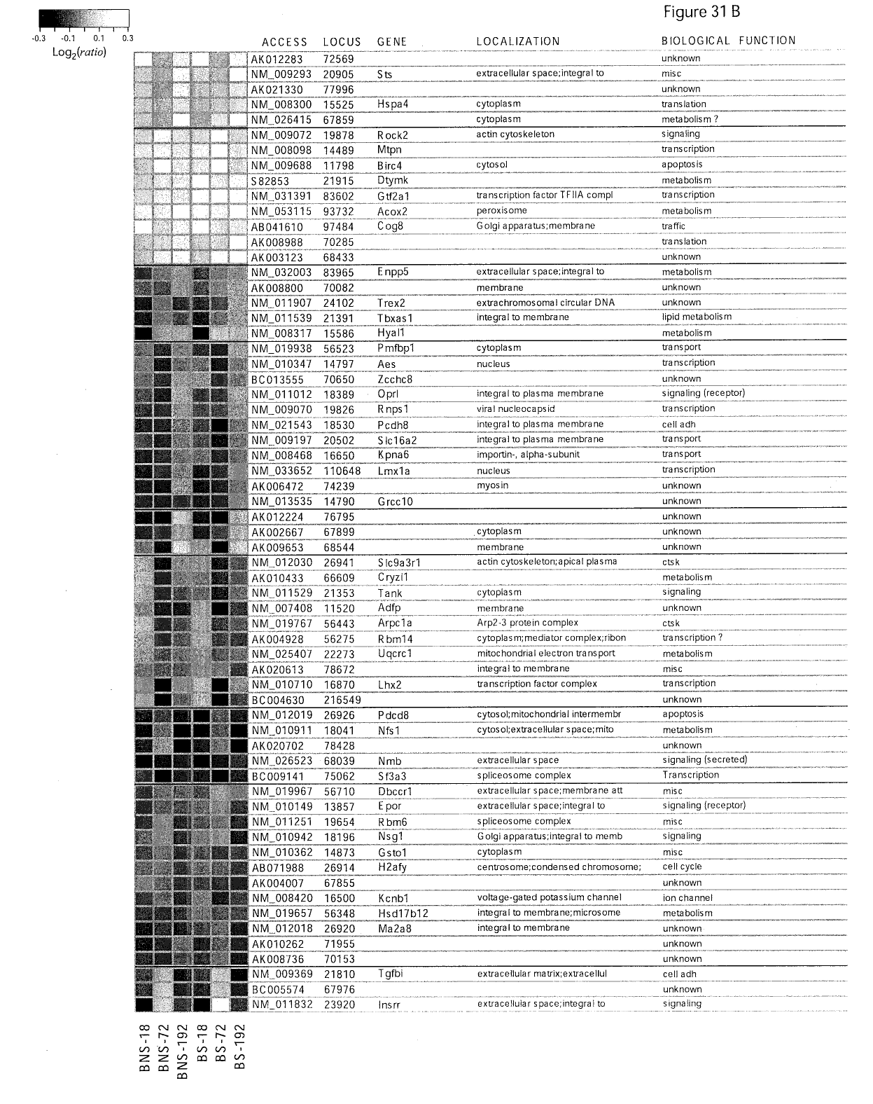

FIGS. 31A & 31B are tables showing differentially expressed genes in mice harboring lung carcinoma after hierarchical cluster analysis. Samples were obtained from prefrontal cortex.

FIG. 31-1 shows down regulated genes in mice harboring lung carcinoma. Samples were obtained from prefrontal cortex. The list of genes corresponds to those that are down regulated at all the time points.

FIG. 31-2 shows down regulated genes in mice harboring lung carcinoma. Samples were obtained from prefrontal cortex. The list of genes corresponds to those that are down regulated at 18 hours, or at 18 hours and 72 hours post tumor injection.

FIG. 31-3 shows down regulated genes in mice harboring lung carcinoma. Samples were obtained from prefrontal cortex. The list of genes corresponds to those that are down regulated at 72 hours and 192 hours post tumor injection.

FIG. 31-4 shows up regulated genes in mice harboring lung carcinoma. Samples were obtained from prefrontal cortex. The list of genes corresponds to those that are up regulated at all the time points.

FIG. 31-5 shows up regulated genes in mice harboring lung carcinoma. Samples were obtained from prefrontal cortex. The list of genes corresponds to those that are up regulated at 18 hours, or at 18 hours and 72 hours post tumor injection.

FIG. 31-6 shows up regulated genes in mice harboring lung carcinoma. Samples were obtained from prefrontal cortex. The list of genes corresponds to those that are up regulated at 72 hours and 192 hours post tumor injection.

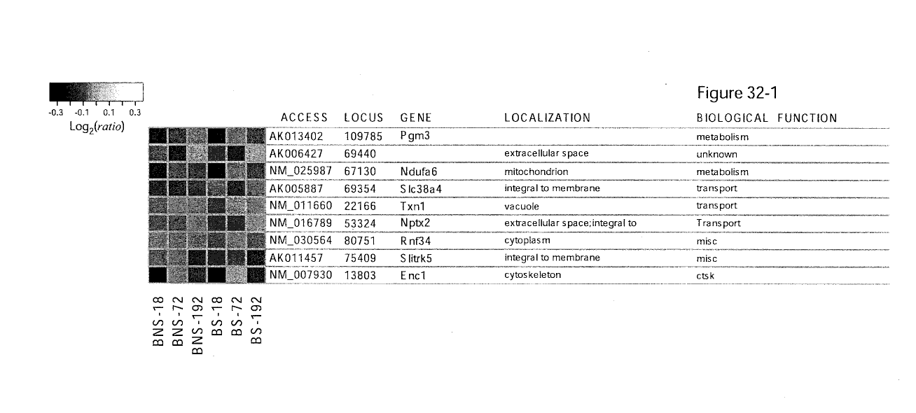

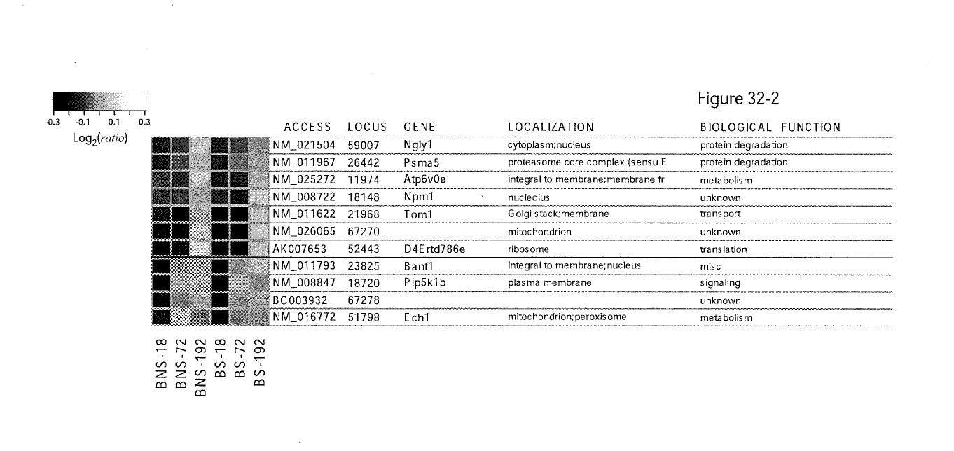

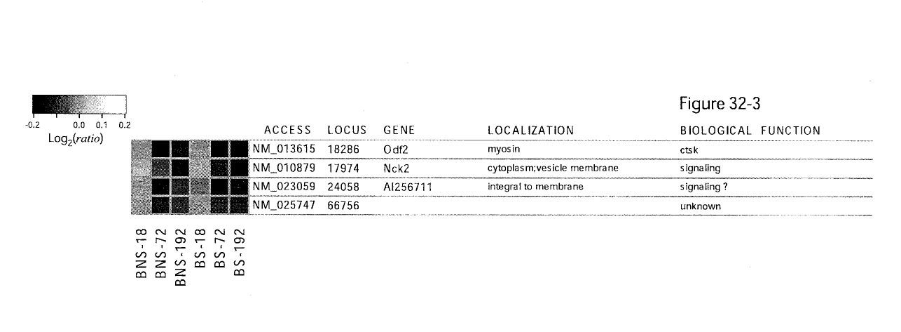

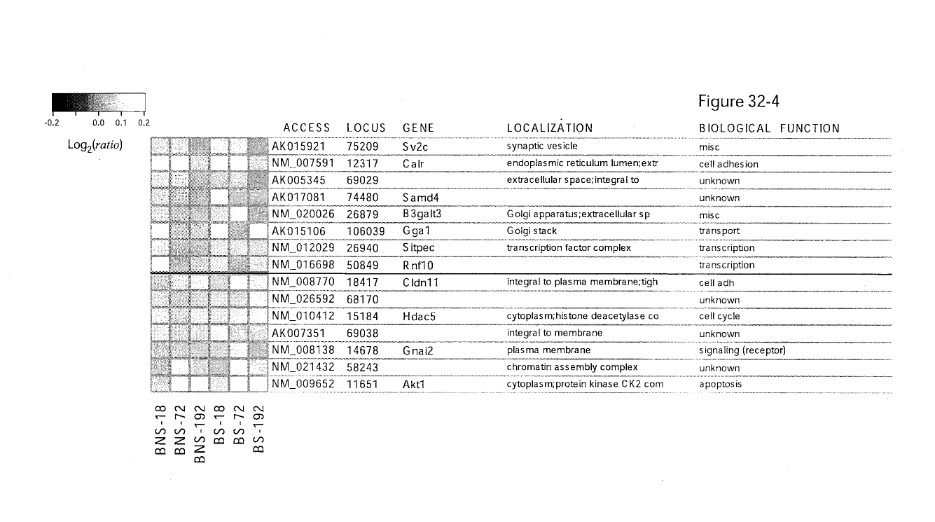

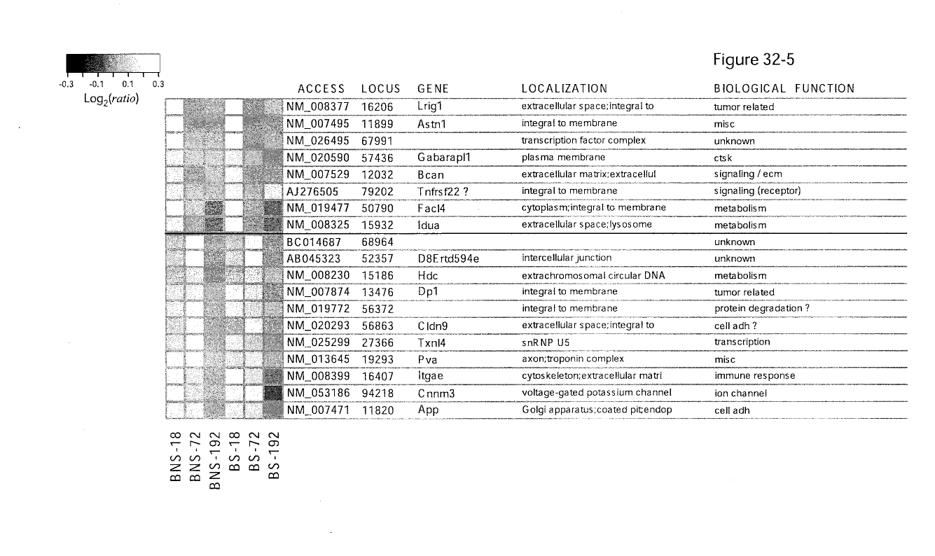

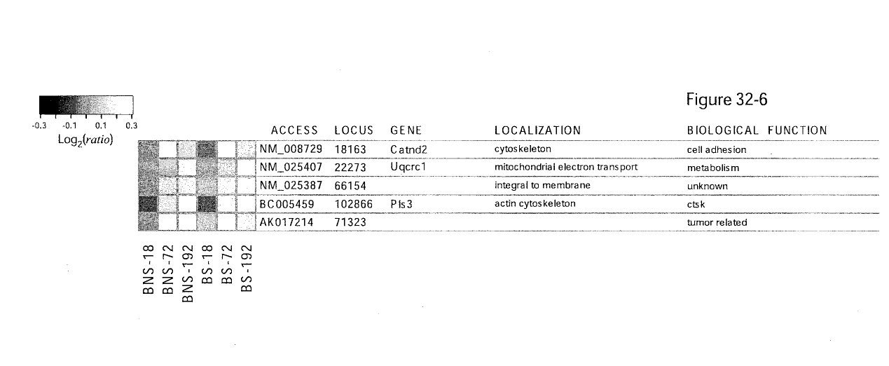

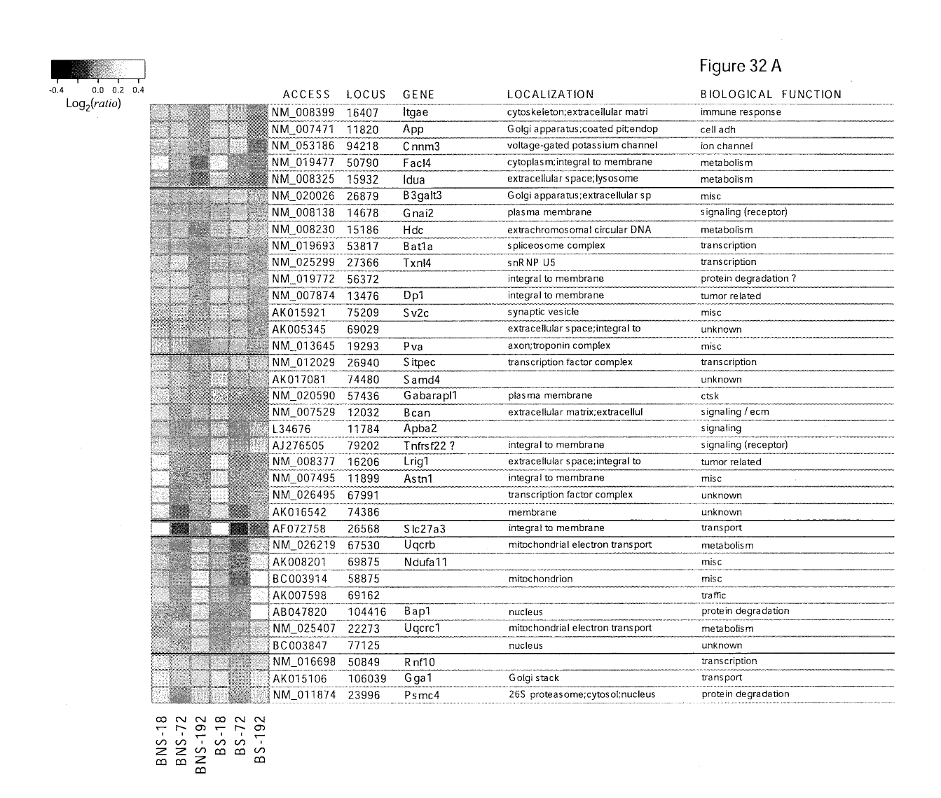

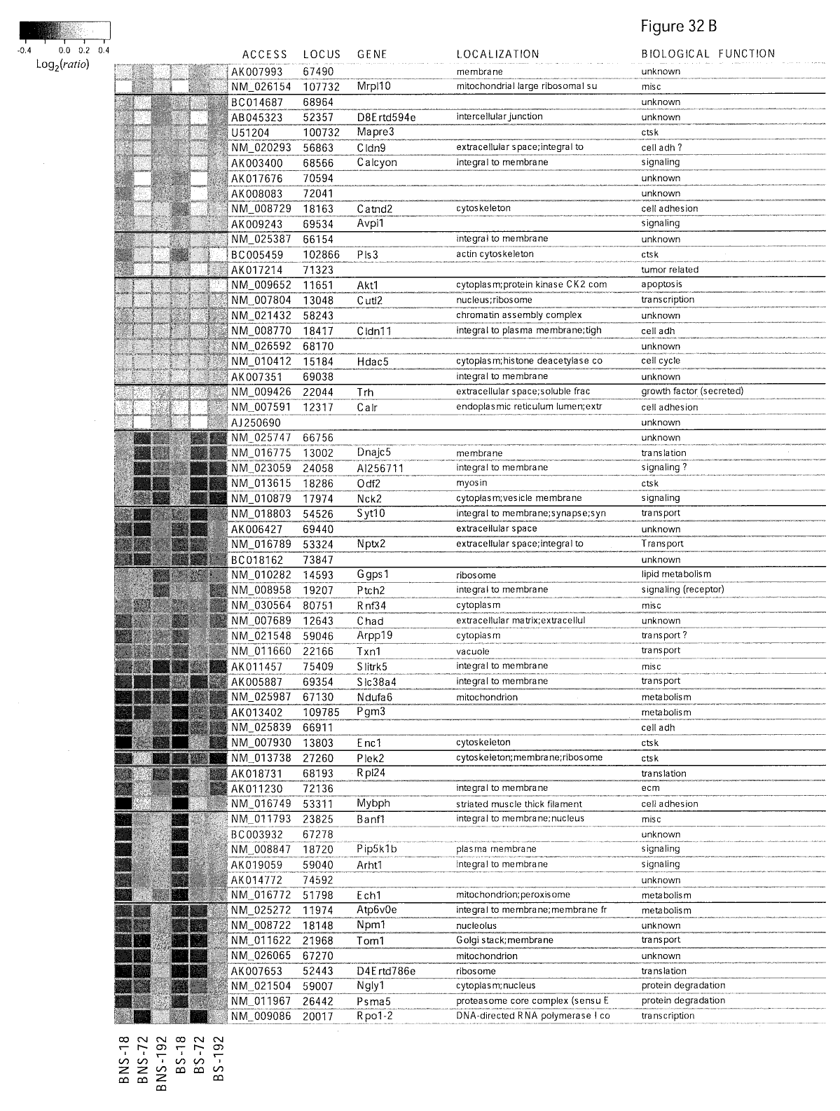

FIGS. 32A & 32B show differentially expressed genes in mice harboring breast carcinoma. Samples were obtained from hypothalamus.

FIG. 32-1 shows down regulated genes in mice harboring breast carcinoma. Samples were obtained from hypothalamus. The list of genes corresponds to those that are down regulated at all the time points.

FIG. 32-2 shows down regulated genes in mice harboring breast carcinoma. Samples were obtained from hypothalamus. The list of genes corresponds to those that are down regulated at 18 hours, or at 18 hours and 72 hours post tumor injection.

FIG. 32-3 shows down regulated genes in mice harboring breast carcinoma. Samples were obtained from hypothalamus. The list of genes corresponds to those that are down regulated at 72 hours and 192 hours post tumor injection.

FIG. 32-4 shows up regulated genes in mice harboring breast carcinoma. Samples were obtained from hypothalamus. The list of genes corresponds to those that are up regulated at all the time points.

FIG. 32-5 shows up regulated genes in mice harboring breast carcinoma. Samples were obtained from hypothalamus. The list of genes corresponds to those that are up regulated at 18 hours, or at 18 hours and 72 hours post tumor injection.

FIG. 32-6 shows up regulated genes in mice harboring breast carcinoma. Samples were obtained from hypothalamus. The list of genes corresponds to those that are up regulated at 72 hours and 192 hours post tumor injection.

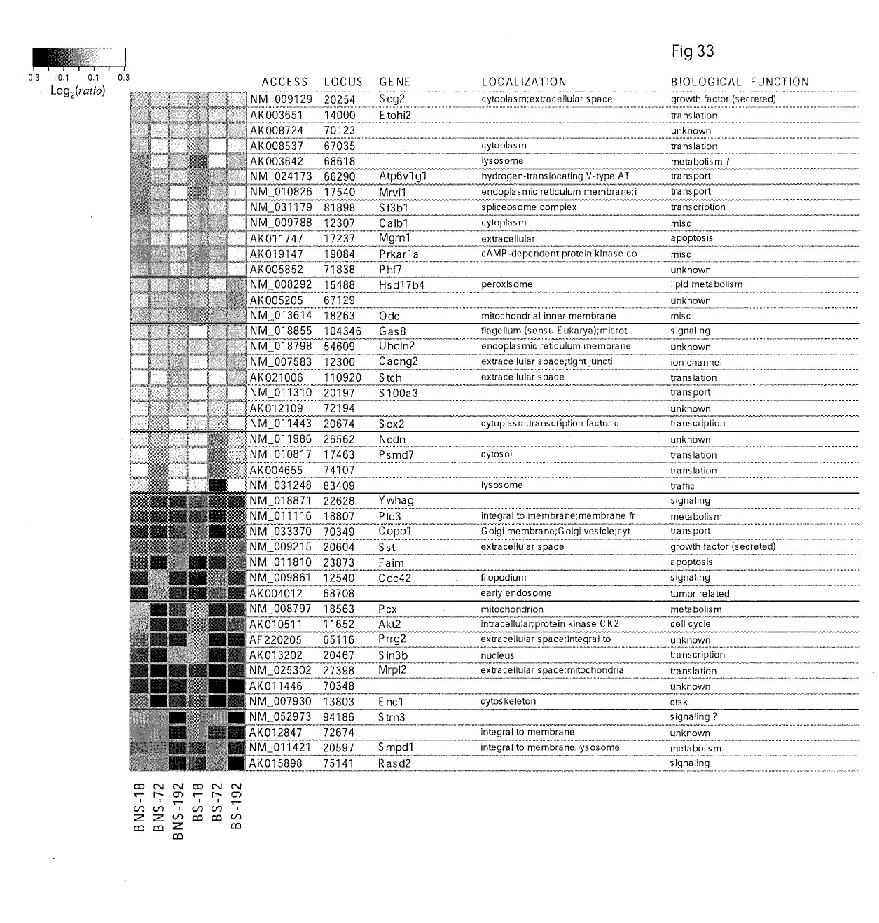

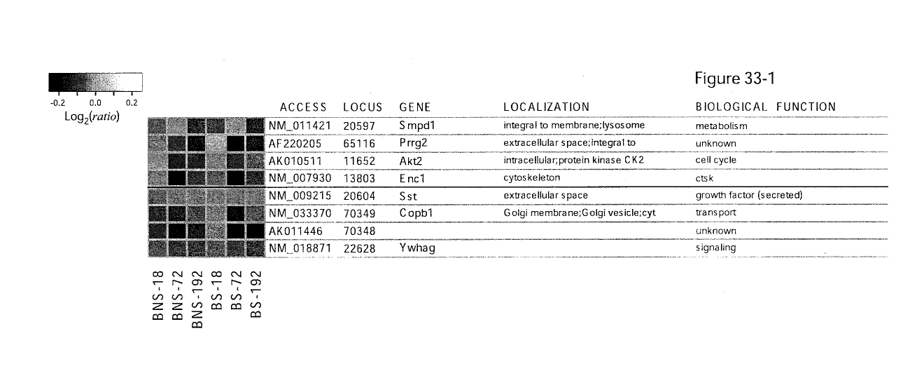

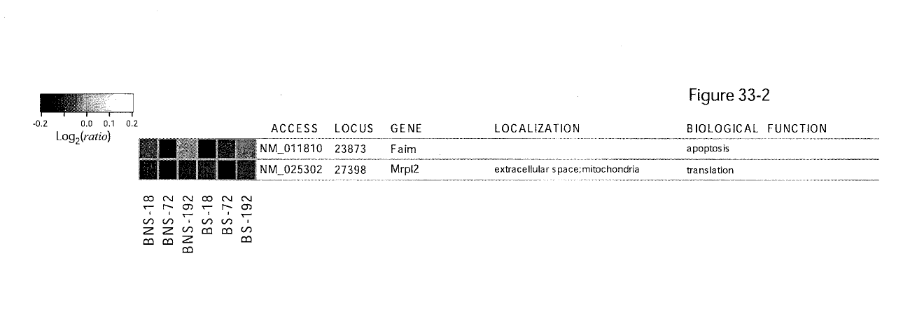

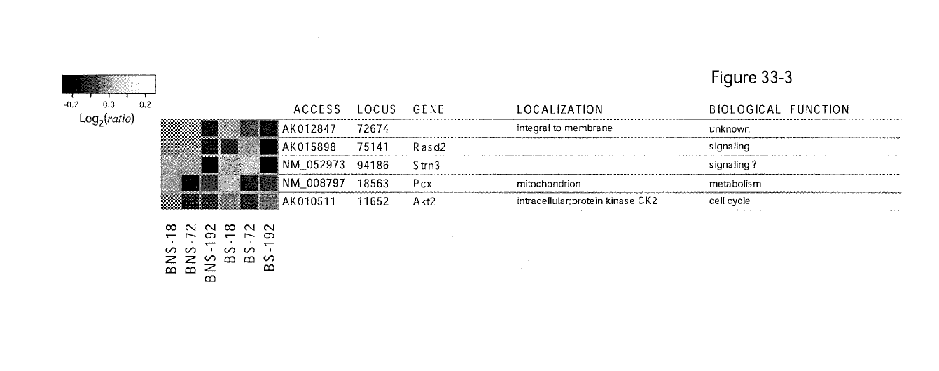

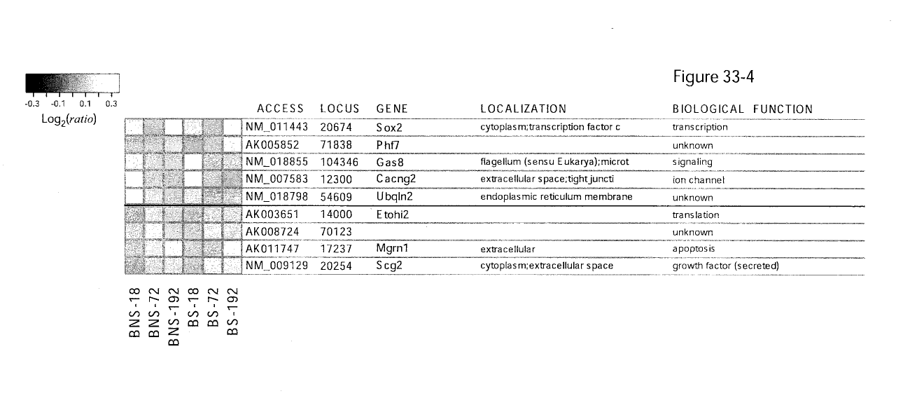

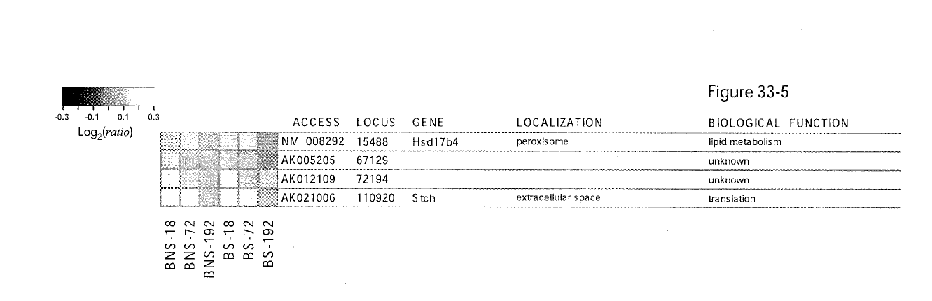

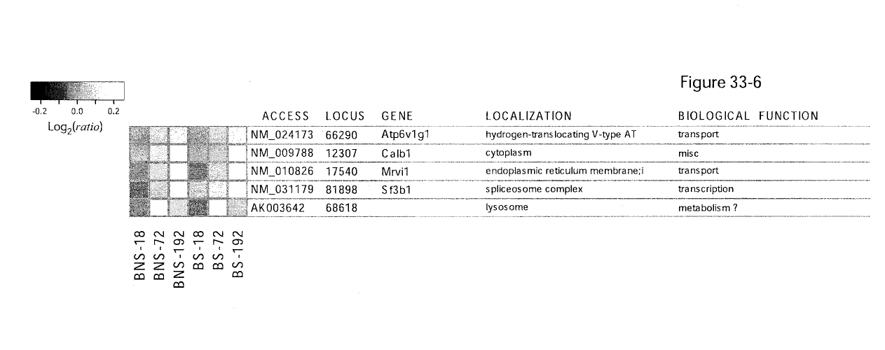

FIG. 33 shows differentially expressed genes in mice harboring colon carcinoma. Samples were obtained from hypothalamus.

FIG. 33-1 shows down regulated genes in mice harboring colon carcinoma. Samples were obtained from hypothalamus. The list of genes corresponds to those that are down regulated at all the time points.

FIG. 33-2 shows down regulated genes in mice harboring colon carcinoma. Samples were obtained from hypothalamus. The list of genes corresponds to those that are down regulated at 18 hours, or at 18 hours and 72 hours post tumor injection.

FIG. 33-3 shows down regulated genes in mice harboring colon carcinoma. Samples were obtained from hypothalamus. The list of genes corresponds to those that are down regulated at 72 hours and 192 hours post tumor injection.

FIG. 33-4 shows up regulated genes in mice harboring colon carcinoma. Samples were obtained from hypothalamus. The list of genes corresponds to those that are up regulated at all the time points.

FIG. 33-5 shows up regulated genes in mice harboring colon carcinoma. Samples were obtained from hypothalamus. The list of genes corresponds to those that are up regulated at 18 hours, or at 18 hours and 72 hours post tumor injection.

FIG. 33-6 shows up regulated genes in mice harboring colon carcinoma. Samples were obtained from hypothalamus. The list of genes corresponds to those that are up regulated at 72 hours and 192 hours post tumor injection.

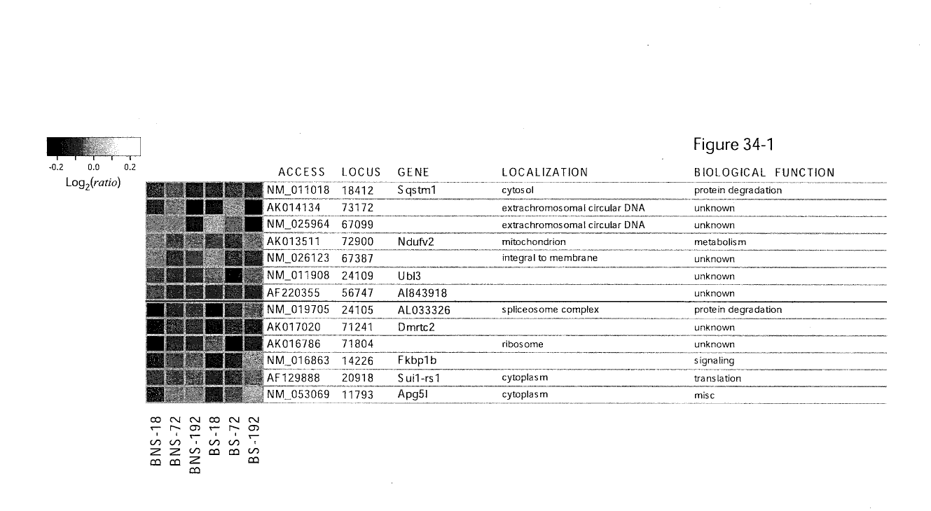

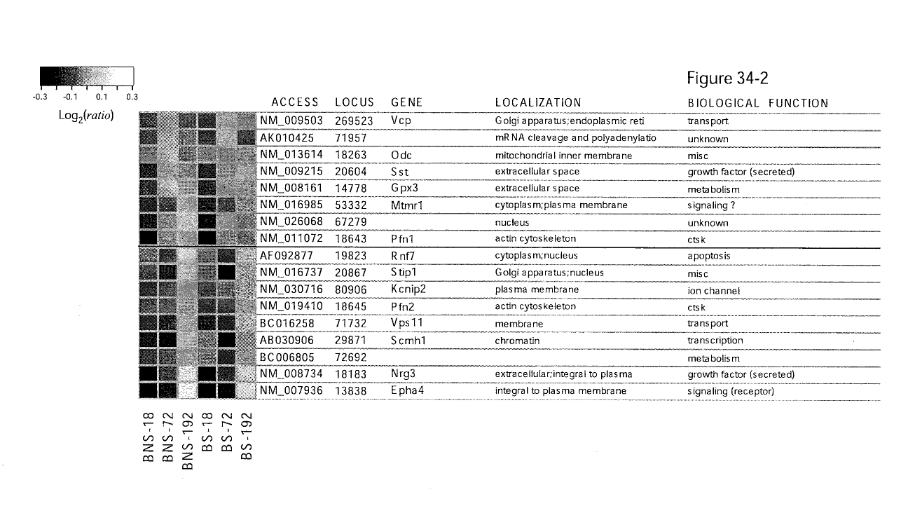

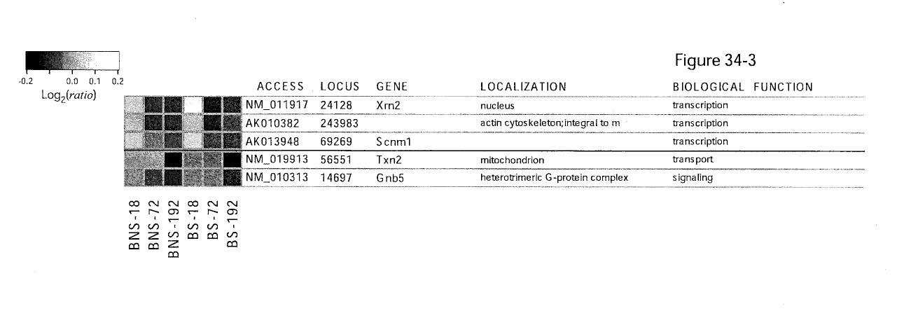

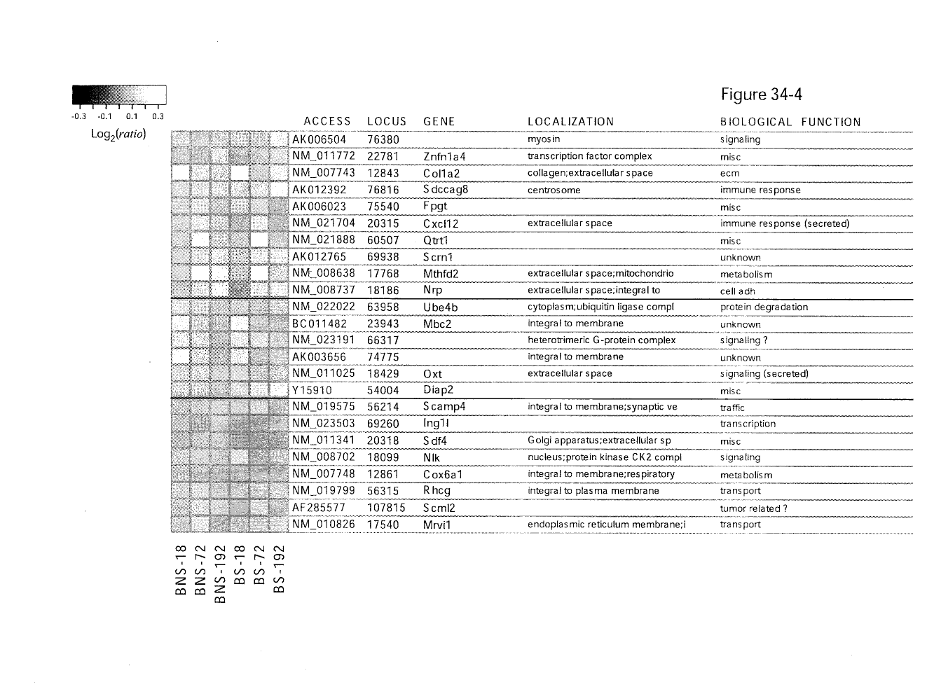

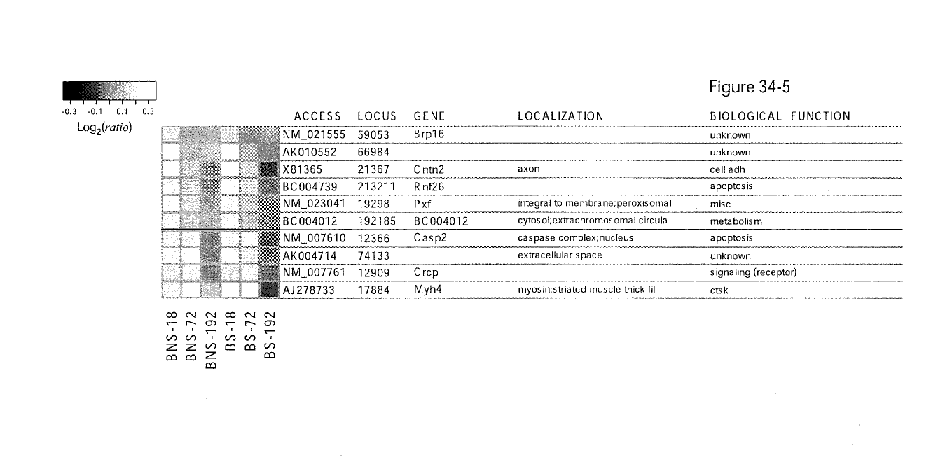

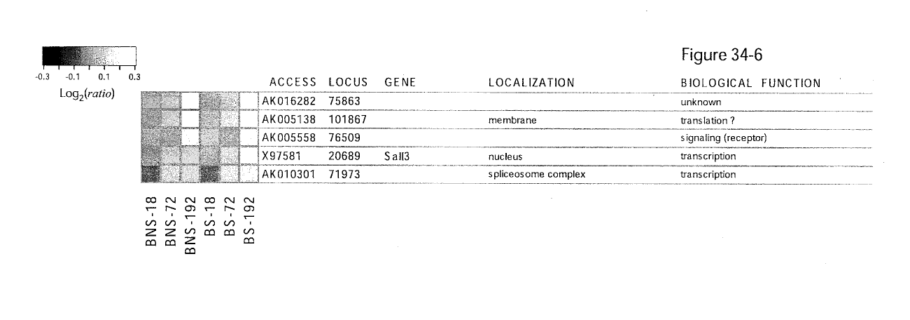

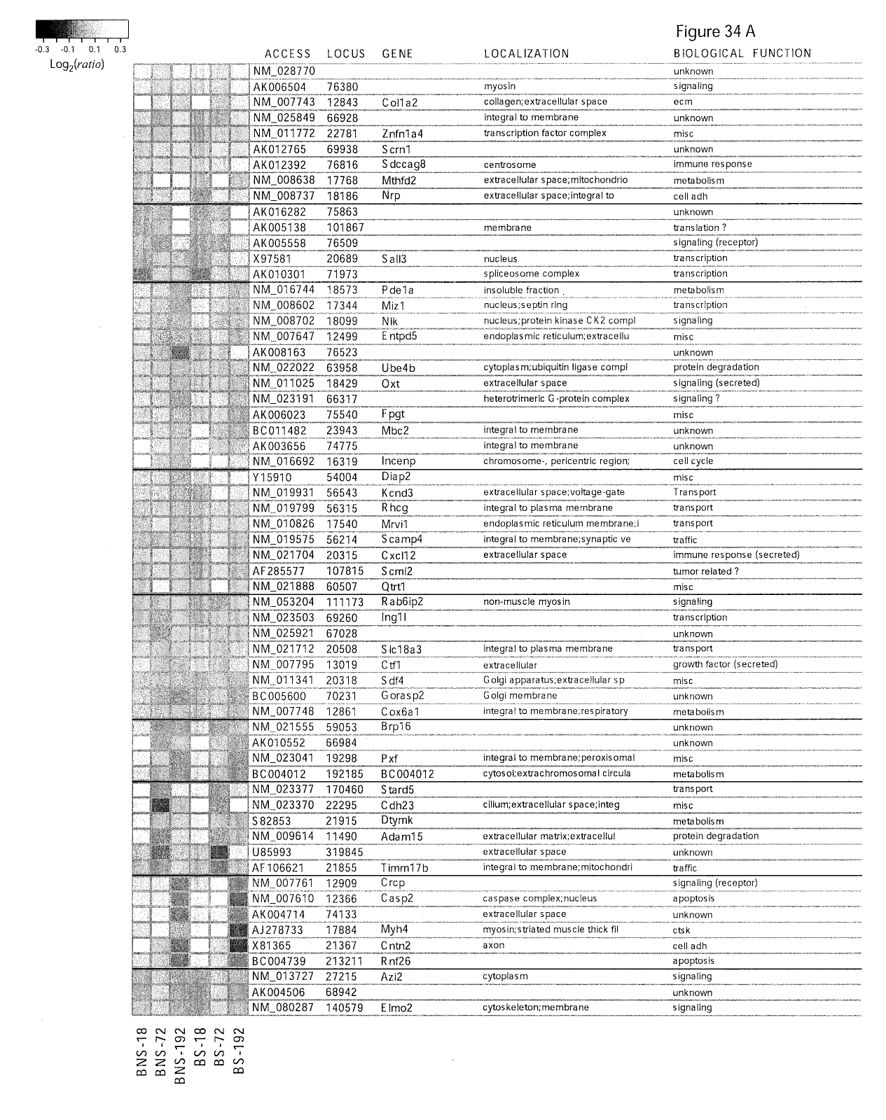

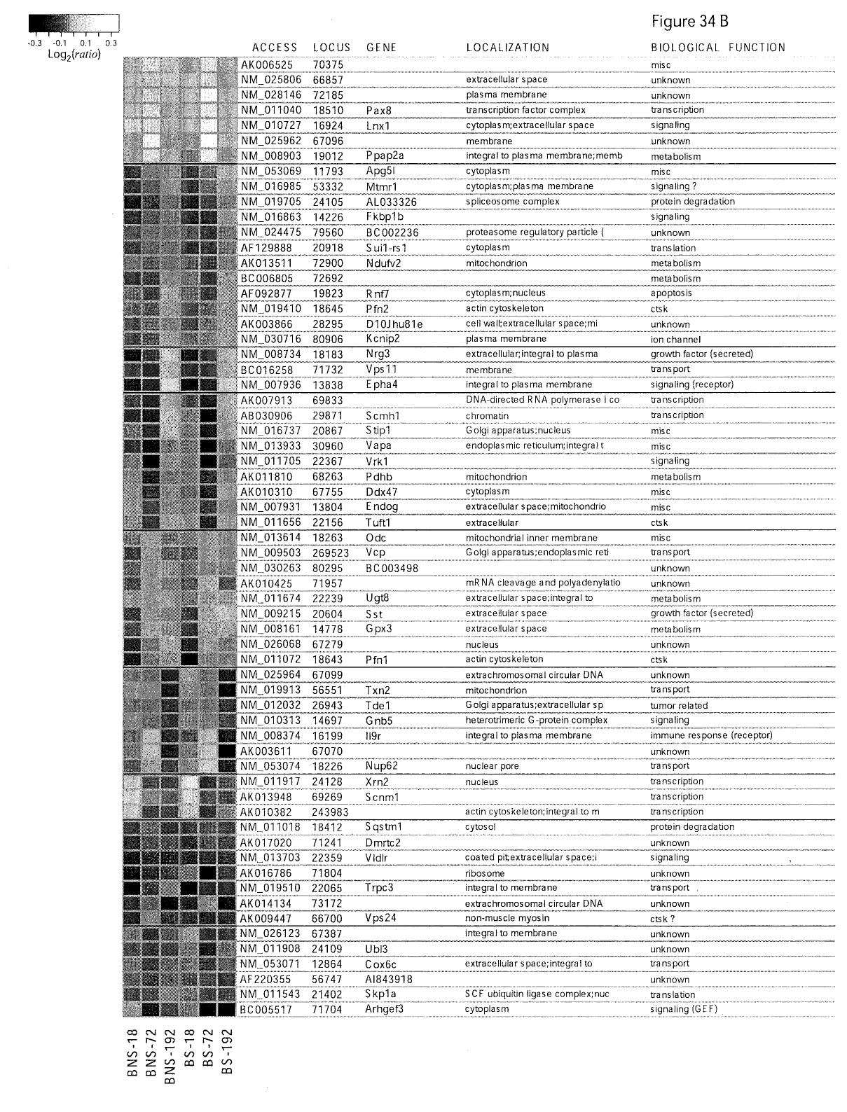

FIGS. 34A & 34B show differentially expressed genes in mice harboring lung carcinoma. Samples were obtained from hypothalamus.

FIG. 34-1 shows down regulated genes in mice harboring lung carcinoma. Samples were obtained from hypothalamus. The list of genes corresponds to those that are down regulated at all the time points.

FIG. 34-2 shows down regulated genes in mice harboring lung carcinoma. Samples were obtained from hypothalamus. The list of genes corresponds to those that are down regulated at 18 hours, or at 18 hours and 72 hours post tumor injection.

FIG. 34-3 shows down regulated genes in mice harboring lung carcinoma. Samples were obtained from hypothalamus. The list of genes corresponds to those that are down regulated at 72 hours and 192 hours post tumor injection.

FIG. 34-4 shows up regulated genes in mice harboring lung carcinoma. Samples were obtained from hypothalamus. The list of genes corresponds to those that are up regulated at all the time points.

FIG. 34-5 shows up regulated genes in mice harboring lung carcinoma. Samples were obtained from hypothalamus. The list of genes corresponds to those that are up regulated at 18 hours, or at 18 hours and 72 hours post tumor injection.

FIG. 34-6 shows up regulated genes in mice harboring lung carcinoma. Samples were obtained from hypothalamus. The list of genes corresponds to those that are up regulated at 72 hours and 192 hours post tumor injection.

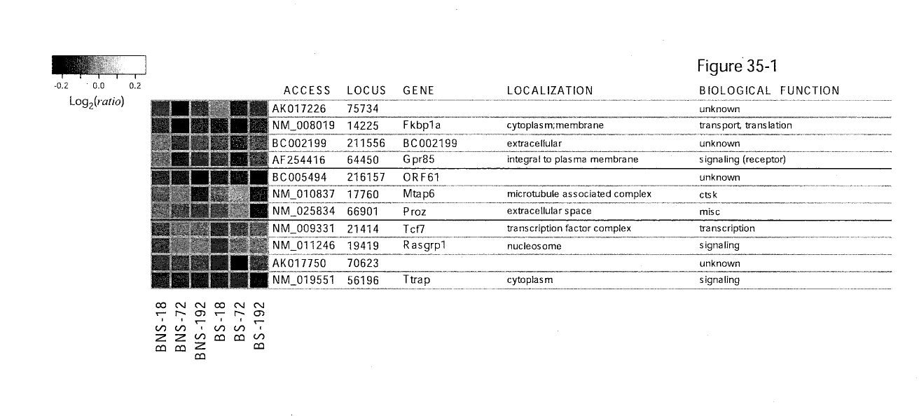

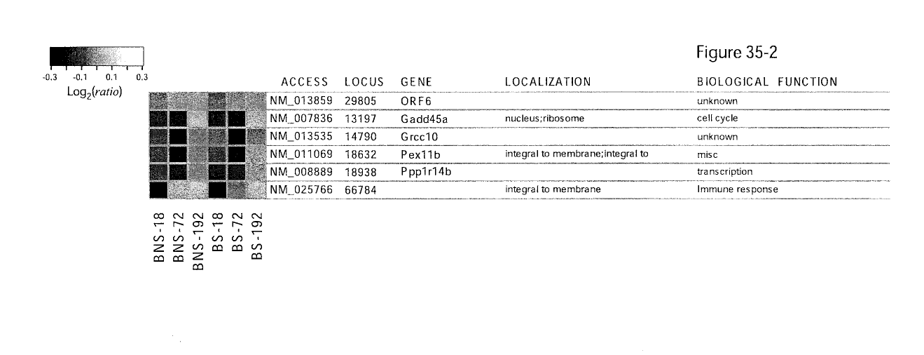

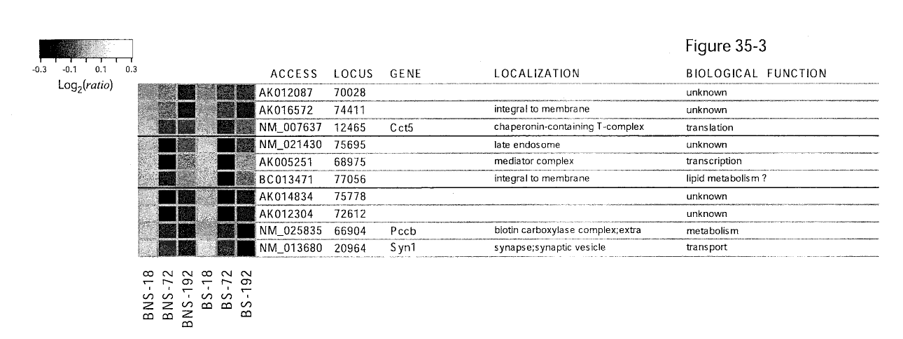

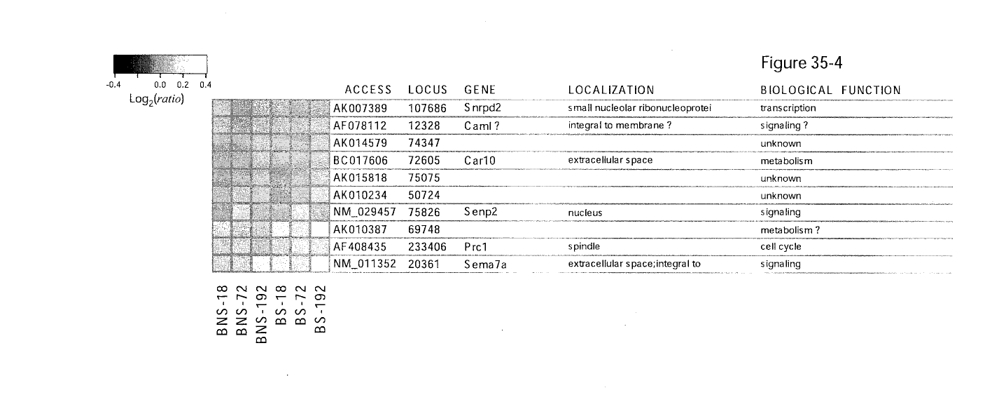

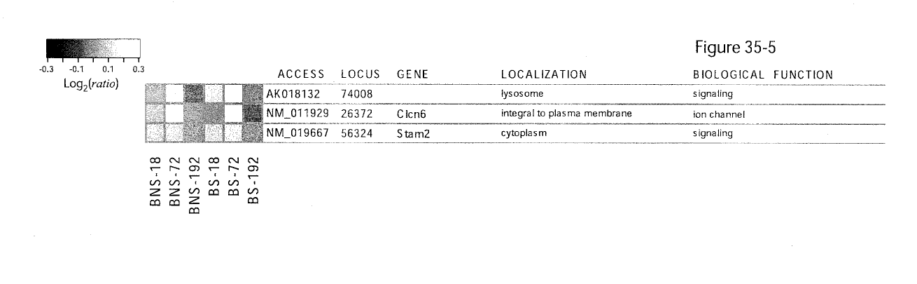

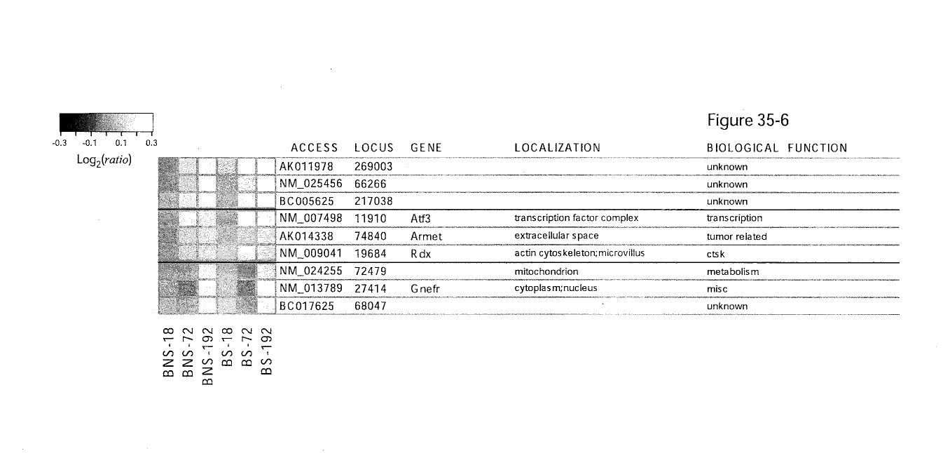

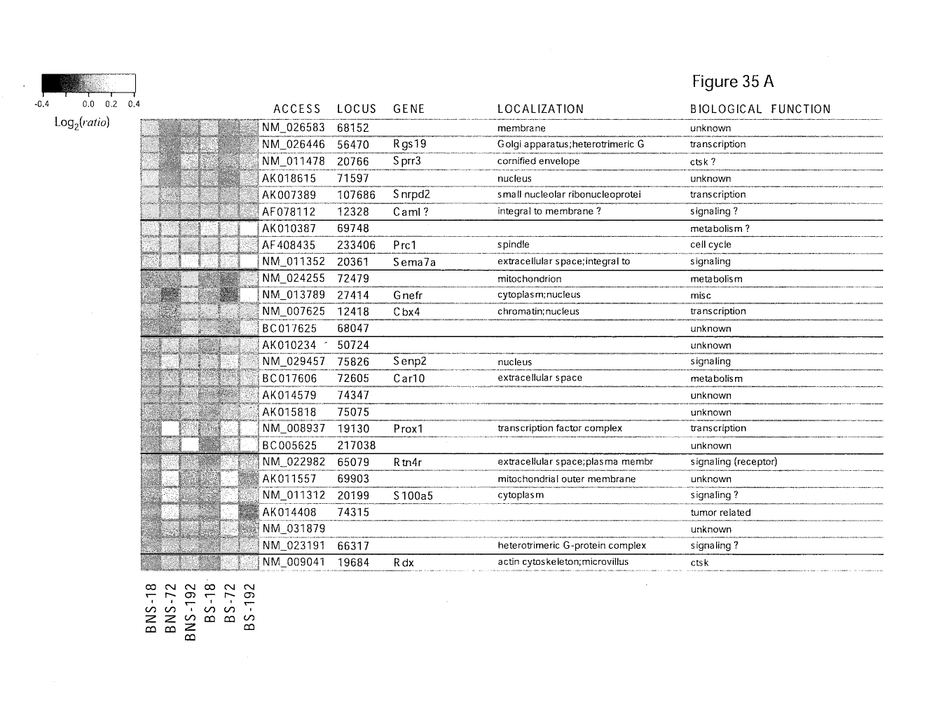

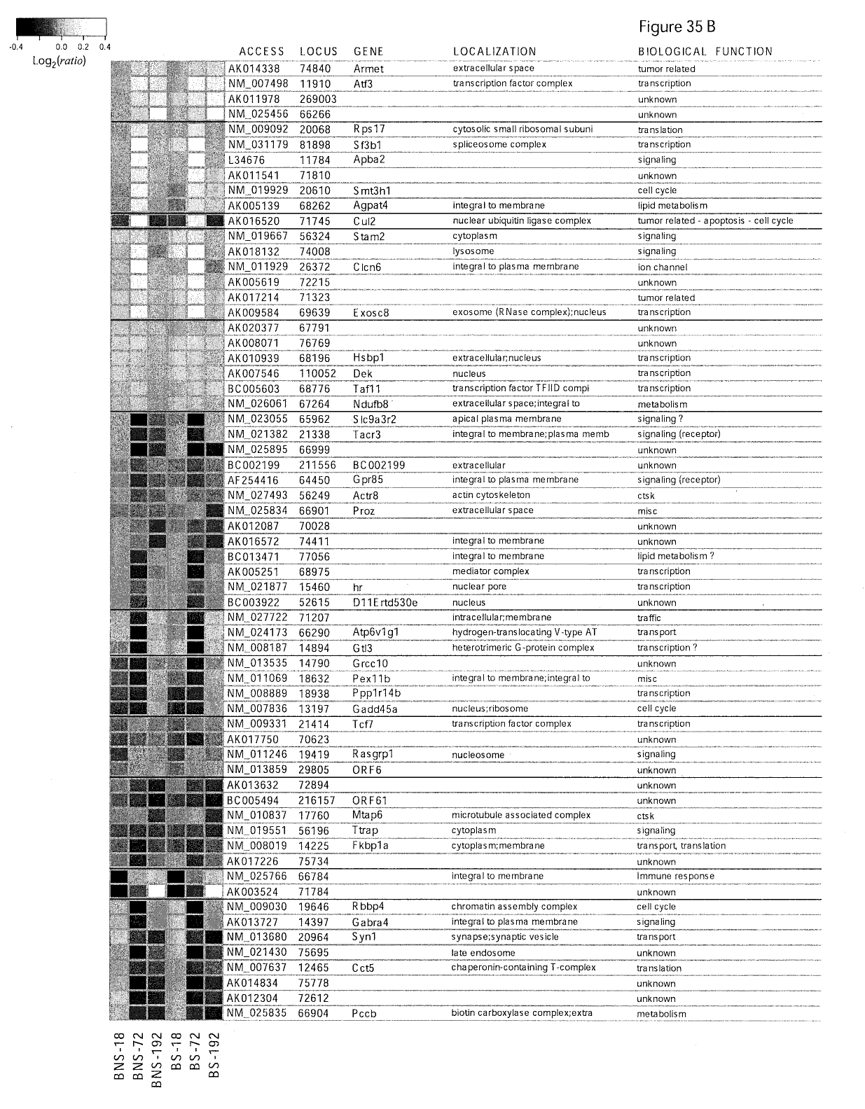

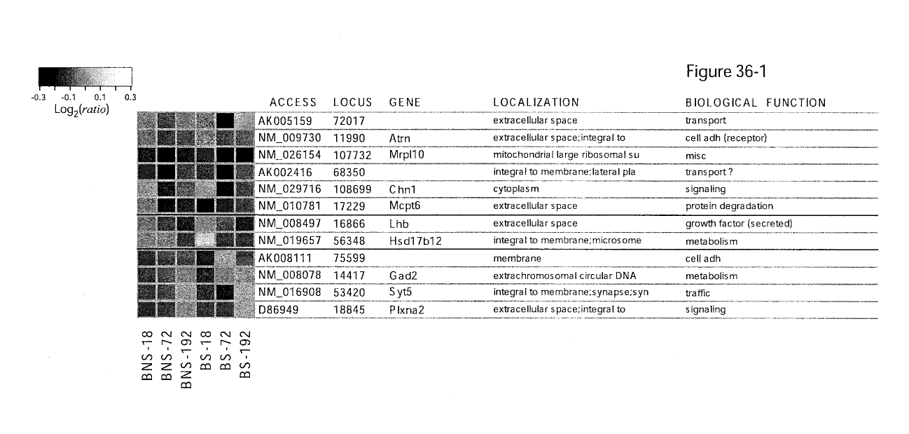

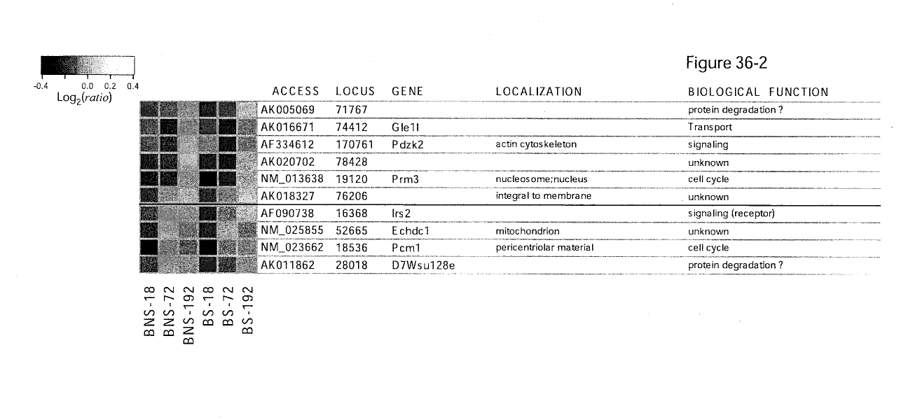

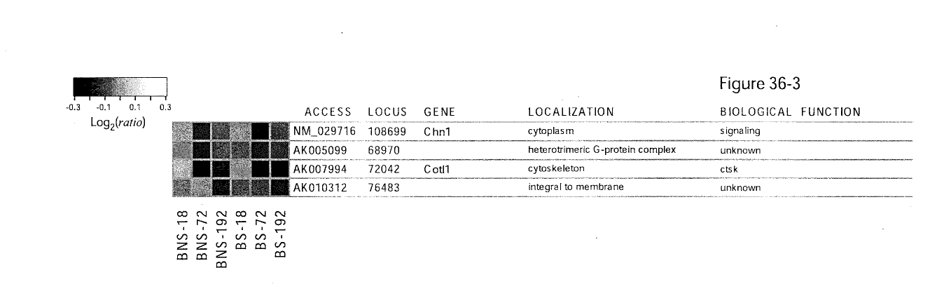

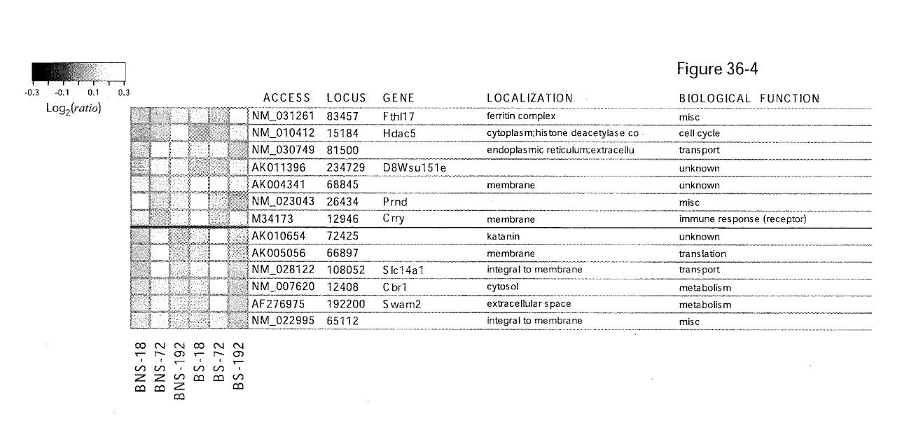

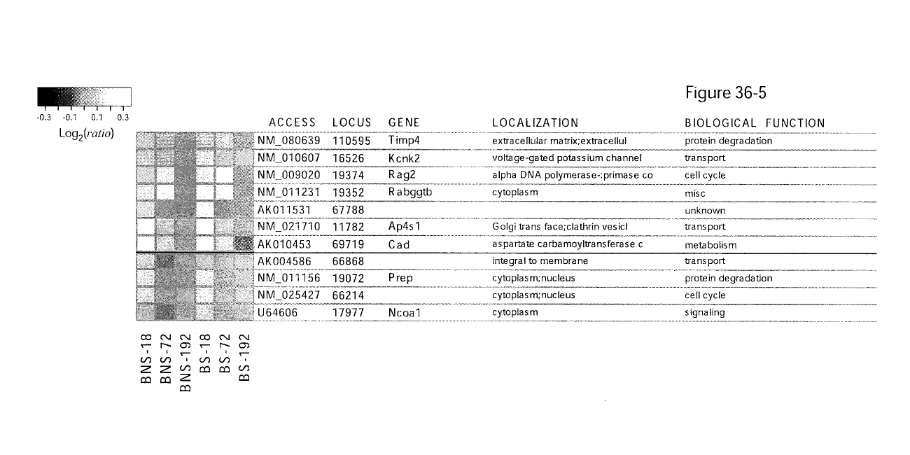

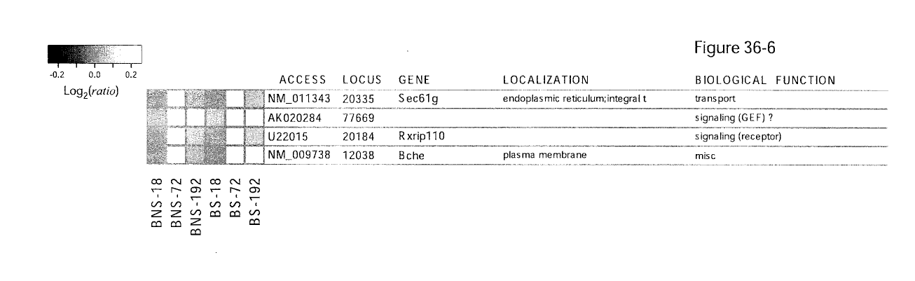

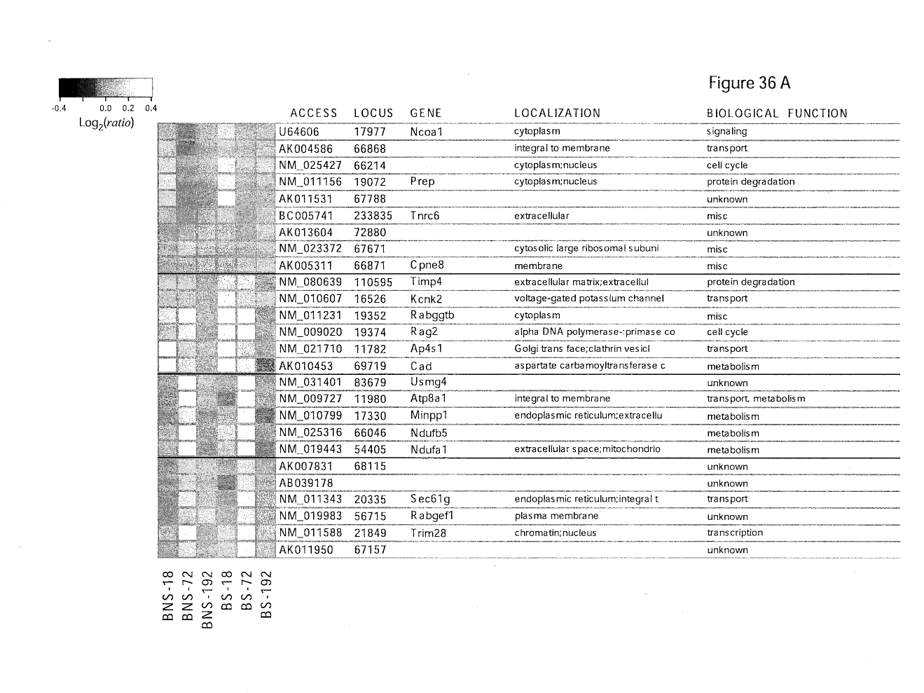

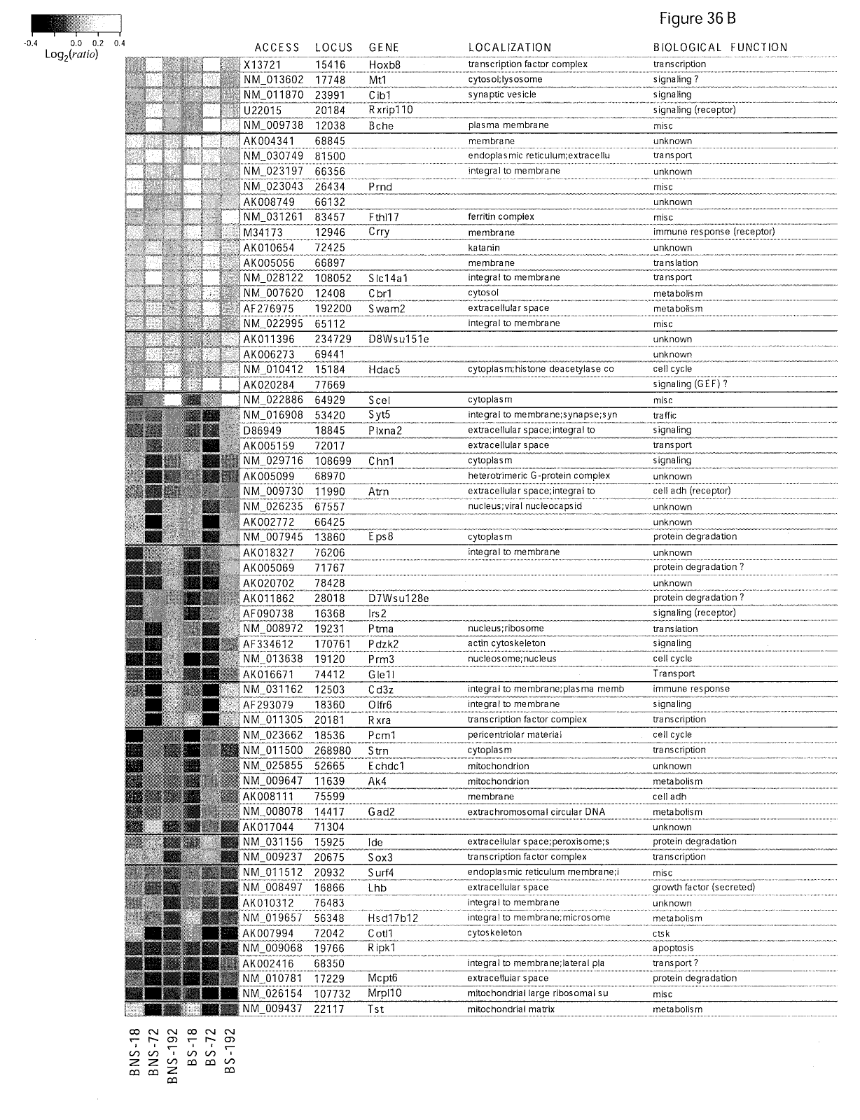

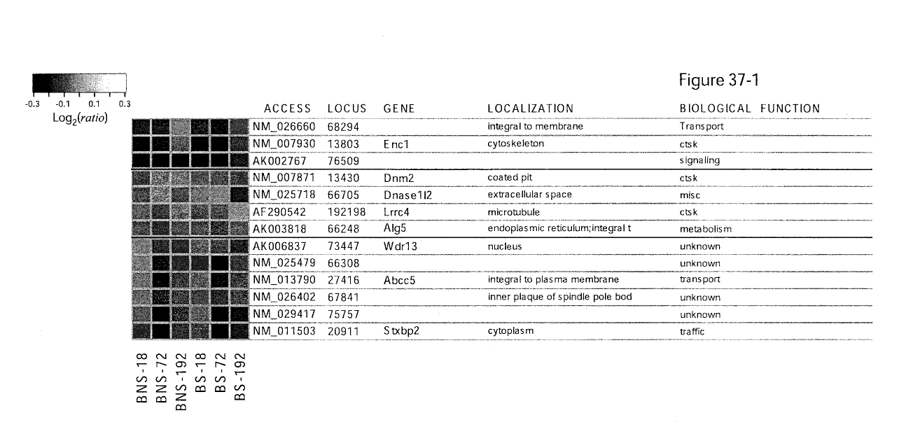

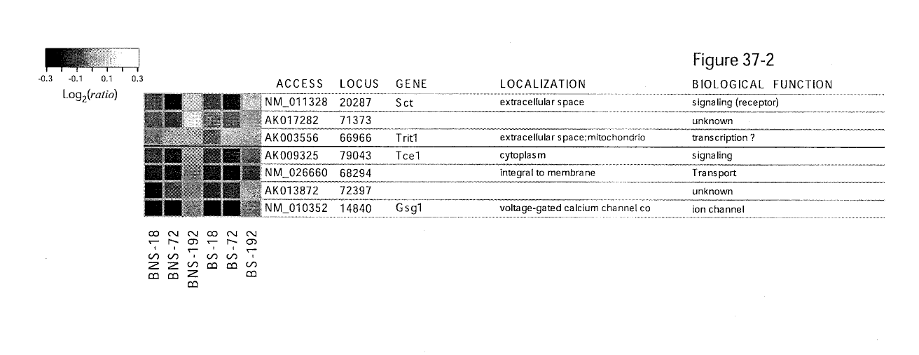

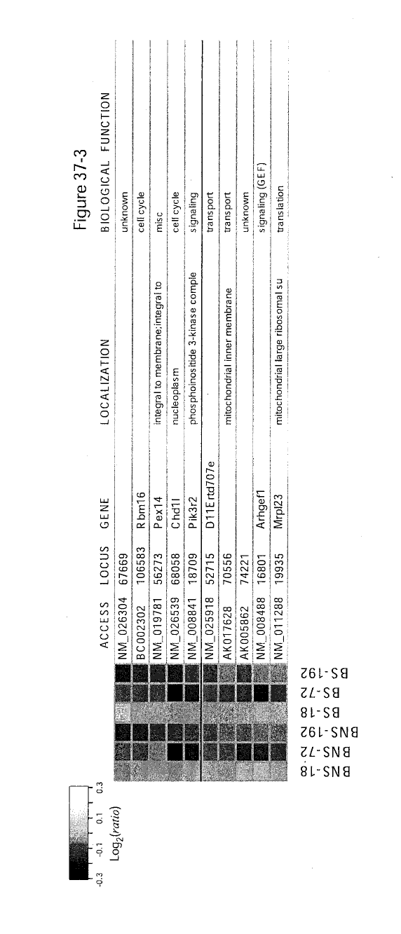

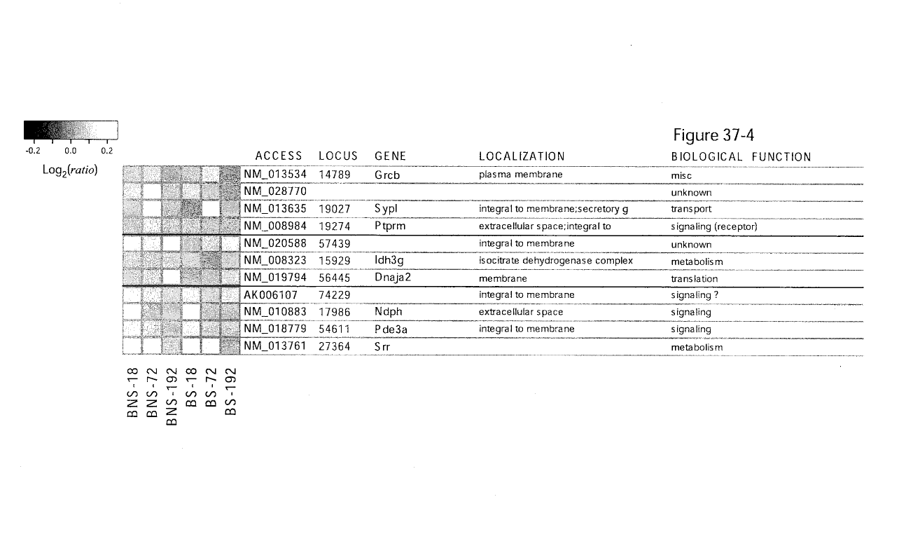

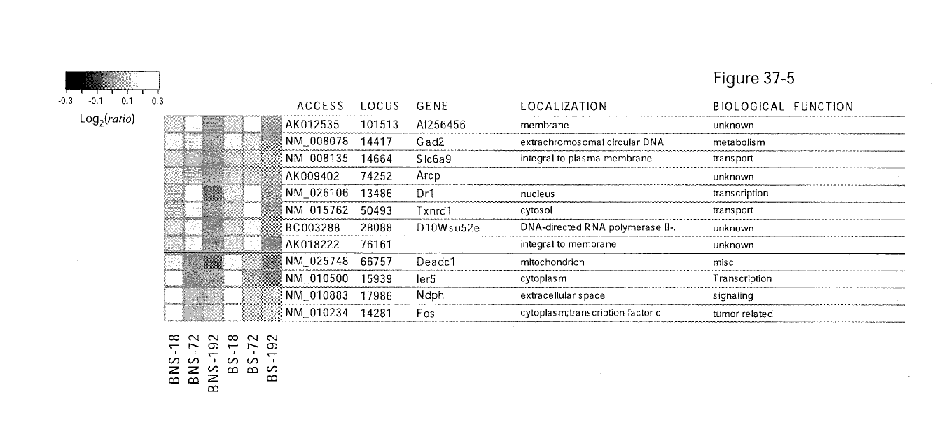

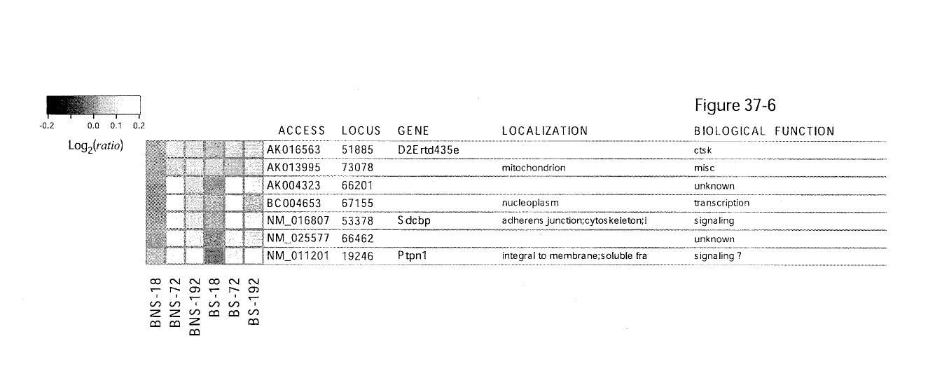

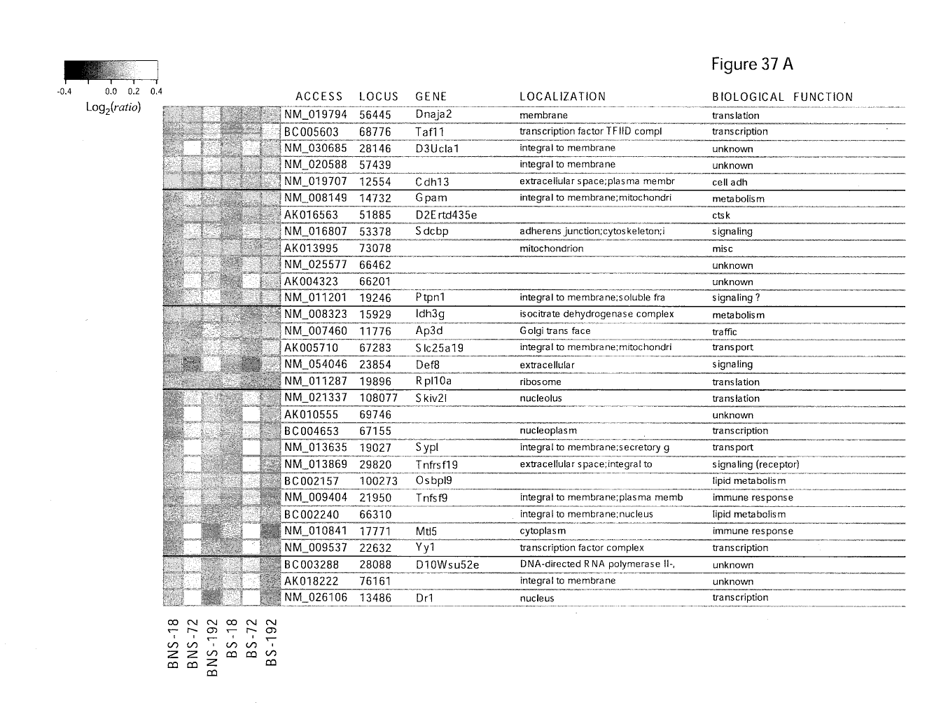

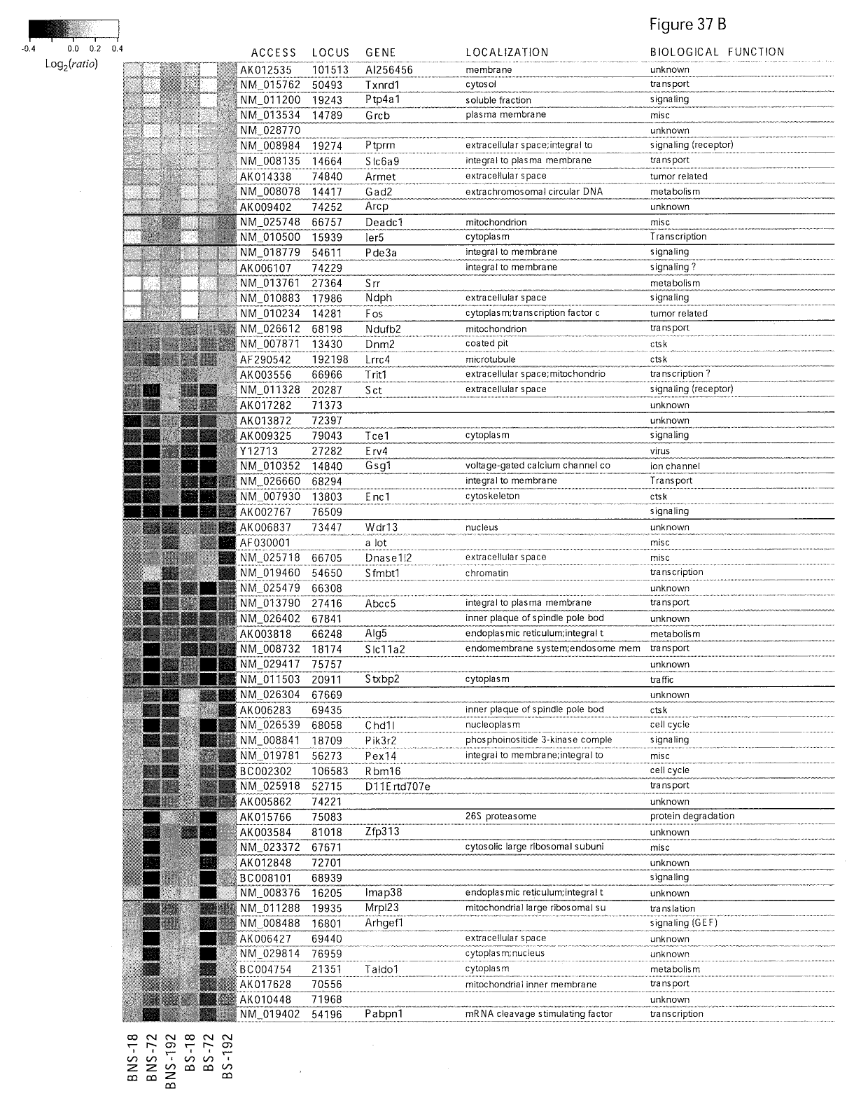

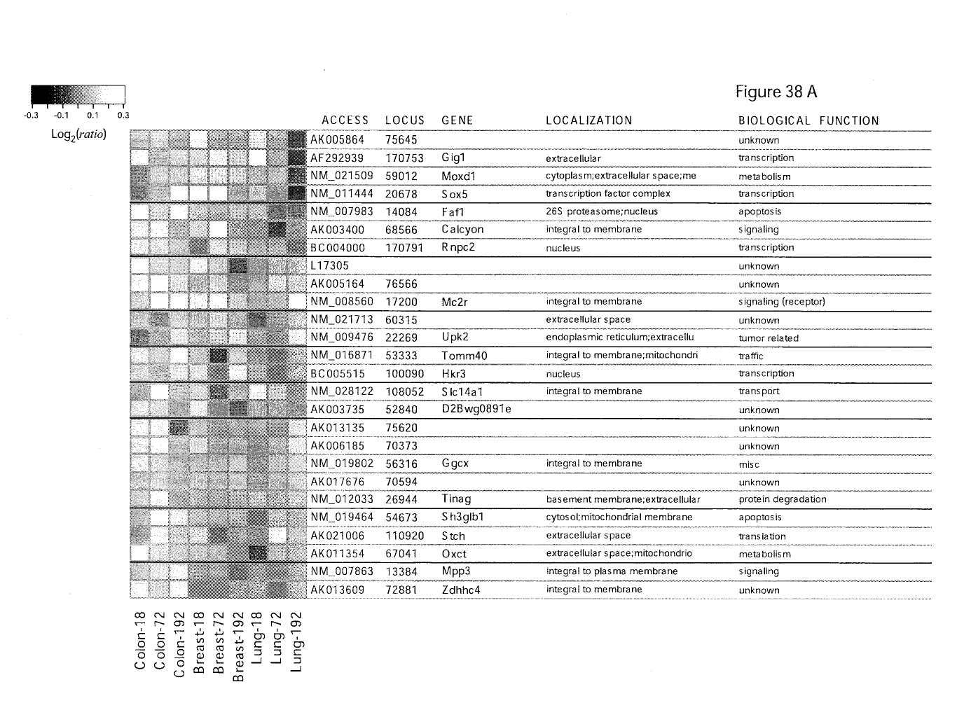

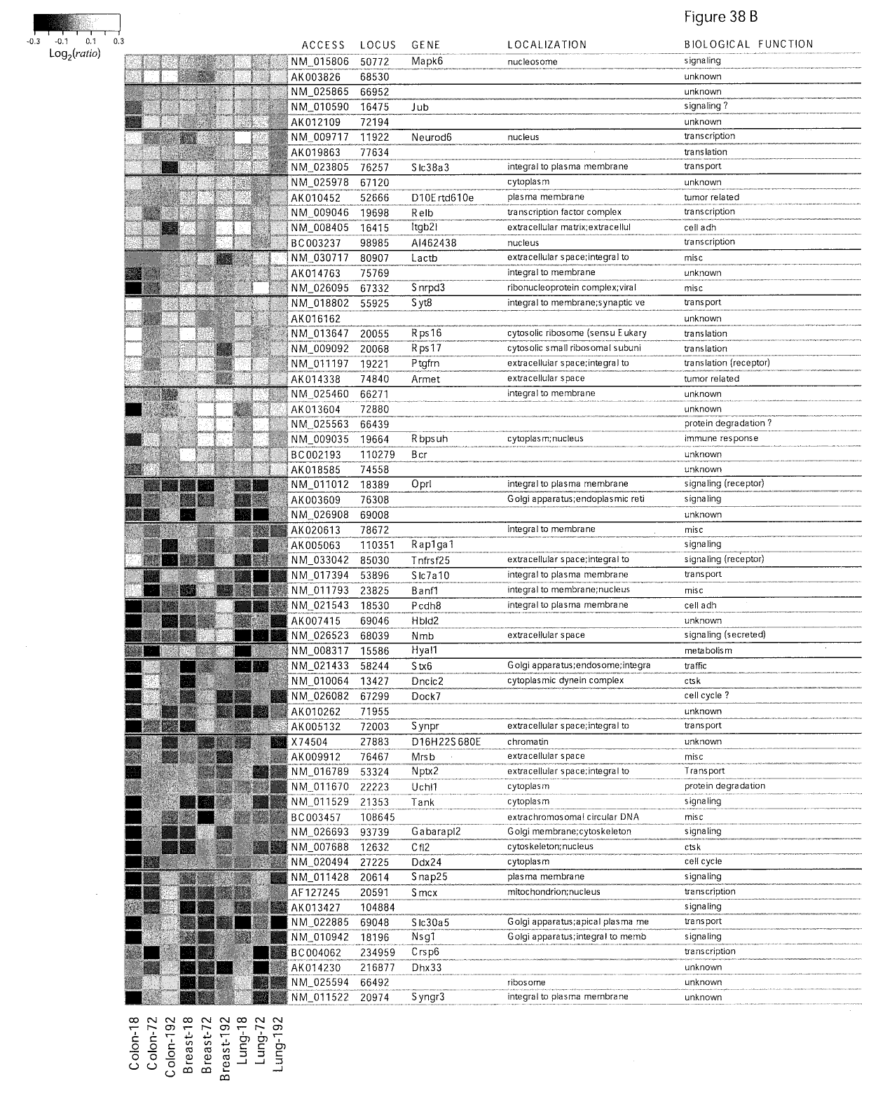

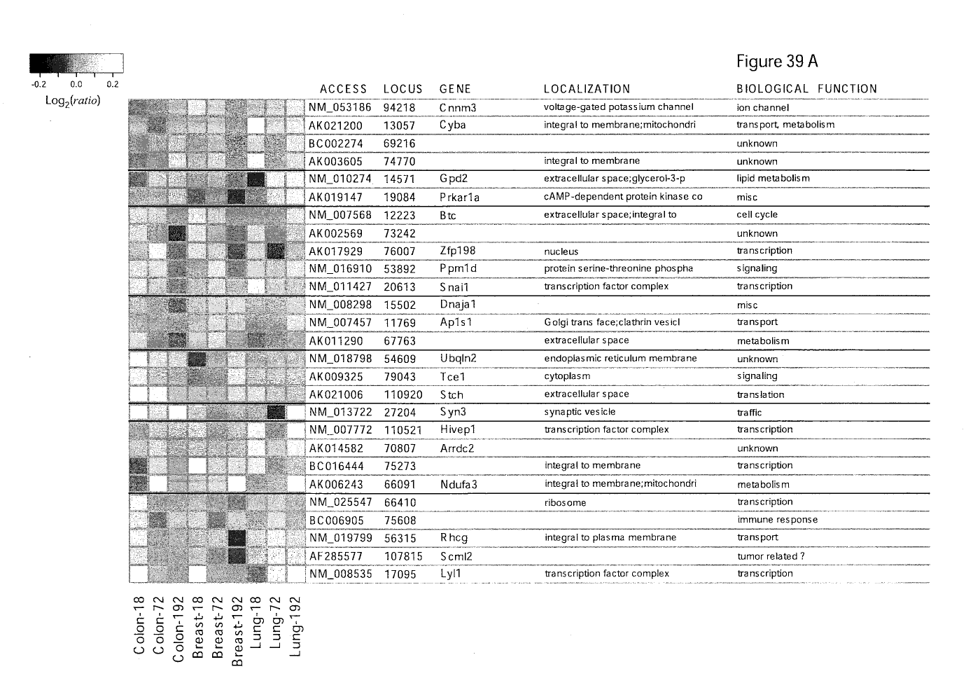

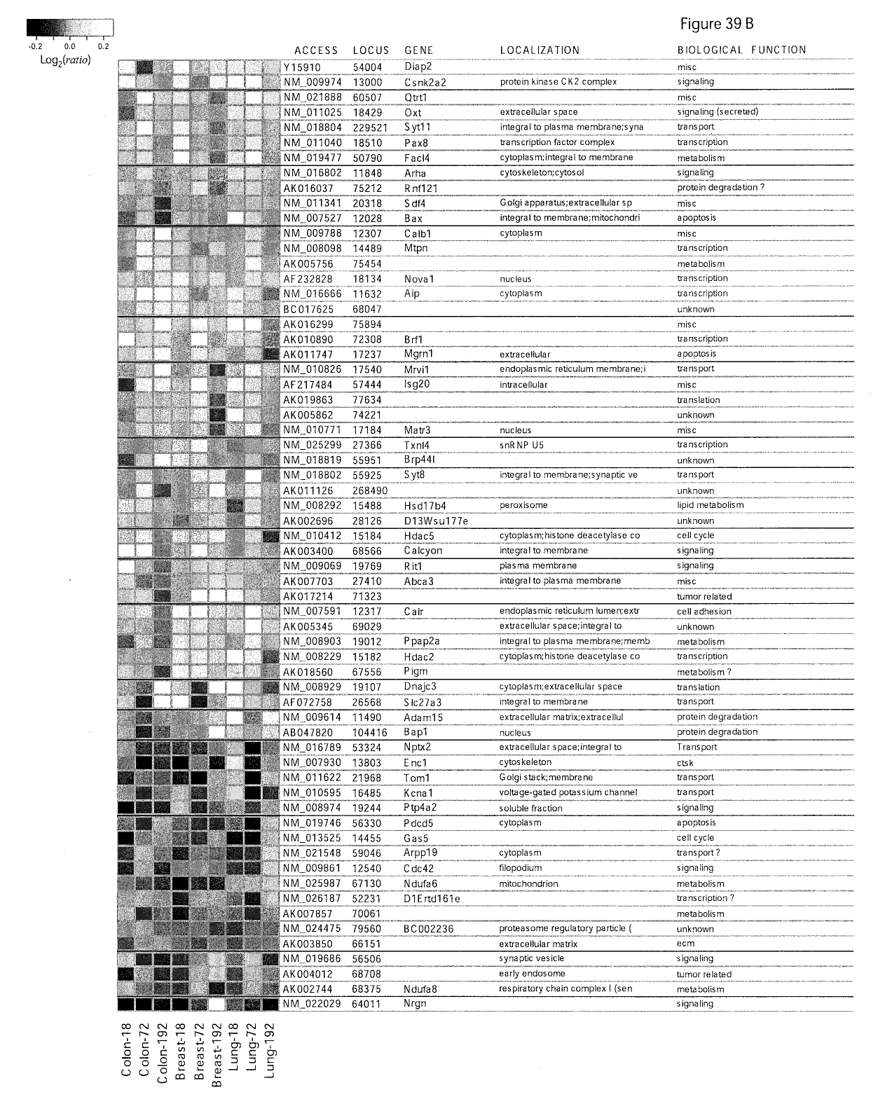

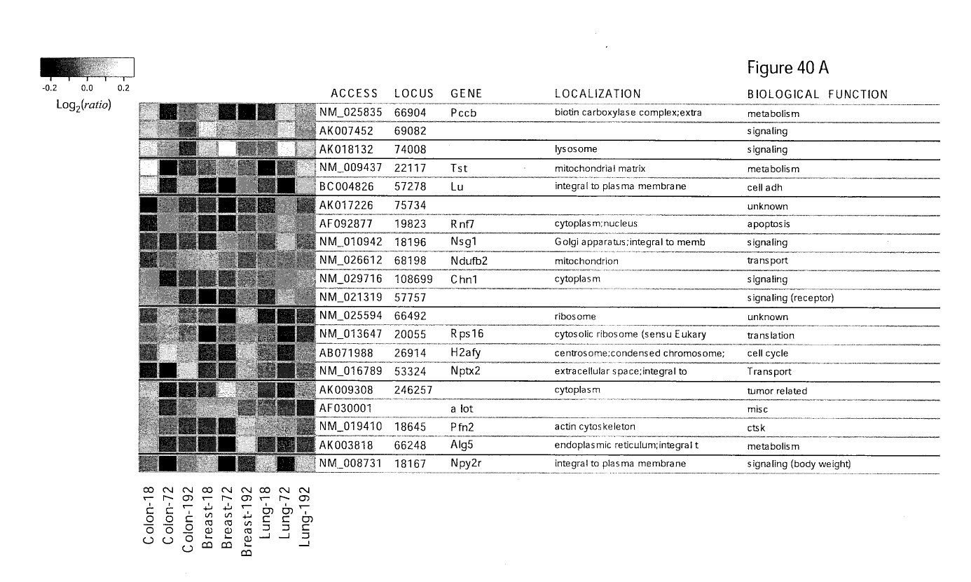

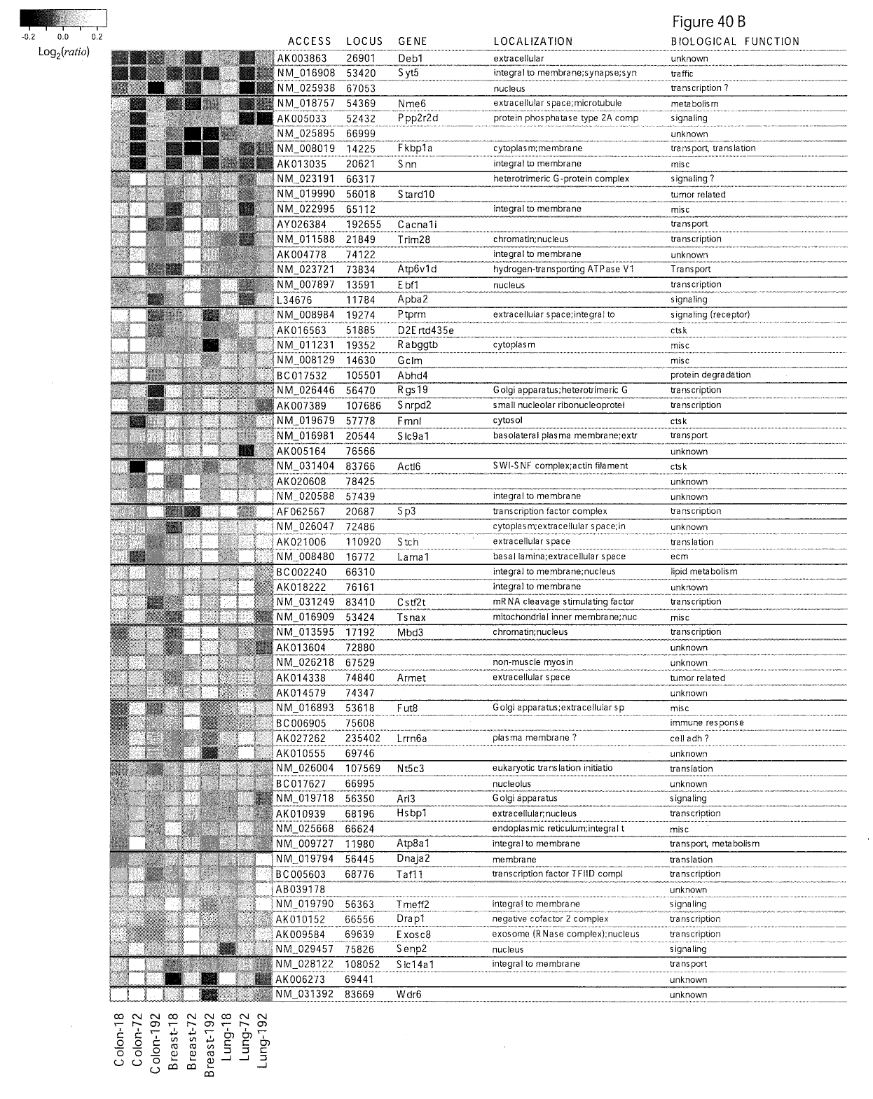

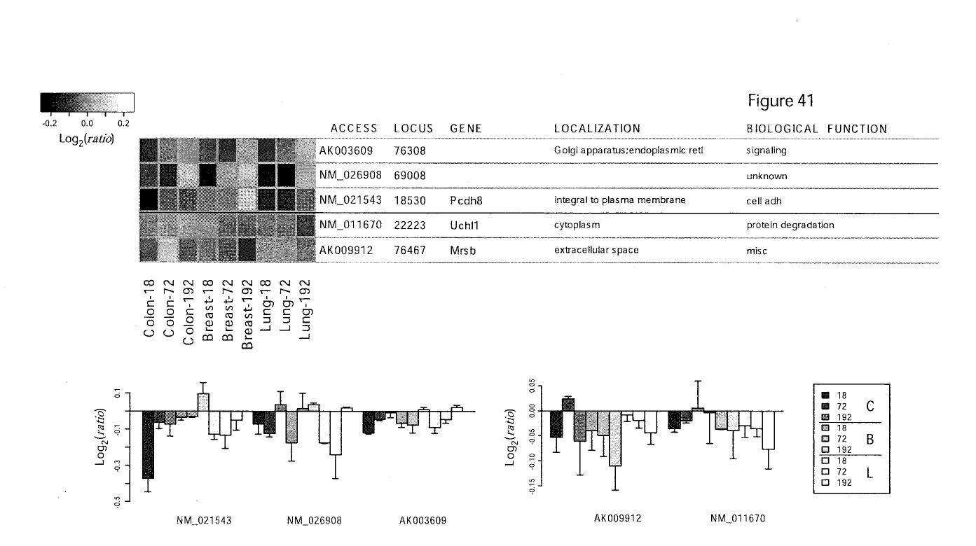

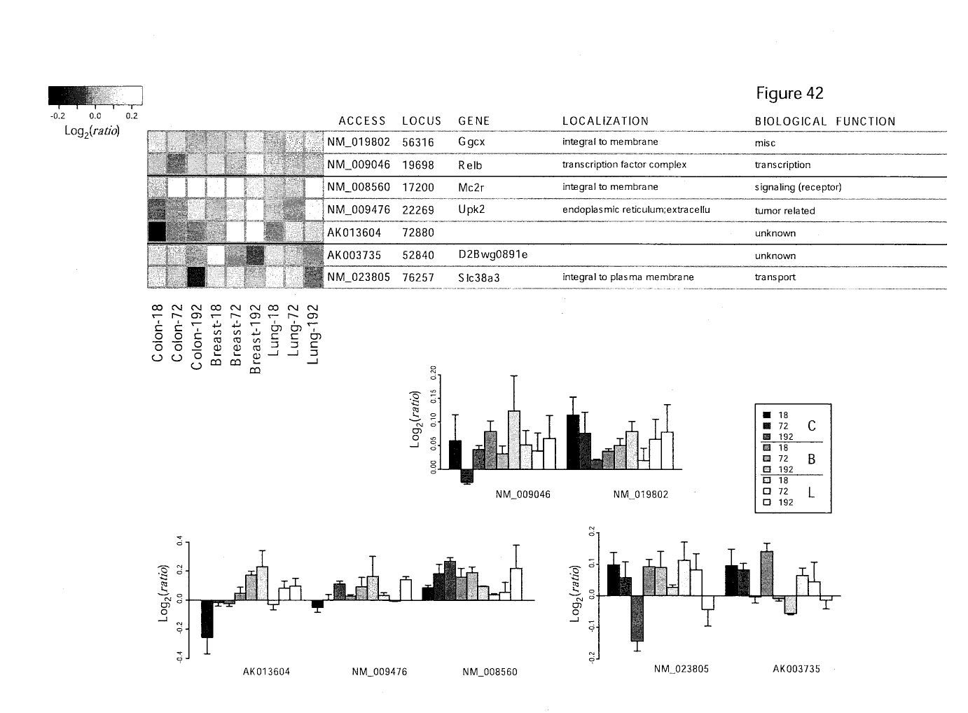

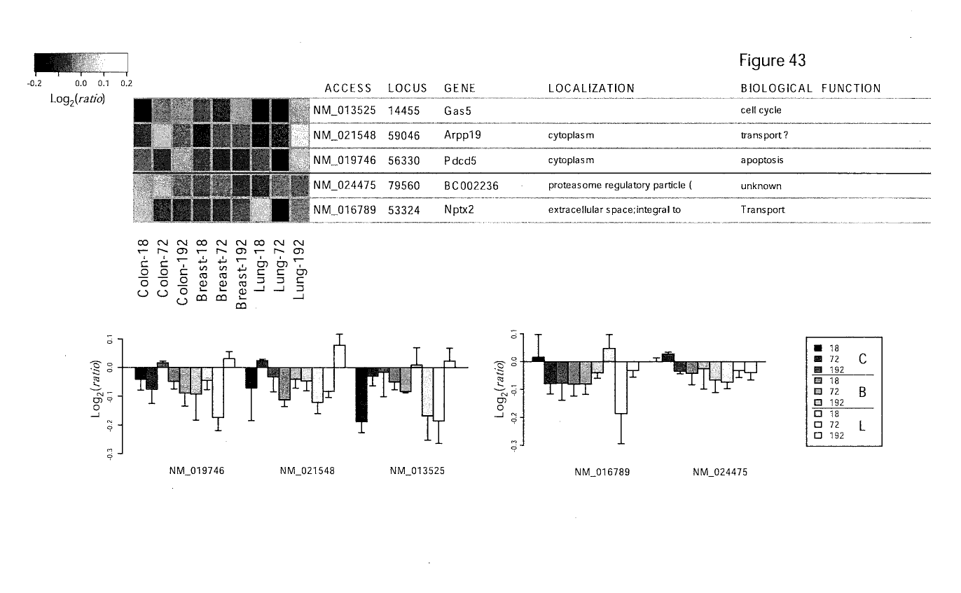

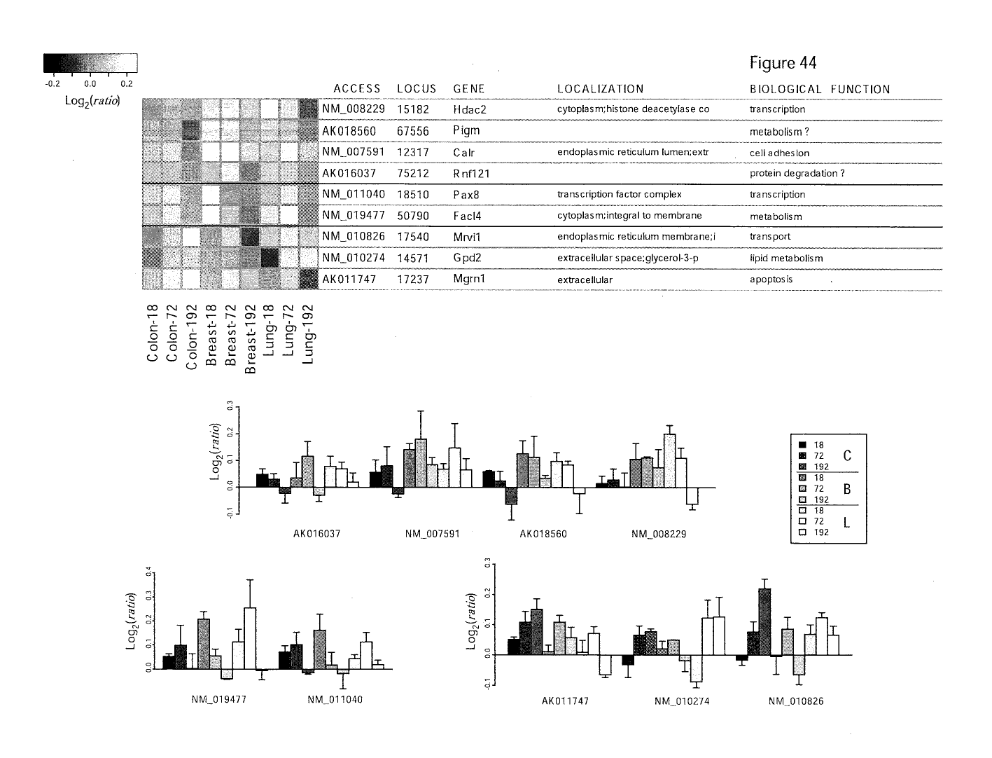

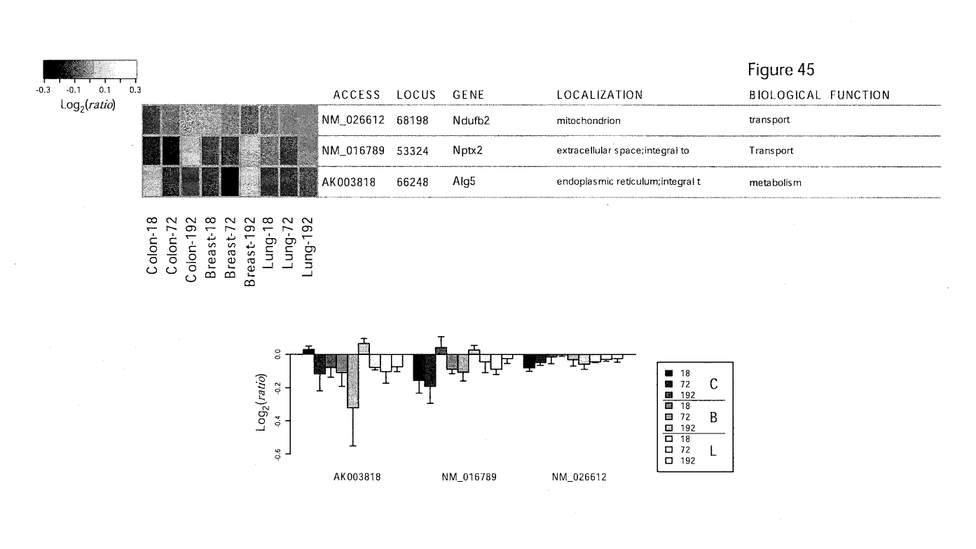

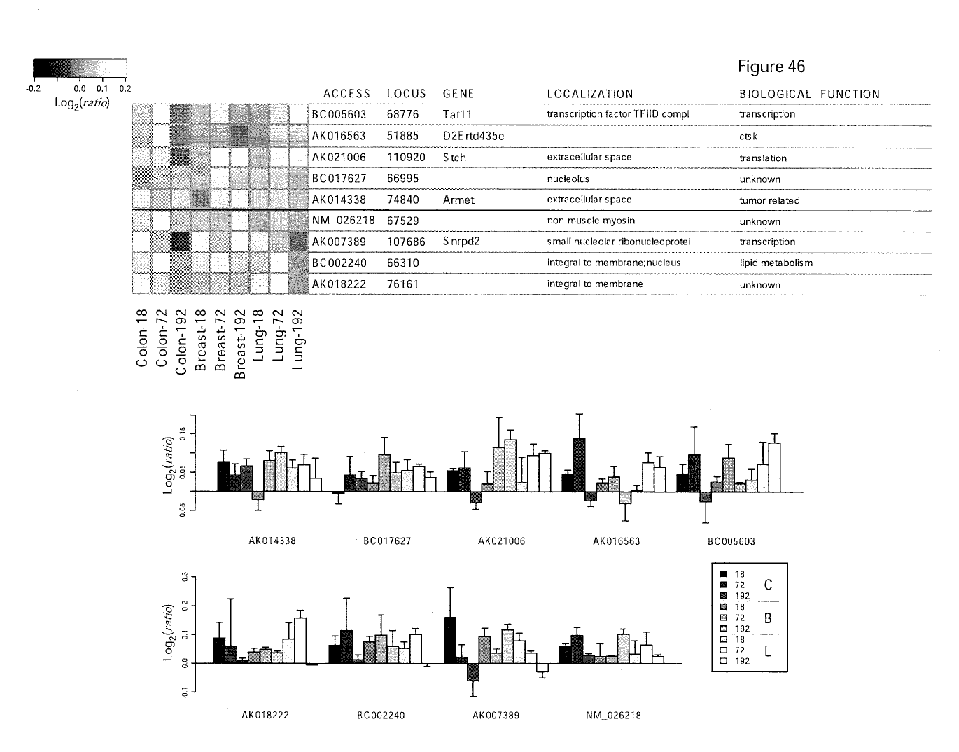

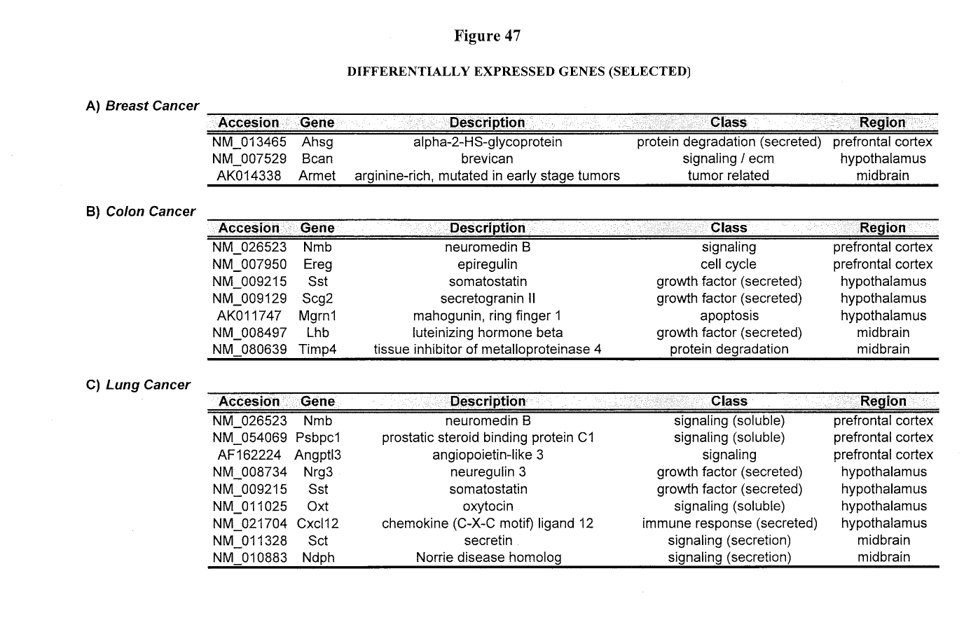

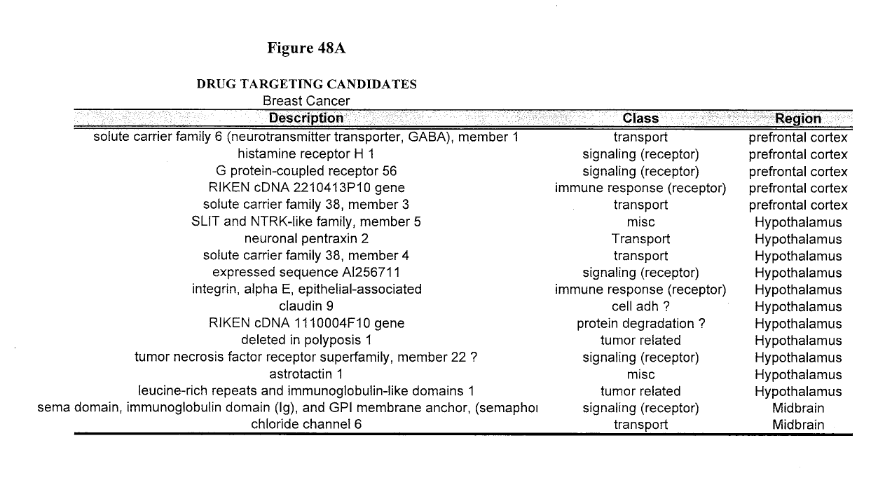

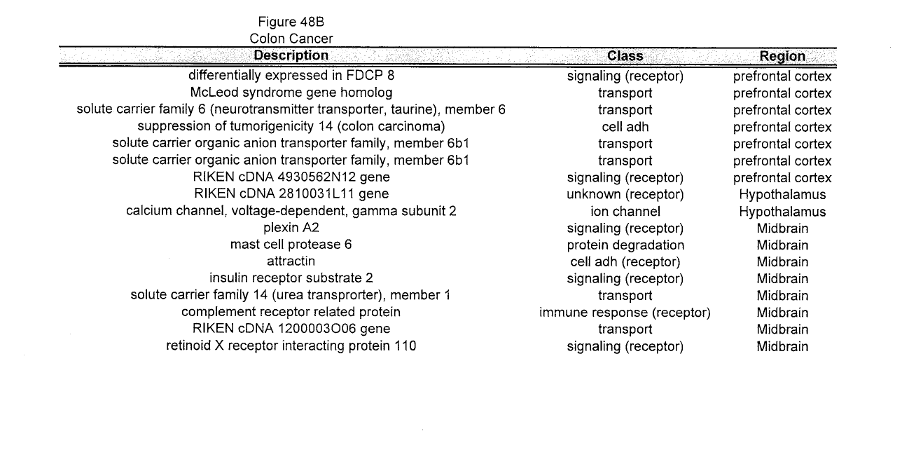

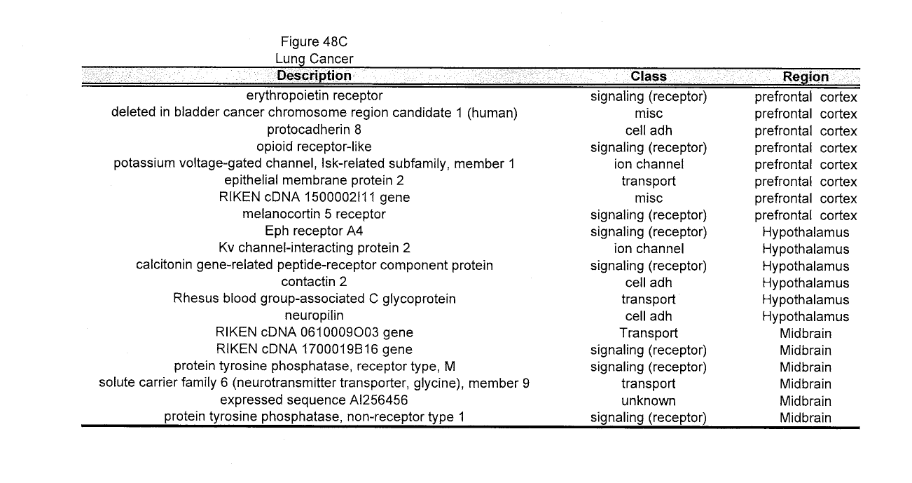

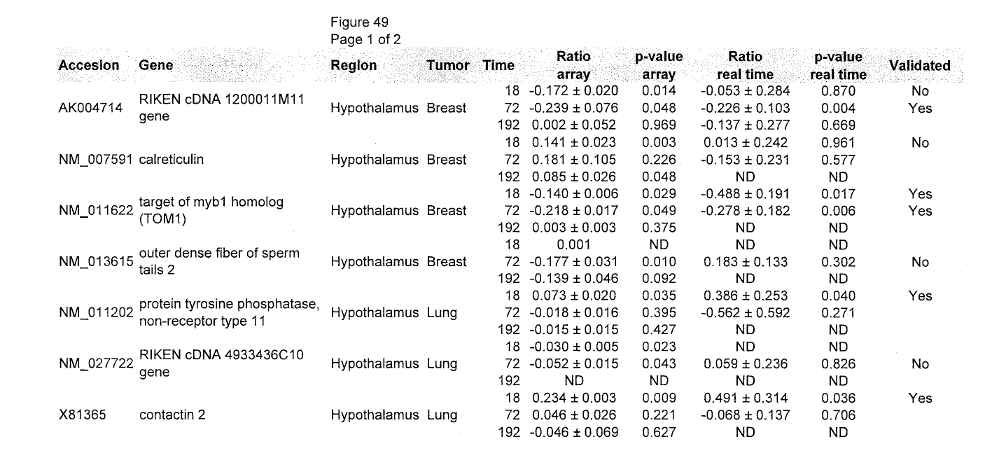

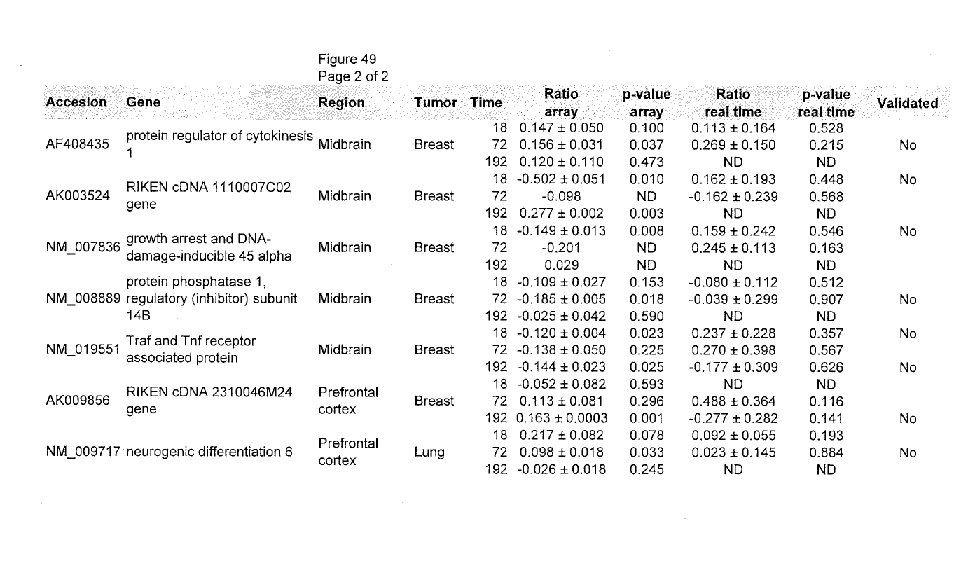

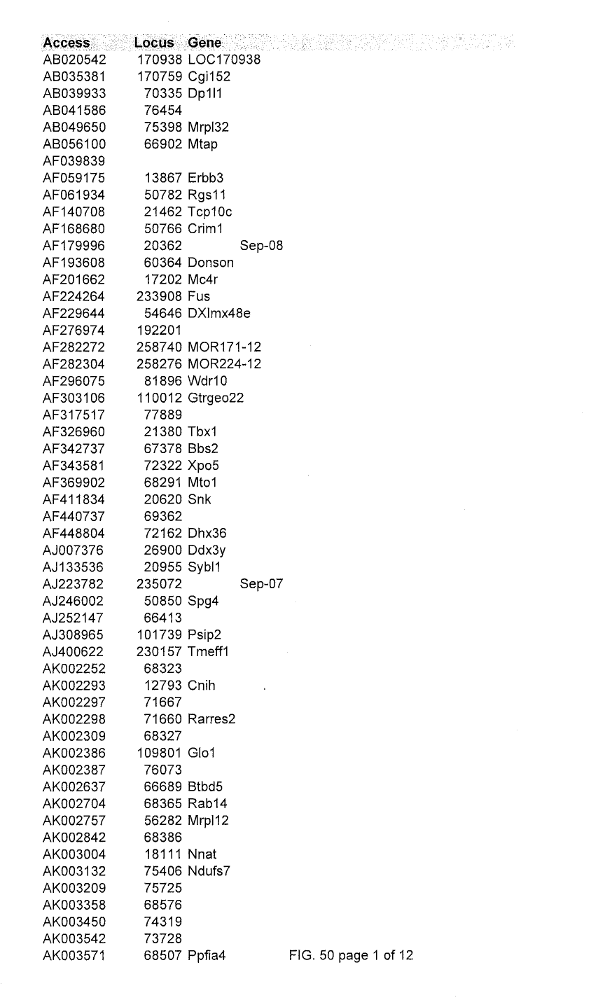

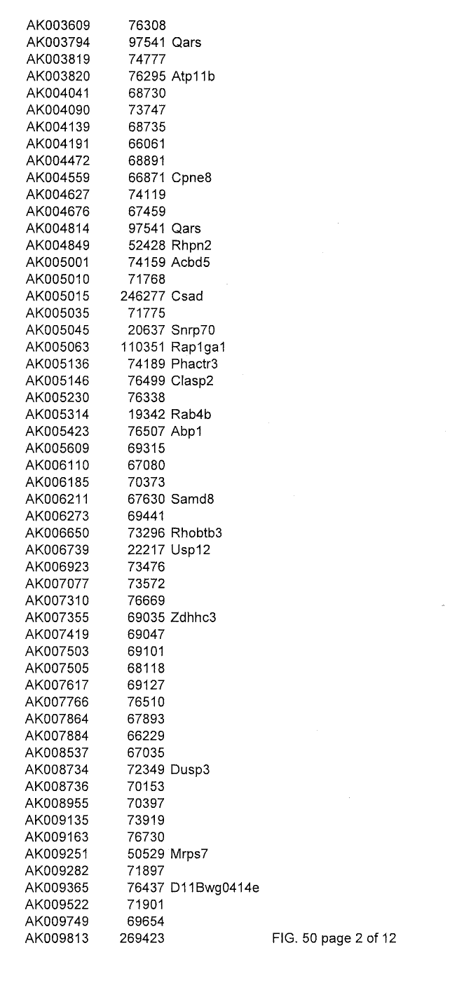

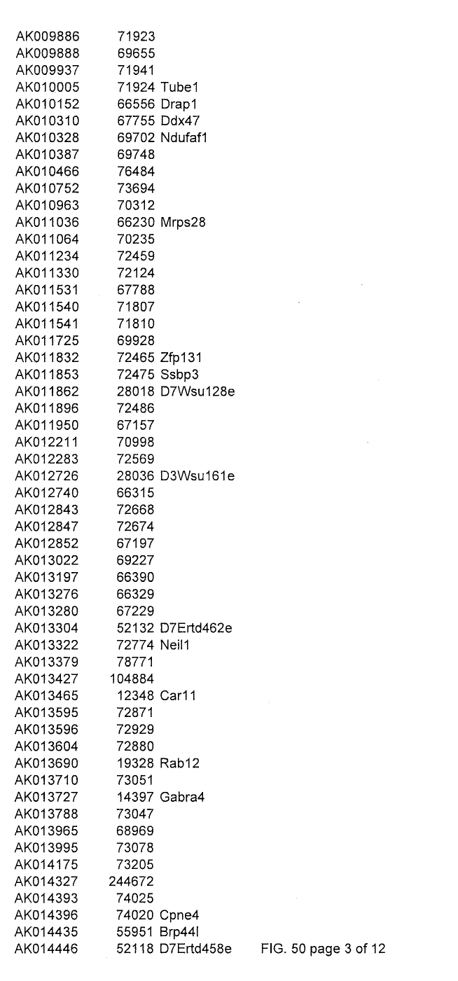

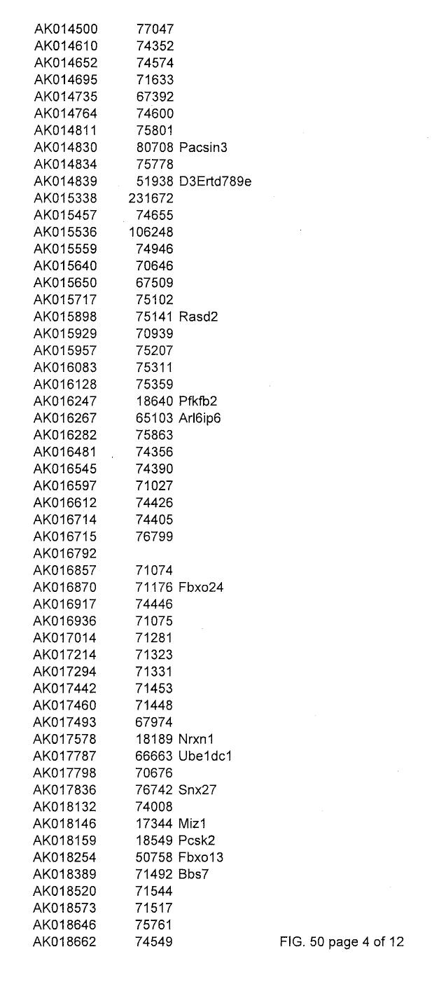

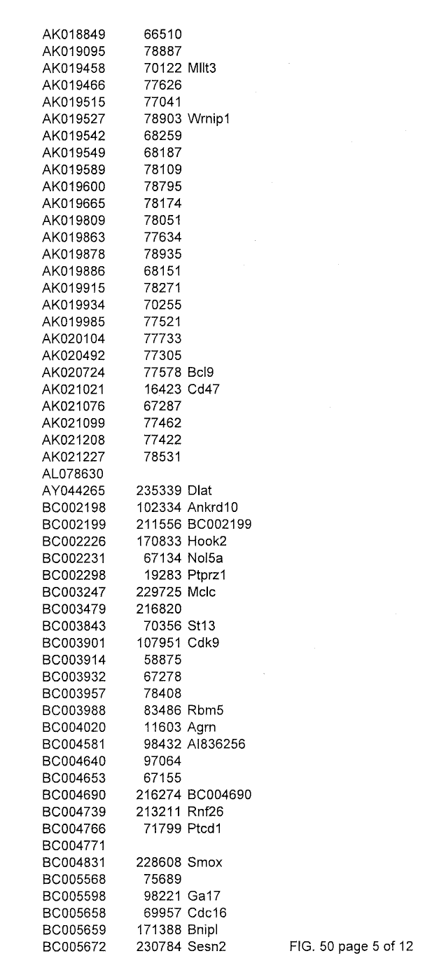

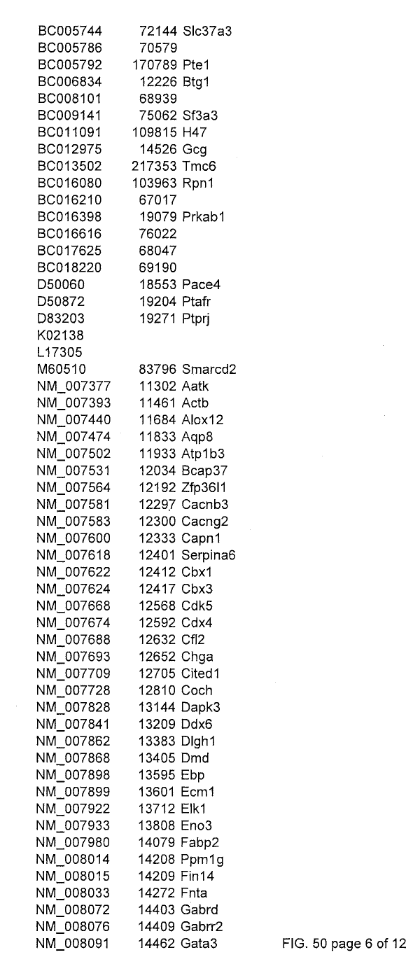

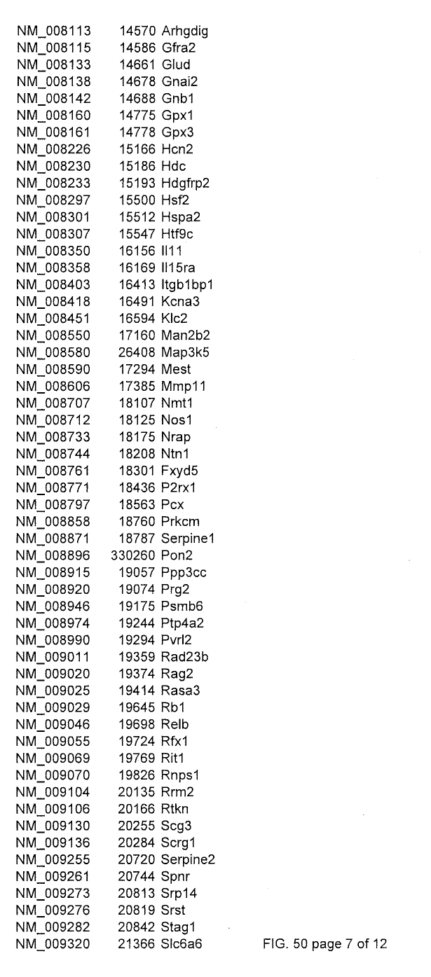

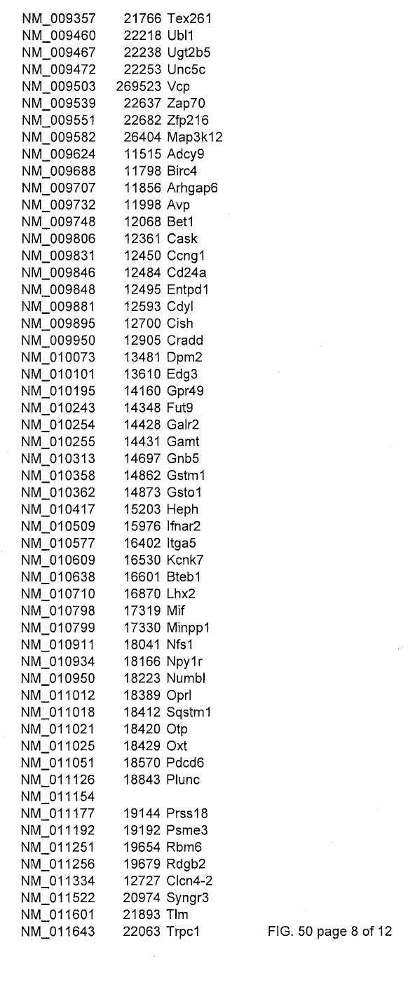

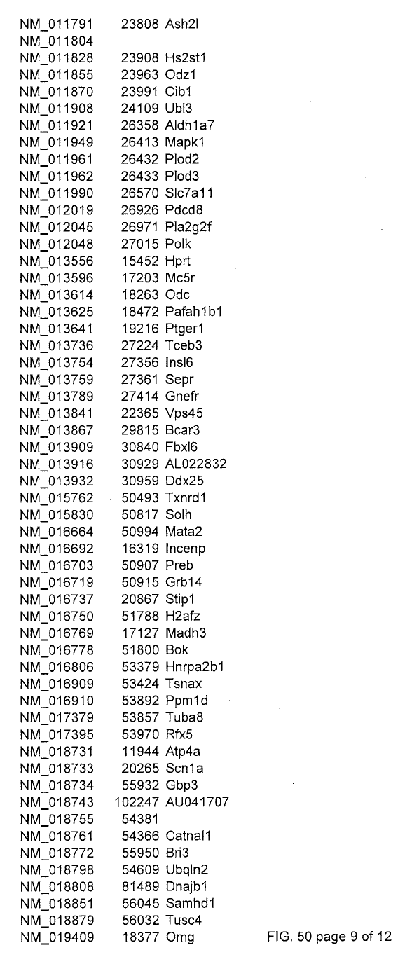

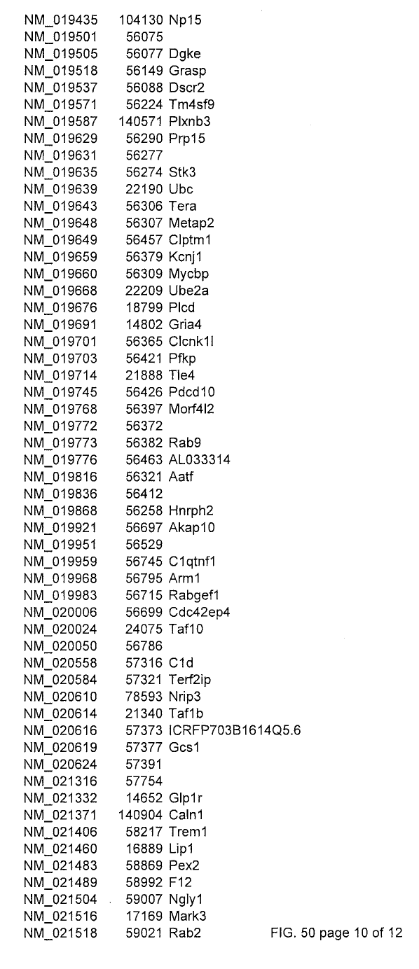

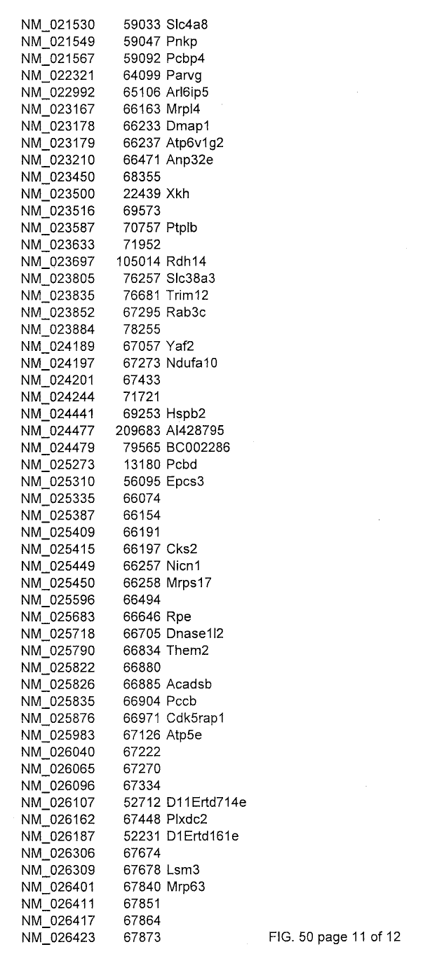

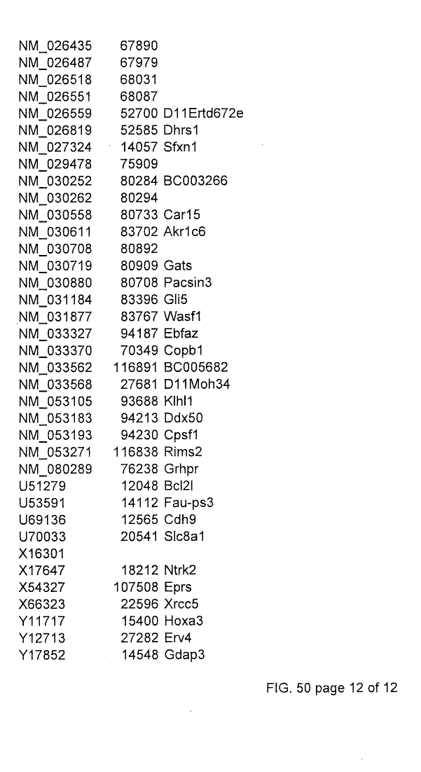

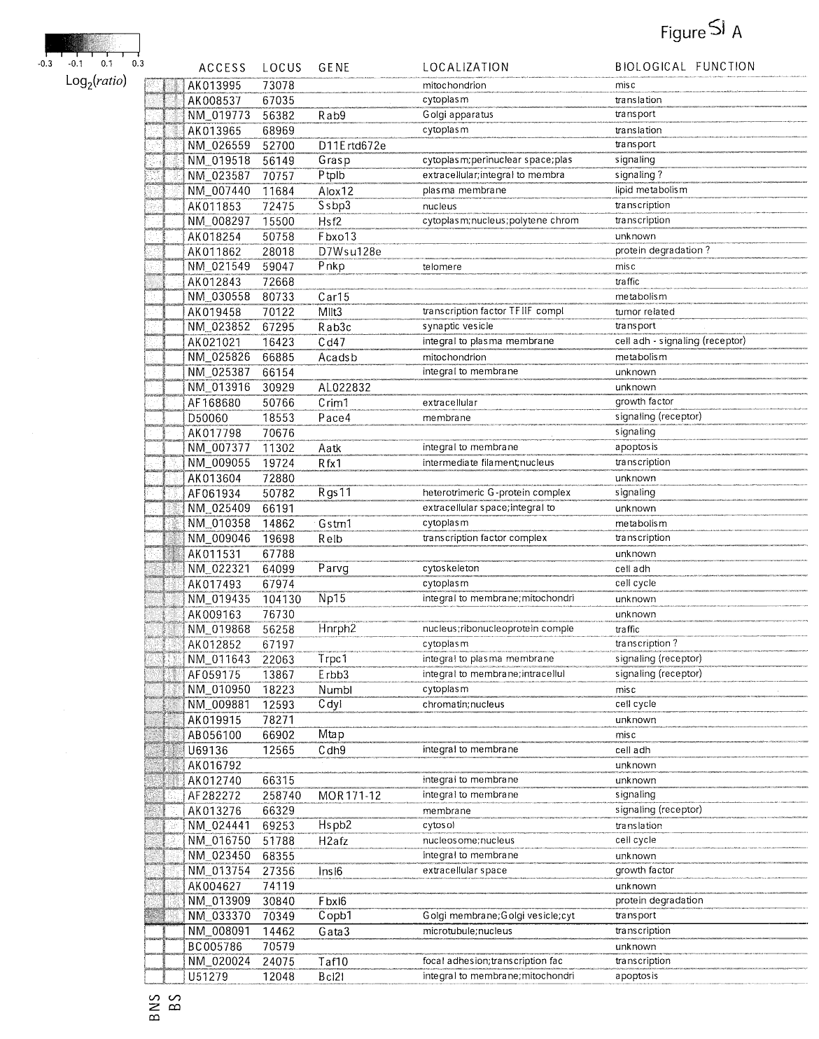

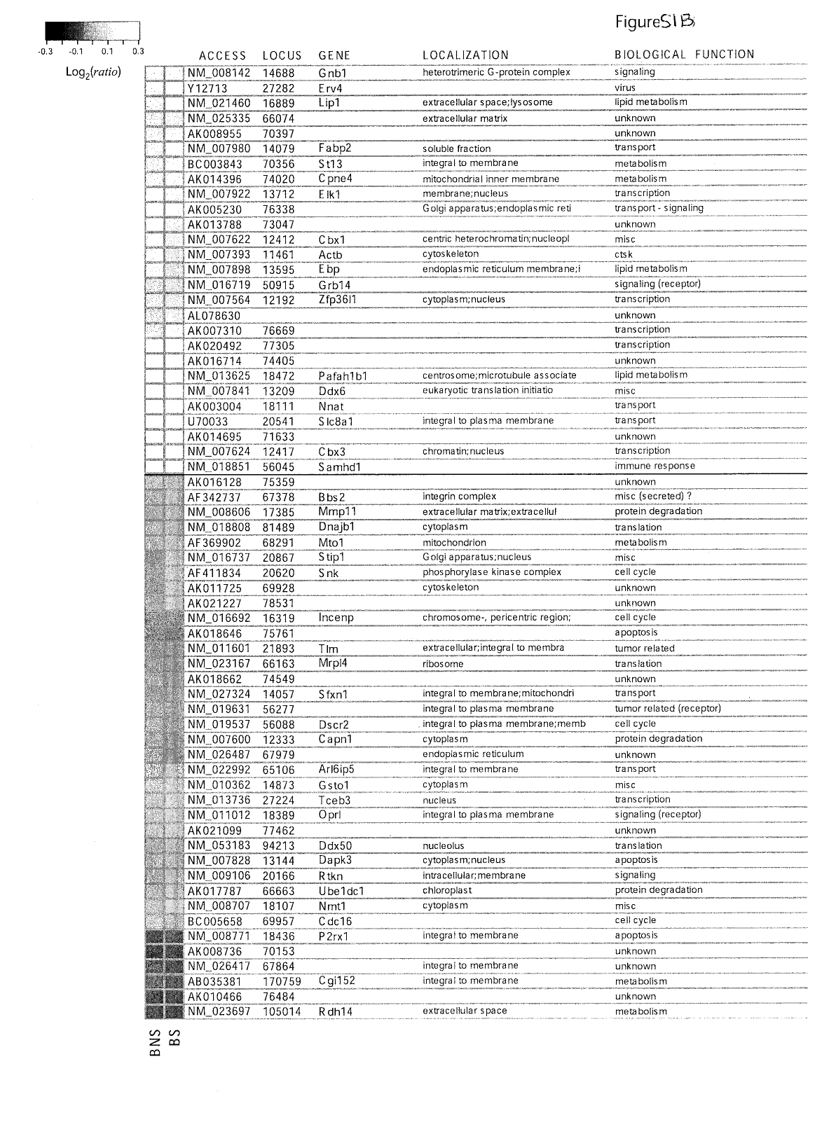

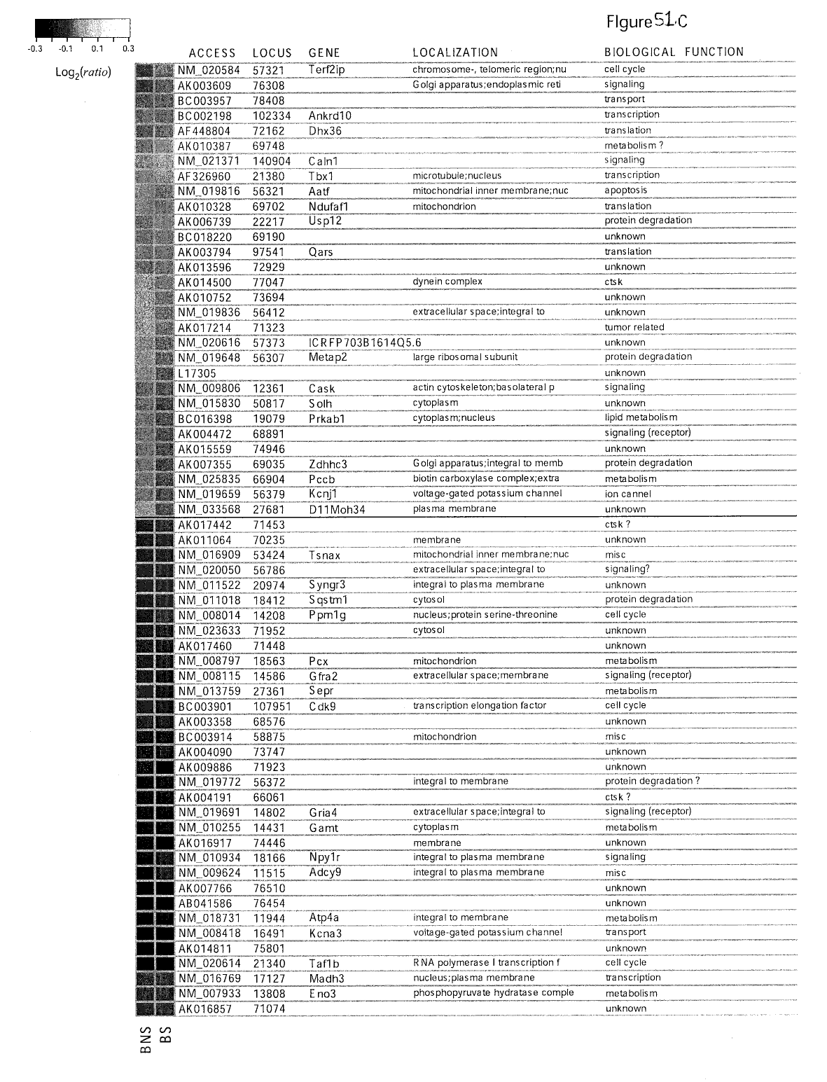

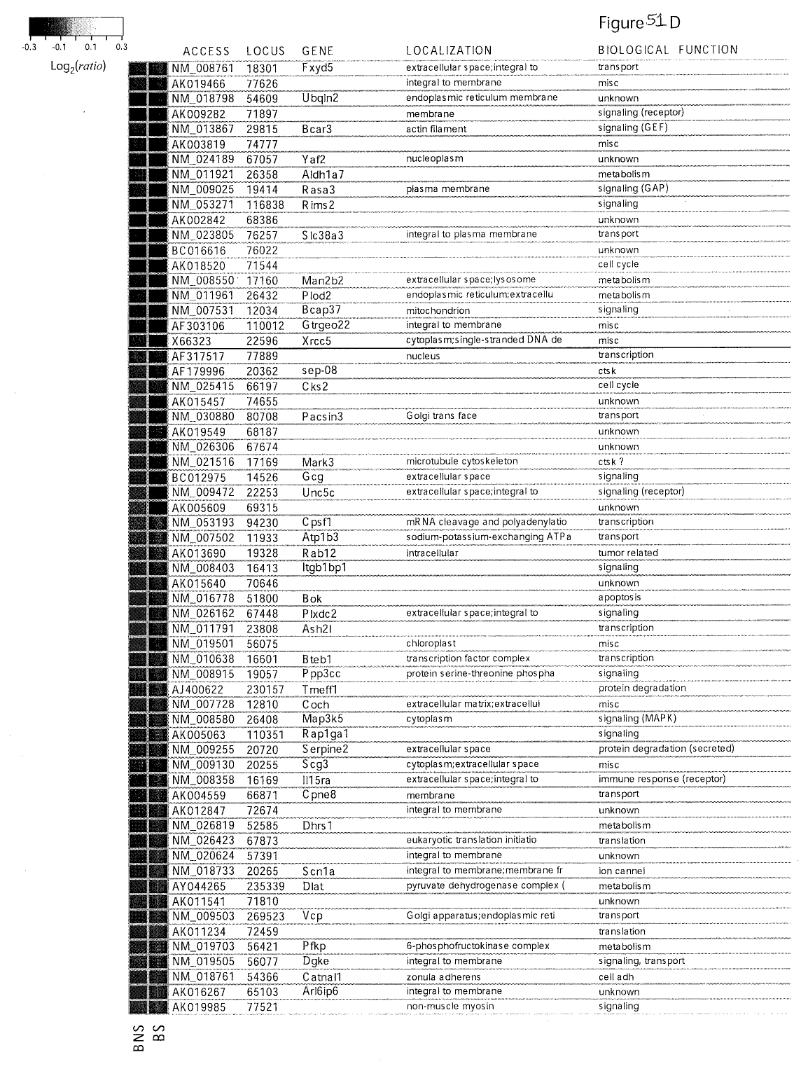

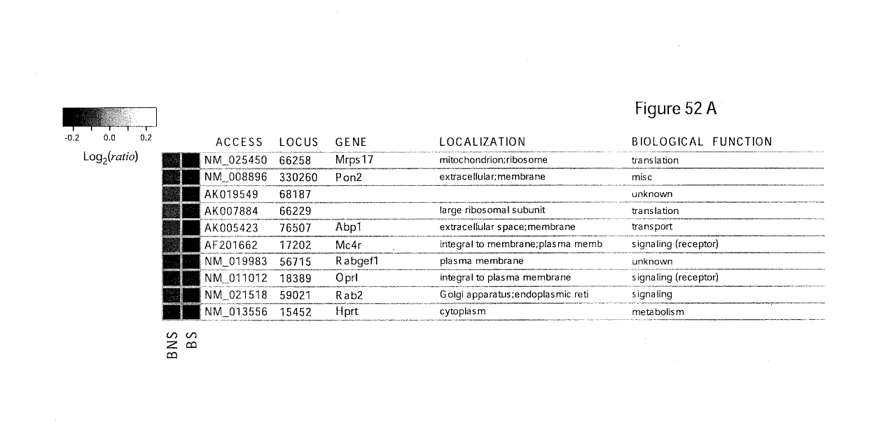

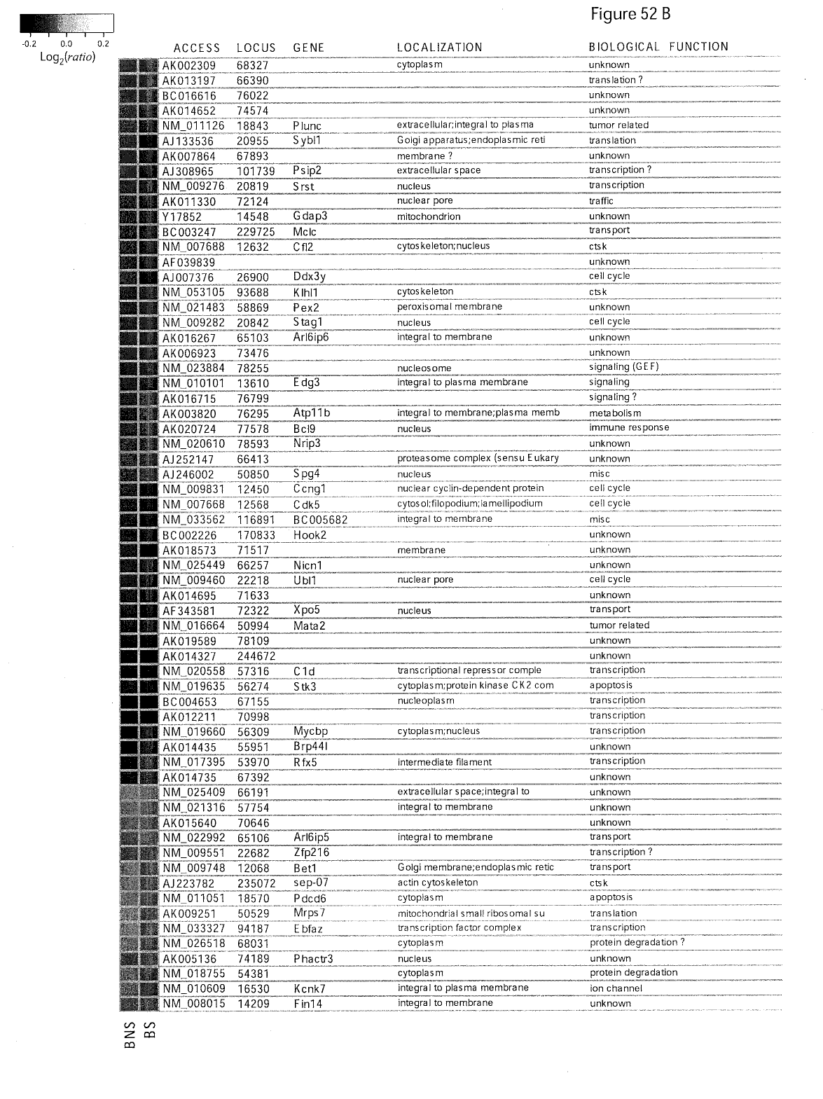

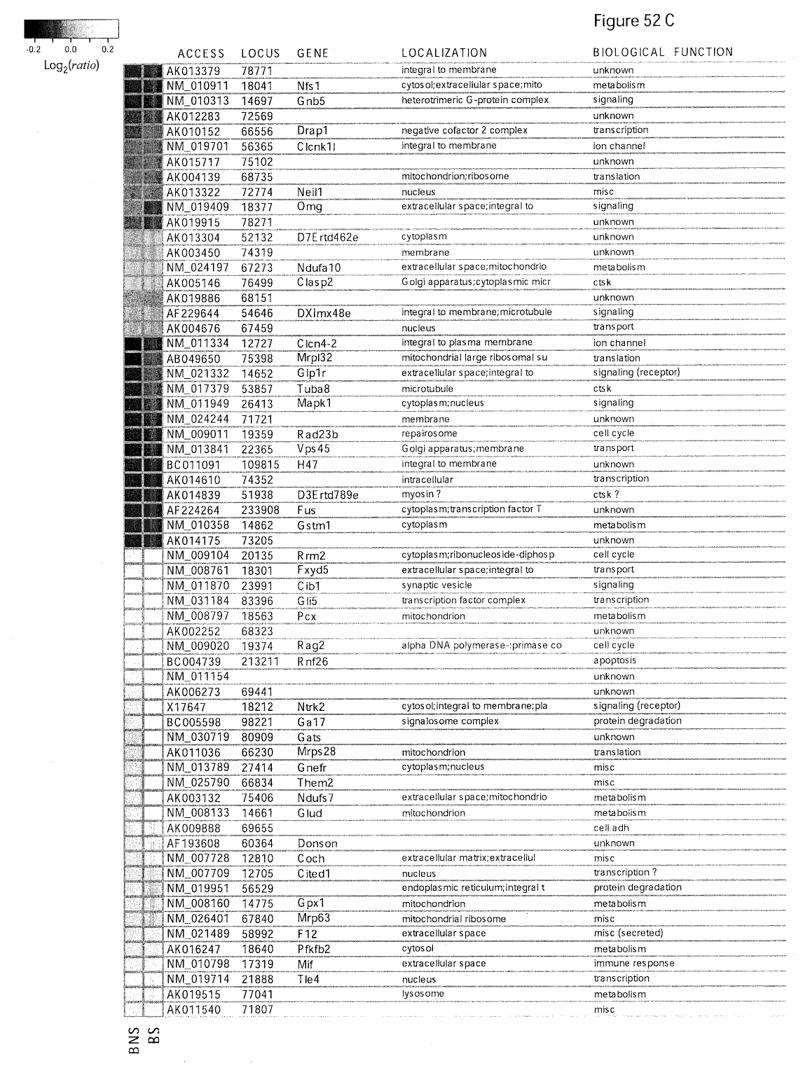

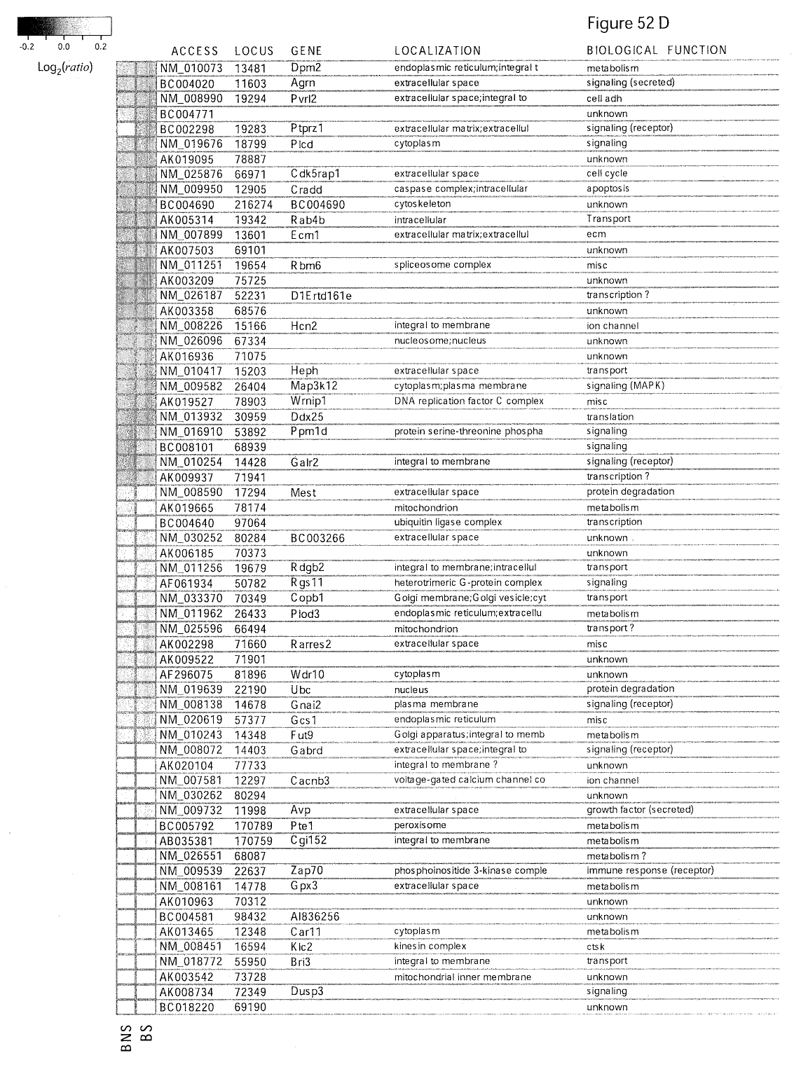

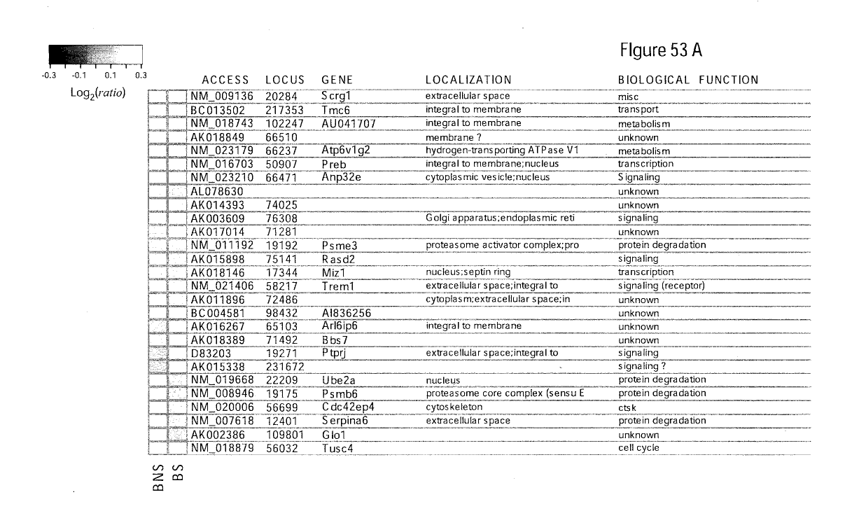

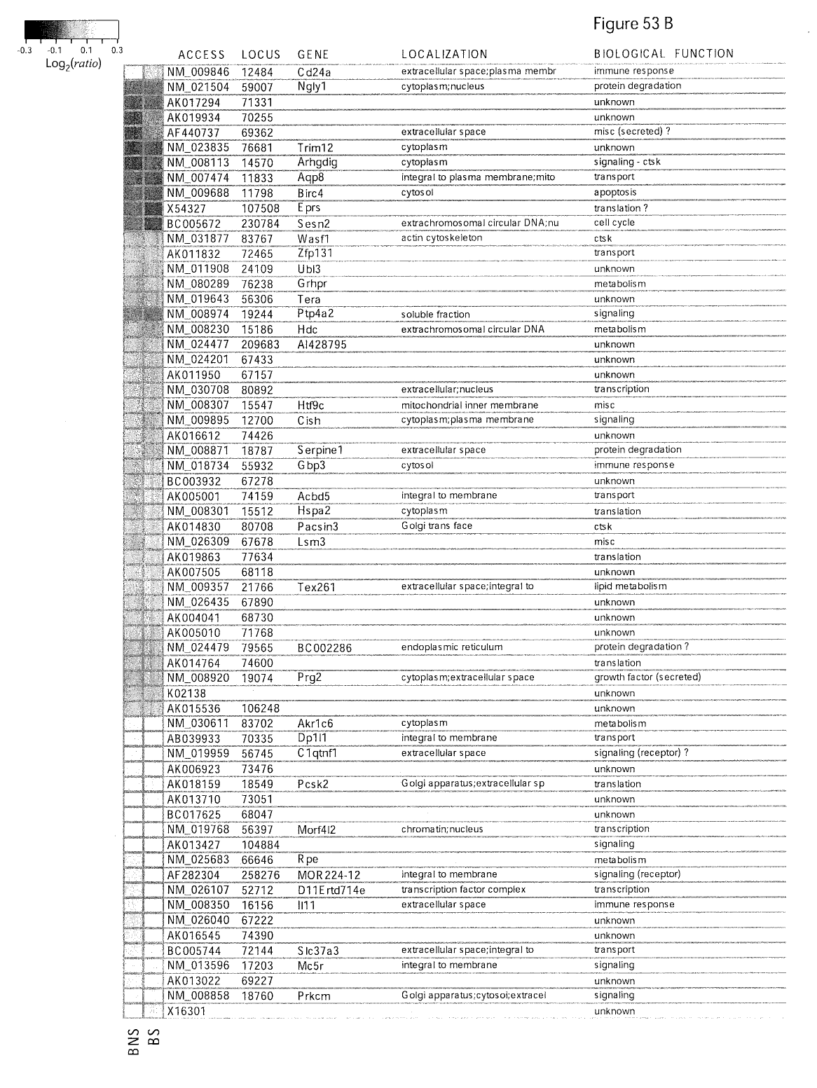

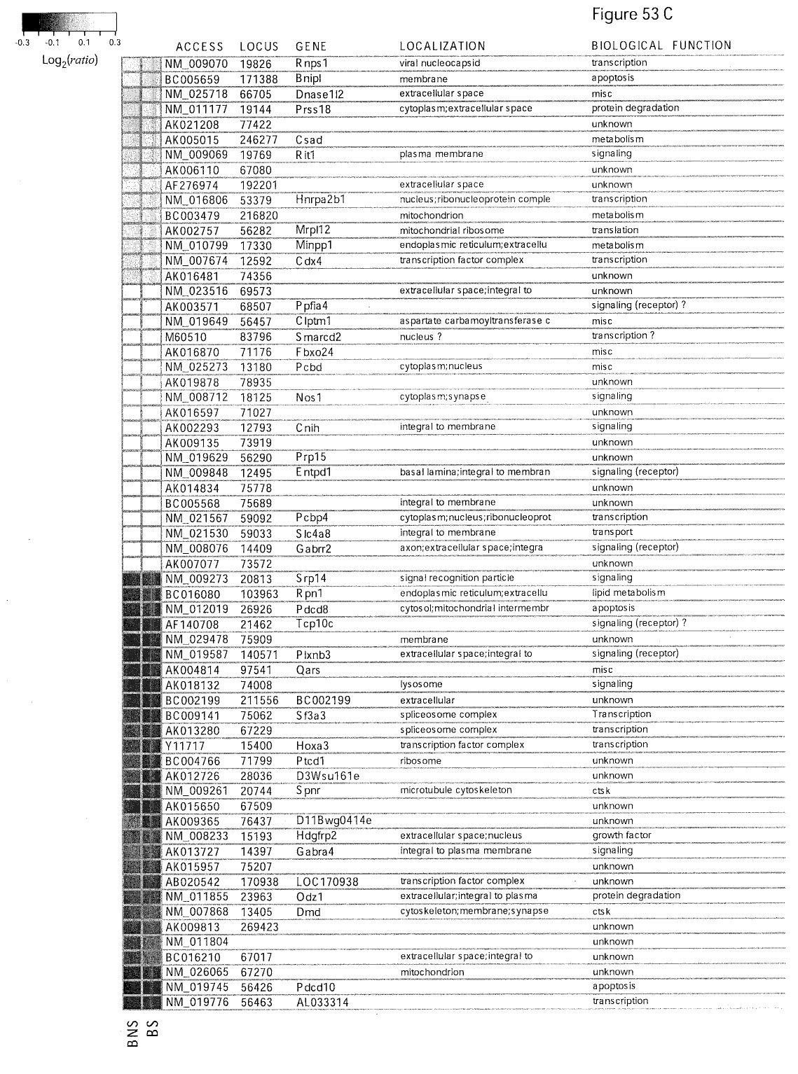

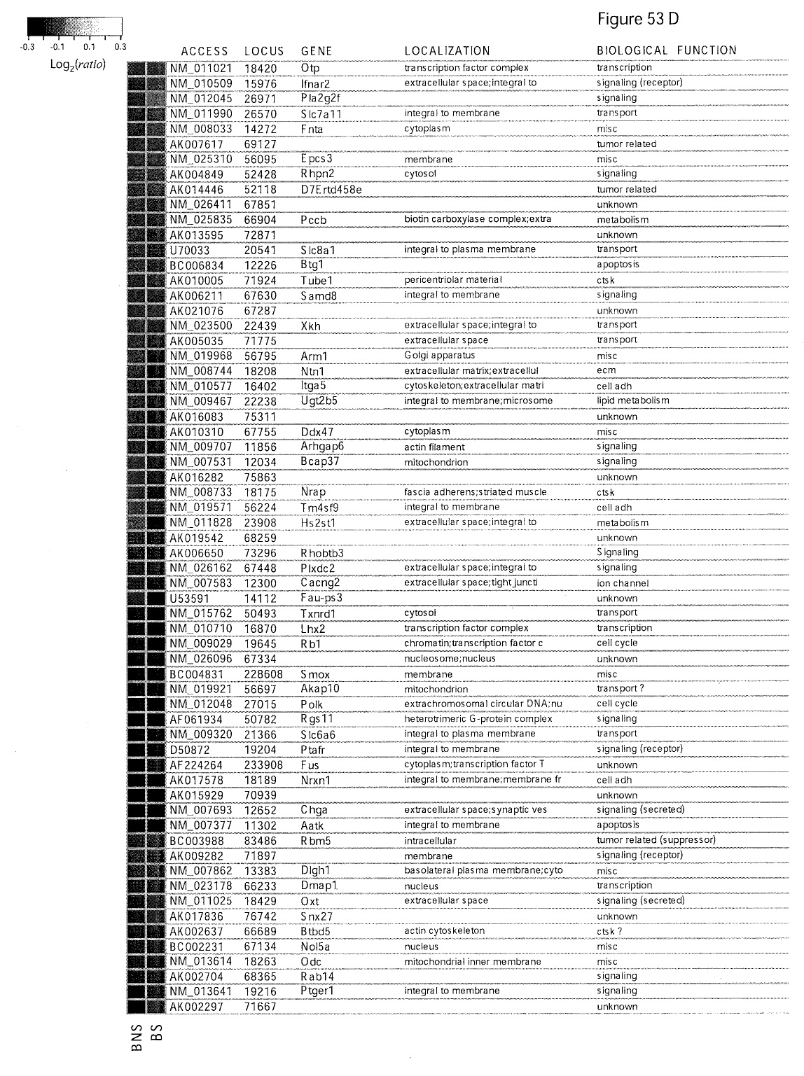

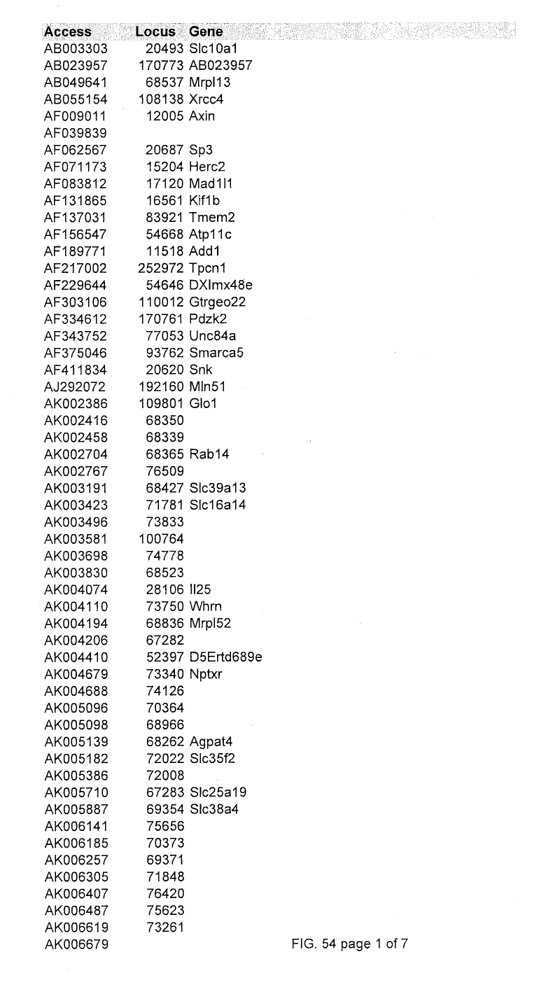

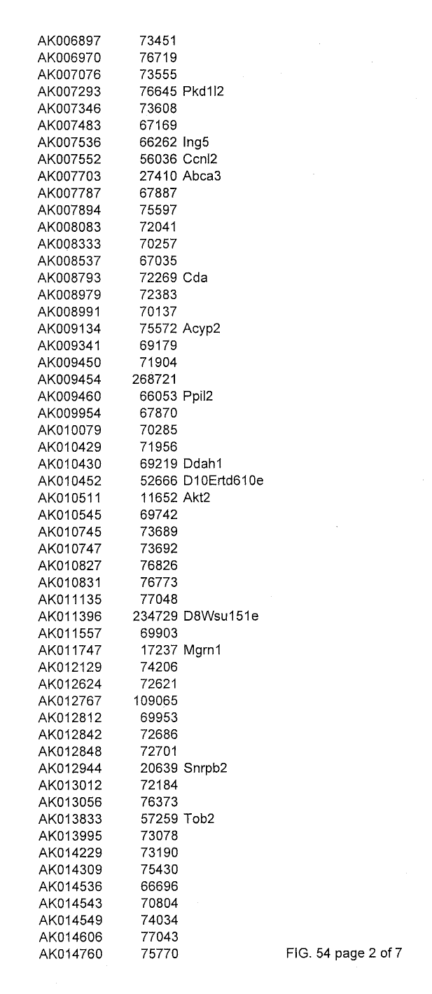

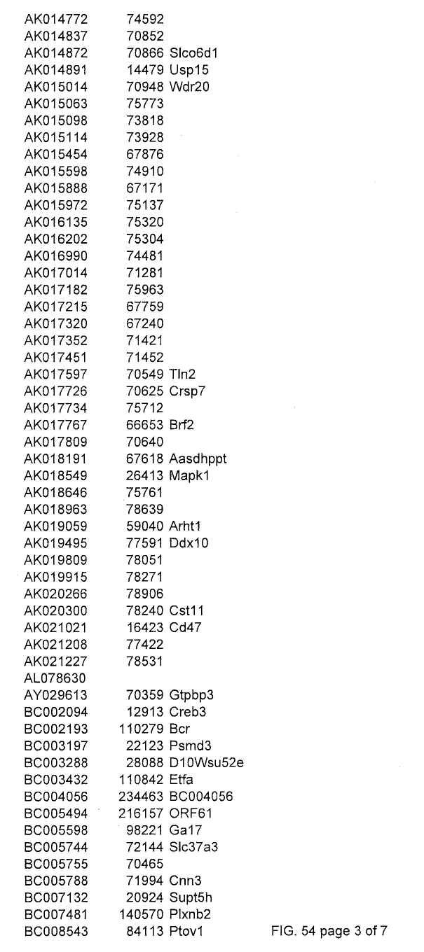

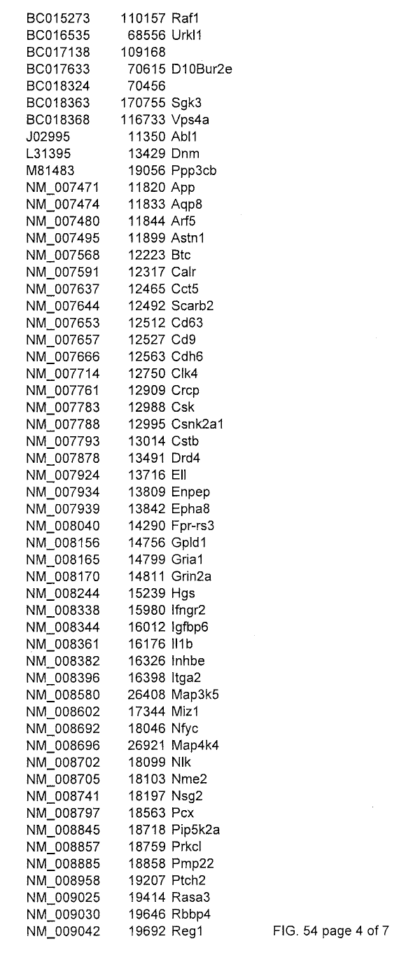

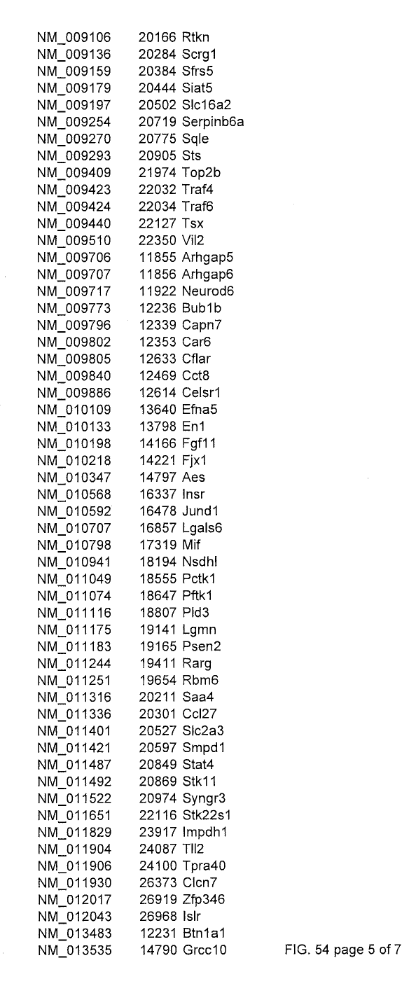

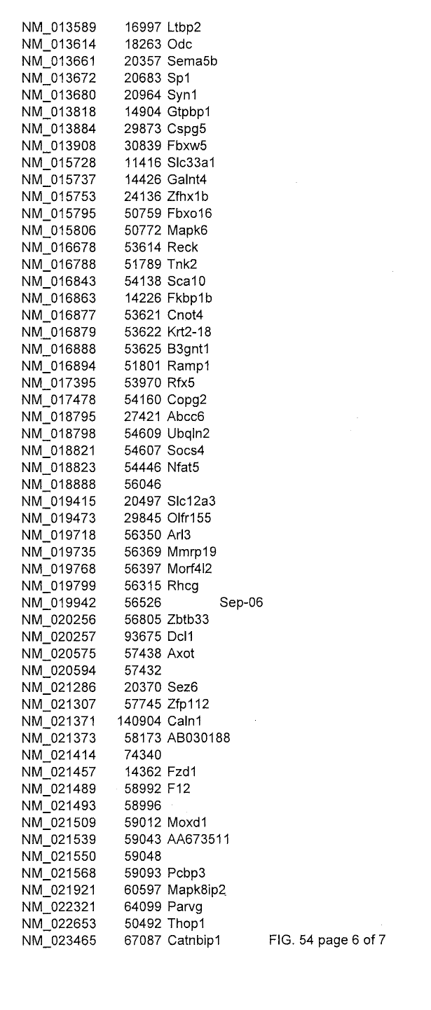

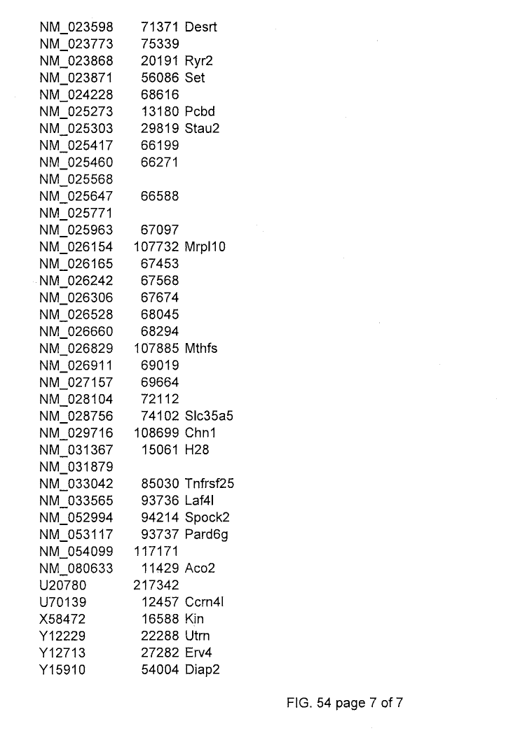

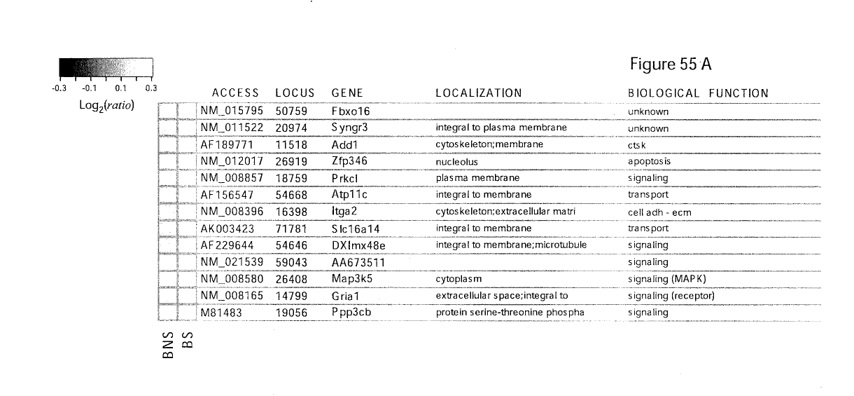

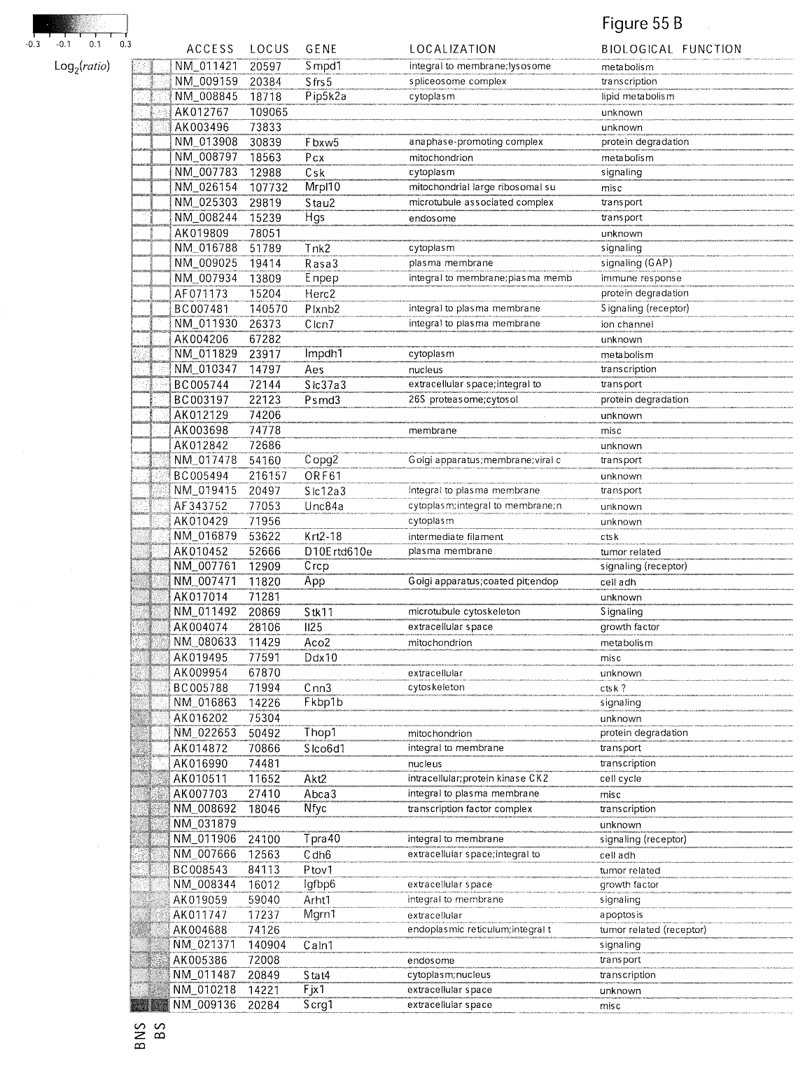

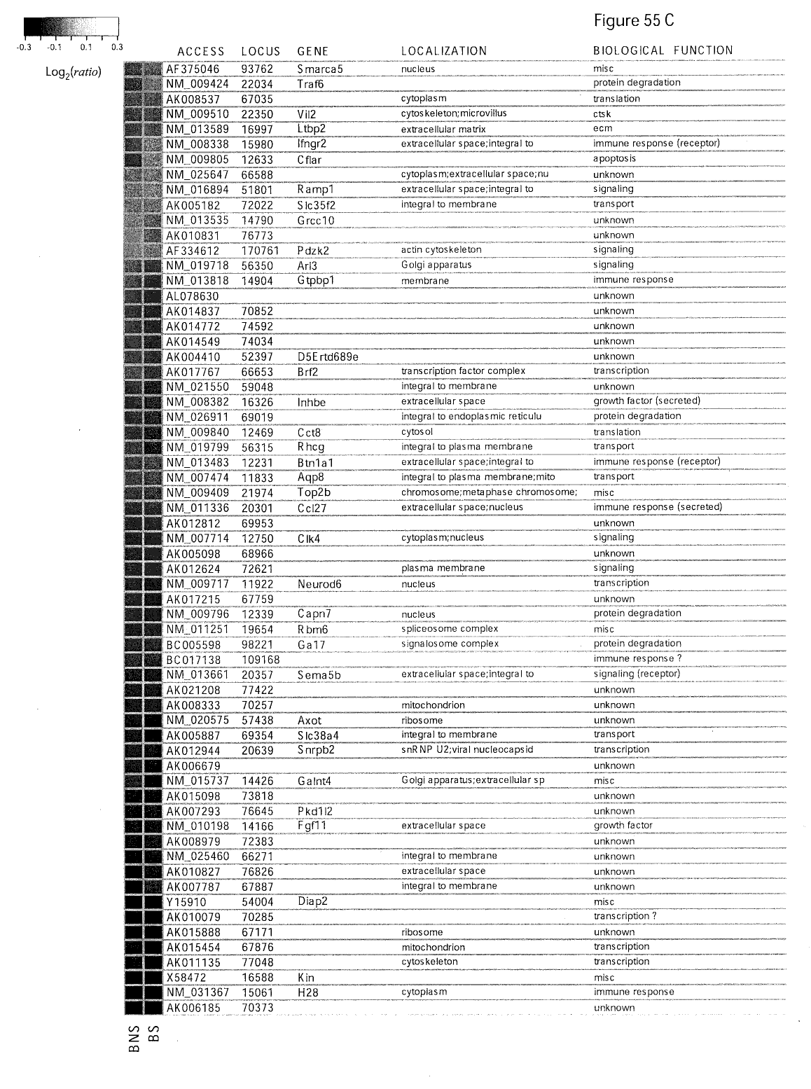

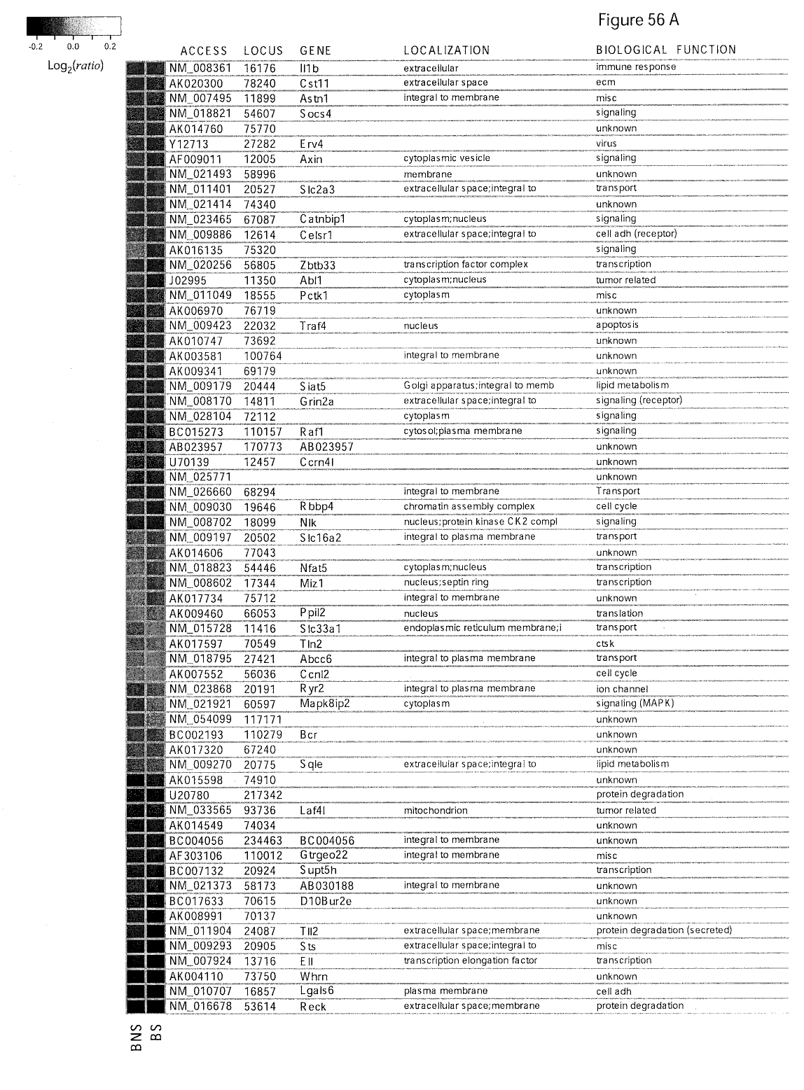

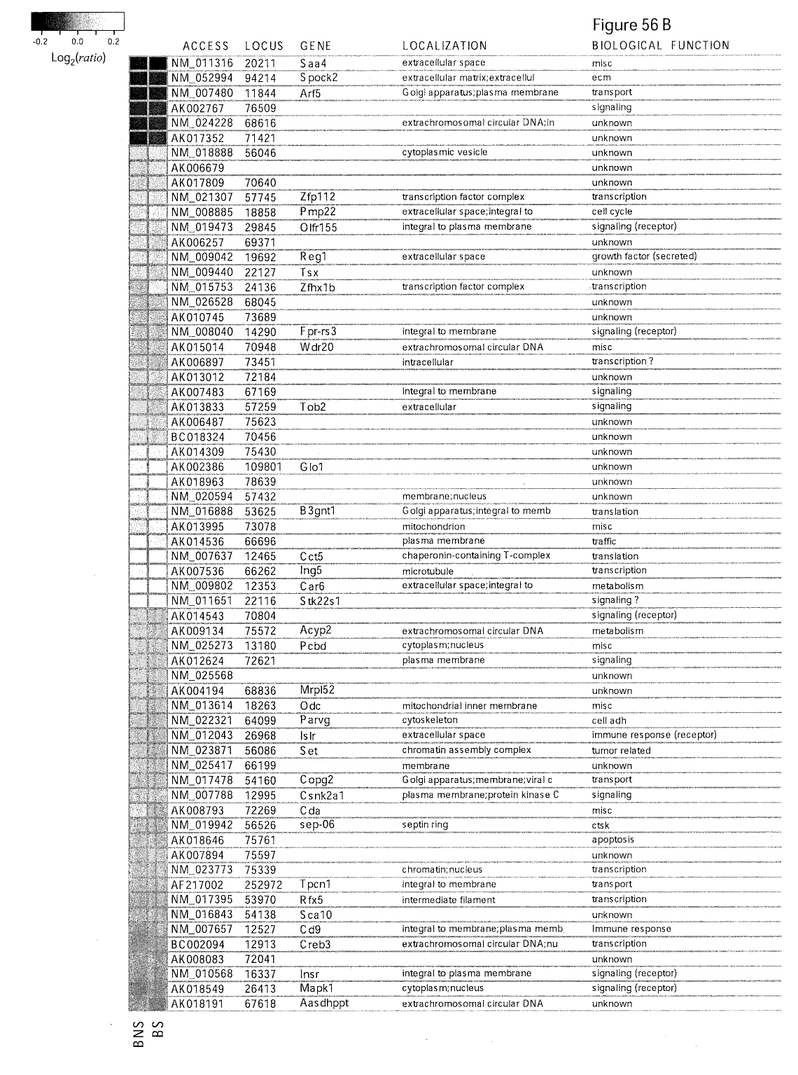

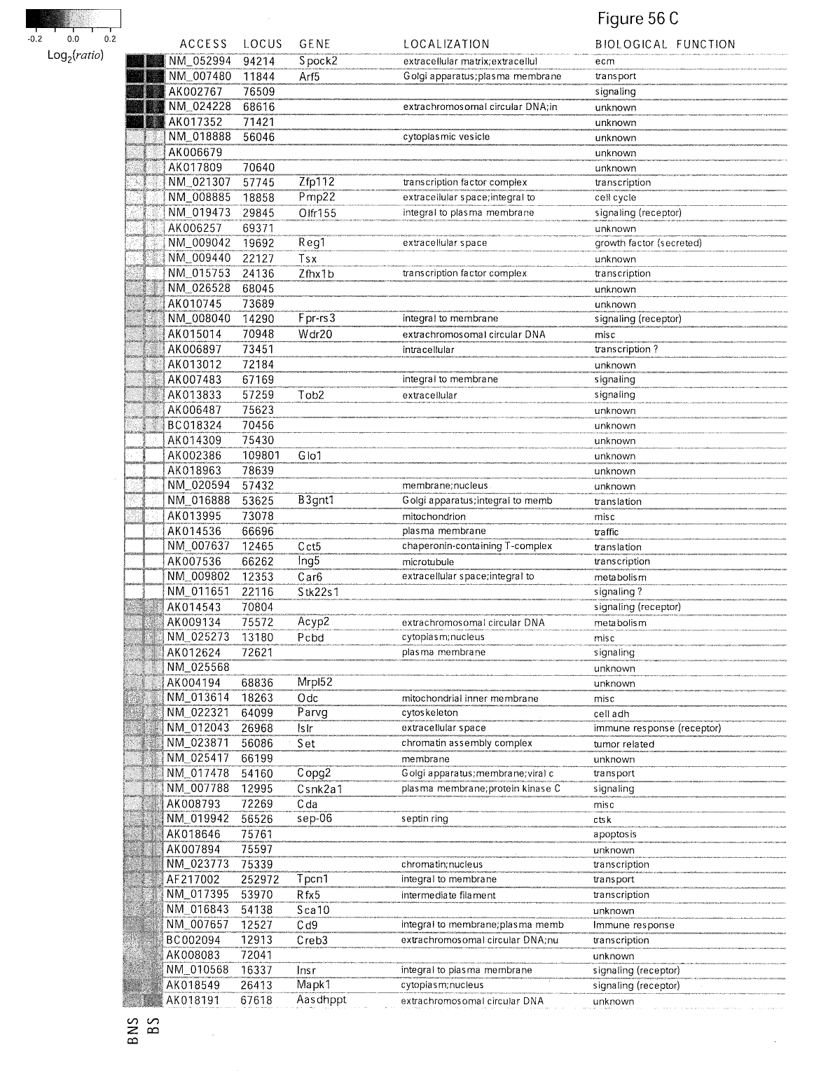

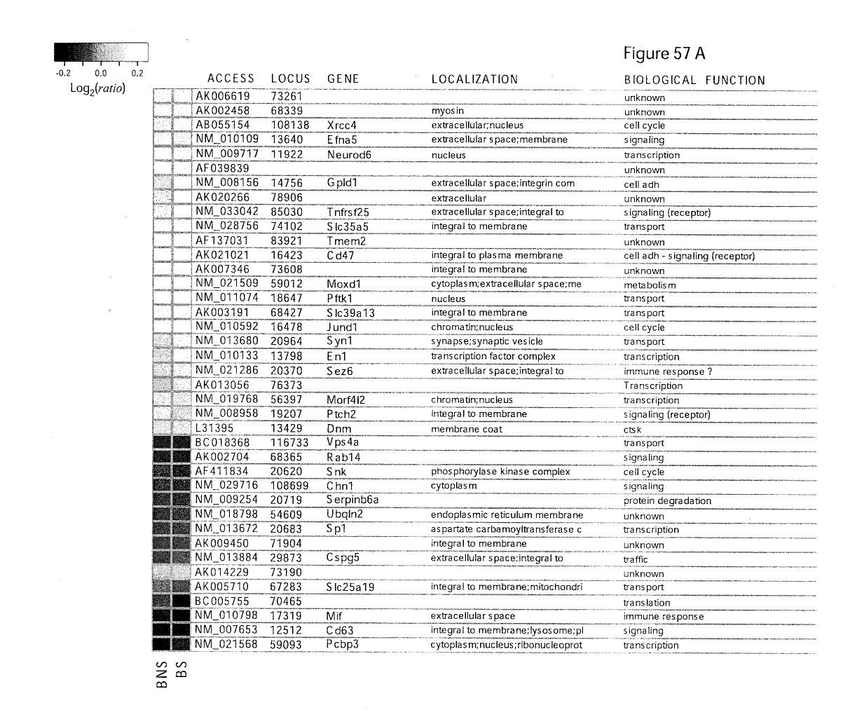

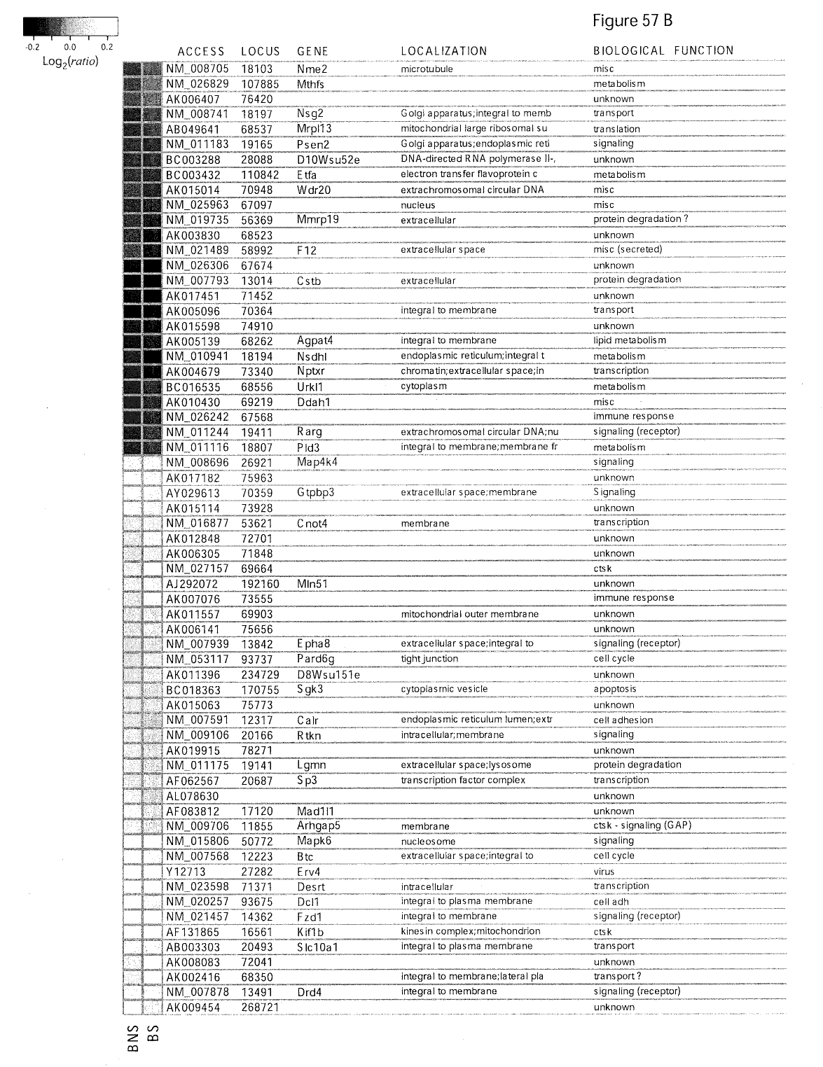

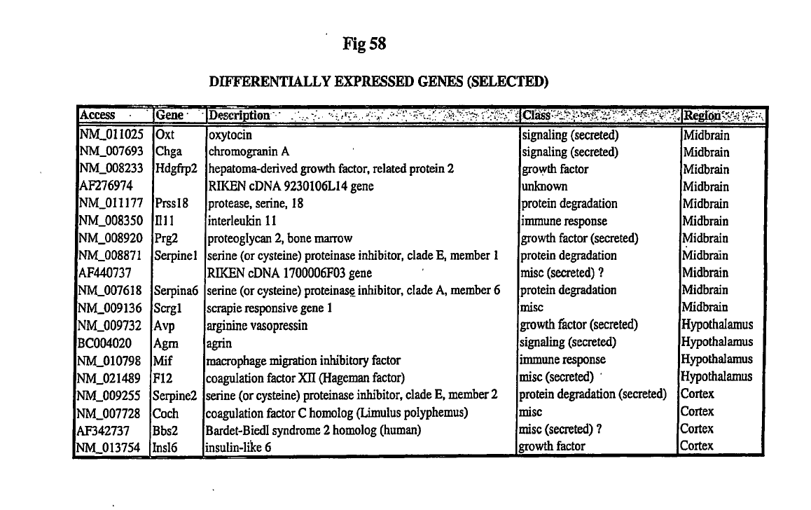

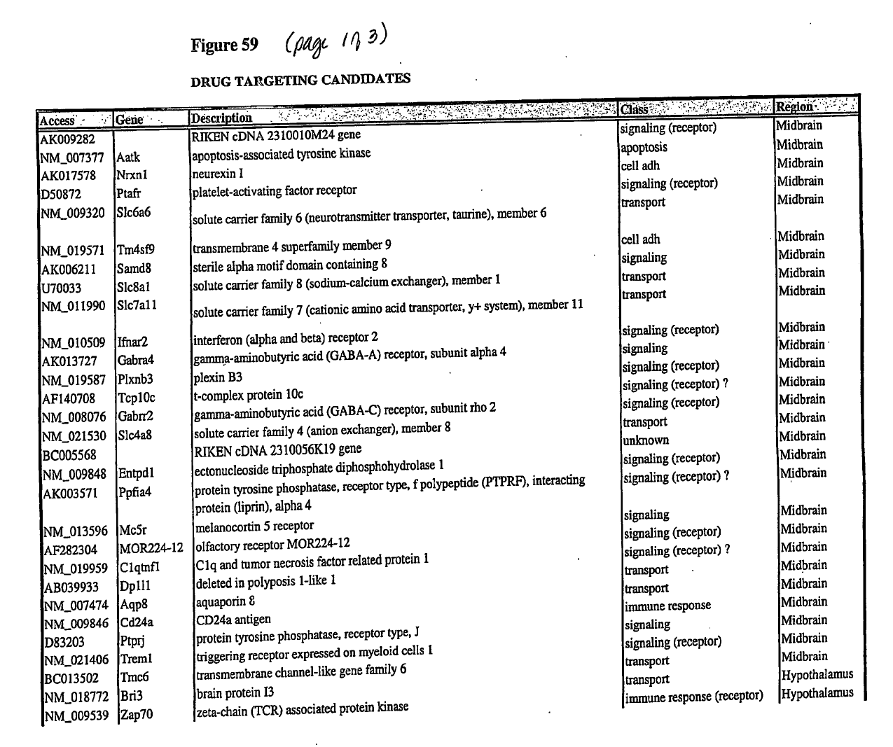

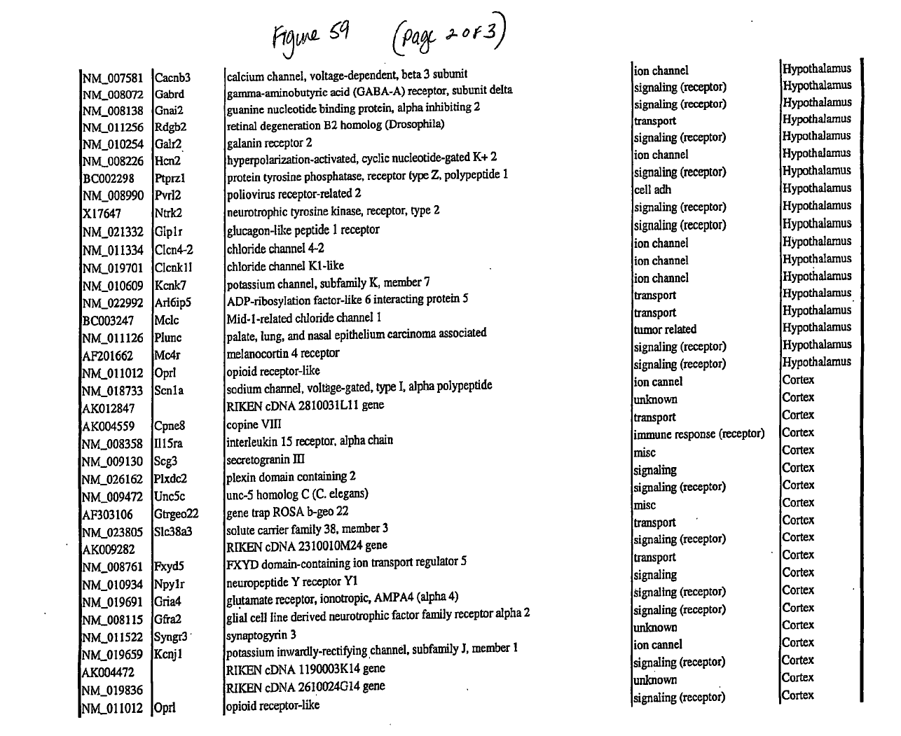

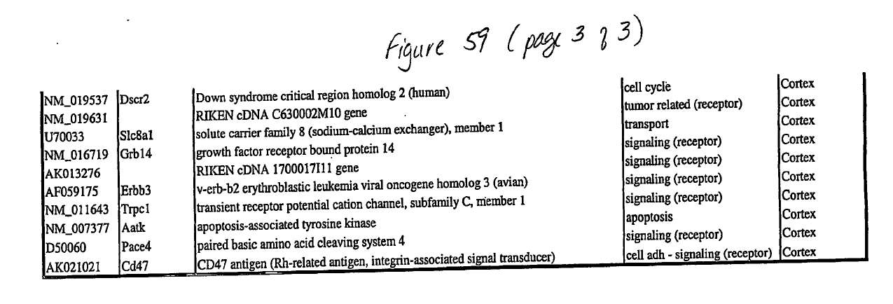

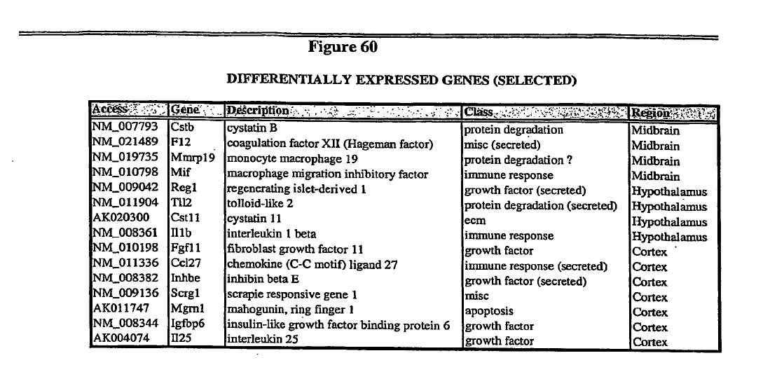

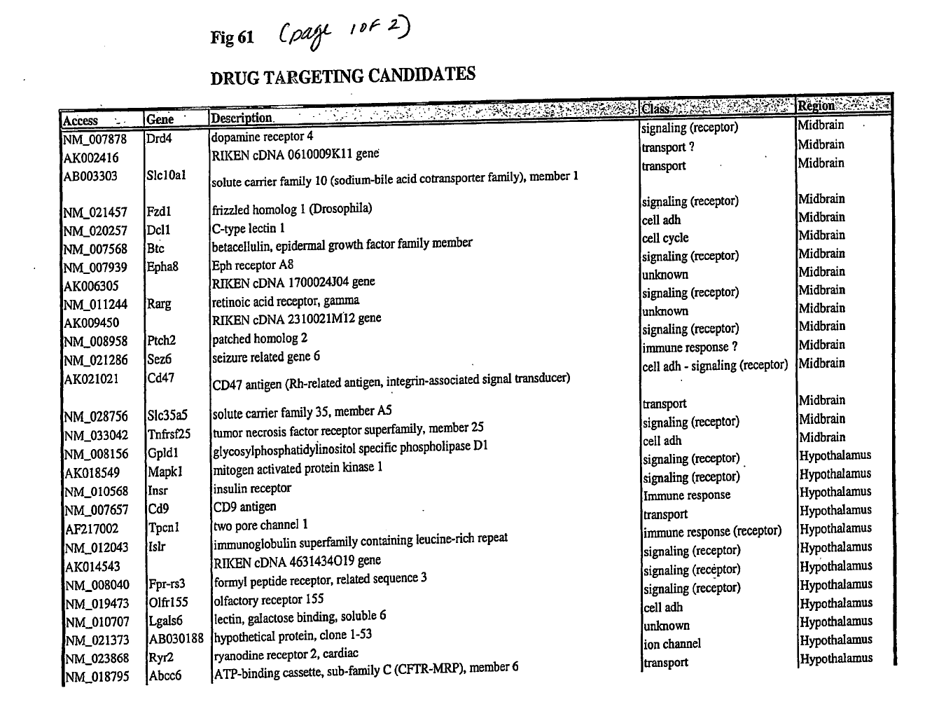

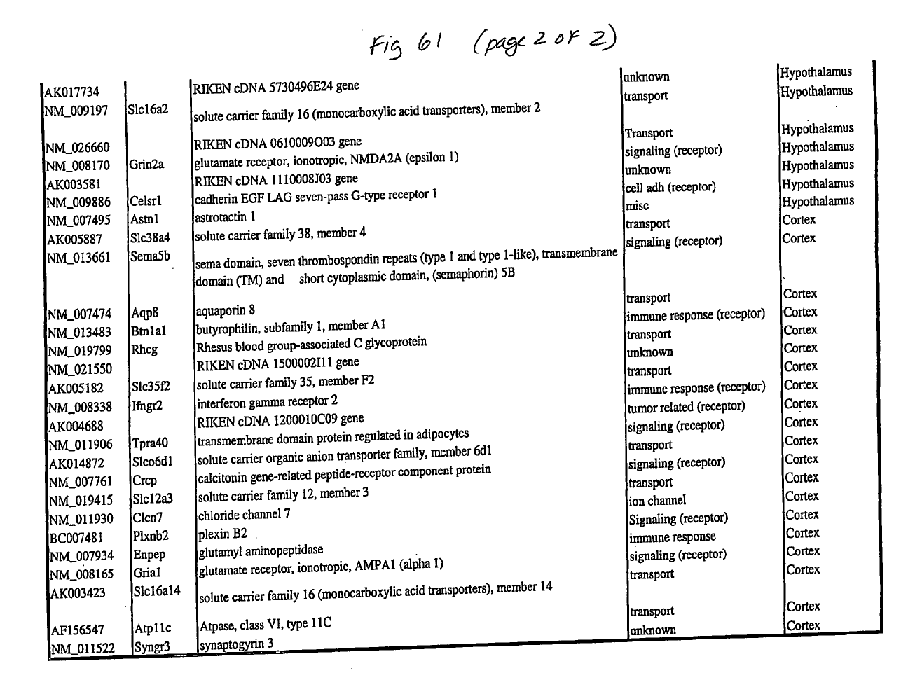

FIGS. 35A & 35B shows differentially expressed genes in mice harboring breast carcinoma after hierarchical cluster analysis. Samples were obtained from midbrain.

FIG. 35-1 shows down regulated genes in mice harboring breast carcinoma. Samples were obtained from midbrain. The list of genes corresponds to those that are down regulated at all the time points.

FIG. 35-2 shows down regulated genes in mice harboring breast carcinoma. Samples were obtained from midbrain. The list of genes corresponds to those that are down regulated at 18 hours, or at 18 hours and 72 hours post tumor injection.

FIG. 35-3 shows down regulated genes in mice harboring breast carcinoma. Samples were obtained from midbrain. The list of genes corresponds to those that are down regulated at 72 hours and 192 hours post tumor injection.

FIG. 35-4 shows up regulated genes in mice harboring breast carcinoma. Samples were obtained from midbrain. The list of genes corresponds to those that are up regulated at all the time points

FIG. 35-5 shows up regulated genes in mice harboring breast carcinoma. Samples were obtained from midbrain. The list of genes corresponds to those that are up regulated at 18 hours, or at 18 hours and 72 hours post tumor injection.

FIG. 35-6 shows up regulated genes in mice harboring breast carcinoma. Samples were obtained from midbrain. The list of genes corresponds to those that are up regulated at 72 hours and 192 hours post tumor injection.

FIGS. 36A & 36B shows differentially expressed genes in mice harboring colon carcinoma after hierarchical cluster analysis. Samples were obtained from midbrain.

FIG. 36-1 shows down regulated genes in mice harboring colon carcinoma. Samples were obtained from midbrain. The list of genes corresponds to those that are down regulated at all the time points.

FIG. 36-2 shows down regulated genes in mice harboring colon carcinoma. Samples were obtained from midbrain. The list of genes corresponds to those that are down regulated at 18 hours, or at 18 hours and 72 hours post tumor injection.

FIG. 36-3 shows down regulated genes in mice harboring colon carcinoma. Samples were obtained from midbrain. The list of genes corresponds to those that are down regulated at 72 hours and 192 hours post tumor injection.

FIG. 36-4 shows up regulated genes in mice harboring colon carcinoma. Samples were obtained from midbrain. The list of genes corresponds to those that are up regulated at all the time points.

FIG. 36-5 shows up regulated genes in mice harboring colon carcinoma. Samples were obtained from midbrain. The list of genes corresponds to those that are up regulated at 18 hours, or at 18 hours and 72 hours post tumor injection.

FIG. 36-6 shows up regulated genes in mice harboring colon carcinoma. Samples were obtained from midbrain. The list of genes corresponds to those that are up regulated at 72 hours and 192 hours post tumor injection.

FIGS. 37A & 37B shows differentially expressed genes in mice harboring lung carcinoma after hierarchical cluster analysis. Samples were obtained from midbrain.

FIG. 37-1 shows down regulated genes in mice harboring lung carcinoma. Samples were obtained from midbrain. The list of genes corresponds to those that are down regulated at all the time points.

FIG. 37-2 shows down regulated genes in mice harboring lung carcinoma. Samples were obtained from midbrain. The list of genes corresponds to those that are down regulated at 18 hours, or at 18 hours and 72 hours post tumor injection.

FIG. 37-3 shows down regulated genes in mice harboring lung carcinoma. Samples were obtained from midbrain. The list of genes corresponds to those that are down regulated at 72 hours and 192 hours post tumor injection.

FIG. 37-4 shows up regulated genes in mice harboring lung carcinoma. Samples were obtained from midbrain. The list of genes corresponds to those that are up regulated at all the time points.

FIG. 37-5 shows up regulated genes in mice harboring lung carcinoma. Samples were obtained from midbrain. The list of genes corresponds to those that are up regulated at 18 hours, or at 18 hours and 72 hours post tumor injection.

FIG. 37-6 shows up regulated genes in mice harboring lung carcinoma. Samples were obtained from midbrain at 18, 72 and 192 hours post tumor cell injection. The list of genes corresponds to those that are up regulated at 72 hours and 192 hours post tumor injection.

FIGS. 38A & 38B shows differentially expressed genes in mice harboring either breast, colon or lung carcinoma compared to control mice (p<0.01) after hierarchical cluster analysis. Samples were obtained from prefrontal cortex at 18, 72 and 192 hours post tumor cell injection. Differentially expressed genes were identified by a mixed model ANOVA, with tumor (or control), tumor model, and time points as fixed effects. Only data obtained without background subtraction was included in the table. The base 2 logarithm of tumor vs. control ratio is shown as a gray scale.

FIGS. 39A & 39B shows differentially expressed genes in mice harboring either breast, colon or lung carcinoma compared to control mice (p<0.01). Samples were obtained from hypothalamus at 18, 72 and 192 hours post tumor cell injection. Differentially expressed genes were identified by a mixed model ANOVA, with tumor (or control), tumor model, and time points as fixed effects. Only data obtained without background subtraction was included in the table. The base 2 logarithm of tumor vs. control ratio is shown as a gray scale.

FIGS. 40A & 40B shows differentially expressed genes in mice harboring either breast, colon or lung carcinoma compared to control mice (p<0.01). Samples were obtained from midbrain at 18, 72 and 192 hours post tumor cell injection. Differentially expressed genes were identified by a mixed model ANOVA, with tumor (or control), tumor model, and time points as fixed effects. Only data obtained without background subtraction was included in the table. The base 2 logarithm of tumor vs. control ratio is shown as a gray scale.

FIG. 41 (A) is a table showing down-regulated genes in mice harboring either breast, colon or lung carcinoma compared to control mice (p<0.01). Samples were obtained from prefrontal cortex at 18, 72 and 192 hours post tumor cell injection. Differentially expressed genes were identified by a mixed model ANOVA, with tumor (or control), tumor model, and time points as fixed effects. Only genes that showed a similar temporal pattern of expression in at least two cancer models were included in the table. Results correspond to data obtained without background subtraction. The base 2 logarithm of tumor vs. control ratio is shown as a gray scale. (B) Base 2 logarithm of tumor vs. control ratio for genes in (A). Bars are the mean±SEM.

FIG. 42 (A) is a table showing up-regulated genes in mice harboring either breast, colon or lung carcinoma compared to control mice (p<0.01). Samples were obtained from prefrontal cortex at 18, 72 and 192 hours post tumor cell injection. Differentially expressed genes were identified by a mixed model ANOVA, with tumor (or control), tumor model, and time points as fixed effects. Only genes that showed a similar temporal pattern of expression in at least two cancer models were included in the table. Results correspond to data obtained without background subtraction. The base 2 logarithm of tumor vs. control ratio is shown as a gray scale. (B) Base 2 logarithm of tumor vs. control ratio for genes in (A). Bars are the mean±SEM.

FIG. 43 (A) is a table showing down-regulated genes in mice harboring either breast, colon or lung carcinoma compared to control mice (p<0.01). Samples were obtained from hypothalamus at 18, 72 and 192 hours post tumor cell injection. Differentially expressed genes were identified by a mixed model ANOVA, with tumor (or control), tumor model, and time points as fixed effects. Only genes that showed a similar temporal pattern of expression in at least two cancer models were included in the table. Results correspond to data obtained without background subtraction. The base 2 logarithm of tumor vs. control ratio is shown as a gray scale. (B) Base 2 logarithm of tumor vs. control ratio for genes in (A). Bars are the mean±SEM.

FIG. 44 (A) is a table showing up-regulated genes in mice harboring either breast, colon or lung carcinoma compared to control mice (p<0.01). Samples were obtained from hypothalamus at 18, 72 and 192 hours post tumor cell injection. Differentially expressed genes were identified by a mixed model ANOVA, with tumor (or control), tumor model, and time points as fixed effects. Only genes that showed a similar temporal pattern of expression in at least two cancer models were included in the table. Results correspond to data obtained without background subtraction. The base 2 logarithm of tumor vs. control ratio is shown as a gray scale. (B) Base 2 logarithm of tumor vs. control ratio for genes in (A). Bars are the mean±SEM.

FIG. 45 (A) is a table showing down-regulated genes in mice harboring either breast, colon or lung carcinoma compared to control mice (p<0.01). Samples were obtained from midbrain at 18, 72 and 192 hours post tumor cell injection. Differentially expressed genes were identified by a mixed model ANOVA, with tumor (or control), tumor model, and time points as fixed effects. Only genes that showed a similar temporal pattern of expression in at least two cancer models were included in the table. Results correspond to data obtained without background subtraction. The base 2 logarithm of tumor vs. control ratio is shown as a gray scale. (B) Base 2 logarithm of tumor vs. control ratio for genes in (A). Bars are the mean±SEM.

FIG. 46 (A) is a table showing up-regulated genes in mice harboring either breast, colon or lung carcinoma compared to control mice (p<0.01). Samples were obtained from midbrain at 18, 72 and 192 hours post tumor cell injection. Differentially expressed genes were identified by a mixed model ANOVA, with tumor (or control), tumor model, and time points as fixed effects. Only genes that showed a similar temporal pattern of expression in at least two cancer models were included in the table. Results correspond to data obtained without background subtraction. The base 2 logarithm of tumor vs. control ratio is shown as a gray scale. (B) Base 2 logarithm of tumor vs. control ratio for genes in (A). Bars are the mean±SEM.

FIG. 47 (A)-(C) is a set of tables listing tumor-specific CNS markers differentially expressed, at any time tested, in three different cancer models: breast cancer, 47A; colon cancer, 47B; and lung cancer, 47C. Criteria for inclusion in this figure were (1) the marker corresponds to a secreted product; and (2) a p value below 0.01 for differential expression.

FIG. 48 (A)-(C) is a set of tables listing genes identified as CNS markers that are also potential targets for therapeutic intervention for each of breast, colon and lung cancer. Criteria for inclusion in this figure were (1) the marker corresponds to a signaling receptor such as a growth factor, hormone, or cytokine; and (2) a p value for differential expression below 0.01

FIG. 49 is a table listing differentially expressed genes (p<0.05) chosen at random for validation. 4 out of 14 (29%) were validated as differentially expressed genes by real time PCR indicating a good level of correlation between microarray and Real Time PCR according to Wurmbach et al., Methods 2003, 31: 306-316. Ratios are expressed as mean±SEM. (ND) No data available. P-value ranks were calculated sorting the genes of microarray results according to their p-values in ascending order.

FIG. 50 is a table showing all the arthritis disease surveillance genes (differentially expressed at p<0.05) identified in prefrontal cortex, hypothalamus and midbrain of relevant animal models. Data corresponds to genes differentially expressed in arthritic mice compared to control mice. Samples were obtained 24 days after the last LPS injection, when animals started to show arthritic symptoms.

FIGS. 51A, 51B, 51C & 51D are tables showing the differentially expressed genes (p<0.05) in prefrontal cortex of arthritic mice. Samples were obtained 24 days after the last lipopolysaccharide injection, when animals started to show arthritic symptoms. Differentially expressed genes were identified by paired samples t-test. The base 2 logarithm of arthritic vs. control ratio is shown as a gray scale. BNS corresponds to data obtained without background subtraction, and BS corresponds to data obtained after background subtraction.

FIGS. 52A, 52B, 52C & 52D are tables showing the differentially expressed genes (p<0.05) in hypothalamus of arthritic mice. Samples were obtained 24 days after the last lipopolysaccharide injection, when animals started to show arthritic symptoms. Differentially expressed genes were identified by paired samples t-test. The base 2 logarithm of arthritic vs. control ratio is shown as a gray scale. BNS corresponds to data obtained without background subtraction, and BS corresponds to data obtained after background subtraction.

FIGS. 53A, 53B, 53C & 53D are tables showing the differentially expressed genes (p<0.05) in midbrain of arthritic mice. Samples were obtained 24 days after the last lipopolysaccharide injection, when animals started to show arthritic symptoms. Differentially expressed genes were identified by paired samples t-test. The base 2 logarithm of arthritic vs. control ratio is shown as a gray scale. BNS corresponds to data obtained without background subtraction, and BS corresponds to data obtained after background subtraction.

FIG. 54 is a table showing all the Asthma disease surveillance genes (differentially expressed at p<0.05) identified in prefrontal cortex, hypothalamus and midbrain of relevant animal models. Data corresponds to genes differentially expressed in asthmatic mice compared to control mice. Samples were obtained two days after the last aerosol ovalbumin exposure.

FIGS. 55A, 55B & 55C are tables showing the differentially expressed genes (p<0.05) in prefrontal cortex of asthmatic mice. Samples were obtained two days after the last aerosol ovalbumin exposure. Differentially expressed genes were identified by paired samples t-test. The base 2 logarithm of asthmatic vs. control ratio is shown as a gray scale. BNS corresponds to data obtained without background subtraction, and BS corresponds to data obtained after background subtraction.

FIGS. 56A & 56B are tables showing the differentially expressed genes (p<0.05) in hypothalamus of asthmatic mice. Samples were obtained two days after the last aerosol ovalbumin exposure. Differentially expressed genes were identified by paired samples t-test. The base 2 logarithm of asthmatic vs. control ratio is shown as a gray scale. BNS corresponds to data obtained without background subtraction, and BS corresponds to data obtained after background subtraction.

FIGS. 57A & 57B are tables showing the differentially expressed genes (p<0.05) in midbrain of asthmatic mice. Samples were obtained two days after the last aerosol ovalbumin exposure. Differentially expressed genes were identified by paired samples t-test. The base 2 logarithm of asthmatic vs. control ratio is shown as a gray scale. BNS corresponds to data obtained without background subtraction, and BS corresponds to data obtained after background subtraction.

FIG. 58 is a table listing arthritis specific CNS markers differentially expressed, at the time tested. Criteria for inclusion in this figure were (1) the marker corresponds to a secreted product; and (2) a p value below 0.05 for differential expression.

FIG. 59 is a table listing genes identified as CNS markers that are also potential targets for therapeutic intervention for arthritis. Criteria for inclusion in this figure were (1) the marker corresponds to a signaling receptor such as a growth factor, hormone, or cytokine; and (2) a p value for differential expression below 0.05.

FIG. 60 is a table listing asthma specific CNS markers differentially expressed, at the time tested. Criteria for inclusion in this figure were (1) the marker corresponds to a secreted product; and (2) a p value below 0.05 for differential expression.

FIG. 61 is a table listing genes identified as CNS markers that are also potential targets for therapeutic intervention for asthma. Criteria for inclusion in this figure were (1) the marker corresponds to a signaling receptor such as a growth factor, hormone, or cytokine; and (2) a p value for differential expression below 0.05

### DETAILED DESCRIPTION

The methods described herein rely, in part, on the detection of gene expression in the CNS to identify (e.g., diagnose or monitor) peripheral (non-CNS) tissues or organs for early stages of disease (e.g., in some cases, within hours, days, weeks or months of the appearance of disease). Early identification and/or diagnosis of disease provides an opportunity for early therapeutic intervention to target the disorder before it becomes overly advanced or aggressive.

### General Methodology

The CNS is involved in the body's response to any internal or external stimulus that by its intensity or functional relevance could alter internal homeostasis. As part of this function, the CNS and the immune system interact to obtain a suitable immune response when necessary.

An immune response impacts the brain via neural and humoral mechanisms. Neural mechanisms primarily involve the activation of the vagal nerve. Humoral mechanisms can include cytokine-mediated action directly on brain structures, e.g., cytokine-mediated increases on neural firing rates (Rothwell and Hopkins, 1995, Trends Neurosci 18(3):130-6; Wang et al., 2003, Nature, 421(6921):384-8). In one example, peripheral cytokines have been shown to bind and activate the vagal nerve, which in turn activates neurons of the nucleus of the tractus solitarius and the hypothalamus in the brain. (Watkins and Maier, 1999, Proc. Natl. Acad. Sci. USA, 96(14):7710-3).

Humoral signals from the periphery act as potent messengers to the brain. Cytokines in the brain can exert their action at a much lower dose than in the periphery. For example, intracerebral administration of interleukin-1 (IL-1) at a dose of 100 pg to 10 ng elicits maximal changes in fever, gastric function, increased metabolism and behavioral changes, while several micrograms of this cytokine are necessary to elicit similar responses when administered to the periphery (Rothwell and Hopkins, supra).

After sensing an internal immune signal, the brain reacts in different ways. A paradigm of CNS response to immune signals is the activation of neuroendocrine axes (I such as the hypothalamus-pituitary-adrenal axis. The activation of this axis results in the liberation of glucocorticoids, which in turn can modulate the ongoing immune response in under 10 minutes. Vagatomy has been shown to blunt the activation of the hypothalamus pituitary adrenal axis after intraperitoneal administration of cytokines (Watkins and Maier, supra). This feedback mechanism is of high physiological relevance; i.e., inhibition of glucocorticoid production after cytokine release in the periphery usually results in the death of the organism (Besedovsky and del Rey, 1996; Endocr. Rev., 17(1):64-102).

The brain can also sense signals that will affect the immune and other systems from the external milieu. For example, the triggering of a stress reaction can result in the release of glucocorticoids and the attenuation of an ongoing immune response. The effects of stress on the immune system are well documented in animal models and humans (Deinzer et al., 2000, Int. J. Psychophysiol., 37(3):219-32; Marshall et al., 1998, Brain Behav. Immun., 12(4):297-307; Benschop et al., 1996, FASEB J., 10(4):517-24; Sheridan et al., 1998, Ann. N.Y. Acad. Sci., 840:803-8). In addition, there is anecdotal and preliminary evidence that mind/body interventions such as meditation or yoga could have an influence on the immune system (Cassileth, 1999, CA Cancer J. Clin., 49(6):362-75).

The new methods harness this natural reaction of the CNS as a way to detect peripheral disease at an early stage. While not limited by any theory, the methods described herein are based, in part, on the discovery that the CNS senses the presence of “alarm signals” from peripheral (non-CNS) disorders at an early stage in the development of disease progression. Thus, the methods described herein relate to diagnosing peripheral disorders by detecting gene expression in the CNS, e.g., in a CNS sample from a subject, such as a human, or from any other bodily fluid where CNS gene products or derivatives thereof could be detected. In one aspect, a non-CNS disorder can be identified based on a profile of gene expression in the CNS (e.g., the brain) within hours, weeks or months after disease progression is initiated in the body. In some embodiments, a non-CNS disorder can be identified based on a profile of gene expression in the CNS (e.g., the brain) within one or more years (e.g., 2, 3, 5, 7, 10 or more years) after disease progression is initiated in the body, but before a disorder is clinically detectable and/or in an advanced stage.

### Cancer Development

It is generally accepted that a clinically detectable tumor mass is composed of cells that, although abnormal, evade immune surveillance and resist immune system attack. During the time of neoplastic progression, cells are characterized by high mutation rates, reflected, inter alia, in phenotypic changes such as down-regulation of histocompatibility antigens. A tumor may thus become resistant to a particular therapeutic by clonal selection and proliferation from the tumor mass of a cell clone having a mutation that allows the cell to resist the given therapeutic. The “natural selection” of tumor cell clones occurs at a given rate leading to the appearance of malignant cells having genetic and epigenetic traits that facilitate growth and escape from the immune system. It is estimated that the average malignancy contains more than 10,000 mutations (Stoler et al., 1999, Proc. Natl. Acad. Sci., USA., 96(26):15121-6). Therefore, it can be concluded that the antigen profile of established cancers by no means reflects the cell genotype and phenotype of very early stage neoplasia. Moreover, it is reasonable to assume that tumor antigens present in the established cancer and the response they can induce in the organism will be different than the antigens and responses induced by early stage neoplastic cells. The new methods described herein can detect such early stage neoplastic cells in spite of these obstacles.

Some neoplasms, e.g., some cancers (e.g., certain types of carcinoma) can grow for long periods (e.g., for 1, 2, 5, 10, 15, 20 or 25 years) before they are clinically detectable using prior known technology and/or before they become malignant. This period provides an extraordinary window of opportunity for detection of cancerous cells before the malignant tumor is clinically detectable by current strategies. During this period tumor cells undergo several modifications at the molecular level as a result of their genomic instability.

Each genetic change is potentially selective for proliferation and/or is capable of triggering a new “alarm signal” to recruit and activate local innate and adaptive immune responses. In a simple view, 10,000 alarm signals are produced during the 10 to 15 years of tumor development before the tumor is clinically detectable.

### Development of Rheumatoid Arthritis

Rheumatoid arthritis (RA) is an acquired autoimmune disease in which genetic factors appear to play a role. RA occurs in 1-2 percent of the general population and is found world-wide. Females with RA outnumber males by 3:1. Onset of the disease in adults is usually between the ages of 40 to 60 years, although it can occur at any age.

RA involves Th1 lymphocytes and macrophage infiltration into joints as well as the presence of rheumatoid factors in patients' serum (Chemajovsky et al., 2000, Genes Immun., 1:295-307). Degradation of cartilage is accompanied by the outgrowth of synovial membrane (pannus). This process is generally regulated by IL-1 and TNF-α, while TGF-β and IL-10 counteract this effect (Chemajovsky et al., ibid). Susceptibility to arthritis has been correlated with MHC class II locus, in particular HLA-DR4 in 70 percent of patients with RA (Chemajovsky et al., ibid). Rheumatoid Factor(s) (RF) are antibodies to IgG, and are present in 60-80 percent of adults with the disease. High titers of RF are usually associated with more severe and active joint disease, greater systemic involvement, and a poorer prognosis for remission.

An unknown antigen is thought to initiate the autoimmune response resulting in RA. It has been suggested that there is a synovial antigen resembling a bacterial lipopolysaccharide (LPS) of arthritogenetic bacteria that initiates the autoimmune response (Kennedy, 2000, Med. Hypotheses, 54(5):723-5). TNF-α appears to be the driving force behind the chronic inflammation characteristic of RA. TNF-α plays also an important role in B cell maturation which appears to participate in disease progression (Chemajovsky et al., ibid). Some data also strongly indicate a role for Suppressor of Cytokine signaling (SOCS) in disease outcome (Egan et al., 2003, J. Clin. Invest. 111(6):915-24).

The initiation of the autoimmune response and/or the initiation of the inflammatory mechanisms in the early development of RA trigger signals detected by changes in gene expression in the CNS.

### Development of Asthma

Asthma is an inflammatory airway disease characterized by the presence of cells such as eosinophils, mast cells, basophils, and CD25+ T lymphocytes in the airway walls. Chemokines attract cells to the site of inflammation and cytokines (Interleukin (IL)-4, IL-5, IL-10 and IL-13) activate them, resulting in inflammation and damage to the mucosa. When asthma becomes chronic, secondary changes occur, such as thickening of basement membrane and fibrosis. IL-4 and other cytokines such as TGF-β may be involved in tissue remodeling and the fibrotic response.

In allergic asthma (also known as extrinsic asthma), the initiation event of airway inflammation is an immunological reaction to allergen. Continued exposure to allergen results in chronic inflammation. Allergic asthma affects about 3 million children (8 to 12 percent of all children) and 7 million adults in the United States at a cost estimated at $6.2 billion a year. It has been suggested that longitudinal studies based on yet unidentified inflammatory markers will guide asthma management in the future (Wilson, 2002, Curr. Opin. Pulm. Med., 8(1):25-32).

In the development of asthma, the initiation of the allergic or inflammatory response, e.g., release of cytokines and/or chemokines, can trigger signals detected by changes in gene expression in the CNS.

### Development of Obesity

Body size and body weight are highly heritable traits. Association studies performed with populations of monozygotic and dizygotic twins, non-twin siblings and adoptive family members indicated that the variance for body mass index (body weight divided by height to the square) is much lower in identical twins that in any other group, indicating that genetic factors rather than environmental effects are the key determinant of human adiposity (Maes et al., 1997, Behav. Genet., 27:325-351; Allison et al., 1996, Int. J. Obes. Relat. Metab. Disord., 20:501-506). Diet-induced obesity is also highly heritable. A pioneer study performed in 12 pairs of young adult identical twins overfed by 1,000 kcal per day during a 100-day period demonstrated that overfeeding induced a variable increase in body weight in all volunteers. However, twin pairs had six times less variance in mass increase than non-twin pairs, indicating that adaptation to long-term overfeeding has important genetic factors (Bouchard et al., 1990, N. Engl. J. Med., 322:1477-1482). The strong genetic predisposition to gain weight after ingesting a fat-rich diet is even more clearly observed in the laboratory when testing mice or rats of different genetic backgrounds (Schaffhauser et al., 2002, Obes. Res., 10:1188-1196). Most strains of mice maintain their body weight throughout relatively long periods of time while being fed ad libitum with low fat diets. However, when fed ad libitum with a high fat diet, some strains develop a considerable increase in body mass and some other strains are resistant to this increase regardless of increase in food consumption (West et al., 1995, Am. J. Physiol., 268:R658-R665; Prpic et al., 2003, Endocrinology, 144:1155-1163).

The regulation of body weight involves a large number of interconnected peripheral and brain circuits that participate in the control of energy balance throughout the entire organism (Spiegelman and Flier, 2001, Cell, 104:531-43). Information about the amount of energy stored in the whole body is transported into the brain by peripheral hormones such as leptin and insulin. The relative variation of the plasma concentration of these hormones is interpreted by central mechanisms to induce signals of appetite or satiety (Friedman and Halaas, 1998, Nature, 395:763-70). Other molecules such as ghrelin and cholecystokinin (CCK) enter into the brain after being released from different portions of the gastrointestinal tract and provide essential information to brain centers about the nutritional status of the organism (Murakami et al., 2002, J. Endocrinol., 174:283-288; Sheng and Moran, 2002, Neuropeptides, 36:171-181).

The hypothalamus, a critical brain area for the complicated control of energy homeostasis, integrates a variety of converging signals within a short time frame. In the ventral hypothalamus a group of appetite-inducing neurons expresses the neuropeptide Y (NPY) gene. As leptin levels drop from circulation NPY is released into the paraventricular nucleus of the hypothalamus to induce food intake (Widdowson et al., 1999, Peptides, 20:367-372). A single intracerebroventricular administration of NPY in mice or rats can dramatically increase food intake for many hours (Zarjevski et al., 1993, Endocrinology, 133:1753-1758). Conversely, another group of neurons located in the arcuate nucleus of the hypothalamus expresses the proopiomelanocortin gene (POMC). These neurons also express the leptin receptor gene. After an excessive intake of fat-enriched food, the levels of triglycerides rise, filling peripheral adipocytes with fat stores. This leads to an increase in production of leptin, which is released into the circulation and eventually enters the brain by a selective uptake mechanism (Hileman et al., 2002, Endocrinology, 143:775-783). Leptin stimulates leptin receptors located in POMC neurons, thereby increasing their firing activity (Cowley et al., 2001, Nature, 411:480-484).

One of the active peptides produced by the POMC precursor is α-melanocyte stimulating hormone (α-MSH). Upon stimulation of leptin receptors, α-MSH is released in the paraventricular nucleus of the hypothalamus to induce satiety. Intracerebroventricular injections of α-MSH in mice or rats induce long lasting anorexia that can promote the death of the animals if they are not forced to feed (Fan et al., 1997, Nature, 385:165-168).

The hormones, neuropeptides and their receptors described above are only a few examples of the many gene products that participate in the central control of energy balance. Regulation of a molecule involved in energy control (e.g., a disruption associated with propensity or presence of obesity) can likely trigger signals that result in changes in gene expression in the CNS.

### Methods of Detecting Gene Expression

Gene expression in the CNS can be detected in vitro, e.g., in an isolated CNS sample, or in vivo, e.g., using in vivo imaging techniques.

Central Nervous System (CNS) Samples

The CNS refers to the brain (including the cranial nerves) and spinal cord. A CNS sample can be, e.g., a cell or tissue from the brain or spinal cord, or a sample of the cerebrospinal fluid (CSF) that fills the ventricles of the brain and the central canal of the spinal cord.

Where the detection of gene expression is to be done in a CNS sample isolated from the subject, a CNS sample can be obtained by any number of methods available to the skilled artisan. For example, a CNS cell or tissue sample can be obtained from the brain, e.g., by needle biopsy or by open surgical incision. Imaging of the brain can be performed to determine the precise positioning of the needle or scalpel to enter the brain.

In one example, known as stereotactic biopsy, a tiny hole is drilled into the skull with the patient under light sedation or general anesthesia, and a needle is inserted into the brain tissue guided by computer-assisted imaging techniques such as computerized tomography (CT) or magnetic resonance imaging (MRI) scans. The needle is used to remove a sample of cells, whose gene expression can then be detected by a routine assay, e.g., a gene expression assay described herein. In another example, a sample of CSF can be obtained by routine methods, such as by lumbar puncture. This procedure can be done on an outpatient basis, e.g., under local anesthetic.

The number of cells or amount of CSF needed to perform a particular gene expression assay on a CNS sample will vary; however, some techniques, such as PCR based techniques, will require a very small number of cells, e.g., as few as 10 to 100 cells (Klein et al., Nat. Biotechnol., 20(4):387-92, 2002). The CNS sample can be used immediately in a diagnostic test described herein, or it can be stored, e.g., cooled or frozen, and/or transported to a facility where the diagnostic test is performed.

Nucleic Acid-Based Methods

In one embodiment, the methods described herein will utilize techniques for detection of gene expression where a polynucleotide (such as an RNA, mRNA, DNA, cDNA, or other nucleic acid corresponding to the gene) is detected. It should be understood by the skilled artisan that many methods for nucleic-acid based detection of gene expression exist and that any suitable method for detection can be used. Typical assay formats utilize nucleic acid hybridization and include, e.g., 1) nuclear run-on assay, 2) slot blot assay, 3) northern blot assay, 4) magnetic particle separation, 5) nucleic acid or DNA arrays or chips (also discussed in more detail below), 6) reverse northern blot assay; 7) dot blot assay, 8) in situ hybridization, 9) RNase protection assay, 10) ligase chain reaction, 11) polymerase chain reaction (PCR), 12) reverse transcriptase (RT)-PCR, and 13) differential display RT-PCR (DDRT-PCR) or any combination of any two or more of these methods. Such assays can employ the use of detectable labels such as radioactive labels, enzyme labels, chemiluminescent labels, fluorescent labels, or other suitable labels, to detect, identify, or monitor the presence or level of a particular nucleic acid being detected. Such techniques and labels are known in the art and widely available to the skilled artisan.

In one embodiment, an RNase protection assay can be utilized in the methods described herein by hybridizing multiple DNA probes corresponding to one or more members of a panel of sequences to mRNA isolated from a CNS sample from a subject to be tested. The expression profile for one or more genes from the CNS sample can be compared to a reference gene expression profile, e.g., a basal pattern of expression, or other negative or positive control (e.g., a profile from a patient known to have no peripheral disease, or a standard or average profile derived from subjects known to not have the particular disorder being tested). In one example, the gene expression profile from the test CNS sample is compared to a reference gene expression profile that is associated with the presence of a non-CNS neoplasia. If the test gene expression profile matches the reference gene expression profile, it indicates that the subject has, or is at risk for developing, the non-CNS neoplastic disorder. As used herein, “matches” means that at least 75% of the genes in a test gene expression profile are either up- or down-regulated in the same manner as the genes in the reference expression profile.

The methods described herein are also well suited for polymerase chain reaction (PCR)-based methods. PCR-based methods include RT-PCR (U.S. Pat. No. 4,683,202), ligase chain reaction (Barany, Proc. Natl. Acad. Sci. USA, 88:189-193, 1991), self-sustained sequence replication (Guatelli et al., Proc. Natl. Acad. Sci. USA, 87:1874-1878, 1990), transcriptional amplification system (Kwoh et al., Proc. Natl. Acad. Sci. USA, 86:1173-1177, 1989), Q-Beta Replicase (Lizardi et al., BioTechnology, 6:1197, 1988), rolling circle replication (Lizardi et al., U.S. Pat. No. 5,854,033), or any other nucleic acid amplification method, followed by the detection of the amplified molecules using techniques known in the art. PCR amplification of mRNAs expressed in a CNS sample can be performed directly from mRNA isolated from the sample, or from cDNA reverse-transcribed from such isolated mRNA. The amplified nucleic acid can then be hybridized to a particular probe of interest, e.g., a probe for a CNS gene as described herein, to determine its expression. The probe can be disposed on an address of an array, e.g., an array described herein. Such methods are routine and are particularly amendable to routine adaptation to automated systems employing computer controlled reagent aliquoting and signal detection. See, e.g., Klein et al., Nat. Biotechnol., 2002, 20(4):387-92.

In another embodiment, in situ methods are used to detect the presence or level of mRNA corresponding to a particular gene. In such methods, a CNS cell or tissue sample can be prepared/processed and immobilized on a support, typically a glass slide, and then contacted with a probe (e.g., a probe for a CNS gene described herein).

In still another embodiment, serial analysis of gene expression, as described in U.S. Pat. No. 5,695,937, is used to detect transcript levels of a CNS gene described herein.

### Polypeptide-Based Methods

In other embodiments, the methods described herein utilize techniques for detection of gene expression where a gene product (polypeptide) encoded by a gene is detected or where an activity of the polypeptide, e.g., an enzymatic activity, is detected. Such methods are particularly advantageous for detecting the expression of genes that encode polypeptides that are secreted from CNS cells, e.g., into the CSF.

A variety of methods can be used to determine the level of protein encoded by a CNS gene. In general, these methods include contacting a CNS sample (such as a brain cell sample or a CSF sample) with an agent, such as an antibody, that selectively binds to the protein of interest. In one embodiment, the antibody bears a detectable label. Antibodies can be polyclonal, or more preferably, monoclonal. An intact antibody, or a fragment thereof (e.g., Fab or F(ab′)2) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Such detection methods can be used to detect a CNS gene product in a CNS sample in vitro as well as in vivo.

In vitro techniques include immunoassays such as enzyme linked immunosorbent assays (ELISAs), immunoprecipitations, immunofluorescence, enzyme immunoassay (EIA), radioimmunoassay (RIA), Western blot analysis, and Luminex™ x MAP™ detection assay. Some immunoassays are “sandwich” type assays, in which a target analyte(s) is “sandwiched” between a labeled antibody and an antibody immobilized onto a solid support. The assay is read by observing the presence and amount of antigen-labeled antibody complex bound to the immobilized antibody.

Another immunoassay useful in the methods described herein is a “competition” type immunoassay, wherein an antibody bound to a solid surface is contacted with a sample (e.g., a CSF sample) containing both an unknown quantity of antigen analyte and labeled antigen of the same type. The amount of labeled antigen bound on the solid surface is then determined to provide an indirect measure of the amount of antigen analyte in the sample. Such immunoassays are readily performed in a “dipstick” format (e.g., a flow-through or migratory dipstick design) for convenient use. A dipstick-based assay optionally includes an internal negative or positive control. Numerous types of dipstick immunoassays are known in the art and are described, e.g., in U.S. Pat. Nos. 5,656,448; 4,366,241; and 4,770,853. In other embodiments, antibody-based assays are performed in an array format. For example, a CNS sample is labeled, e.g., biotinylated, and then contacted to an antibody, e.g., an antibody positioned on an antibody array. The sample can be detected, e.g., with avidin coupled to a fluorescent label.

In vivo techniques include, e.g., introducing into a subject (e.g., into the CSF) a labeled antibody that binds to the gene product to be detected. The antibody can be labeled, e.g., with a radioactive marker, whose presence and location in a subject can be detected by standard imaging techniques.

Polyclonal and monoclonal antibodies to be used to detect a particular CNS gene product will, in certain cases, be available. For example, commercially available antibodies exist for many of the CNS marker genes described herein. Alternatively, a skilled artisan can make a suitable antibody for use in a diagnostic assay using routine techniques. Methods of making and using polyclonal and monoclonal antibodies to detect a particular target are described, e.g., in Harlow et al., Using Antibodies: A Laboratory Manual: Portable Protocol I. Cold Spring Harbor Laboratory (Dec. 1, 1998). Methods for making modified antibodies and antibody fragments (e.g., chimeric antibodies, reshaped antibodies, humanized antibodies, or fragments thereof, e.g., Fab′, Fab, F(ab′)2 fragments); or biosynthetic antibodies (e.g., single chain antibodies, single domain antibodies (DABs), Fv, single chain Fv (scFv), and the like), are known in the art and can be found, e.g., in Zola, Monoclonal Antibodies: Preparation and Use of Monoclonal Antibodies and Engineered Antibody Derivatives, Springer. Verlag (Dec. 15, 2000; 1st edition).

Imaging of CNS Gene Expression

In one embodiment, the methods described herein utilize techniques for imaging of gene expression, e.g., non-invasive imaging of gene expression, in the CNS. For example, a labeled probe that is capable of detecting the expression of a target gene can be delivered into the brain through the blood-brain barrier (BBB) by targeting the labeled probe to the brain via endogenous BBB transport systems, such as carrier-mediated transport systems that exist for the transport of nutrients across the BBB. Similarly, receptor-mediated transcytosis systems operate to transport circulating peptides across the BBB, such as insulin, transferrin, or insulin-like growth factors. These endogenous peptides can act as “transporting peptides,” or “molecular Trojan horses,” to ferry a labeled diagnostic probe as described herein, across the BBB. The label can then be detected by known brain imaging techniques. Such an approach is described, e.g., in U.S. Pat. No. 6,372,250. In other embodiments, Shi et al., Proc. Natl. Acad. Sci. USA, 2000, 97(26):14709-14 and Lee et al., J. Nucl. Med. 2002, 43(7):948-56 describe imaging of gene expression in the brain in vivo using an antisense radiopharmaceutical combined with drug-targeting technology to traverse the BBB.

Other methods of delivering into the brain a labeled probe that is capable of detecting the expression of a target gene are described, e.g., in U.S. Pat. No. 5,720,720. This patent describes methods of delivering agents (such as labeled antibodies for imaging gene products) into the brain by high-flow microinfusion.

Detection of Changes in CNS Gene Expression in Bodily Fluids

In some cases, gene activation in the CNS can result in a measurable alteration in a gene product at a distant site, e.g., in a fluid such as blood, urine or semen. It is known, e.g., that the cerebral cortex, hippocampus, entorrhinal cortex, parts of the thalamus, basal ganglia, cerebellum, and the reticular formation influence the output of the autonomic nervous system (Kandel et al, Principles of Neural Science, Third Edition, Appleton & Lange). These influences can result in measurable alterations of gene expression at the mRNA or protein level in autonomic ganglia or in innervated organs. An example of this type of interaction is the immunomodulatory action of the activation of the vagus nerve after cytokine release in the periphery (Tracey, Nature, 420:853-9, 2002).

In addition, gene activation in the CNS can be detected by measuring changes in blood proteins in some cases. For example, neurons in the CNS can trigger the release of hormones in blood via the activation of several neuroendocrine axes such as the hypothalamus-pituitary-adrenal, -gonadal, or thyroid axes (Besedovsky and del Rey, Endocrine Reviews, 17:1-39, 1996). Moreover, brain extracellular fluid drains into blood and deep cervical lymph (Cserr et al, Brain Pathol., 2(4):269-76, 1992). Cerebral extracellular fluids drain from brain to blood across the arachnoid villi and to lymph along certain cranial nerves (primarily olfactory) and spinal nerve root ganglia. A minimum of 14 to 47% of protein injected into different regions of brain or cerebrospinal fluid passes through lymph. Thus, CSF markers drain into, and can be detected in, lymph, blood, or serum. Such markers found in blood may also be enriched, and thereby detectable, in urine, due to selective filtration of blood components by the kidneys.

The CNS is connected to the testis via the autonomic nervous system as well as the endocrine system. If a change in gene activity in the brain results in modifications in the activity of the hypothalamus-pituitary-gonadal axis or in the innervation of the testes, these changes could be then detected in fluids related to the testes, such as semen. For example, patients with spinal cord injury have been shown to have alterations in the composition of their semen (See Naderi and Safarinejad, Clin. Endocrinol., 58(2):177-84, 2003).

Routine methods can be used to identify gene products in peripheral tissues, such as peripheral bodily fluids, which are the result of changes in gene expression in the CNS. For example, a candidate marker gene can be disrupted in the brain of an experimental animal. A change in the expression of a candidate gene in a peripheral tissue in the experimental animal, compared to a wild type animal (i.e., an animal not disputed for the candidate marker gene) indicates that the expression of the candidate molecule in the peripheral tissue is tied to changes in gene expression in the CNS.

Arrays

The methods described herein are readily adapted for nucleic acid or protein arrays, e.g., nucleic acid and/or protein “chips,” following the methods known in the art. In a typical embodiment, an array chip includes multiple probes (e.g., DNA probes and/or antibody probes) for detection of expression of multiple CNS genes. In one embodiment, the probes on a specific chip are chosen to detect the members of one or more specific panels or “clusters” of genes, each cluster being associated with a specific gene expression profile if a non-CNS neoplasia or other disorder is present in the subject from whom the CNS sample was taken. A chip can contain tens, hundreds, or thousands of individual probes immobilized (tethered) at discrete, predetermined locations (addresses or “spots”) on a solid, planar support, e.g., glass, metal, or nylon. An array can be a macroarray or microarray, the difference being in the size of the spots. Macroarrays contain spots of about 300 microns in diameter or larger and can be imaged using gel or blot scanners. Microarrays contain spots less than 300 microns, typically less than 200 microns, in diameter.

For analysis and comparison of profiles of gene expression in the methods described herein, a nucleic acid array can be constructed using nucleic acid probes for at least four, e.g., at least 10, 20, 40, 60, 80 or 100 CNS genes. Such an array can include control probes (i.e., probes for genes whose expression is expected to remain unaffected in a negative sample, e.g., a sample from a subject not having a non-CNS disorder). Typically, such controls or “normal” non-disease samples are obtained from healthy volunteers. Longitudinal studies of healthy volunteers can be performed to confirm that the control samples are from individuals that remained disease free. Such studies provide the raw data for a database of control gene expression profiles. Such a database provides a source of normal or control “reference” profiles that can be used in the present methods. Control samples can also be obtained post-mortem from individuals who died for a reason unrelated to the disorder being diagnosed (e.g., individuals who died from an accidental trauma). In such cases, post-mortem samples should be taken as soon as possible after death, e.g., no later than 3 hours after death.

A population of labeled cDNA representing total mRNA from a sample of a tissue of interest, e.g., brain, spinal cord, or CSF, is contacted with the DNA array under suitable hybridization conditions. Hybridization of cDNAs with sequences in the array is detected, e.g., by fluorescence at particular addresses on the solid support. Thus, a pattern of fluorescence representing a gene expression pattern in the CNS sample of a particular subject or group of subjects is obtained. These patterns of gene expression can be digitized and stored electronically for computerized analysis and comparison. For example, an array can be used to compare expression of CNS genes in individuals being tested with one or more reference gene expression profiles stored electronically, e.g., in a digital database, where the reference gene expression profile is associated with either the presence (positive control) or absence (negative control) of a peripheral neoplasia or other disorder.

In some embodiments, cDNAs are used as probes to form the array. Suitable cDNAs can be obtained by conventional polymerase chain reaction (PCR) techniques, as described above. The length of the cDNAs can be from 20 to 2,000 nucleotides, e.g., from 100 to 1,000 nucleotides. Other methods known in the art for producing cDNAs can be used. For example, reverse transcription of a cloned sequence can be used (for example, as described in Sambrook et al., eds., Molecular Cloning: A Laboratory Manual. 2nd ed., Cold Spring Harbor Laboratory, Cold Spring Harbor Laboratory Press, Cold Spring Harbor, N.Y., 1989). The cDNA probes are deposited or placed (“printed” or “spotted”) onto a suitable solid support (substrate), e.g., a coated glass microscope slide, at specific, predetermined locations (addresses) in a two-dimensional grid. A small volume, e.g., 5 nanoliters, of a concentrated DNA solution is used in each spot. Spotting can be carried out using a commercial microspotting device (sometimes called an arraying machine or gridding robot) according to the vendor's instructions. Commercial vendors of solid supports and equipment for producing DNA arrays include BioRobotics Ltd., Cambridge, UK; Corning Science Products Division, Acton, Mass.; GENPAK Inc., Stony Brook, N.Y.; SciMatrix, Inc., Durham, N.C.; and TeleChem International, Sunnyvale, Calif.

The cDNAs can be attached to the solid support by any suitable method. In general, the linkage is covalent. Suitable methods of covalently linking DNA molecules 30, to the solid support include amino cross-linking and UV crosslinking. For guidance concerning construction of cDNA arrays, see, e.g., DeRisi et al., Nature Genetics, 1996, 14:457-460; Khan et al., Electrophoresis, 1999, 20:223-229; Lockhart et al., Nature Biotechnol., 1996, 14:1675-1680.

In some embodiments of the new methods, the immobilized DNA probes in the array are synthetic oligonucleotides. Preformed oligonucleotides can be spotted to form a DNA array, using techniques described herein with regard to cDNAs. In general, however, the oligonucleotides are synthesized directly on the solid support. Methods for synthesizing oligonucleotide arrays are known in the art. See, e.g., Fodor et al., U.S. Pat. No. 5,744,305. The sequences of the oligonucleotides represent portions of the sequences of a particular gene to be detected. Generally, the lengths of oligonucleotides are 10 to 50 nucleotides, e.g., 15, 20, 25, 30, 35, 40, or 45 nucleotides.

Also useful in the methods are aptamer arrays. Aptamers are nucleic acid molecules that bind to specific target molecules based on their three-dimensional conformation rather than hybridization. The aptamers are selected, for example, by synthesizing an initial heterogeneous population of oligonucleotides, and then selecting oligonucleotides within the population that bind tightly to a particular target molecule. Once an aptamer that binds to a particular target molecule has been identified, it can be replicated using a variety of techniques known in biological and other arts, e.g., by cloning and polymerase chain reaction (PCR) amplification followed by transcription. The target molecules can be nucleic acids, proteins, peptides, small organic or inorganic compounds, and even entire micro-organisms.

The synthesis of a heterogeneous population of oligonucleotides and the selection of aptamers within that population can be accomplished using a procedure known as the Systematic Evolution of Ligands by Exponential Enrichment or SELEX. The SELEX method is described in, e.g., Gold et al., U.S. Pat. Nos. 5,270,163 and 5,567,588; Fitzwater et al., (“A SELEX Primer,” Methods in Enzymology, 267:275-301, 1996); and in Ellington and Szostak (“In Vitro Selection of RNA Molecules that Bind Specific Ligands,” Nature, 346:818-22). Briefly, a heterogeneous DNA oligomer population is synthesized to provide candidate oligomers for the in vitro selection of aptamers. This initial DNA oligomer population is a set of random sequences 15 to 100 nucleotides in length flanked by fixed 5′ and 3′ sequences 10 to 50 nucleotides in length. The fixed regions provide sites for PCR primer hybridization and, in one implementation, for initiation of transcription by an RNA polymerase to produce a population of RNA oligomers. The fixed regions also contain restriction sites for cloning selected aptamers. Many examples of fixed regions can be used in aptamer evolution. See, e.g., Conrad et al. (“In Vitro Selection of Nucleic Acid Aptamers That Bind Proteins,” Methods in Enzymology, 267:336-83, 1996); Ciesiolka et al., (“Affinity Selection-Amplification from Randomized Ribooligonucleotide Pools,” Methods in Enzymology, 267:315-35, 1996); Fitzwater, supra.

Aptamers are generally selected in a 5 to 100 cycle procedure. In each cycle, oligomers are bound to the target molecule, purified by isolating the target to which they are bound, released from the target, and then replicated by 20 to 30 generations of PCR amplification.

Aptamer selection is similar to evolutionary selection of a function in biology. Subjecting the heterogeneous oligonucleotide population to the aptamer selection procedure described above is analogous to subjecting a continuously reproducing biological population to 10 to 20 severe selection events for the function, with each selection separated by 20 to 30 generations of replication.

Heterogeneity is introduced, e.g., only at the beginning of the aptamer selection procedure, and does not occur throughout the replication process. Alternatively, heterogeneity can be introduced at later stages of the aptamer selection procedure.

Various oligomers can be used for aptamer selection, including, e.g., 2′-fluoro-ribonucleotide oligomers, NH2-substituted and OCH3-substituted ribose aptamers, and deoxyribose aptamers. RNA and DNA populations are equally capable of providing aptamers configured to bind to any type of target molecule. Within either population, the selected aptamers occur at a frequency of 109 to 1013, see Gold et al., (“Diversity of Oligonucleotide Functions,” Annual Review of Biochemistry, 64:763-97, 1995), and most frequently have nanomolar binding affinities to the target, affinities as strong as those of antibodies to cognate antigens. See Griffiths et al., (EMBO J., 13:3245-60, 1994).

Using 2′-fluoro-ribonucleotide oligomers is likely to increase binding affinities ten to one hundred fold over those obtained with unsubstituted ribo- or deoxyribo-oligonucleotides. See Pagratis et al. (“Potent 2′-amino and 2′ fluoro 2′deoxyribonucleotide RNA inhibitors of keratinocyte growth factor” Nature Biotechnology, 15:68-73). Such modified bases provide additional binding interactions and increase the stability of aptamer secondary structures. These modifications also make the aptamers resistant to nucleases, a significant advantage for real world applications of the system. See Lin et al. (“Modified RNA sequence pools for in vitro selection” Nucleic Acids Research, 22:5229-34, 1994); Pagratis, supra.

In the present invention, aptamers can be used to detect, e.g., mRNAs, cDNAs, or proteins corresponding to CNS marker genes.

In some embodiments of the invention, probes (e.g., nucleic acid probes, antibodies, or aptamers) for the human homologs of animal model CNS genes are used in the detection method. In other embodiments, the probe used for detection consists of highly conserved regions of a gene, e.g., a sequence that is highly conserved between homologous mouse and human sequence.

Sample Preparation and Analysis

In the new methods, the transcription level of one or more CNS genes is assumed to be reflected in the amount of its corresponding mRNA present in cells of an assayed CNS sample. In general, mRNA from the CNS cells or tissue is copied into cDNA under conditions such that the relative amounts of cDNA produced representing specific genes reflect the relative amounts of the mRNA in the sample. Comparative hybridization methods involve comparing the amounts of various, specific mRNAs in two tissue samples, as indicated by the amounts of corresponding cDNAs hybridized to sequences from the genes of interest.

The mRNA used to produce cDNA is generally isolated from other cellular contents and components. One useful approach for mRNA isolation is a two-step approach. In the first step, total RNA is isolated. The second step is based on hybridization of the poly(A) tails of mRNAs to oligo(dT) molecules bound to a solid support, e.g., a chromatographic column or magnetic beads. Total RNA isolation and mRNA isolation are known in the art and can be accomplished, for example, using commercial kits according to the vendor's instructions. Similarly, synthesis of cDNA from isolated mRNA is known in the art and can be accomplished using commercial kits according to the vendor's instructions. Fluorescent labeling of cDNA can be achieved by including a fluorescently labeled deoxynucleotide, e.g., Cy5-dUTP or Cy3-dUTP, in the cDNA synthesis reaction. For guidance concerning isolation of mRNA and synthesis of fluorescently labeled cDNA for analysis on a DNA array, see, e.g., Ross et al., Nature Genetics, 2000, 24:227-235.

Conventional techniques for hybridization and washing of DNA arrays, detection of hybridization, and data analysis can be employed in the new methods without undue experimentation. Commercial vendors of hardware and software for scanning DNA arrays and analyzing data include Cartesian Technologies, Inc. (Irvine, Calif.); GSI Lumonics (Watertown, Mass.); Genetic Microsystems Inc. (Woburn, Mass.); and Scanalytics, Inc. (Fairfax, Va.).

In other embodiments, the expression level of one or more CNS genes is reflected in the presence and/or level of protein present in cells of a CNS sample to be assayed. The presence or level of protein in a CNS sample can be detected by routine methods. For example, a CNS sample (e.g., a CSF sample) can be analyzed by gel electrophoresis techniques such as 2-dimensional (2D) PAGE. Once protein spots are separated on a 2D-PAGE gel, differentially expressed spots can be identified, e.g., by matrix assisted laser desorption ionization time of flight (MALDI-TOF) and electrospray ionization (ESI). This method can also be used for peptide analysis to provide the fingerprint of a particular protein in a sample.

A second proteomic approach can involve obtaining a proteomic spectrum by directly analyzing a CNS sample, such as a CSF sample, by mass spectroscopy. For example, surface enhanced laser desorption ionization time of flight (SELDI-TOF) analysis can be performed to generate a proteomic pattern from a CNS sample. SELDI-TOF analysis has been shown to be able to identify a cluster pattern that differentiates between normal and disease patients. See, Paweletz et al., Dis. Markers, 17(4):301-7, 2001.

### Generating Gene Expression Profiles

A gene expression profile used in the methods described herein is a pattern of expression of two or more CNS genes. In some cases, an expression profile can be a pattern of expression of 5, 10, 25, 50, 100, 200, 500, or more genes. A “reference gene expression profile” as used herein is a characteristic pattern (dataset) of expression (e.g., up or down regulated and/or level of expression) of two or more CNS genes, where the pattern of expression is associated with risk or presence of a particular disorder (e.g., a ratio of the level of expression associated with a particular disorder to the level of expression in a person without the disorder). The association between the characteristic profile and the particular disorder is determined through the generation and analysis of CNS gene expression data to identify correlations between particular patterns of CNS gene expression (e.g., relative increases and/or decreases of gene expression of particular genes compared to a negative control) and particular clinical states. For example, a reference gene expression profile can be data for a set of genes (also referred to herein as a “panel” or “cluster” of genes), where each gene of the set is either down-regulated or up-regulated when associated with a specific peripheral disorder or any peripheral disorder.

A reference profile can also include a value, e.g., a relative value, of gene expression for two or more genes in a panel, where at least one gene of the panel is down-regulated and at least one gene is up-regulated. An example of such a gene expression profile is a profile that includes a value for the relative differential expression of at least 2, e.g., between 5 and 50, of the genes shown in any of the tables of FIGS. 47A-C or any number of the genes listed in FIGS. 58 and 60. Such a reference profile is associated with the presence of early stage carcinoma, arthritis or asthma. Other examples are provided by each of the figures disclosed herein. For example, FIG. 31-4 provides a profile or panel of genes that are significantly up-regulated in the cortex in response to the presence of lung cancer.

Exemplary gene expression profiles associated with non-CNS carcinoma (or particular types of non-CNS carcinoma, such as breast, lung or colon carcinoma) are shown in FIGS. 2-46. A reference gene expression profile can include data from at least a portion of the genes or gene products shown in these figures. For example, a reference gene expression profile associated with lung carcinoma can include a value for the differential expression of 1, 2, 5, 10, 20, 30, 40, 50, or more, genes or gene products listed as CNS markers for lung carcinoma in FIGS. 8, 9, 10, 17, 18, 19, 26, 27, and 28. The reference profiles that can be used with the methods of the invention are not limited by the CNS markers described herein.

Reference profiles can be generated by detecting changes in patterns of gene expression in the CNS in response to the presence of non-CNS disease in an experimental animal, and identifying the human homologs of the genes and gene clusters that are differentially expressed in a certain pattern in the experimental samples, as exemplified in Examples 1-3 described herein.

A reference gene expression profile can also be obtained by evaluating human CNS gene expression data. For example, a database is created and maintained where CNS gene expression data is obtained and stored, e.g., electronically e.g., digitally, for tens, hundreds, or thousands of individuals. The individuals can be followed and evaluated with regard to, e.g., cancer clinical state longitudinally (e.g., over at least 5 years, 10 years, 15 years, 20 years, 30 years, 50 years or a lifetime). The expression profiles of individuals who developed a particular disease, e.g., 5, years, 10 years, 15 years, 20 years, 30 years, or 50 years after the CNS gene expression data was obtained, are compared with the expression profiles of individuals who remained disease free. Similar comparison is made between individuals who developed one clinical type of the disorder compared to another, or individuals who developed the disease at an early age versus a late age. These analyses provide specific reference CNS gene expression profiles that are associated with different stages of disease, e.g., different stages of neoplasia, or different types of tumors. A “control gene expression profile” is a profile of a given set of genes in a healthy (normal) individual or animal model.

Both reference and control gene expression profiles are typically stored in electronic digital form, e.g., on a computer-readable medium, such as a CD, diskette, DVD, hard drive, computer memory, or memory cards, along with identifying information such as gender, type and stage of disorder, age group, and race of the subject.

A “test gene expression profile” is obtained from a CNS sample of a subject to be tested for the presence of peripheral disease. First, a CNS sample, e.g., a brain cell sample or CSF sample, is obtained from the subject by routine means such as brain needle biopsy (for a brain cell sample) or a lumbar puncture (for CSF), as described herein. The sample is then prepared for use in a method of detecting gene expression, e.g., any method of detecting gene expression described herein. In one embodiment, total RNA can be prepared from the sample, and reverse transcribed into cDNA for use in a nucleic acid array assay described herein. In another embodiment, total protein is prepared from the sample for use in an antibody assay described herein. The prepared sample can then be contacted with an array (e.g., an antibody or nucleic acid array) that can detect expression levels (or protein levels in the case of an antibody array) of at least one cluster or panel of CNS genes or gene products corresponding to the cluster or panel of CNS genes or gene products of one or more particular reference gene expression profiles to which the test sample will be compared. For example, a prepared CNS sample from the test subject can be contacted with a nucleic acid array containing nucleic acid probes or an antibody array containing antibody probes for two or more, e.g., between 2 and 150, between 10 and 50, or between 20 and 30, of the genes shown in FIGS. 2-46. In one embodiment, the array can contain probes for each of the marker genes in a particular cluster disclosed in any of FIGS. 2-46.

The results of the array assay are obtained by routine techniques, such as fluorescence detection and measurement of bound antibody or hybridized nucleic acid for each position (each probe) on the array. A dataset of the values for the level of each polypeptide or gene detected in the CNS sample by each antibody or probe on the array can then be generated. The dataset can contain information such as patient identifier, and actual and/or relative levels of expression or protein detected. Such a dataset can be used directly as the “test” or “sample” gene expression profile or the dataset can be converted into a format comparable to the format of the reference profile.

Once the test expression profile is generated, a test profile can be compared to a reference expression profile as described herein.

### Analyzing Gene Expression Profiles

The new methods any systems enable one to of evaluate a test subject by comparing a test gene expression profile from the test subject with a reference gene expression profile associated with the presence of a particular disorder and/or a control (“normal”) gene expression profile associated with the absence of a particular non-CNS disorder. Longitudinal studies of CNS gene expression in multiple volunteers are performed to identify and confirm control gene expression profiles that are associated with individuals who remain disease free or reference profiles individuals who get the disease. Such studies provide the raw data for a database of negative and positive control gene expression profiles that can be used in the present methods.

Subject “test” and “reference” profiles can be obtained by methods described herein. In one embodiment, the methods include obtaining a CNS sample from a subject (either directly or indirectly from a caregiver or other party), creating an expression profile from the sample, and comparing the subject's expression profile to one or more control and/or reference profiles and/or selecting a reference profile most similar to that of the subject.

As with other detection methods, profile-based assays can be performed prior to the onset of symptoms (in which case they are diagnostic), prior to treatment (in which case they are prognostic) or during the course of treatment (in which case they serve as monitors.)

A variety of routine statistical measures can be used to compare two gene expression profiles. One possible metric is the length of the distance vector that is the difference between the two profiles. Each of the test and reference profile is represented as a multi-dimensional vector, wherein each dimension is a value in the profile, e.g., a value for the expression of a particular gene in a panel. A test profile and reference or control profile can be said to “match” if at least 75% of the genes in a test gene expression profile are either up- or down-regulated in the same manner as the genes in the reference expression profile. A “high level match” would mean that at least 75% of the genes come within at least plus or minus 50% of the expression level (or Log2 ratio of expression level) of the gene in the reference expression profile.

In one embodiment, test and reference profiles are said to match if their respective multi-dimensional vectors, as described above, have a 30% or lower variance with respect to each other. If the test and reference profile match, the test subject can be identified as having the peripheral disorder with which the reference profile is associated. If the test and normal control profile match, the subject is likely to be free of the peripheral disorder.

In one embodiment, pattern recognition software is used to identify matching profiles. For example, unsupervised clustering algorithms, such as hierarchical clustering, K-means clustering, and SOM (self-organizing maps) for pattern discovery can be used. Supervised techniques such as SVM (support vector machines) and SPLASH (structural pattern localization analysis by sequential histograms) algorithms implemented in the Genes @Work software package (IBM Corp.) can also be used.

In another embodiment, gene expression profiles are analyzed by quantitative pattern comparison performed by applying a nearest neighbor classifier (see Jelinek et al., Mol. Cancer Res., 1:346-61, 2003). Based on the nearest neighbor classifier, a score is defined which, together with a permutations-derived distribution, can be used to estimate the probability of each test profile of belonging to a class defined by a reference gene expression pattern (see Jelinek, supra).

The result of the diagnostic test, which can be transmitted in paper or electronic form to the subject, a caregiver, or another interested party, can be the subject expression profile per se, a result of a comparison of the subject expression profile with another profile, a most similar reference profile, or a descriptor of any of these. Transmission can occur across a computer network (e.g., in the form of a computer transmission such as a computer data signal embedded in a carrier wave). The new systems also include a computer-readable medium (such as a CD, diskette, or hard drive) having executable code for effecting the following steps: receive a subject expression profile; access a database of reference expression profiles; and either i) select a matching reference profile most similar to the subject expression profile, or ii) determine at least one comparison score for the similarity of the subject expression profile to at least one reference profile. The subject expression profile and the reference expression profile each include a value representing the level of expression of one or more of the identified genes or gene products or the proteins they encode.

### Predictive Medicine

The methods described herein are generally useful in the field of predictive medicine and, more specifically, are useful in diagnostic and prognostic assays, in monitoring progression of a disease, e.g., neoplasia, or monitoring of response to treatment, e.g., in clinical trials. For example, one can determine whether a subject has a very early stage neoplasia, in the absence of other, e.g., clinical, indications of neoplasia. In another example, one can determine whether a subject is at risk for developing rheumatoid arthritis or whether the subject has early stage RA, in the absence of clinical indications of RA such as joint inflammation. The methods are particularly useful, e.g., for patients who have had surgery or treatment for the disease (e.g., to remove cancer), in which case the methods could be used to monitor recurrence or metastasis, for persons living in regions of high incidence of cancer due, e.g., to environmental factors, or for individuals who have a family history of a disease (e.g., diabetes, asthma or cancer) or are carriers of a disease susceptibility gene, e.g., a cancer susceptibility gene (e.g., BRCA1 or BRCA2, hMSH2, MLH1, MSH2, or MSH6). Other cancer susceptibility genes are described in The Genetic Basis of Human Cancer, 2nd edition (Vogelstein and Kinzler, Eds.), McGraw-Hill Professional (2002). Such individuals can be evaluated using the methods described herein.

In some cases, for example, where the risk of developing a disease is high (e.g., where an individual has a strong family history of asthma or cancer, or where an individual carries a cancer susceptibility gene or lives in a high risk area for cancer), an individual can be evaluated periodically (e.g., every 10 years, every 5 years, or every year) during his lifetime.

The “subject” referred to here, and that is referred to in the context of any of the methods, is a vertebrate animal, typically a mammal, or a human. The subject can be an experimental animal (e.g., an experimental rodent such as a rat or mouse), a domesticated animal (e.g., a dog or cat); an animal kept as livestock (e.g., a pig, cow, sheep, goat, or horse); a non-human primate (e.g., an ape, monkey, or chimpanzee). The animal or human can be unborn (accordingly, the methods of the invention can be used to carry out genetic screening or to make prenatal diagnoses).

### A System for Diagnosing a Non-CNS Disorder

A system for diagnosing a non-central nervous system (non-CNS) disorder in a subject can include the following elements: a sampling device to obtain a CNS sample, a gene expression detection device, a reference gene expression profile, and a means for comparing gene expression (e.g., a comparator) of one or more genes in the CNS sample with the reference gene expression profile.

A sampling device obtains a CNS sample by a minimally invasive technique, e.g., a form of neurosurgery. Minimally invasive neurosurgery techniques include computer-assisted stereo-taxis, intra-operative ultrasound, brain mapping and neuro-endoscopy, among other techniques. Stereo-taxis refers to a system of navigating to any area within the brain, with the aid of imaging techniques that display external reference landmarks and neural structures.

Alternatively, a “sample” can be taken by imaging gene expression, e.g., in the brain, rather than taking an actual sample. Brain imaging can be performed by Computer Tomography Scan (CT), Magnetic Resonance Imaging (MRI) or Positron Emission Tomography (PET), among other methods. Signals originated from these methods provide reference points from which a computer can calculate and present trajectories and depths to any target point within the brain. The latest generation of stereo-tactic systems, which includes the Cosman-Roberts-Wells (CRW) system, can be used with MRI and cerebral angiographic localization. Intra-operative ultrasound can be used either alone or in combination with stereo-taxis. Intra-operative ultrasound is used to identify structures such as the ventricles prior to dural opening. The ultrasound probe can also be used to guide a needle biopsy of a deep-seated lesion to obtain the CNS sample. Both the rigid and fiber-optic flexible endoscopes can be used to obtain a brain sample using minimally invasive techniques. Lasers and various other instruments (including biospy instruments) can be attached and used. A sampling device to obtain cerebrospinal fluid by lumbar puncture can be also guided by any of the imaging methods listed above.

Gene expression detection devices include those described herein under the subheading Nucleic Acid-Based Methods, Array, and, sample preparation and analysis. The comparator can be a computer loaded with pattern recognition software, as described herein.

### Computer-Readable Medium

In another aspect, the new systems feature a computer-readable medium having a plurality of digitally encoded data records or data sets. Each data record or data set includes a value representing the level of expression of a CNS gene, and a descriptor of the sample. The descriptor can be, e.g., an identifier (e.g., an identifier for the patient from which the sample was obtained, e.g., a name or a reference code that can be matched with patient information only by those having access to a decoding table), a diagnosis made, or a treatment to be performed in the event the level of expression reaches a certain level or falls below a certain level. The data record can also include values representing the level of expression of related genes (e.g., the data record can include values for each of a plurality of genes in a gene “cluster,” where a particular reference gene expression for the genes in the cluster is associated with a non-CNS disorder). The data record can also include values for control genes (e.g., genes whose expression is not changed in control samples or whose expression is not diagnostically correlated with a non-CNS disorder). The data record can be structured in various ways, e.g., as a table (e.g., a table that is part of a database such as a relational database (e.g., a SQL database of the Oracle or Sybase database environments) or as a list.

### Non-CNS Diseases

The methods described herein are not limiting in that they can be used to diagnose and monitor various non-CNS disorders, such as a neoplasia (e.g., tumor or cancer); immune disorders (e.g., an autoimmune disorder such as rheumatoid arthritis, multiple sclerosis, systemic lupus erythematosus, psoriasis, scleroderma); allergic or inflammatory disorders (e.g., asthma, inflammatory bowel disease, Crohn's disease); metabolic or endocrine disorders (e.g., diabetes, obesity, Addison's disease); pathogenic infections (e.g., a viral, parasitic or fungal infection, e.g., HIV infection); and cardiovascular disorders.

As used herein, “neoplasia” refers to the uncontrolled and progressive proliferation of cells under conditions that would not elicit, or would cause cessation of, proliferation of normal cells. Neoplasia results in the formation of a “neoplasm,” which is defined herein to mean any new and abnormal growth, particularly a new growth of tissue, in which the growth is uncontrolled and progressive. Neoplasm, as used herein, is synonymous with “tumor.” Malignant neoplasms or tumors are distinguished from benign in that the former show a greater degree of anaplasia, or loss of differentiation and orientation of cells, and have the properties of invasion and metastasis. Thus, neoplasia includes “cancer,” which herein refers to a proliferation of cells having the unique trait of loss of normal controls, resulting in unregulated growth, lack of differentiation, local tissue invasion, and metastasis. The methods described herein can be used to diagnose neoplasia from any non-CNS cell or tissue type, such as neoplasia derived from epithelial or endocrine tissue, mesenchymal tissues, or hematopoietic tissue.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the colon, lung, prostate, breast, cervix, head and neck, and ovary. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. The disorders can arise from poorly differentiated acute leukemias, e.g., erythroblastic leukemia and acute megakaryoblastic leukemia. Exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

### Identification of Disease Surveillance Genes for Non-CNS Disorders

The new methods also include methods of identifying disease surveillance genes for non-CNS disorders in a subject, as well as lists (in the Figures) of those CNS marker genes that have already been discovered. Generally, such methods involve detecting changes in gene expression in the CNS in response to the presence of a particular non-CNS disease condition in a subject, e.g., an experimental animal. The methods will generally involve inducing a disease condition or disorder in a test experimental animal; and comparing the expression of at least one gene in a CNS sample from the test experimental animal to expression of the gene in a CNS sample from a control experimental animal. A gene (or a human homolog of a gene) that is differentially expressed in the CNS sample from the test experimental animal compared to the CNS sample from the control experimental animal is a CNS diagnostic marker for a non-CNS disorder. Such markers are referred to herein as CNS “marker genes” or “disease surveillance genes” for non-CNS disease. It is understood, however, that the gene product of the marker gene can also serve as a diagnostic marker. In most cases, a plurality of differentially expressed markers are identified (e.g., a “profile” or “cluster” of markers is identified). The experimental animal is preferably an experimental mammal, and can be, e.g., an experimental rodent (e.g., a rat, mouse or guinea pig) or non-human primate (e.g., an ape, e.g., a monkey or chimpanzee).

The methods of detection of gene expression described herein, and particularly array and chip technology, are useful for methods of identifying Disease surveillance genes for non-CNS neoplasia. CNS samples are prepared from experimental and control animals (e.g., brains are biopsied or removed, or CSF samples are taken) and RNA, cDNA, or protein is prepared from the samples as described herein. A single chip (e.g., a commercially available chip having probes for a large number of genes in the genome of the experimental animal species) can allow measurement of the level at which hundreds, thousands, or even tens of thousands of genes are expressed in the CNS sample of a test experimental animal compared to a control experimental animal. Typically, clustering methodology or other bioinformatics tools are used to mine the data obtained from such large scale experiments and identify the genes or clusters of genes that are statistically significantly differentially expressed in an experimental sample compared to a control sample. Many such tools and programs are available to the skilled artisan. An exemplary method of data analysis is described herein and exemplified in the Examples below.

Disease Surveillance Genes for Neoplasia

In one embodiment, CNS diagnostic markers for non-CNS neoplastic disorders are identified by detecting changes in gene expression in the CNS in response to the presence of a non-CNS neoplasm in an experimental animal. For example, a neoplasm is induced in an experimental animal and gene expression in the CNS of the experimental animal is evaluated compared to a control animal. Methods for inducing growth of a non-CNS neoplasm, e.g., a cancer, in an experimental animal, are known in the art and include, e.g., chemical or radiation mutagenesis, or transplantation of a neoplastic cell (e.g., a neoplastic cultured cell or cell line) to the experimental animal. CNS genes or gene products whose expression is altered in the experimental animal compared to a control animal are identified as CNS markers or surveillance genes for neoplasia. Examples of CNS marker genes for cancer, particularly for carcinoma, are provided herein by FIGS. 2-48 and Examples 1-3.

In various embodiments, the diagnostic markers for breast cancer include Nedd8 (FIG. 29-1), Col4a3 bp (FIG. 29-2), Bgn (FIG. 29-4), Sox5 (FIG. 29-5), Slc38a4 (FIG. 32-1), Tom1 (FIG. 32-2), Calr (FIG. 32-4), Itgae (FIG. 32-5), Ttrap (FIG. 35-1), P ex11b (FIG. 35-2), Sema7a (FIG. 35-4), Stam2 (FIG. 35-5).

In other embodiments, the diagnostic markers for colon cancer include Nmb (FIG. 30-1), Ryr2 (FIG. 30-2), Trfr (FIG. 30-4), Mfap5 (FIG. 30-5), Prrg2 (FIG. 33-1), Faim (FIG. 33-2), Mgrn1 (FIG. 33-4), Stch (FIG. 33-5), Lhb (FIG. 36-1), Prm3 (FIG. 36-2), Crry (FIG. 36-4), Timp4 (FIG. 36-5).

Diagnostic markers for lung cancer include Nmb (FIG. 31-1), Pcdh8 (FIG. 31-2), Rock2 (FIG. 31-4), Angpt13 (FIG. 31-5), Sqstm1 (FIG. 34-1), Kcnip2 (FIG. 34-2), Oxt (FIG. 34-4), Myh4 (FIG. 34-5), Enc1 (FIG. 37-1), Gsg1 (FIG. 37-2), Srr (FIG. 37-4), Ndph (FIG. 37-5).

Any one of these disease surveillance genes can be used alone or in a set, e.g., of 2, 5, or 10 genes to create probes useful in the methods described herein to diagnose specific cancers.

Disease Surveillance Genes for Rheumatoid Arthritis

In another embodiment, identifying CNS diagnostic markers for rheumatoid arthritis (RA) can be identified by detecting changes in gene expression in the CNS in an animal model of RA compared to a wild type animal. For example, the art-recognized rodent collagen induced arthritis (CIA) model can be used. In this model, arthritis is induced in a rodent, e.g., a DBA/1 mouse, by intradermal injection of purified collagen. 100 μg of purified type II collagen emulsified in complete adjuvant is typically injected at the base of the tail. Onset of arthritis is macroscopically visible as paw swelling or redness approximately three weeks after immunization (Williams et al., 1992, Proc. Natl. Acad. Sci. (USA), 89:9784-9788). Clinical features of arthritis are monitored by quantitatively assessing paw swelling (e.g., with calipers) over a period of time. Severity of arthritis is assessed according to established clinical scores (Williams et al., 1995, Eur. J. Immunolo., 25:763-769). CNS genes or gene products whose expression is altered in the CIA animal compared to a control animal are identified as CNS markers or surveillance genes for RA.

Given the involvement of Th1 lymphocytes and B cells, pro-inflammatory cytokines, and a possible mimicry of bacterial LPS in disease evolvement, it is likely that genes that regulate these processes are candidates to be involved in early RA surveillance in the CNS. For example, pro-inflammatory cytokines produced in the brain such as IL-1β, TNF, IL-18, IFN-γ, IL-12, gp130; cytokines such as IL-6 and leukemia inhibitory factor (LIF); neurotransmitters and neurotrophic factors such as N-methyl-D-aspartate (NMDA), brain-derived neurotrophic factor (BDNF), glial cell line-derived neurotrophic factor (GDNF), nerve growth factor (NGF); inhibitors of cytokines such as prostaglandin E2 (PGE2) and SOCS-1 and -3; SOCS regulators such as cAMP-inducing central peptides; brain molecules that are produced as a result of cytokine action, such as pentraxin 3 (PTX3); hormone releasing factors such as cortocotropin; corticotropin-releasing hormone (CRH) and other hormones involved in the regulation of the HPA axis; pituitary corticotroph proteins such as POMC; molecules involved in NF-κB-mediated signaling of inflammatory response; and other members of the families of these genes, as well as inducers and stimulators of these proteins, may be disease-surveillance genes for RA. See, e.g., See, e.g., Blond et al., 2002, Brain Res., 958(1):89-99; Suk et al., 2001, Immunol. Lett., 77(2):79-85; Losy et al., 2001, Acta Neurol. Scand., 104(3):171-3; Opp et al., 2001, Neuroendocrinology, 73(4):272-84; Chesnokova et al., 2002, Endocrinology, 143(5):1571-4; Bousquet et al., 2002, Mol. Endocrinol., 15(11):1880-90; Polentarutti et al., 2000, J. Neuroimmunol., 106(1-2):87-94; Bayas et al., 2003, Neurosci. Lett. 335(3):155-8; Xu et al., 2000, Acta Pharmacol. Sin. 21(7):600-4; Fang et al., 2000, Neuroreport, 11(4):737-41).

In various embodiments, the diagnostic markers for rheumatoid arthritis include Bcl21 (FIG. 51A), P2rx1 (FIG. 51B), Pafah1b1 (FIG. 51B), Kcna3 (FIG. 51C), Taf1b (FIG. 51C), Slc38a3 (FIG. 51D), Hprt (FIG. 52A), C1d (FIG. 52B), Car11 (FIG. 52D), Dusp3 (FIG. 52D), Gabrr2 (FIG. 53C), Aatk (FIG. 53D.

Disease Surveillance Genes for Asthma

In another embodiment, CNS diagnostic markers for asthma can be identified by detecting changes in gene expression in the CNS in an animal model of asthma compared to a wild type animal. Several experimental models of asthma are known in the art, including rodent, sheep, and non-human primate models (for a review, see Isenberg-Feig et al., 2003, Curr. Allergy Asthma Rep. 3(1):70-8). Any of these can be used in the present methods. In one embodiment, the experimental model of asthma is performed according to Komai et al. (2003, Br. J. Pharmacol., 138(5):912-20). In brief, Balb/c mice are sensitized by intraperitoneal administration of 50 μg of ovalbumin combined with 1 mg of alum (Al(OH)3) on day 0 and 12. From day 22 to 43 animals are exposed to daily aerosol challenges of 1% w/v of ovalbumin for 30 minutes. Control animals can include saline-injected animals and animals sensitized with ovalbumin and alum and challenged with saline. Airway function is evaluated by measuring one or more of: airway responsiveness to acetylcholine; IL-4, IL-5, and/or IL-13 levels; interferon-γ levels; eosinophil numbers in bronchoalveolar fluids; specific IgG1 and IgG2a levels in sera; lung histology; and rectal temperature. CNS markers or surveillance genes for asthma are those whose expression is altered in the asthma model animal compared to a control animal, or those whose expression is altered after aerosol challenge compared to before aerosol challenge.

Several gene products associated with the CNS have been shown to influence the Th-2 response and are candidates as disease-surveillance genes. These include glucocorticoid, one of the main hormonal mediators of stress, which acts on antigen-presenting cells to suppress the production of IL-12 in vitro and ex vivo; neurotransmitters norepinephrine or epinephrine; β-adrenoreceptor (ARs) agonists and antagonists (e.g., propranolol); modulators of neurotransmission such as adenosine and adenosine analogues; opiod system components, which influence the immunological response in general and the Th-1/Th-2 balance in particular; mediators of allergic reactions, such as histamine; neuropeptides such as substance P, vasoactive intestinal peptide and somatostatin, which increase the release of histamine from mast cells. See Blotta et al., 1997, J. Immunol. 158: 5589-5595; Elenkov et al., 1996; Proc. Assoc. Am. Physicians, 108: 374-381; Cooper et al., The Biochemical Basis of Neuropharmacology, Oxford University Press, 1996, p. 123; Link et al., 1999, J. Immunol. 164: 436-442; Loizzo et al., 2002, Br. J. Pharmacol., 135(5):1219-26; Lowman et al., 1988, British Journal of Pharmacology, Vol 95:121-130; and Elenkov et al., Annals of the New York Academy of Sciences, 2000, 917:94-105.

In one embodiment, the diagnostic markers for asthma are Rasa3 (FIG. 55B), Tnk2 (FIG. 55B); H28 (FIG. 55C), Diap2 (FIG. 55C), Lgals6 (FIG. 56A), Reck (FIG. 56A), Whrn (FIG. 56A), Stk22s1 (FIG. 56B), CD47 (FIG. 57A), Jund1 (FIG. 57A), Cstb (FIG. 57B), and Desrt (FIG. 57B).

Disease Surveillance Genes for Diabetes

In another embodiment, CNS diagnostic markers for diabetes can be identified by detecting changes in gene expression in the CNS in an animal model of diabetes compared to a wild type animal. Several experimental models of diabetes are known in the art, e.g., spontaneous models such as the NOD Mouse and BB Rat, and inducible models such as streptozotocin-induced (STZ) Diabetic Rats. These are reviewed in Cheta, 1998, J. Pediatr. Endocrinol. Metab., 11(1):11-9. CNS markers or surveillance genes for diabetes are those whose expression is identified to be altered in an induced animal compared to an uninduced animal (e.g., a streptozotocin-fed STZ rat compared to a control fed STZ rat), or those whose expression is altered in the early stages of spontaneous progression of disease.

Disease Surveillance Genes for Obesity

In yet another embodiment, CNS diagnostic markers for a propensity for obesity can be identified by detecting changes in gene expression in the CNS in an animal model of obesity, e.g., comparing CNS gene expression in an obesity-prone animal before and after obesity develops or is clinically detectable. The method can involve comparing differences in CNS gene expression between mouse strains that are either prone to obesity or resistant to obesity after being exposed to a fat-rich diet. For example, the method can employ the C57BL/KsJ(KsJ) or A/J strain of mice, both of which are resistant to the development of dietary obesity, or the obesity-prone strain C57BL/6J (B6).

Possible disease-surveillance genes for obesity or loss or body weight control include leptin, leptin receptor, ghrelin, cholecystokinin (CCK), CCK-A receptor, neuropeptide Y (NPY), proopiomelanocortin (POMC), α-melanocyte stimulating hormone (α-MSH), and other molecules that participate in the central control of energy balance. Given the fact that so many gene products orchestrate behaviors related to food intake, genetic deficiencies or the presence of particular polymorphic alleles in one or more of these genes may induce disorders in the control of energy homeostasis leading to obesity. Such a deficiency or disruption in the normal signaling of such molecules can likely trigger an early signal that alters CNS gene expression.

Isolating Homologous Sequences from Other Species

The human homologs of the genes listed in FIGS. 1, 50, & 54 can be found on public databases such as GenBank and others that are available on the Internet.

The human homologs of CNS marker genes and their products (e.g., human homologs of CNS marker genes identified by experiments in non-human experimental animals) are useful for various embodiments of the methods described herein. Human homologs are known for most of the CNS marker genes provided herein. In those cases where a human homolog is not identified, several approaches can be used to identify such genes. These methods include low stringency hybridization screens of human libraries with a mouse marker gene nucleic acid sequence, polymerase chain reactions (PCR) of human DNA sequence primed with degenerate oligonucleotides derived from a mouse marker gene, two-hybrid screens, and database screens for homologous sequences.

Therapeutic Methods

The methods described herein can be used to identify or diagnose the presence of a non-CNS disorder in a subject at an early stage in the pathogenic process. As such, the methods allow for early intervention, which can be the key to successful treatment and/or management of many disorders. For example, if a propensity for obesity or diabetes can be diagnosed at an early stage using the methods described herein, simple lifestyle or nutritional changes may be sufficient to stop or slow the progress of the disease, where such changes would not be sufficient if the disease were diagnosed at a later, more progressive stage. Similarly, a neoplasia that is detected at an early stage is more likely to be treated with less toxic therapeutic agents, or lower doses of a therapeutic agent, than would be used at a stage of advanced neoplasia, e.g., cancer.

Chemotherapeutic Agents

In one embodiment, the methods described herein can identify or diagnose the presence of a non-CNS neoplasia in a subject at an early stage, e.g., before a neoplasm has formed, before a neoplasm is clinically detectable, and/or before a tumor has become malignant. As such, a neoplasm detected by a method described herein is amenable to treatment by an agent that targets neoplastic cells in general or targets specific neoplastic cells in particular. In one embodiment, a subject may be treated with a chemotherapeutic agent. Chemotherapeutic agents, as used herein, refer to chemical therapeutic agents or drugs used in the treatment of neoplasia. This term is used for simplicity notwithstanding the fact that other compounds may be technically described as chemotherapeutic agents in that they exert an anti-cancer effect. A number of exemplary chemotherapeutic agents are described below.

Suitable chemotherapeutic agents include: antitubulin/antimicrotubule drugs, e.g., paclitaxel, taxol, tamoxifen, vincristine, vinblastine, vindesine, vinorelbin, taxotere; topoisomerase I inhibitors, e.g., topotecan, camptothecin, doxorubicin, etoposide, mitoxantrone, daunorubicin, idarubicin, teniposide, amsacrine, epirubicin, merbarone, piroxantrone hydrochloride; antimetabolites, e.g., 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate=PALA, pentostatin, 5-azacitidine, 5-Aza 2′-deoxycytidine, ara-A, cladribine, 5-fluorouridine, FUDR, tiazofurin, N-[5[N-(3,4-dihydro-2-methyl-4-oxoquinazolin-6-ylmethyl)-N-methylamino]-2-thenyl]-L-glutamic acid; alkylating agents, e.g., cisplatin, carboplatin, mitomycin C, BCNU=Carmustine, melphalan, thiotepa, busulfan, chlorambucil, plicamycin, dacarbazine, ifosfamide phosphate, cyclophosphamide, nitrogen mustard, uracil mustard, and pipobroman, 4-ipomeanol; estrogen modulators, e.g., raloxifene; piroxicam; 9-cis retinoic acid.

Suitable dosages for the selected chemotherapeutic agent are known to those of skill in the art. For example, where the agent is doxorubicin, suitable dosage may include 30 mg/m² of patient skin surface area, administered intravenously, twice at 1 week intervals. However, one of skill in the art can readily adjust the route of administration, the number of doses received, the timing of the doses, and the dosage amount, as needed. Bearing in mind these considerations, generally, a suitable dose for a given chemotherapeutic agent is between 10 mg/m² to about 500 mg/m², and more preferably, between 50 mg/m² to about 250 mg/m² of patient skin surface area (the skin surface of an average sized adult human is about 1.8 m²). Such a dose, which may be readily adjusted depending upon the particular drug or agent selected, may be administered by any suitable route, including, e.g., intravenously, intradermally, by direct site injection, intraperitoneally, intranasally, or the like. Doses may be repeated as needed.

In one embodiment, because a method described herein can identify or diagnose the presence of a non-CNS neoplasia in a subject at an early stage, e.g., before a neoplasm has formed, before a neoplasm is clinically detectable, and/or before a tumor has become malignant, the dose of a chemotherapeutic agent may be lower than that typically used after a neoplasm, e.g., a cancer, is detected or diagnosed by clinical methods, such as visualization or palpation of a tumor mass.

Therapeutic Targets

A CNS marker gene for a non-CNS disorder, e.g., a CNS marker gene described herein, may not only “sense” the presence of the disorder, but also actively participate in responding to the presence of the disorder by generating a response, e.g., an antitumor response. Alternatively, a CNS marker gene may respond to the presence of non-CNS disorder by promoting progression of the disorder, e.g., inducing growth of a neoplasm or promoting malignant transformation of a neoplasm. As a therapeutic strategy, one would want to promote the expression or activity of the former type of gene, and/or inhibit the expression of activity of the latter type of gene, in the CNS. Thus, regardless of whether a CNS marker gene generates a response to curb or promote a specific disorder, its identification can provide a target for inhibiting progression of the disorder.

One way to identify such CNS marker genes that are also potential therapeutic targets is to identify CNS genes that are differentially expressed in animals that exhibit an inhibitory response against a disease compared to animals that do not exhibit an inhibitory response. For example, experimental animals can be injected with tumor inducing cells (e.g., colon cancer cells such as CT26) that express an interleukin (IL), e.g., IL-12. Injection of tumor cells genetically modified to express IL-12 is known to induce Th1 immune-mediated tumor rejection (Adris et al., 2000, Cancer Res., 60(23):6696-703). Control mice can be injected with tumor cells that do not express IL-12. At different times after injection, gene expression in the CNS is analyzed in the animals, as described herein, e.g., by microarray analysis. Thus, genes that “turn off” and “turn on” specifically in the CNS (e.g., brain) of the animals can be identified. Some of these genes will respond to the presence of the IL. Others will correspond to genes actively engaged in the “stimulation” of the antitumor immune response. This strategy can be used for any interleukin gene that may be involved in the stimulation of an antitumor immune response. Identification of brain genes actively involved in “stimulating” an antitumor response will provide a target for therapeutic intervention, e.g., by direct use of the gene or its gene product, or by screening for agents that block or stimulate their activity.

A second strategy for identifying CNS genes that are potential therapeutic targets is by using transgenic animals (e.g., knockout mice) having brain-specific disruptions (e.g., knockouts) in specific genes. A great number of CNS-specific knockout mice are currently available to the skilled artisan (see, e.g., the Jackson Laboratory web site, describing numerous JAX® mice models used in neurobiology), and many more can be expected to become routinely available. A role in the CNS response to non-CNS disease can be established for any particular gene for which a brain knockout animal can be obtained or produced, by inducing the disorder in the knockout mice (e.g., as described herein for cancer, RA, asthma or obesity), and evaluating disease outcome.

CNS marker genes and gene products that are also potential therapeutic targets are listed in FIGS. 48A-C, 59, and 61. These genes are or encode molecules involved in cell signaling, (e.g., growth factors, hormones, cytokines and their receptors) and are also differentially expressed markers in each of the tumors studied.

Vaccines

The methods described herein also provide targets for preventive vaccination. A set of brain genes that “senses” a disease may include receptors for known or unknown ligands. A disease cell might produce these ligands to inhibit the induction of a brain-derived anti-disease response. In such an instance, identifying a CNS gene that is involved in an anti-disease response can lead to the identification of a gene product secreted by the diseased cell that might impact in the brain to inhibit disease response. A genetic vaccine targeting these products could be a viable therapeutic strategy.

One approach to identify CNS targets for preventive vaccination in the treatment of non-CNS disorders is the following: obtain a CNS gene expression profile (using techniques such as those described herein above) from animals that exhibit an anti-disease response, e.g., in the case of a tumor, an IL-12 mediated antitumor response, in an experimental tumor model. It is expected that from the cluster of genes “sensing” the tumor, some will change their expression levels in the presence of IL-12. This subset of genes will likely be those involved in “generating” the antitumor response. This subset of genes is likely to have predictable modulators. For example, if a CNS gene that changes its expression profile in response to a non-CNS gene in the presence of IL-12 is a receptor, one could predict that the change in gene expression of such a receptor could be brought about by its ligand. Thus, a preventive genetic vaccine could be designed to generate a memory response to such a ligand.

A second experimental approach can involve identifying those CNS genes that change their activity in response to a non-tumorigenic dose of tumor cells (e.g., a condition where neoplasia exists in the body, but no neoplasm is yet formed). From this subset of CNS genes one can predict the modulating genes responsible for their changes in activity, as explained above. Such modulating genes, which may be derived from the neoplastic cells, are likely to be initial tumor-derived signals of alarm in the peripheral body. Thus, a preventive genetic vaccine could be designed to generate a memory response to such genes.

A vaccine can be, e.g., a polypeptide or nucleic acid corresponding to the gene to be targeted. Vaccines described herein can be administered, or inoculated, to an individual in physiologically compatible solution such as water, saline, Tris-EDTA (TE) buffer, or in phosphate buffered saline (PBS). They can also be administered in the presence of substances (e.g., facilitating agents and adjuvants) that have the capability of promoting uptake or recruiting immune system cells to the site of inoculation. Vaccines have many modes and routes of administration. They can be administered intradermally (ID), intramuscularly (IM), and by either route, they can be administered by needle injection, gene gun, or needleless jet injection (e.g., Biojector™, Bioject Inc., Portland, Oreg.). Other modes of administration include oral, intravenous, intraperitoneal, intrapulmonary, intravitreal, and subcutaneous inoculation. Topical inoculation is also possible, and can be referred to as mucosal vaccination. These include, for example, intranasal, ocular, oral, vaginal, or rectal topical routes. Delivery by these topical routes can be by nose drops, eye drops, inhalants, suppositories, or microspheres.

The following examples are illustrative only and not intended to be limiting.

### EXAMPLES

### Example 1

### CNS Gene Expression Profiles Associated with Colon Carcinoma

CNS gene expression profiles associated with the presence of a peripheral tumor were identified using gene expression microarray analysis on brain tissue from experimental animals implanted peripherally with tumor cells. This example describes the identification of brain gene expression profiles associated with colon carcinoma.

Male BALB-C mice were injected subcutaneously with 5×10⁵ CT-26 WT cells, a murine colon carcinoma cell line (ATCC cat #: CRL-2638), resuspended in 300 μl of PBS, as described below. Control mice were injected with the corresponding volume of PBS following the same procedure. After a specified time, the animals were sacrificed, their brains dissected, and first strand cDNA was synthesized from total or polyA+RNA prepared from different brain regions, as described in detail below. Gene expression microarray analysis was performed with the first strand cDNA by hybridizing to preprinted slides (Corning's CMT-GAPT™ II Coated Slides) containing Pan® Mouse 10K Oligo set A (MWG Biotech). This slide set contains probes for 9,769 genes selected from mouse genes that have been functionally defined.

The data from the microarray experiments was analyzed with a Biorad Versarray chip reader 5 μm system, laser scanner (Biorad, Waterloo, ON, Canada) using then Versarray Analizer software, as described in more detail below.

Experimental Methodology

Cell Lines: The experimental work was based on the following murine cell lines: CT26WT colon carcinoma (ATCC cat #: CRL-2638), LL/2 (LLC1) lung carcinoma (ATCC cat #: CRL-1642) and 4T1 breast carcinoma (ATCC cat #: CRL-2539). All cell lines were grown in P-100 plates with 10 ml of the corresponding medium. All culture media were sterilized by filtration using 0.22 μm CA filter. CT-26 cells were grown in DMEM containing 1.5 g/L Sodium Bicarbonate, 10 mM Hepes, and 1 mM Sodium pyruvate, supplemented with 10% Fetal Bovine Serum at 37° C. with 5% CO₂-LL/2 (LLC1) cells were grown in DMEM containing 4.5 g/L Glucose, 1.5 g/L Sodium Bicarbonate, 10 mM Hepes, and 1 mM Sodium pyruvate, supplemented with 10% Fetal Bovine Serum at 37° C. with 5% CO₂. 4T1 cells were grown in RPMI 1640 containing 4.5 g/L Glucose, 1.5 g/L Sodium Bicarbonate, 10 mM Hepes, and 1 mM Sodium pyruvate, supplemented with 10% Fetal Bovine Serum at 37° C. with 5% CO₂.

In vivo studies: Six week-old animals were housed in an Hepa filtered air rack, 5 animals per cage (both tumor and control animals in the same cage) with food and water ad libitum for two weeks. At the age of 8 weeks Balb-C males were injected subcutaneously with 5×10⁵ CT-26 WT cells resuspended in 300 μl of PBS. BALB-C female mice were injected subcutaneously with 1×10⁵ 4T-1 cells resuspended in 100 μl of PBS. C-571BL6 male were injected subcutaneously with 1×10⁶ LL/2 (LLC1) cells resuspended in 300 μl of PBS. Control animals were injected with the corresponding volume of PBS following the same procedure.

For each tumor type 4 different experiments were performed and 3 time points evaluated in quadruplicate. Each single time point corresponded to 30 mice (15 Tumor bearing mice and 15 control mice). All injections were done using a 27-G syringe. At the corresponding time, mice were killed by cervical dislocation. Mice were immediately decapitated, the brain extracted and dissected using the following procedure: the hypothalamus and the cerebellum were dissected, the brain was cut with a surgical razor blade leaving the right and left hemispheres separated, and two persons dissected the midbrain, the hippocampus, the prefrontal cortex and the striatum from each brain hemisphere. All brain regions were immediately frozen in dry ice and stored at −80° C. until RNA extraction.

Isolation of Total RNA: Frozen tissue samples were homogenized in the presence of 6 ml of Trizol Reagent (Invitrogen, life technologies, Carlsbad, Calif., USA), for Hypothalamus and Prefrontal Cortex and 10 ml for Mid Brain Total RNA was obtained following manufacturers instructions. The RNA was DNase treated with 10 μl of DNase I (2 U/μl) (Ambion, Inc. Austin, Tex., USA) for the hypothalamus and pre-frontal cortex and with 40 μl for the mid brain in the presence of RNase Out (Invitrogen, Life Technologies, Carlsbad, Calif., USA) at 37° C. for 30 min. DNA-free RNA was extracted with phenol-chloroform, and resuspended in RNase-free Milli-Q water.

Preparation of Poly A+ RNA: Poly A+ RNA was obtained from total RNA using the MicroPoly(A) Pure® kit from Ambion. In general, starting material was 400 μg total RNA to which a volume of 5M NaCl was added up to a final concentration of 0.45 M NaCl. After mixing, samples were transferred to an RNase-free microfuge tube. After adding binding buffer provided by the manufacturer, the RNA was heated for 5 minutes at 65° C. and immediately chilled on ice for 1 minute. Oligo (dT) Cellulose was added to the sample, mixed by inversion and incubated for 60 minutes at room temperature with gentle agitation. This was followed by centrifugation at 4,000 rcf for 3 minutes. After the supernatant was removed, the pellet was treated with 1 ml binding buffer, mixed and spun down by centrifuging at 4,000 rcf for 3 minutes. After removing the supernatant, the pellet was washed 3 times with binding buffer followed by 4 washes with wash buffer. The Oligo(dT) Cellulose was then dissolved in 400 μl of wash buffer provided by the manufacturer and transferred to a spin column when the resin was washed 4 more times. When the flow-through of the column reached an absorbance of <0.05 OD at A260, the mRNA was eluted from the Oligo(dT) Cellulose with 200 μl of Elution Buffer (provided by the manufacturer) pre-warmed at 65° C. The eluted polyA+ RNA was concentrated with a mixture containing 20 μl of 5 M Ammonium Acetate, 1 μl Glycogen and 550 μl of 100% ethanol. After overnight precipitation at −20° C. samples were centrifuged at 14,000 rcf for 20 minutes at 4° C. After careful removal of the supernatant the pellet containing the polyA+ RNA was resuspended in 10 μl of DEPC treated Water/EDTA.

Labeling of probes for microarray hybridization: Labeling was performed by an indirect method. The first method used aminoallyl labeled nucleotides via first strand cDNA synthesis using SuperScript Reverse Transcriptase followed by coupling of the aminoallyl to either Cyanine 3 or 5 (Cy3/Cy5) fluorescent molecules (Amersham Pharmacia). To 3 μg of poly(A+) RNA were added 0.6 μl Random Primers (pd (N)6, Invitrogen) (3 μg/μl) and 1.2 μl Oligo (dT) 12-18 (0.5 μg/μl). Milli-Q H₂O was added up to a final volume of 15.5 μl. The mixture was heated to 65° C. for 5 minutes, chilled on ice and spun down. 12.5 μl of a master mix containing: 6 μl of 5× First Strand Buffer, 3 μl of 100 mM DTT, 0.6 μl of 50× aminoallyl (Sigma Co)-dNTP mix (Amersham Pharmacia), 1.5 μl of RNase Out (40 units/μl, Invitrogen), 1.4 μl Milli-Q H₂O were added to each tube, incubated at 37° C. for 2 minutes, followed by the addition of 2 μl of SuperScript II Reverse Transcriptase (Invitrogen). After incubation for 2 hours at 37° C., the tubes were incubated for 15 minutes at 70° C. and then were spun down. RNA was degraded by the sequential addition of 3 μl of 2.5 M NaOH incubated at 37° C. for 15 minutes, then 15 μl of 2 M HEPES free acid, 4.8 μl of 3 M NaAcO (pH 5.2) and finally 150 μl of 100% EtOH. After mixing, tubes were incubated at −20° C. for 1 hour. Tubes were centrifuged for 30 minutes at 4° C., the supernatant was removed and the pellet was washed twice in 70% ethanol. The pellet was dissolved in 2.25 μl Milli-Q H₂O. Coupling of fluorescent Cy3 and Cy5 was performed by adding to the 4.5 μl cDNA sample 2.25 μl of 0.2 M NaHCO₃ (pH 9.0) and then 4.5 μl of the DMSO/dye mixture. Tubes were mixed well and incubated for 1 hour at room temperature in the dark. For probe purification 500 μl of loading buffer were added to the sample and mixed. A SNAP Column (Invitrogen) was placed on a collection tube and the sample loaded on the column and incubated at room temperature for 2-5 minutes. The SNAP Column was centrifuged at maximum speed for 1 minute and the flow-through was discarded. After two more washes the SNAP column was put back in the collection tube and centrifuged at maximum speed for 30 seconds to remove residual wash buffer from the membrane filter. cDNA was eluted by adding 60 μl TE buffer to the SNAP column, incubated for 2-5 minutes and centrifuged at maximum speed at room temperature for 1-2 minutes. After saving the first eluate, the elution was repeated and both samples were combined.

Quantification of the levels of incorporation of dyes and total DNA: The extent of dye incorporated was obtained by the absorbance at 550 nm and 650 nm for Cy3- and Cy5-probes, respectively. The amount of DNA was obtained by the absorbance at 260 nm. The percentage of dye incorporation was 3-5%.

Microarrays and Data Analysis

Prehybridization: The prehybridization buffer (5 ml of 20×SSC Buffer, 0.25 ml of 20% SDS, 5 ml of 10% BSA and 24.75 ml of Milli-Q H₂O) was preheated at 42° C. The printed slide was put in a 50 ml-Falcon polypropylene tube containing the preheated prehybridization buffer and incubated at 42° C. for 40 min. After washing the slide five times, 1 minute each time, with Milli-Q H₂O preheated at 42° C. in a Wash Station, slides were washed four or five times in 2-propanol. The slide was dried by centrifugation for 1 minute using a Microarray Centrifuge. Cover glasses were washed with Milli-Q H₂O and 2-propanol and dried. Slides were used immediately for hybridization.

Hybridization. All hybridization was done in dye swap manner. Each hybridization mix contained: 0.15% SDS, 30% formamide, 3% SSC; 1 μg/μl Salmon Sperm DNA. To this mix 70 pmoles of Cy3 containing probe and 35 pmoles of Cy5 containing probe were added to give a total volume of 60 μl. The mixture incubated at 95° C. for 3 minutes, snap cooled on ice for 1 minute, and centrifuged at 16.000 g for 1 minute. A pre-hybridized microarray slide (array side up) was placed in a hybridization chamber. The probe mixture was placed carefully on the top of the slide surface and covered by a cover slip. The edges of the cover slip were circumscribed with Immedge pen (Vector Laboratories Inc., Burlingame, Calif., USA). 10 μl of Milli-Q H₂O (20 μl total) was added to the small wells at each end of the chamber to seal the chamber. Slides were incubated at 42° C. for 16-20 hours in a 3D-rotator. At the end of the hybridization, the slide was carefully removed and washed with washing buffer (2×SSC, 0.1% SDS) preheated at 42° C. for 5 minutes with agitation. Slides were washed twice more in different chambers, each time for 5 minutes (first in 1×SSC and then in 0.1×SSC). The slide was dried by centrifugation for 1 minute in a microarray centrifuge and placed in a light tight slide box until scanning.

Data acquisition and image processing: The slides were scanned with a Virtek ChipReader laser scanner model A0-B0-05 (Virtek Vision Corp, Waterloo, ON, Canada) using the VersArray ChipReader software v3.0 build 1.63 (BioRad). Three images were obtained for each of the Cy3 and Cy5 channels with different detector sensitivity values for each image, with a resolution of 10 μm and a pixel depth of 16 bits. The images were stored as 16 bit TIFF files (Tagged Image File Format) and analyzed with VersArray Analyzer software v4.5 (BioRad). Image segmentation was performed with the “cross-correlated” algorithm, and “local corners” were used for background determination. The results were stored in plain text files with the following fields separated by tabulations: Grid, Row, Column, Signal Average for each channel, Signal Median for each channel, Background average for each channel, Area in pixels, and Quality score. The quality score (QS) was defined as the geometric mean between spot shape QS and signal-to-noise QS scores. Signal-to-noise QS was calculated as the percentage of pixels in a spot with values higher than 2*median (local background). Spot shape QS is defined as ratio of spot area to spot perimeter scaled to be in a range between 0 and 1.

Data filtration and normalization: All the data processing was performed under the R System v1.8.1 (The R Development core Team). To maximize the working dynamic range of the data, the nine possible combinations of channels were analyzed. The data was filtered to eliminate dust derived data points (spots with size less than 75 pixels or with a mean to median correlation less than 80% (Tran et al., Nucleic Acids Res. 30(12), e54, 2002), to eliminate saturated data points (spots with a proportion of saturated pixels greater than 20%), and to eliminate low signal data points (spots with signal to noise ratio below 1.2). Since spot intensity was not correlated to background, and in most images we observed that spot background was lower than slide background (Fang et al., Nucleic Acid Res. 31(16):e96), we decided to perform data analysis in two parallel ways, depending on whether background was subtracted (BS) or not (BNS) from spot intensity data. The base 2 logarithm of the ratio and the product between Cy5 and Cy3 was calculated as:

M=log₂(Cy5/Cy3) (1)

A=½·log₂(Cy5×Cy3) (2)

Data for each of the nine replicates was globally normalized by subtraction of its own median value. Outlier data points were eliminated from the nine replicated data with a leave-one-out algorithm. Briefly, a data point was discarded as being outlier if it was outside the confidence interval defined by the remaining data points with a confidence level of 95% estimated from a t-student distribution with n−1 degrees of freedom. Here, n is the number of the remaining data points.

A gene expression dataset was then generated with the average of non-outlier data points.

For data normalization we assumed the following model:

Mⱼₖ=mⱼ+cₖ+eₖ(Fⱼ)+eₖ(Aⱼₖ·Pⱼ)+εⱼₖ (3)

Where mⱼ (j=1, 2, . . . , g) represents the true ratio of expression levels for the gene measured by spot j, and Mⱼₖ (j=1, 2, . . . , g; k=1, 2, . . . , n) represents the measured ratio of expression levels for spot j on replicate k. This model states that the measured ratio M of replicate k is affected by a global measurement bias between the two channels cₖ, a spot (or gene) specific bias eₖ(Fⱼ), a spot intensity-dependent bias eₖ(Aⱼₖ), a spot location-related bias eₖ(Pⱼ), and a zero mean random error εⱼₖ. Since our experimental results showed that eₖ(Pⱼ) and eₖ(Aⱼₖ) were not independent, we modeled the intensity-dependent and location-dependent bias as eₖ(Aⱼₖ·Pⱼ)=f (xⱼ, yⱼ, Aⱼₖ), where xⱼ and yⱼ define the coordinate of spot j in the slide. Data was corrected for global measurement bias between channels (cₖ) by global median normalization. The gene specific bias (eₖ(Fⱼ)) was corrected by dye-swap analysis (see below). Finally, the intensity-dependent and location-dependent bias (eₖ(Aⱼₖ·Pⱼ)) was corrected by a locally weighted 3D-polynomial surface regression of M vs. xⱼ, yⱼ and Aⱼₖ for the entire slide, followed by a 3D-polynomial surface regression for each grid, to correct for grid-specific intensity-dependent and location-dependent systematic bias. Locally weighted 3D-polynomial surface regression was carried out with the loess function of R system (modern regression package).

Data Integration Between Replicated Slides (Dye-Swap Analysis):

Each labeled probe was hybridized at least twice in a dye-swap protocol (technical replicate). Genes that do not correlate in a dye-swap experiment were eliminated. Non-correlated genes were identified as follows: the product between the two ratios was calculated and sorted. The data points corresponding to the lower ratios were eliminated iteratively until the first quantile (in a total of 100 quantiles) was equal or greater than the 99^{th} quantile.

If the scale (i.e., variance) between all the replicates of an experiment was different (p<0.05, Fligner-Killeen test for homogeneity of variances), data was transformed to be equally scaled. Assuming that the ratios follow a normal distribution with mean zero and variance αᵢ²σ², we estimated αᵢ as follows:

$${a}_{i} = \frac{{MAD}_{i}}{\sqrt[1]{{\prod }_{i = 1}^{1} {MAD}_{i}}} . \qquad ( 4 )$$

with I denoting the total number of slides, and the median absolute deviation (MAD) defined by,

MAD=medianⱼ|Mᵢⱼ−medianⱼ(Mᵢⱼ)| (5)

where Mᵢⱼ denotes the j^{th} spot in the i^{th} slide.

An integrated data set was obtained as the average of A values from technical replicates weighted by their mean quality score, and the average of M values from technical replicates weighted by their mean quality score.

Analysis and integration of biological replicates: At least four biological replicates were prepared. The arithmetic mean (Mn) and SD were estimated from the integrated data for technical replicates. Differentially expressed genes for each experiment were identified (p<0.05, t-student test for paired data).

Multivariate Analysis:

Time analysis: A mixed-model design with two fixed effects (tumor cell injection or control treatment, and time points) and one random effect (biological replicates) without repetition was analyzed by Analysis of Variance (ANOVA) between groups (Pavlidis P, Methods, 31:282-289, 2003). Such a design allowed for the estimation of p-values for treatment, time points and their interaction.

Tumor and time analysis: A mixed-model design with three fixed effects and one random effect without repetition design was analyzed by ANOVA. For such a design, biological replicates were analyzed as random effects, and fixed factors were treatment (tumor vs. control), tumor model (breast, colon and lung cancer), and time (18, 72 and 192 hours). Such a design allowed for the estimation of p-values for treatment, tumor model, time, and interactions of treatment with tumor model and time.

Cluster analysis: Only genes differentially expressed were included in cluster analyses. A given gene was considered differentially expressed if its expression ratio was significantly different from zero for the two analyzed data sets (BNS and BS). Thus, genes differentially expressed (p<0.01) in dataset BNS that were also differentially expressed (p<0.05) in dataset BS were included in the cluster analysis. Similarly, genes differentially expressed in dataset BS (p<0.01) that were also differentially expressed (p<0.05) in dataset BNS were included in cluster analysis. FIGS. 5, 6, and 7 list the genes that were considered differentially expressed in the prefrontal cortex at 18 hours, 72 hours, and 192 hours, respectively, after tumor cell injection. FIGS. 14, 15, and 16 list the genes that were considered differentially expressed in the hypothalamus at 18 hours, 72 hours, and 192 hours, respectively. Similarly, FIGS. 23, 24, and 25 list the genes that were considered differentially expressed in the midbrain at 18 hours, 72 hours, and 192 hours, respectively.

Before cluster analysis, the data was scaled as follows: Ms=(M−Mn(M))/SD(M). A figure of merit algorithm (Yeung et al., Bioinformatics 17(4):309-18, 2001) was used to identify the clustering algorithm and the number of clusters that minimized the intra-cluster variability. After examining the figure of merit of all the datasets analyzed with seven different clustering algorithm and different variations of such algorithms that led to a total of 51 different clustering methods, we decided to perform a hierarchical algorithm using Euclidean distance between gene expression patterns and a Ward's minimum variance agglomeration method (Hartigan, Clustering Algorithms. Wiley, New York, 1975).

FIGS. 30A and 30B show the results of a clustering analysis that included data on genes that were differentially expressed at the 18, 72, and 192 hour time points in the prefrontal cortex. FIG. 30-1 shows the result of a clustering analysis that included genes that were down-regulated in the prefrontal cortex at all time points. FIG. 30-2 shows the result of a clustering analysis that included genes that were down-regulated at the 18 hour, or at the 18 hour and 72 hour time points. FIG. 30-3 shows the result of a clustering analysis that included genes that were down-regulated at the 192 hour, or 72 and 192 hour time points. FIG. 30-4 shows the result of a clustering analysis that included genes that were up-regulated at all time points. FIG. 30-5 shows the result of a clustering analysis that included genes that were up regulated at the 18 hour, or the 18 and 72 hour time points. FIG. 30-6 shows the result of a clustering analysis that included genes up-regulated at the 192 hour, or 72 and 192 hour time points. FIGS. 33 and 33-1 through 33-6 show the same kind of data except that the samples come from the hypothalamus. FIGS. 36A, 36B and 36-1 through 36-6 show the same kind of data except that the samples come from the midbrain.

Secreted markers: FIG. 47B lists the genes that were differentially expressed at any time (p<0.01) and is predicted or known to be a secreted product related to colon cancer. Secreted markers are particularly useful in that their expression can be detected in cerebral or cerebrospinal fluid, avoiding the need for a solid tissue biopsy.

Gene annotation: Gene information was obtained from:

Entrez Gene (on the internet at ncbi.nlm.nih.gov/entrez),
LocusLink (on the internet at ncbi.nlm.nih.gov/LocusLink),
UniGene (on the internet at ncbi.nlm.nih.gov/UniGene), and
Mouse Genome Informatics (on the internet at informatics.jax.org).

Fields for annotation are “locus” (LocusLink number), “gene” (gene name), “description”, “localization” (component), “biochemical function” (function), “biological function” (process), and “class.”

### Example 2

### CNS Gene Expression Profile Associated with Breast Carcinoma

This example describes the identification of brain gene expression profiles associated with breast carcinoma.

BALB-C mice were injected subcutaneously with 1×10⁵ 4T-1 breast carcinoma cells (ATCC cat #: CRL-2539) resuspended in 100 μl of PBS. All experimental methods, microarrays and data analysis were otherwise performed as described above for Example 1.

Results

Quality filtering, normalization, and analysis of the microarray data were performed as discussed above.

Cluster analysis: Only genes differentially expressed were included in cluster analyses. A given gene was considered differentially expressed if its expression ratio was significantly different from zero for the two analyzed data sets (BNS and BS). Thus, genes differentially expressed (p<0.01) in dataset BNS that were also differentially expressed (p<0.05) in dataset BS were included in the cluster analysis. Similarly, genes differentially expressed in dataset BS (p<0.01) that were also differentially expressed (p<0.05) in dataset BNS were included in cluster analysis. FIGS. 2, 3, and 4 list the genes that were considered differentially expressed in the prefrontal cortex at 18 hours, 72 hours, and 192 hours, respectively, after tumor cell injection. FIGS. 11, 12, and 13 list the genes that were considered differentially expressed in the hypothalamus at 18 hours, 72 hours, and 192 hours, respectively. Similarly, FIGS. 20, 21, and 22 list the genes that were considered differentially expressed in the midbrain at 18 hours, 72 hours, and 192 hours, respectively.

FIG. 29 shows the results of a clustering analysis that included data on genes that were differentially expressed at the 18, 72, and 192 hour time points in the prefrontal cortex. FIG. 29-1 shows the result of a clustering analysis that included genes that were down-regulated in the prefrontal cortex at all time points. FIG. 29-2 shows the result of a clustering analysis that included genes that were down-regulated at the 18 hour, or at the 18 hour and 72 hour time points. FIG. 29-3 shows the result of a clustering analysis that included genes that were down-regulated at the 192 hour, or 72 and 192 hour time points. FIG. 29-4 shows the result of a clustering analysis that included genes that were up-regulated at all time points. FIG. 29-5 shows the result of a clustering analysis that included genes that were up-regulated at the 18 hour, or the 18 and 72 hour time points. FIG. 29-6 shows the result of a clustering analysis that included genes up-regulated at the 192 hour, or 72 and 192 hour time points. FIGS. 32A, 32B, and 32-1 through 32-6 show the same kind of data except that the samples come from the hypothalamus. FIGS. 35A, 35B, and 35-1 through 35-6 show the same kind of data except that the samples come from the midbrain.

Secreted markers: FIG. 47A lists the genes that were differentially expressed at any time (p<0.01) and is predicted or known to be a secreted product related to breast cancer. Secreted markers are particularly useful in that their expression can be detected in cerebral or cerebrospinal fluid, avoiding the need for a solid tissue biopsy.

### Example 3

### CNS Gene Expression Profile Associated with Lung Carcinoma

This example describes the identification of brain gene expression profiles associated with lung carcinoma.

Male C-57/BL6 mice were injected subcutaneously with 1×10⁶ lung carcinoma LL/2(LLC1) cells (ATCC cat #: CRL-1642) resuspended in 300 μl of PBS. All experimental methods, microarray and data analysis were otherwise performed as described above for Example 1.

Results

Quality filtering, normalization, and analysis of the microarray data was performed as discussed above.

Cluster analysis: Only genes differentially expressed were included in cluster analyses. A given gene was considered differentially expressed if its expression ratio was significantly different from zero for the two analyzed data sets (BNS and BS). Thus, genes differentially expressed (p<0.01) in dataset BNS that were also differentially expressed (p<0.05) in dataset BS were included in the cluster analysis. Similarly, genes differentially expressed in dataset BS (p<0.01) that were also differentially expressed (p<0.05) in dataset BNS were included in cluster analysis. FIGS. 8, 9, and 10 list the genes that were considered differentially expressed in the prefrontal cortex at 18 hours, 72 hours, and 192 hours, respectively, after tumor cell injection. FIGS. 17, 18, and 19 list the genes that were considered differentially expressed in the hypothalamus at 18 hours, 72 hours, and 192 hours, respectively. Similarly, FIGS. 26, 27, and 28 list the genes that were considered differentially expressed in the midbrain at 18 hours, 72 hours, and 192 hours, respectively.

FIGS. 31A and 31B show the results of a clustering analysis that included data on genes that were differentially expressed at the 18, 72, and 192 hour time points in the prefrontal cortex. FIG. 31-1 shows the result of a clustering analysis that included genes that were down-regulated in the prefrontal cortex at all time points. FIG. 31-2 shows the result of a clustering analysis that included genes that were down-regulated at the 18 hour, or at the 18 hour and 72 hour time points. FIG. 31-3 shows the result of a clustering analysis that included genes that were down-regulated at the 192 hour, or 72 and 192 hour time points. FIG. 31-4 shows the result of a clustering analysis that included genes that were up-regulated at all time points. FIG. 31-5 shows the result of a clustering analysis that included genes that were up regulated at the 18 hour, or the 18 and 72 hour time points. FIG. 31-6 shows the result of a clustering analysis that included genes up regulated at the 192 hour, or 72 and 192 hour time points. FIGS. 34A, 34B, and 34-1 through 34-6 show the same kind of data except that the samples come from the hypothalamus. FIGS. 37A, 37B, and 37-1 through 37-6 show the same kind of data except that the samples come from the midbrain.

Secreted markers: FIG. 47C lists the genes that were differentially expressed at any time (p<0.01) and is predicted or known to be a secreted product related to lung cancer. Secreted markers are particularly useful in that their expression can be detected in cerebral or cerebrospinal fluid, avoiding the need for a solid tissue biopsy.

### Example 4

### CNS Gene Expression Profile Associated with Carcinoma

This example describes the identification of brain gene expression profiles associated with any two of the following three types of cancer: lung carcinoma, breast carcinoma, and colon carcinoma.

All experimental methods, microarray and data analysis were otherwise performed as described above for Examples 1, 2, & 3.

In a final analysis, the filtered data was re-clustered to select sequences that were differentially expressed in any two of the three tumors analyzed and showed a similar expression pattern for these two tumor models. FIG. 41 shows genes that were down-regulated in any two of the three cancer models analyzed from prefrontal cortex samples. FIG. 42 shows genes that were up-regulated in any two of the three cancer models analyzed from prefrontal cortex samples. FIG. 43 shows genes that were down-regulated in any two of the three cancer models analyzed from hypothalamus samples. FIG. 44 shows genes that were up-regulated in any two of the three cancer models analyzed from hypothalamus samples. FIG. 45 shows genes that were down-regulated in any two of the three cancer models analyzed from midbrain samples. FIG. 46 shows genes that were up-regulated in any two of the three cancer models analyzed from midbrain samples.

### Example 5

### Real-Time PCR Validation of the Microarray Data Real-Time RT-PCR Conditions

Reverse Transcription Reaction: 0.5 μg of mRNA were reverse-transcribed using 0.5 μg oligo(dT)₁₂₋₁₈ (Invitrogen) and 200 U of Superscript II RNaseH⁻ Reverse Transcriptase (Invitrogen). mRNA and oligo(dT) were mixed first, heated at 65° C. for 5 minutes, and placed on ice until addition of remaining reaction components. The reaction was incubated at 42° C. for 50 minutes, and terminated by heat inactivation at 70° C. for 15 minutes. For mRNA degradation, 2 μl of 2.5 M NaOH were added to each cDNA reaction and incubated at 37° C. for 15 minutes. Reactions were neutralized with 10 μl of 2 M HEPES free acid, and cDNA was ethanol precipitated using 1 μl of 20 mg/ml glycogen as carrier. The amount of cDNA was quantified using Oligreen ssDNA Quantitation Reagent (Invitrogen) according to manufacturer instructions.

Reaction Setup and Cycling Conditions: Primers were designed using Primer3 program (available free on the internet at genome.wi.mitedu/cgi-bin/primer/primer3.cgi/primer3_www.cgi), and purchased from Invitrogen. Each gene analyzed for validation was analyzed by comparing the gene with two housekeeping genes (beta2-microglobulin and beta-actin) using SYBR Green I (Invitrogen) in 96-well optical plates on an iCycler IQ Real-Time Detection System (Bio-Rad). For each 25 μl reaction, 1 μl cDNA dilution, 2.5 μl 10×PCR Buffer, 1.5 μl 50 mM MgCl₂, 0.75 μl 10 mM dNTP Mix, 0.5 μl of each primer (19 μM), 0.75 μl SYBR Green I (1:1000 dilution), 0.25 μl 10 mg/ml BSA, 0.25 μl mM fluorescein dye (Bio-Rad), 0.25 μl glycerol, 16.55 μl, and 0.2 μl Platinum Taq DNA Polymerase (Invitrogen) were employed. PCR conditions were set as follows: 2.5 minutes at 94° C., and 40 cycles of 45 seconds at 94° C., 30 seconds at 58° C. and 15 seconds at 72° C.

Calculations: All samples were assayed by triplicate (n=3), and each experiment performed by duplicate (n=2). For analysis, first corrected Tm of each PCR product was checked. Then, efficiency of each reaction was tested using LinRegPCR program (available free on the internet at bioinfo@amc.uva.nl) according to Ramakers et. al., 2003, Neurosci. Lett., 339:62-66. Efficiencies between 85% and 100% were considered appropriate. One standard curve was constructed for each gene (generally 1000 ng, 100 ng, and 10 ng of cDNA dilution were employed), and the relative level of expression calculated according to Rajeevan et. al., 2001, Methods, 25(4):443-51. Normalization versus housekeeping expression were performed using geNorm program (available free on the internet at medgen31.ugent.be/jvdesomp/genorm) according to Vandesompele et. al., 2002, Genome Biol., 18; 3(7):RESEARCH0034.

Real-time PCR: The results obtained from a microarray experiment are influenced by each step in the experimental procedure, from array manufacturing to sample preparation and application to image analysis Brazma et al., 2000, FEBS Lett, 480:17-24. These factors affect the representation of transcripts in the sample, creating the need for validations by complementary techniques. Different techniques may be used for validation. Traditionally, measurements of mRNA levels have been achieved using hybridization-based techniques such as Northern blot, in situ hybridization and ribonuclease protection assay (RPA). However, these approaches are limited by hybridization kinetics and require large amounts of RNA. Additionally, the number of samples that can be handled simultaneously is very limited.

The accuracy of quantitative RT-PCR combined with its potential for high sample throughput makes it an ideal complement to microarray analysis. Real time quantitative PCR is a technique optimized to monitor the progress of the reaction by measuring the accumulation of the amplification products during each cycle via a change in fluorescence, (Gibson U E et al. Genome Res 1996, 6:995-1001; Heid C A et al. Genome Res 1996, 6:986-994). SYBR Green was used for detection of PCR products. In solution, SYBR Green I exhibits very little fluorescence, however, fluorescence is greatly enhanced upon binding to the minor groove of the DNA double helix.

The analysis of gene expression in the brain is very complex. Although the brain has a few primary cell types, these show immense phenotypic diversity, and gene expression changes may affect only small cell subpopulations. Consequently, even profound transcriptome changes in a small subpopulation of brain cells may not be detected; more abundant sources of transcripts can mask these changes. As a result, the magnitude of expression changes found with microarray is often only modest and hard to separate from experimental noise (Mimics K et al., 2004, Nature Neurosc, 7:434-439). For example, Wurmbach et al., 2003, Methods, 31:306-316 have shown that in mouse cerebral cortex after hallucinogens treatment, there was a 43% gene validation when microarray fold difference was greater than 1.6, but only 14.3% gene validation when the fold difference was between 1.3 and 1.6.

We started validating our results by using Real Time PCR analysis. As a first approach for validation we chosen ad random 14 differentially expressed genes. FIG. 49 shows a table comparing the fold difference obtained by microarray analysis versus the fold change obtained by real time PCR. Four genes out of 14 (29%) were validated, when microarray folds were between 1.15 and 1.35. The genes that were validated were the following:

a) TOM1 (target of myb1 homolog) which has been reported to function in inflammatory cytokine-dependent signaling pathways induced by IL-1 beta and TNF-alpha (Yamakami M, 2004, Biol. Pharm. Bull., 27:564-566)

b) Ptpn11 (protein tyrosine phosphatase, non-receptor type 11) which has been reported to be involved in several signal transduction pathway, among them, a pathway required for neurite growth (Chen B. et al., 2002, Dev Biol., 15; 252(2):170-87).

c) Cntn2 (Contactin 2) which has been reported to be involved in organization of mielydated fibers. (Traka M. et al., 2003, J. Cell Biol., 15; 162(6):1161-72).

d) RIKEN cDNA 1200011M11, a novel gene with unknown function

### Example 6

### CNS Gene Expression Profile Associated with Asthma

This example describes the identification of brain gene expression profiles associated with asthma.

Eight week Balb-c males were intraperitoneally injected with 50 μg of ovalbumin (250 μl of a 200 μg/ml solution of ovalbumin in physiologic saline) for seven consecutive days. Negative control animals were injected with the corresponding volume of physiologic saline alone. All injections were done using a 27-G syringe. Three weeks after the last injection, the animals were exposed to repeated ovalbumin (2 mg/ml) aerosols for the asthma group or physiologic saline alone for the negative control group, once a day for 8 days. The aerosol was applied in one cage for each experimental group coupled to a nebulizer. Exposure was performed in groups of 5 animals for 5 minutes.

ELISA for Detection of Ovalbumin-specific Antibodies in Serum: Blood samples were obtained after the last nebulization, stored 1 hour at room temperature centrifuged at 10,000 g for 10 minutes at room temperature. The supernatant (serum) was stored at −80° C. until use. 100 μl of Rat anti-mouse IgE 2 μg/ml in PBS (pH 7.5) was added to a 96 well plate and incubated overnight at 4° C. with agitation. The plate was washed 3× with 100 μl of wash buffer (PBS pH 7.5; 0.05% Tween 20). Blocking was done with 100 μl of blocking buffer (PBS pH 7.5; 1% BSA), and incubated 30 minutes at room temperature with agitation, then washed 3× with 100 μl of Wash buffer. Serum was added in appropriate dilution series in PBS (pH 7.5) and incubated overnight at 4° C. with agitation. The next day 100 μl of a solution containing ovalbumin coupled to Digoxigenin (4 μg/ml) in blocking buffer was added and incubated 2 hours and 30 minutes with agitation at room temperature. The plate was washed 3× with 100 μl of wash buffer and 100 μl of anti-Digoxigenin-POD, Fab Fragments, diluted 1:1000 from the stock solution in wash buffer was added, and incubated 1 hour and 30 minutes at room temperature with agitation. The plate was washed 3× with 100 μl of wash buffer. Developing was done by adding 100 μl of developing solution (Citric Acid 48.8 mM; Sodium Phosphate basic 0.102 M; one O.P.D. pill to 7 ml of solution, H2O2 150× to make it 1×). The reaction was stopped with 100 μl of sulphuric acid 4N and read on an ELISA reader at 420 nm. Animals from the asthma group with levels of anti-ovalbumin IgE similar to controls animals were not included for dissection.

Methods for isolating total RNA, for labeling probes, for microarray hybridization and for data analysis were otherwise performed as described above for Example 1.

### Results

Quality filtering, normalization and analysis of the microarray data were performed as discussed above.

A given gene was considered differentially expressed if its expression ratio was significantly different from zero for the two analyzed data sets (BNS and BS). Thus, genes differentially expressed (p<0.05) in dataset BNS that were also differentially expressed (p<0.1) in dataset BS were included in the cluster analysis. Similarly, genes differentially expressed in dataset BS (p<0.05) that were also differentially expressed (p<0.1) in dataset BNS were included in cluster analysis. FIG. 55 lists the genes that were considered differentially expressed in the prefrontal cortex 2 days after exposure to ovalbumin. FIG. 56 lists the genes that were considered differentially expressed in the hypothalamus 2 days after exposure to ovalbumin. Similarly, FIG. 57 lists the genes that were considered differentially expressed in the midbrain 2 days after exposure to ovalbumin.

Secreted Markers: FIG. 60 lists the genes that were differentially expressed at any time (p<0.05) and is predicted or known to be a secreted product related to asthma. Secreted markers are particularly useful in that their expression can be detected in cerebral or cerebrospinal fluid, avoiding the need for a solid tissue biopsy.

### Example 7

### CNS Gene Expression Profile Associated with Arthritis

This example describes the identification of brain gene expression profiles associated with arthritis.

Ten weeks C57BL/6J mice were intradermal injected at the base of the tail with 0.1 ml of chicken collagen type II (CII) emulsified with complete Freund's adjuvant at a final concentration of 2 mg/ml. Twenty-one days later, a booster (0.1 ml) consisting of CII emulsified with incomplete Freunds adjuvant (2 mg/ml) was injected intradermally too. A further three days later animals were injected with lipopolysacharide (40 mg in 0.1 ml phosphate-buffered saline (PBS); E. coli serotype 055:B5) intra-peritoneally.

Clinical assessment of arthritis: The development and progression of arthritis was monitored and a clinical score was assigned based on visual signs of arthritis (0.5=swelling in the digits, difficulty to walk or pain (paw retraction); 1=swelling of the paw; 2=swelling of the paw and the ankle; 3=complete inflammation). After three weeks, mice were killed by cervical dislocation, immediately decapitated, and the brain extracted and dissected as described below.

Methods for isolating total RNA, for labeling probes, for microarray hybridization and for data analysis were otherwise performed as described above for Example 1.

Results

Quality filtering, normalization and analysis of the microarray data were performed as discussed above.

A given gene was considered differentially expressed if its expression ratio was significantly different from zero for the two analyzed data sets (BNS and BS). Thus, genes differentially expressed (p<0.05) in data set BNS that were also differentially expressed (p<0.1) in data set BS were included in the cluster analysis. Similarly, genes differentially expressed in data set BS (p<0.05) that were also differentially expressed (p<0.1) in data set BNS were included in cluster analysis. FIG. 51 lists the genes that were considered differentially expressed in the prefrontal cortex 24 days after the last lipopolysacharide injection. FIG. 52 lists the genes that were considered differentially expressed in the hypothalamus 24 days after the last lipopolysacharide injection. Similarly, FIG. 53 lists the genes that were considered differentially expressed in the midbrain 24 days after the last lipopolysacharide injection.

Secreted Markers: FIG. 58 lists the genes that were differentially expressed at any time (p<0.05) and are predicted or known to be a secreted product related to arthritis. Secreted markers are particularly useful in that their expression can be detected in cerebral or cerebrospinal fluid, avoiding the need for a solid tissue biopsy.

### Example 8

### Diagnosis of Breast Cancer in a Human by Detecting a Gene Product Profile

This example describes a diagnostic test for non-CNS carcinoma performed on a human subject. The subject is a carrier of the BRCA1 breast cancer susceptibility gene.

A CSF sample is obtained from the subject by means of a lumbar puncture. This procedure is done on an outpatient basis under local anesthetic. The CSF sample is used immediately in the diagnostic assay, or is cooled or frozen and stored or transported to a facility where the diagnostic test is performed.

The diagnostic test involves contacting the CSF sample to an antibody array containing a panel of 3 antibodies that can detect a set (cluster) of CNS gene products that are associated with the presence of breast cancer when secreted in a characteristic profile in the CSF. The panel includes antibody probes for the three CNS markers for breast carcinoma listed in FIG. 47(A). Thus, in this example, the characteristic profile is the CNS “reference profile” for breast carcinoma.

The results of the antibody array are obtained by routine techniques, such as fluorescence detection and measurement of bound antibody vs. unbound antibody for each position (each antibody) on the array. A dataset of the value for the level of each polypeptide detected in the CSF sample by each antibody on the array is generated. The dataset is used directly as the test expression profile. A control expression profile is generated from the average results from antibody arrays of persons without breast carcinoma.

Once the test expression profile is generated, the test profile is compared to the reference expression profile and the control profile. In this example, the reference profile is a dataset that includes relative values of expression for a panel of 3 CNS gene products secreted into the CSF, all of which are known to be up-regulated in subjects who have early stage breast cancer. The Log2 ratios for those three genes are depicted as grey-scale levels in FIGS. 29, 32A, and 35B respectively. If the test profile shows a match, as defined herein, with the reference profile and the subject is determined to have (or be at risk for) early stage breast cancer.

### Example 9

### Diagnosis of Colon Cancer in a Human by Detecting a Gene Product Profile

This example describes a diagnostic test for colon carcinoma performed on a human subject. The subject is a person who has early stage colon cancer. Methods for obtaining a CSF sample from a subject is the same as in Example 8.

The diagnostic test involves contacting the CSF sample to an antibody array containing a panel of 3 antibodies that can detect a set (cluster) of CNS gene products that are associated with the presence of breast cancer when secreted in a characteristic profile in the CSF. The panel includes antibody probes for three of the seven CNS markers for colon carcinoma listed in FIG. 47(B). Thus, in this example, the characteristic profile is the CNS “reference profile” for colon carcinoma.

The results of the antibody array are obtained by routine techniques, such as fluorescence detection and measurement of bound antibody vs. unbound antibody for each position (each antibody) on the array. A dataset of the value for the level of each polypeptide detected in the CSF sample by each antibody on the array is generated. The dataset is used directly as the test expression profile. A control expression profile is generated from the average results from antibody arrays of persons without colon carcinoma.

Once the test expression profile is generated, the test profile is compared to the reference expression profile and the control profile. In this example, the reference profile is a dataset that includes relative values of expression for Ereg, Mgrn1, and Lhb), all of which are known to be up-regulated in subjects who have early stage breast cancer. The Log2 ratios for those three genes are depicted as grey-scale levels in FIGS. 30, 33, and 36 respectively (Ereg ratio=0.67, Cortex 18 hr; Mgrn1 ratio=1.095, average of 1.08 and 1.11, hypothalamus at 72 hr and 192 hr respectively; Lhb ratio=0.92, average of 0.94 and 0.90, midbrain at 72 hr and 192 hr respectively. If the test profile shows a match, as defined herein, with the reference profile and the subject is determined to have (or be at risk for) early stage colon cancer.

### Other Embodiments

A number of embodiments of the invention have been described. Nevertheless, it will be understood that various modifications may be made without departing from the spirit and scope of the invention. Accordingly, other embodiments are within the scope of the following claims.

1-46. (canceled)

47. A method of diagnosing a non-central nervous system (non-CNS) disorder in a subject, the method comprising:
detecting expression of one or more genes in a CNS sample of the subject, wherein the CNS sample comprises a cell from the brain of the subject, a cell from the spinal cord of the subject, or cerebrospinal fluid (CSF), and the gene expression data corresponds to the presence or level of the protein in the sample;
generating gene expression data from the detected gene expression;
obtaining a reference gene expression profile for a specific non-CNS disorders; and
comparing the gene expression data with the reference gene expression profile, wherein a match of the CNS sample gene expression data to the reference gene expression profile indicates the subject has or will develop the non-CNS disorder.

48. The method of claim 47, wherein the CNS sample comprises cerebrospinal fluid (CSF), and the gene expression data corresponds to a protein in the CSF.

49. The method of claim 47, wherein the gene expression is detected using a microarray assay.

50. The method of claim 47, wherein the protein is selected from the group consisting of a hormone, a growth factor, an immune system component, and a cytokine.

51. The method of claim 47, wherein the protein is encoded by a gene listed in any of FIGS. 1, 50, and 54, or a human or other mammalian homolog thereof.

52. The method of claim 47, wherein the gene encodes a gene product selected from the group consisting of hepatocyte growth factor (HGF), apherin A3, chemokine (C—C motif) ligand 4, growth differentiation factor-9b (GDF-9b); bone morphogenetic protein 15 (BMP 15), neuroblastoma suppressor of tumorigenicity 1, melanocyte proliferating gene 1, and fibroblast growth factor 22 (FGF 22).

53. The method of claim 47, wherein the CNS sample is a sample of one or more cells from the brain, and the gene expression data corresponds to a nucleic acid molecule or protein in the sample.

54. The method of claim 53, wherein the brain cells are selected from the group consisting of cells from the hypothalamus, the midbrain, the prefrontal cortex, and the striatum.

55. The method of claim 53, wherein the nucleic acid molecule comprises mRNA corresponding to the gene.

56. The method of claim 47, wherein two or more reference gene expression profiles are used, each specific for a different non-CNS disorder.

57. The method of claim 47, wherein the non-CNS disorder is selected from the group consisting of cancer, rheumatoid arthritis, asthma, diabetes, and obesity.

58. The method of claim 47, wherein the non-CNS disorder is cancer.

59. The method of claim 47, wherein the non-CNS disorder is a solid tumor less than 0.5 cm in diameter.

60. The method of claim 47, wherein the gene expression data comprises data for a plurality of genes in the CNS sample, and comprises a gene expression profile.

61. The method of claim 47, further comprising
obtaining a control gene expression profile corresponding to one or more healthy subjects; and
comparing the gene expression data with the control gene expression profile, wherein a match of the CNS sample gene expression data to the control gene expression profile indicates the subject does not have and will not likely develop the non-CNS disorder.

62. The method of claim 47, wherein the subject is a human.

63. The method of claim 62, wherein the subject has a family history of the disorder.

64. The method of claim 47, wherein the subject lacks a clinical sign of a disorder as evaluated by imaging analysis.

65. The method of claim 47, wherein the subject is a carrier of a gene associated with an increased risk of developing the disorder.

66. The method of claim 65, wherein the subject is a carrier of the BRCA1, BRCA2, hMSH2, hMLH1, or hMSH6 gene.
